(19) United States
(12) Patent Application Publication
(10) Pub. No.: US 2025/0084480 A1
(43) Pub. Date: Mar. 13, 2025

(54) METHODS OF TREATING EOSINOPHILIC COLITIS

(71) Applicant: Children's Hospital Medical Center, Cincinnati, OH (US)

(72) Inventors: Marc E. Rothenberg, Cincinnati, OH (US); Tetsuo Shoda, Cincinnati, OH (US)

(21) Appl. No.: 18/728,969

(22) PCT Filed: Jan. 17, 2023
(86) PCT No.: PCT/US23/10932

§ 371 Date: Jul. 15, 2024

Related U.S. Application Data
(60) Provisional application No. 63/302,386, filed on Jan. 24, 2022.

(51) Int. Cl. C12Q 1/6883; C12Q 1/6851; G01N 33/68

(52) U.S. Cl. CPC C12Q 1/6883; C12Q 1/6851; G01N 33/6875; C12Q 2600/106; C12Q 2600/158; G01N 2474/20

(57) ABSTRACT

Disclosed are methods for treating active eosinophilic colitis (EoC), or in certain aspects, inflammatory bowel disease (IBD), in an individual in need thereof. In one aspect, the methods may comprise a) assaying a tissue sample obtained from a colon of an individual who may be in need of such treatment, wherein the assaying comprises detecting expression of one or more gene of a transcriptome gene set; b) calculating a score based on the expression of one or more gene of a transcriptome gene set; and c) selecting a tissue sample that exhibits a score indicative of EoC or IBD. The methods may further comprise administering an EoC or IBD therapy to the individual whose tissue sample exhibited a score indicative of having EoC or IBD.

### CROSS-REFERENCE TO RELATED APPLICATIONS

This application claims priority to and benefit of U.S. Provisional Patent Application Ser. No. 63/302,386, filed on Jan. 24, 2022, the contents of which are incorporated in their entirety for all purposes.

### STATEMENT REGARDING FEDERALLY-SPONSORED RESEARCH

This invention was made with government support under AI117804 awarded by the National Institutes of Health. The government has certain rights in the invention.

### BACKGROUND

Eosinophilic colitis is a poorly understood disease process. Classification of eosinophilic colitis as part of a spectrum of eosinophilic gastrointestinal disorders or inflammatory bowel disease has not been determined. Eosinophilic gastrointestinal diseases (EGIDs) are clinicopathologically characterized by marked eosinophilic infiltration of the gastrointestinal (GI) tract with related symptoms and are classified according to the site of infiltration: eosinophilic esophagitis (EoE), eosinophilic gastritis (EoG), eosinophilic duodenitis, eosinophilic gastroenteritis, and eosinophilic colitis (EoC). Among EGIDs, EoC represents the least frequent manifestation (1.6-2.1 per 100,000 persons) and least well-understood disorder; however, patients with EoC have a higher disease burden of symptoms and comorbidities than patients with EoE, the most common EGID. Because of a lack of agreed-on diagnostic criteria, EoC is currently defined as a clinicopathologic disorder that primarily affects the colon with eosinophil-rich inflammation in the absence of known causes of eosinophilia. However, this diagnostic definition is problematic because there are numerous more common diseases associated with colonic eosinophilia, most notably inflammatory bowel disease (IBD), and the relationship between IBD and eosinophilic infiltration in GI biopsies is unclear. Thus, there is a need for improved methods of diagnosis and treatment for the reduction of disease burden.

### BRIEF SUMMARY

Disclosed are methods for treating active eosinophilic colitis (EoC), or in certain aspects, inflammatory bowel disease (IBD), in an individual in need thereof. In one aspect, the methods may comprise a) assaying a tissue sample obtained from a colon of an individual who may be in need of such treatment, wherein the assaying comprises detecting expression of one or more gene of a transcriptome gene set; b) calculating a score based on the expression of one or more gene of a transcriptome gene set; and c) selecting a tissue sample that exhibits a score indicative of EoC or IBD. The methods may further comprise administering an EoC or IBD therapy to the individual whose tissue sample exhibited a score indicative of having EoC or IBD.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1. Distinct, conserved pattern of gene expression in active EoC colonic tissue. (A) Volcano plot (red, upregulated; blue, downregulated) of expression profiles of differentially dysregulated genes between NL and subjects with active EoC (EoC, FDRP<0.05, 1.5-fold change). (B) Clustering analysis based on 987 differentially expressed genes (EoC transcriptome). (C) Venn diagram of the number of genes dysregulated in EoC and CD transcriptomes (FIG. 6). (D) Colonic transcriptome data on NL (blue), subjects with inflamed CD (CD, yellow), and subjects with active EoC (red) reduced to 3-dimensional presentation by multidimensional scaling analysis for visual presentation of the expression distance between samples. (E) Heat map (red, upregulated; blue, downregulated) and clustering analysis of the expression profiles of the 1847 genes from the EoC and/or CD transcriptomes with differentially dysregulated expression in active EoC and/or inflamed CD vs NL (FDR P<0.05, 1.5-fold change). (B and E) Each column represents an individual subject or control.

FIG. 2. EoC transcriptome associates with colonic eosinophilia and distinguishes EoC from NL and other EGIDs. (A-C) Correlation plots for peak colonic eosinophil count and colonic expression of CLC and CCL11, the genes that most correlated with EoC eosinophil count. (D) Correlation of peak colonic eosinophil counts with each of the EoC and CD transcriptomes. ****P<0.0001, using the c2 test. (E) Venn diagram of the number of genes dysregulated in EGID transcriptomes (EoE, EoG, EoC). (F-H) Comparisons of type 2-related gene expression by reverse-transcription quantitative PCR in active EGIDs (esophagus [EoE n=82, NL n=50], stomach [EoG n=21, NL n=20], colon [EoC n=12, NL n=16]), (F) eosinophil and mast cells genes, (G) eosinophil chemotactic chemokines, and (H) type 2 cytokines. Data presented as median with interquartile range. Markers represent individual samples. *P<0.05, **P<0.01, and ****P<0.0001, using Mann-Whitney U test. TPM, transcripts per kilobase million.

FIG. 3. Functions and cell types enriched in EoC transcriptome. (A and B) Functional annotation enrichment analyses of 410 downregulated (A) and 577 upregulated (B) genes of EoC transcriptome using CluGO overview charts and showing the 5 most significant terms in biological process by ToppGene (full list; Table 9). (C and D) Decreased cell proliferation and increased apoptosis in patients with EoC. Representative photographs and quantitative evaluation of Ki-67b (proliferating) and cleaved caspase-3b (apoptotic) colonic cells from NL, inflamed CD, and active EoC. Ki-67b: left, 4; right, 20. Cleaved caspase-3+: left, 10; right, 20. *P<0.05, vs NL. (E and F) Specific increase of gene expression-estimated proportion of cell types in EoC (E) and CD (F). Data presented as mean±SEM. *P<0.05, **P<0.01, ***P<0.001, ****P<0.0001, using Kruskal-Wallis test followed by Dunn multiple-comparison test. aDC, activated dendritic cells; CD4b Tem, CD4b effector memory T cells; MPP, multipotent progenitors; SEM, standard error of mean.

FIG. 4. Colonic histologic features and associations with colonic transcripts. (A) Hematoxylin and eosin-stained colon biopsy specimen of a representative subject with EoC (200 magnification). Eosinophils densely populate crypts (arrow) and pericryptal circumferential collars (arrowhead). (B) Histologic feature clustering in colon biopsies with features arranged to ensure that members of the same cluster are adjacent in the correlation plot and in the same order as in the cluster members. Color map shows correlations among histologic features; darker red shades indicate stronger positive correlations. (C) Comparison of histologic features among NL, inflamed CD, and active EoC. Data are mean±SEM. *P<0.05, **P<0.01, and ****P<0.0001, using Kruskal-Wallis test followed by Dunn multiple-comparison test. (D) Spearman r correlations of eosinophilic histologic features with cell proliferation/apoptosis in the epithelium. *P<0.05. (E) Hierarchic relationships between histologic features on the basis of EoC transcriptome gene expression profile correlations, showing a Spearman r-based heat diagram for gene-level correlations. Darker red shades indicate stronger positive correlations, whereas darker blue shades indicate stronger negative correlations. SEM, standard error of mean.

FIG. 5. EoC transcriptome as a function of disease activity and differential diagnosis. (A) Schematic summary of EoC score generation based on dimensionality reduction of the EoC transcriptome to distinguish active EoC vs NL and quantify EoC disease severity. (B) Discovery and replication of the EoC score with independent patients and from different colon sites (discovery: ascending, replication: descending/sigmoid colon). Peak colonic eosinophil count (left) and EoC score (right) are shown. Data are mean±SEM. ***P<0.001, using Mann-Whitney U test. (C) EoC score as a function of disease activity in EoC. Peak colonic eosinophil count (left) and the EoC score (right) are shown. Data are mean±SEM. **P<0.01, ***P<0.001, and ****P<0.0001, using Kruskal-Wallis test followed by Dunn multiple-comparison test. (D) Unsupervised principal component analysis of the EoC transcriptome showed complete separation of active EoC from inactive EoC and controls, whereas controls and inactive EoC overlapped. (E) Comparison between active EoC and the challenge cases of IBD (CD and UC) with high colonic eosinophil count (High eos). Peak colonic eosinophil count (left) and the EoC-IBD differential score (right) are shown. The dashed line indicates 65 eosinophils/HPF. Data are mean±SEM. NS, not significant, ***P<0.001, and ****P<0.0001, using Mann-Whitney U test. (F) A receiver operating characteristic curve analysis showing utility of the EoC-IBD differential score to differentiate active EoC from IBD (CD and UC) (High eos). AUC, area under the curve; SEM, standard error of mean.

FIG. 6. Molecular comparisons among active EoC by clinical heterogeneity. A-D, Heatmap based on the EoC transcriptome by indicated groups. Heatmap was depicted by each individual (left) and averaged (right) for Age [children (C) vs. adults (A)](A), Atopy [subjects having atopy (+atopy) vs. subjects not having atopy (−atopy)](B), EoE [subjects having EoE (+EoE) vs. subjects not having EoE (−EoE)](C), and treatment [treated subjects (+Tx) vs. untreated subjects (−Tx)](D). E, EoC score by indicated groups. Data are mean±SEM. NS, not significant, using Mann-Whitney U test. EoC, eosinophilic colitis; EoE, eosinophilic esophagitis; Treatment, Tx.

FIG. 7. Identification of a conserved pattern of gene expression from colonic tissue of subjects with CD. A, Volcano plot (red, upregulated; blue, downregulated) of expression profiles of differentially dysregulated genes between normal controls (NL) and subjects with inflamed CD (FDR P<0.05, ≥1.5-fold change). B, Clustering analysis was performed on the basis of 996 differentially expressed genes (CD transcriptome); each column represents an individual subject or control. C-D, Unsupervised analyses [principal component analysis (C), clustering analysis (D)] of the CD transcriptome showed separation of subjects with noninflamed CD (gray) and subjects with inflamed CD (red). CD, Crohn disease; EoC, eosinophilic colitis; FDR, false discovery rate.

FIG. 8. Functional enrichment analysis of the CD transcriptome. A, Functional enrichment analysis in the Disease category using ToppGene (https://toppgene.cchmc.org/). CD transcriptome shows enrichment in Inflammatory Bowel Disease (IBD)-relevant disease pathways (red). B-E, Shown are the 10 most significant terms identified for the CD transcriptome by functional enrichment analysis in the following categories: Pathway (B), Molecular Function (C), Biological Processes (D), and Cellular Component (E). CD, Crohn disease; FDR, false discovery rate.

FIG. 9. Comparisons of EoC and CD to several different UC datasets. A-N, Venn diagrams of the number of genes dysregulated in EoC (blue) and CD (red) transcriptomes to several different pediatric UC transcriptome data from the previously published studies (green). A, GSE9452; B, GSE6731; C, GSE10714; D, GSE10191; E, GSE13367; F, GSE10616; G, GSE14580; H, GSE38713; I, GSE36807; J, GSE47908; K, GSE59071; L, GSE87473; M, GSE87473; N, GSE109142. O, Comparison of EoC and CD transcriptomes to publicly available UC transcriptomes. ****P<0.0001, using Wilcoxon matched-pairs signed rank test. EoC, eosinophilic colitis; CD, Crohn disease; UC, ulcerative colitis

FIG. 10. Immunofluorescence staining of colonic biopsy. Representative images of colonic biopsy sections for Ki-67 (cyan) and Phospho-Histone H3 (red) with DAPI-stained nuclei (blue); sections from normal control individuals (NL) (N=7), patients with inflamed CD (N=6), and patients with active EoC (N=8). CD, Crohn disease; DAPI, 4′,6-diamidino-2-phenylindole; EoC, eosinophilic colitis; NL, normal controls.

FIG. 11. EoC scores by several cut-offs. A-B, Discovery and replication of the EoC score with independent patients and from different colon sites (discovery: ascending, replication: descending/sigmoid colon). EoC score based on FC>3 (A) and FC>5 (B) are shown. Data are mean±SEM. ***P<0.001, using Mann-Whitney U test. C-D, EoC score as a function of disease activity in EoC. EoC score based on FC>3 (C) and FC>5 (D) are shown. Data are mean±SEM. **P<0.01, ***P<0.001, and ****P<0.0001, using Kruskal-Wallis test followed by Dunn multiple-comparison test. E, Spearman correlation plots for peak colonic eosinophil count and EoC scores. EoC, eosinophilic colitis; CD, Crohn disease; NL, normal; HPF, high-power microscopic field; SEM, standard error of mean, FC; fold change.

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting. The methods may comprise, consist of, or consist essentially of the elements of the compositions and/or methods as described herein, as well as any additional or optional element described herein or otherwise useful in the diagnosis or treatment of EoC and/or other diseases as disclosed herein.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

Abbreviations: CCHMC, Cincinnati Children's Hospital Medical Center; CCL, C-C Motif Chemokine Ligand; CD, Crohn's disease; CEGIR, Consortium of Eosinophilic Gastrointestinal Disease Researchers; CLC, Charcot-Leyden crystal; EGID, eosinophilic gastrointestinal diseases; EoC, eosinophilic colitis; EoE, eosinophilic esophagitis; EoG, eosinophilic gastritis; FDR, false-discovery rate; GI, gastrointestinal; HPF, high-power microscopic field; IBD, inflammatory bowel disease; IL, interleukin; NL, normal; PCR, polymerase chain reaction; UC, ulcerative colitis.

Applicant identified a conserved colonic transcriptome in patients with eosinophilic colitis, which was proportional to the degree of colonic eosinophilia, markedly distinct from other gastrointestinal diseases, and uniquely associated with mechanistic processes distinct from other eosinophilic gastrointestinal disorders. Applicant identified eosinophilic colitis as a disease markedly distinct from other eosinophilic gastrointestinal disorders and inflammatory bowel disease, with a disease mechanism that does not involve allergic inflammation, thereby providing a foundation for understanding the disease and improving diagnosis and treatment.

Applicant identified 987 differentially expressed genes (EoC transcriptome) between EoC and NL (>1.5-fold change, P<0.05). Colonic eosinophil count correlated with 31% of EoC transcriptome, most notably with CCL11 and CLC (r=0.78 and 0.77, P<0.001). Among EoC and other EGIDs, there was minimal transcriptomic overlap and minimal evidence of a strong allergic type 2 immune response in EoC compared with other EGIDs. Decreased cell cycle and increased apoptosis in EoC compared with NL were identified by functional enrichment analysis and immunostaining using Ki-67 and cleaved caspase-3. Pericryptal circumferential eosinophil collars were associated with the EoC transcriptome (P<0.001). EoC transcriptome-based scores were reversible with disease remission and differentiated EoC from IBD, even after controlling for colonic eosinophil levels (P<0.0001).

In one aspect, a method of treating active eosinophilic colitis (EoC) in an individual is disclosed. The method may comprise:
a) assaying a tissue sample obtained from a colon of said individual for expression of one or more genes of the EoC transcriptome gene set of Table 12;
b) calculating an EoC score based on the assaying of the EoC transcriptome gene set;
c) selecting a tissue sample that exhibits an EoC score indicative of active EoC; and, optionally,
d) administering an EoC therapy to the individual whose tissue sample was selected in (c).

In one aspect, (a) and (b) above, and optionally (c), may be carried out for the purpose of diagnosing an individual with active EoC, wherein the assaying of a sample and calculating of an EoC score based on the expression of one or more genes of the EoC transcriptome gene set may be used to identify an individual having active EoC. Following identification of an individual having active EoC, a therapy suitable for treatment of EoC may be administered to such individual.

In one aspect, the EoC transcriptome gene set may comprise at least 80%, or at least 85%, or at least 90%, or at least 95%, or at least 96%, or at least 97%, or at least 98%, or at least 99%, or 100% of the gene set of Table 12. In one aspect, the EoC transcriptome gene set may comprise the genes of Table 12 having at least a 2-fold change as compared to a normal control, or at least a 2.5-fold change as compared to a normal control, or at least a 3-fold change as compared to a normal control, or at least a 3.5-fold change as compared to a normal control, or at least a 4-fold change as compared to a normal control, or at least a 4.5-fold change as compared to a normal control, or at least a 5-fold change as compared to a normal control, or at least a 5.5-fold change as compared to a normal control, or at least a 6-fold change as compared to a normal control.

In one aspect, the EoC therapy may be an anti-inflammatory therapy. In one aspect, the method may be performed prior to treatment with a therapy for active EoC. In one aspect, the method may be performed after treatment with a therapy for active EoC. In one aspect, the method may be performed during disease progression or clinical relapse on a therapy for active IBD. In one aspect, the method may be performed after suspension of a therapy for active EoC.

The EoC score may be calculated as described herein. For example, the EoC score may be calculated by summing the normalized expression values of genes dysregulated in the EoC transcriptome. Any suitable normalization method may be used. In one aspect, the expression is normalized to the housekeeping gene glyceraldehyde-3-phosphate dehydrogenase (GAPDH).

In one aspect, the method may be performed prior to treatment with a therapy for active EoC. In one aspect, the method may be performed after treatment with a therapy for active EoC. In one aspect, the method may be performed during disease progression or clinical relapse on a therapy for active EoC. In one aspect, the method may be performed after suspension of a therapy for active EoC.

In one aspect, disclosed is a method of treating inflammatory bowel disease (IBD), for example IBD with high colonic eosinophilia, in an individual, the method comprising
a) assaying a colonic tissue sample obtained from said individual for expression of at least one gene, or at least two genes, or at least two genes, or at least two genes, or at least three genes, or at least four genes, or at least five genes, or at least six genes, or at least seven genes, or at least eight genes, or at least nine genes, or at least ten genes, or at least 11 genes, or at least 12 genes, or at least 13 genes, or at least 14 genes, or at least 15 genes, or at least 16 genes, or all genes of the EoC transcriptome as listed in Table 11;
b) calculating an EoC-IBD differential score based on the data obtained in (a);
c) selecting a sample that exhibits an EoC-IBD differential score indicative of IBD; and
d) administering a therapy for IBD to the individual whose sample was selected in (c).

In one aspect, (a) and (b) above, and optionally (c), may be caried out for the purpose of diagnosing an individual with IBD, wherein the assaying of a sample and calculating of an EoC-IBD differential score based on the expression of one or more genes of the transcriptome gene set may be used to identify an individual having active IBD. Following identification of an individual having active EoC, a therapy suitable for treatment of IBD may be administered to such individual. In one aspect, the individual may be one which exhibits symptoms which may be indicative of either IBD or EoC, said method being used to distinguish the disease types for appropriate treatment thereof.

In one aspect, the IBD therapy may be an anti-inflammatory therapy. In one aspect, the method may be performed prior to treatment with a therapy for active IBD. In one aspect, the method may be performed after treatment with a therapy for active IBD. In one aspect, the method may be performed during disease progression or clinical relapse on a therapy for active IBD. In one aspect, the method may be performed after suspension of a therapy for active IBD.

In one aspect, the tissue sample of any of the aforementioned methods may be obtained from the colon. For example, the tissue sample may be obtained from a site selected from ascending colon, descending colon, sigmoid colon, or a combination thereof.

Exemplary anti-inflammatory therapies that may be used with the aforementioned methods may be any treatment that indicated for EoC and/or IBD as determined to be appropriate for such disease state. Exemplary anti-inflammatory therapies may include, for example, a glucocorticoid therapy, such as a low-dose corticosteroid. Non-limiting examples of corticosteroids include prednisone, budesonide, hydrocortisone, prednisilone, methylprednisilone (Solumedrol®), dexamethasone (Decadron®), betamethasone (Celestone®), fluticasone (e.g., fluticasone propionate). The glucocorticoid therapy may be administered in an amount of from about 0.1 mg to about 20 mg, or from about 0.15 to about 15 mg, or from about 0.2 to about 10 mg, or from about 0.25 to about 8 mg, or from about 0.3 to about 5 mg. The glucocorticoid dose may be administered daily, every other day, every third day, every fourth day, every fifth day, every sixth day, or weekly, or may be administered twice a day, three times a day, or in an amount determined to be effective in the individual in need thereof.

In one aspect, the anti-inflammatory therapy may be a food restriction or diet therapy. For example, exemplary diet therapy may include a “targeted elimination diet” (TED). Alternatively, where many or no allergens are identified, the diet therapy may be an “empiric elimination diet” or “elemental diet” (EED).

In one aspect, the anti-inflammatory therapy may be a leukotriene inhibitor, for example montelukast (Singulair @), which selectively blocks the action of leukotriene D4 (LTD4). In one aspect, montelukast may be administered at a dose of about 10 to about 40 mg for at least one week, or at least two weeks, or at least three weeks, or monthly, or for at least two months, or at least three months, in an interval sufficient to reduce the symptoms of the disease.

In one aspect, the anti-inflammatory therapy may be a mast cell stabilizer, for example oral cromolyn sodium, (e.g., administered at a dose of about 200 mg daily, or twice a day), or ketotifen, a 2nd-generation H1-antihistamine agent that also modulates the release of mast cell mediators (e.g., administered at a dose of 1-2 mg daily, or twice daily). In another aspect, the mast-cell stabilizer may be sodium cromoglycate, alone or combined with ketotifen or cromolyn.

In one aspect, the anti-inflammatory therapy may be an immunosuppressive drug. Exemplary immunosuppressive drugs include, but are not limited to, azathioprine and 6-mercaptopurine.

In one aspect, the anti-inflammatory therapy may be a biological therapy, for example an adhesion molecule antagonists such as natalizumab or vedolizumab, an anti-interleukin 5 antibody such as benralizumab, mepolizumab, reslizumab, and/or vedolizumab), an anti-IgE monoclonal antibody such as omalizumab, anti-IL-12 antibody (such as Ustekinumab, sold under the brand name Stelara®), HUMIRA® (adalimumab), REMICADE® (infliximab), SIMPONI ARIA® (golimumab), STELARA® (ustekinumab)). In one aspect, the biological therapy may be an anti-TNF agent, for example infliximab, adalimumab, golimumab, certolizumab, or a combination thereof.

In one aspect, the anti-inflammatory therapy may be an intravenous interferon-alpha therapy.

In one aspect, the anti-inflammatory therapy may be a fecal microbiota transplantation.

In one aspect, the anti-inflammatory therapy may be selected from mast cell depleting drug (e.g., masitinib, nilotinib, bezuclastinib, avapritinib, ripretinib, nintedanib, midostaurin, imatinib, or other WT KIT-targeting drug), an eosinophil depleting drug (such as benralizumab, mepolizumab, reslizumab), an anti-eotaxin agent (such as bertilimumab, a recombinant human IgG4 monoclonal antibody), an anti-CCR3 agent or antibody, and combinations thereof.

In one aspect, the anti-inflammatory therapy may be a combination thereof of any of the aforementioned therapies.

In one aspect, any of the methods above may further comprise assaying for one or more pathologic changes selected from the presence of eosinophil sheets, cryptitis, crypt abscesses, muscular involvement, and combinations thereof, wherein one or more pathological change weighs in favor of a diagnosis of EoC. In one aspect, any of the methods above may further comprise assaying for one or more pathologic changes selected from a lack of acute inflammation and cryptitis, wherein a lack of said features is indicative of EoC.

In one aspect, the assaying of any of the methods described herein may comprise whole transcriptome sequencing, antibody-based protein quantifications, mass spectrometry based protein quantification, targeted mRNA sequencing and quantification, Nanostring determination, and/or real-time RT-PCR.

In one aspect, the assaying of any of the methods described herein may comprise Sanger sequencing, targeted sequencing and/or whole exome/genome sequencing and/or quantification.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

### Example 1

With regard to the molecular causes of EGIDs, substantial progress has been made using whole-genome transcript expression profiling (transcriptome) of tissue biopsies from patients with EoE, and, more recently, from patients with EoG. Cumulative evidence has elucidated specific molecular, cellular, and immune mechanisms involved in EoE and EoG pathogenesis, including overproduction of type 2 cytokines (e.g., interleukin [IL]5, IL-13) and IL-13-induced gene products (e.g., CCL26/eotaxin-3, CAPN14). In contrast, EoC pathogenesis remains poorly understood because of the relative rarity of EoC and its challenging differential diagnosis.

The differential diagnosis for increased eosinophil density in colonic mucosa is clinically problematic because colonic eosinophils are present during homeostasis, unlike esophageal eosinophils; the eosinophil level is highest in the ascending colon, tapers to lower levels in the recto-sigmoid colon and increases during inflammation in many conditions. As eosinophil-rich inflammation is not exclusive to EoC, primary EoC is a diagnosis that can be made only after all other known causes for increased colonic mucosal eosinophils have been eliminated. Distinguishing EoC from other causes of GI eosinophilia (e.g., hypereosinophilic syndrome, IBD, infection, and autoimmune disorders) is important because the therapeutic strategy may substantially differ. If EoC were similar to other EGIDs, elimination diets and anti-type 2 cytokine therapy would be appropriate therapies; conversely, if EoC were similar to IBD, distinct anti-inflammatory and/or biologic (eg, anti-TNF) therapy would be preferred. The lack of a way to accurately differentiate these colonic states is increasingly recognized as a clinical conundrum.

Applicant examined pediatric and adult patients with EoC across multiple sites associated with the Consortium of Eosinophilic Gastrointestinal Disease Researchers (CEGIR) and subjected colonic biopsies to genome-wide transcriptomic profiling and parallel histological analysis, followed by pathway interrogation, and comparison of the derived findings with other EGIDs and IBD.

### Materials and Methods

### Study Design and Participants

This study was conducted in CEGIR, a national collaborative network of academic centers caring for and researching adults and children with EGIDs. The CEGIR observational study, Outcome Measures in Eosinophilic Gastrointestinal disorders Across the ages (OMEGA), is a longitudinal cohort study investigating the natural history of EoE, EoG, eosinophilic duodenitis, eosinophilic gastroenteritis, and EoC during routine clinical care. Demographic, clinical, endoscopic, and histologic data and GI tissue were prospectively collected starting from 2015; all samples from any CEGIR site that contributed subjects with EoC were used (n=5 sample-providing institutions) (Table 2). The clinical features of subjects were determined during a standard-of-care evaluation using standardized intake forms. All subjects' clinical data were stored at the Rare Diseases Clinical Research Network Data Management and Coordinating Center (University of South Florida in Tampa, FL [2015-2019], and Cincinnati Children's Hospital Medical Center [CCHMC; 2020-2024]).

Pediatric subjects were defined as having an age of less than 18 years. Atopy was defined on the basis of self-report of allergic rhinitis, atopic dermatitis, asthma, or food allergy. Subjects were defined as having EoC if they had a history of colonic eosinophilia (ascending colon≥100 eosinophils/high-power field [HPF], descending colon≥85 eosinophils/HPF, and/or sigmoid colon≥65 eosinophils/HPF) without other known causes of GI eosinophilia; negative tests typically included stool culture for pathogenic bacteria or parasites, viral antibody titers and/or PCR, and Celiac and inflammatory bowel disease serology. A 2× the upper limit of normal for each anatomic site in normal biopsies was used as the thresholds for the definitions of colonic eosinophilia. Active EoC was defined as colonic biopsies that met the above criteria, and inactive EoC was defined as <100 eosinophils/HPF for ascending colon, <85 eosinophils/HPF in descending colon and/or <65 eosinophils/HPF for sigmoid colon in subjects with a previous history of EoC. Subjects with EoC with GI eosinophilia outside of the colon (esophagus: ≥15 eosinophils/HPF, stomach: ≥30 eosinophils/HPF in at least 5 HPF) were included.

An EoC diagnosis was made using a combination of the following: (1) presence of symptoms; symptoms include (but are not limited to) hematochezia, bloody/nonbloody diarrhea, tenesmus, abdominal pain; (2) a history of clinical features indicative of colonic inflammation, such as anemia, peripheral eosinophilia, hemoccult positive stool, EGID, and/or allergic diseases (allergic rhinitis, asthma, food allergy, eczema, or other allergic features suggestive of atopic disease); and (3) colonic mucosal eosinophilia (ascending colon 100 eosinophils/highpower field [HPF], descending colon 85 eosinophils/HPF, and/or sigmoid colon 65 eosinophils/HPF) based on 2× the upper limit of normal for each anatomic site in normal biopsies. Inclusion and exclusion criteria are detailed in Table 3. In each case, alternative causes of mucosal eosinophilia were ruled out, including proctocolitis in infancy; negative tests typically included stool culture for pathogenic bacteria or parasites, viral antibody titers and/or polymerase chain reaction (PCR), and celiac and IBD serology.

For diagnosed EoC cases, EoC disease activity was defined by colonic biopsy eosinophil counts meeting (active EoC) or being lower than the above colonic eosinophilia criteria (inactive EoC). The patients with inactive EoC showed colonic eosinophilia more than the threshold level in the past but less than the threshold level when the biopsy samples were analyzed. Subjects with EoC and concomitant EGID involving other GI segments (esophagus: 15 eosinophils/HPF, stomach: eosinophils/HPF in 5 HPFs) were not excluded.

Non-EoC control subjects (normal [NL], Crohn's disease [CD] as an IBD-representative/spectrum disease) from the Cincinnati Center for Eosinophilic Disorders EGID database between 2015 and 2019 included children and adults who had undergone endoscopy, had no history of EoC or pathologic evidence of EoC surveyed during the index endoscopy, and had colonic biopsies collected for research purposes during the index endoscopy. NLs were patients who underwent endoscopic examination due to digestive symptoms but did not show colonic eosinophilia. NL subjects having treatments because of concomitant diseases (eg, gastroesophageal reflux disease and immunoglobulin E-mediated food allergy) were not excluded. A CD diagnosis was made using previously published guidelines. Features include a variable combination of the following: (1) clinical signs and symptoms including abdominal pain, diarrhea, rectal bleeding, growth delay, and pubertal delay; (2) physical findings including abdominal tenderness, perirectal skin tags, perirectal fistula, and erythema nodosum; (3) endoscopic findings of aphthous, linear or stellate ulcerations, cobble stoning, skip lesions, and strictures in the ileum or colon; (4) histologic findings including ulceration, crypt abscesses, noncaseating granuloma, focal changes within biopsy, and patchy inflammation; and (5) cross-sectional imaging findings including mural thickening, hyperemia, abnormal luminal caliber, altered peristalsis, fibro-fatty proliferation, regional lymphadenopathy, and sinus tracts/fistulae.

CD diagnosis and disease activity were based on a combination of the clinical, endoscopic, and histologic characteristics by gastroenterologists and pathologists at CCHMC. The inflammation status (inflamed, noninflamed) of subjects was defined by assessing histologic features of chronicity and quantifying acute inflammation. A subset of patients with CD who also had a high peak colonic eosinophils/HPF (65 eosinophils/HPF) was defined as CD-high colonic eosinophils.

### Molecular Evaluation

Fresh biopsy specimens collected from subjects with EoC and controls were stored in RNAlater until they were subjected to RNA isolation using the miRNeasy kit (Qiagen, Valencia, Calif) per the manufacturer's instructions. The RNA concentration was measured by Nanodrop, and the RNA integrity number (RIN) was determined by the Gene Expression Core at CCHMC using the Agilent Bioanalyzer. Samples for RNA sequencing were selected from the total cohort on the basis of RNA quality and quantity. RNA sequencing was performed with high-quality RNA (RIN>8) using the QuantSeq 3′ mRNA Seq Library Prep Kit FWD for Illumina (Lexogen). Libraries were subjected to quality control and concentration measurements at the Gene Expression Core at CCHMC. Libraries were diluted to a final concentration of 5 nM and sequenced on a HiSeq 4000 Illumina sequencing machine at the Genomics & Cell Characterization Core Facility at the University of Oregon with single reads of 100-150 bp. Data were aligned to the GRCh37 build of the human genome using the Ensembl annotations. Data analyses, including principal component analysis (PCA) and hierarchical clustering, were performed using DESeq2 in CLC Genomics Workbench software (CLC bio, Waltham, MA, USA) and GeneSpring software ver. 14.9 (Agilent Technologies). Transcripts per kilobase million (TPM) were assessed for statistical significance using a Welch t test with Benjamini-Hochberg false-discovery rate (FDR), threshold of P<0.05, and 1.5-fold-change cut-off filter. Data are available at EGIDExpress (https://egidexpress.research.cchmc.org/data/).

Gene ontology enrichment analysis was performed with the ToppGene suite and CluGO. Cell type enrichment analysis was performed with xCell. EoC score was calculated by summing the normalized expression values of the dysregulated genes of the EoC transcriptomes, respectively. Of note, the EoC score calculated from the EoC transcriptome positively correlated with disease severity.

A real-time reverse-transcription quantitative polymerase chain reaction (RT-qPCR) array platform was performed to determine mucosal expression of genes associated with type 2 inflammation in patients with EGIDs. As type 2 inflammation, 7 genes [eosinophils (CLC), mast cells (HPGDS), chemokines/cytokines (CCL11, CCL26, IL13, IL4, IL5)] were assessed. Patients' biopsies [esophagus (EoE n=82, NL n=50),¹⁰ stomach (EoG n=21, NL n=20), colon (EoC n=12, NL n=16)] were assessed by the EoE Diagnostic Panel (EDP) or EoG Diagnostic Panel (EGDP) with normalization to the housekeeping gene glyceraldehyde-3-phosphate dehydrogenase (GAPDH).

As another relevant disease control, publicly available colonic transcriptome datasets from patients with ulcerative colitis (UC) having active colitis and patient clinical data were comprehensively searched and obtained by the BaseSpace correlation engine (Illumina Inc., San Diego, CA, USA).

RNA sequencing was performed using the QuantSeq 3′mRNA Seq Library Prep Kit FWD for Illumina (Lexogen, Vienna, Austria). Briefly, total RNA was extracted with the miRNeasy kit (Qiagen, Valencia, CA) and evaluated with an Agilent (Santa Clara, CA) Bioanalyzer by the CCHMC Gene Expression Core. Data analyses were performed using DESeq2 in CLC Genomics Workbench software (CLC bio, Waltham, MA) and GeneSpring software version 14.9 (Agilent Technologies). Transcripts per kilobase million were assessed for statistical significance. Data are available at EGIDExpress (https://egidexpress.research.cchmc.org/data/). Functional enrichment analyses were performed with the ToppGene suite and CluGO. Cell type enrichment analysis was performed with xCell. EoC score was calculated by summing the normalized expression values of genes dysregulated in the EoC transcriptomes. A real-time reverse-transcription quantitative PCR was performed to determine mucosal expression of genes associated with type 2 inflammation in patients with EGIDs. As another relevant disease control, publicly available colonic transcriptome datasets from patients with ulcerative colitis (UC) having active colitis and patient clinical data were comprehensively searched and obtained by the BaseSpace correlation engine (Illumina Inc., San Diego, CA). One dataset having colonic eosinophil counts (GSE109142) were also used for EoC score analysis.

### Histologic Features

Colonic biopsies were assessed for the peak eosinophil counts and other histologic features of EoC. Hematoxylin and eosin (H&E)-stained biopsy slides from NL, CD, and EoC were blindly reviewed by CEGIR pathologists (M.H.C., K.E.C., G.Y.). Standardization across centers was performed. CEGIR central review pathologists reviewed images of slides that had been scanned (Aperio scanner) at 40× magnification. Each pathologist used the same annotation to count eosinophils/hpf. The annotation was created for the purpose of counting eosinophils in a view finder that mimicked a round high-power field and measured 0.27 mm², an area that is commonly covered at 40× magnification. Histologic features in images of all submitted colon biopsies were as follows: acute crypt abscess, acute cryptitis, acute inflammation, crypt architectural abnormalities, crypt dropout/loss, crypt epithelial injury, crypts partly destroyed by eosinophilic inflammation, eosinophil crypt abscess, eosinophil cryptitis, eosinophils in muscularis mucosa/submucosa, eosinophils in surface epithelium, granulomas, lamina propria eosinophil sheets, lymphocytes in surface epithelium, overall eosinophilic inflammation, pericryptal circumferential eosinophil collars, subcryptal eosinophil aggregates, subcryptal lymphoplasmacytes, and surface epithelial injury. Each feature was scored using a 3-point scale (0=absent, 1=mild/moderate, 2=marked) (Table 4).

### Immunostaining of Biopsy

Immunohistochemical stains with the Ki-67 (a proliferation marker, 790-4286, Roche) or cleaved caspase-3 (an apoptotic marker, ab2302, Abcam) in colonic biopsies were performed at the Pathology Research Core at CCHMC using the Ventana BenchMark XT automated immunostainer (Ventana Medical Systems, Inc., Tucson, AZ). No signal was observed in biopsies stained with negative control IgG antibodies. Stained biopsy slides from NL, CD, and EoC were blindly reviewed by an expert pathologist (M.H.C.). Immunofluorescent staining was performed, using the following primary antibodies (1:100 dilution): Ki67 (MA5-14520; Invitrogen) and phospho-histone H3 (#9706; Cell Signaling Technology). The nuclei were stained with DAPI. The slides were blocked with PBS with 10% donkey serum. The secondary antibodies (1:400 dilution) used were donkey anti-mouse Alexa Fluor 570 or donkey anti-rabbit Alexa Fluor 488 (Invitrogen). Imaging was performed with a Nikon A1 inverted confocal microscope.

### Statistical Analysis

Statistical analyses were performed using the JMP v13.2.1 (SAS Institute, Cary, NC), CLC Genomics Workbench software (CLC bio, Waltham, MA, USA), GeneSpring GX 14.9 (Agilent Technologies, Santa Clara, CA), and GraphPad Prism 9 (GraphPad Software, Inc., San Diego, CA). Data are presented as n (%) or median (interquartile range [IQR]) unless otherwise stated. Missing data were excluded from all formal statistical analyses. Nonparametric correlation analysis was performed using Spearman's rank correlation coefficient. For continuous data, statistical significance comparing 2 different groups was determined by the Mann-Whitney U test (nonparametric test, 2 groups) or the Kruskal-Wallis test followed by a Dunn multiple-comparison test (nonparametric test, 3 groups or more). Benjamini-Hochberg correction was applied for multiple testing to control the FDR. For categorical data, the chi-square test was used to ascertain differences. A significant P value was defined as less than 0.05.

### Results

### Subject Characteristics

Eighty-seven colonic biopsies (n=31 EoC [12 active, 19 inactive], 27 CD [16 inflamed, 11 noninflamed], 29 NL) from 61 subjects (n=27 EoC, 14 CD, 20 NL) were analyzed, with instances of multiple biopsies (n=3 EoC, 13 CD, 8 NL subjects) being obtained from different colon sites during a single endoscopy. Demographic and clinical characteristics of the study cohort stratified by group (EoC, CD, NL) are detailed in Table 1 and Table 5.

Ages ranged from 4 to 64 years, with 43 pediatric (70.5%) and 18 adult (29.5%) subjects. There was a similar proportion of both genders, with 29 male (47.5%) and 32 female (52.5%) subjects. Most subjects self-identified as White (93.4%). Many subjects had a history of atopy (any allergic disease, 62.3%), such as asthma, allergic rhinitis, atopic dermatitis, and food allergy (24.6%, 47.5%, 32.8%, and 19.7%, respectively). Peak colonic eosinophil counts ranged from 2 to 187 eosinophils/HPF (active EoC 69-187, inactive EoC 9-44, CD 9-110, NL 2-52 eosinophils/HPF).

Focusing on subjects with EoC (n=27), 15 (56%) had concurrent eosinophilia in the esophagus, 5 (19%) in the stomach, and 1 (0.4%) in both the esophagus and stomach. Demographic features (age at biopsy, gender, race) were similar at baseline among EoC, CD, and NL subjects, whereas EoC subjects had significantly higher peak colonic eosinophil counts (P=0.025) and a higher percentage of treatment (proton pump inhibitor therapy at time of biopsy, mainly for concurrent eosinophilia in the esophagus) than CD and NL subjects.

### Identification of EoC Transcriptome

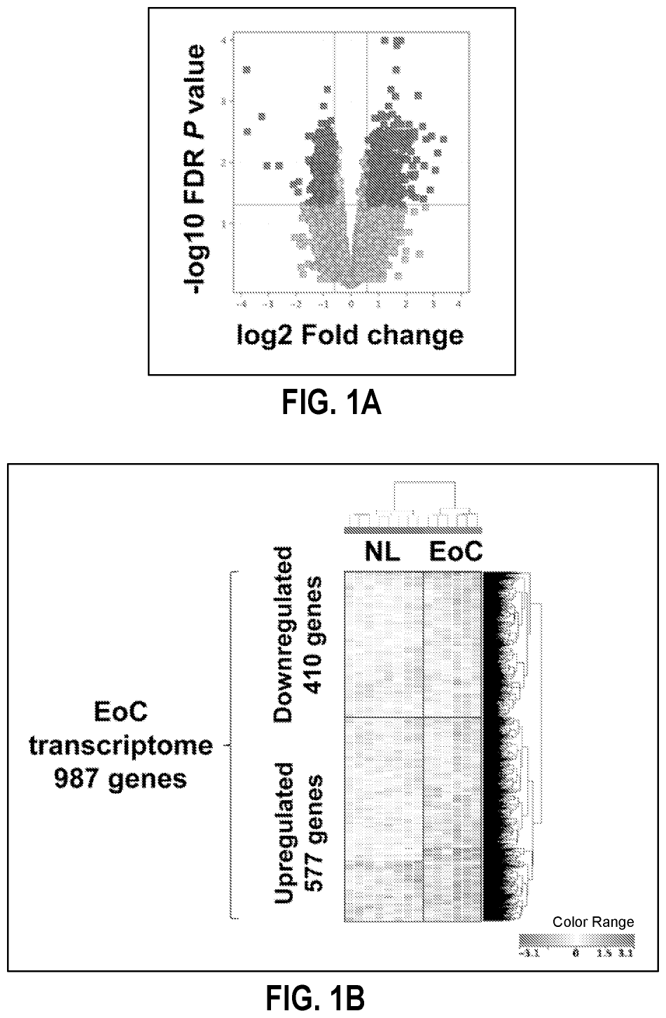

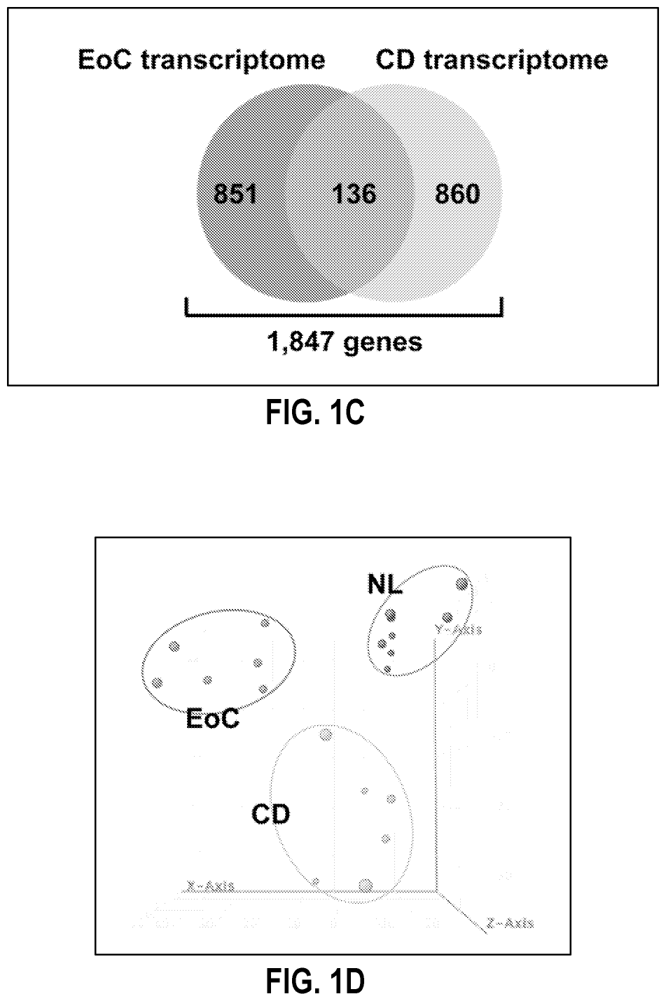

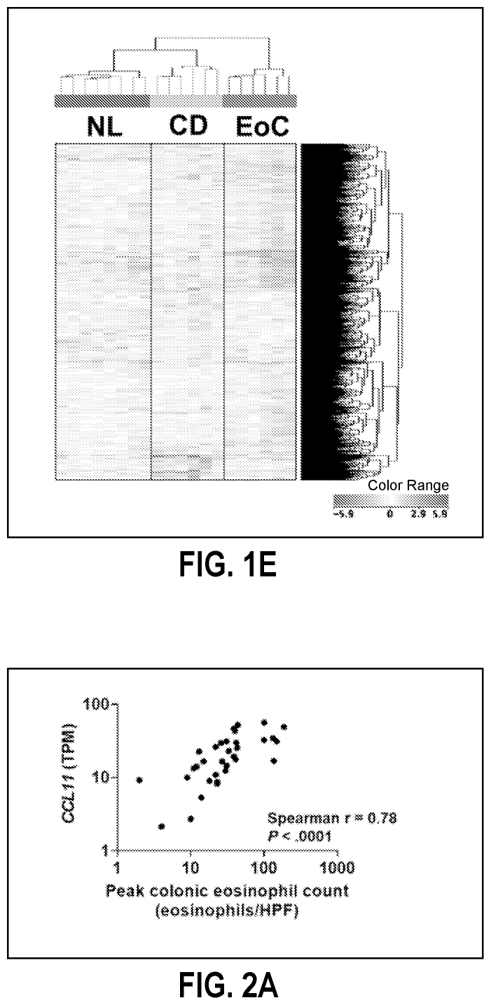

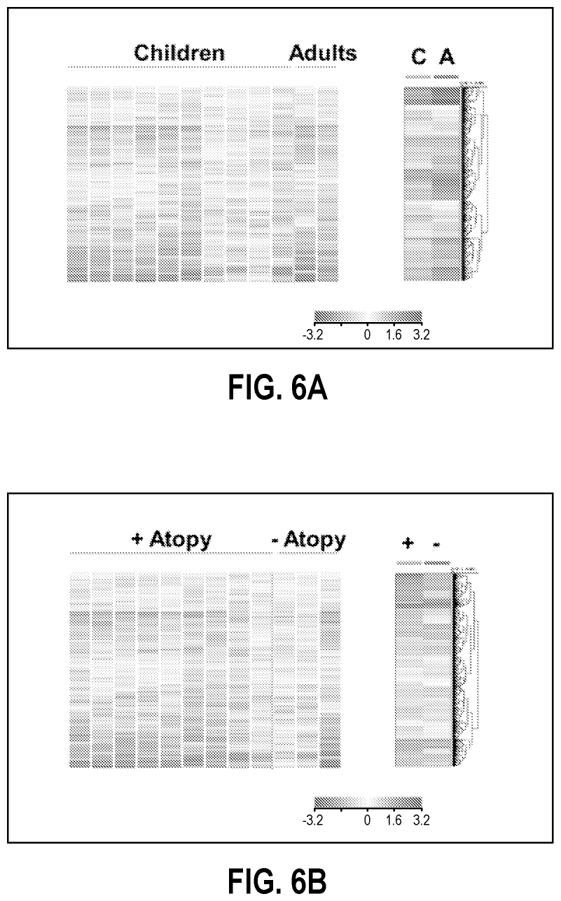

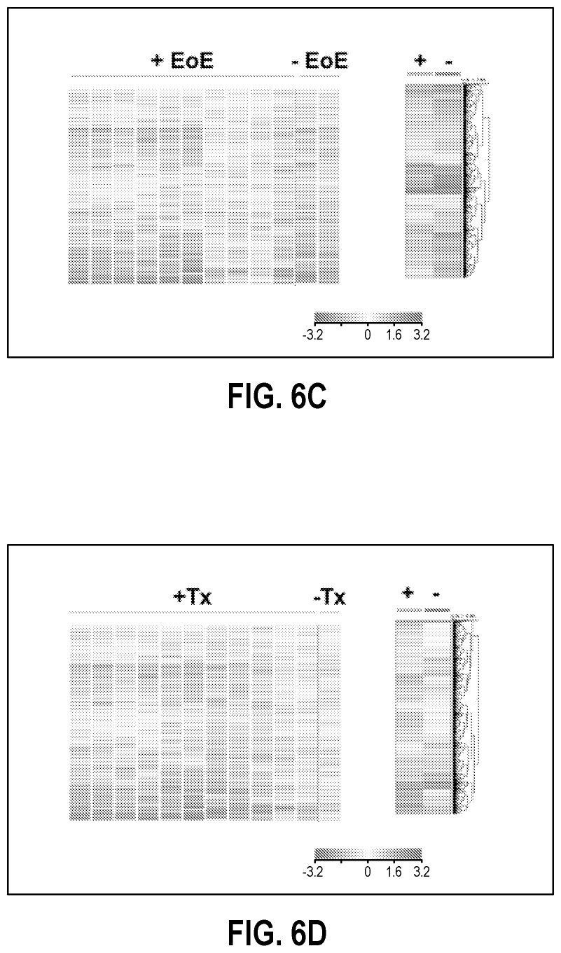

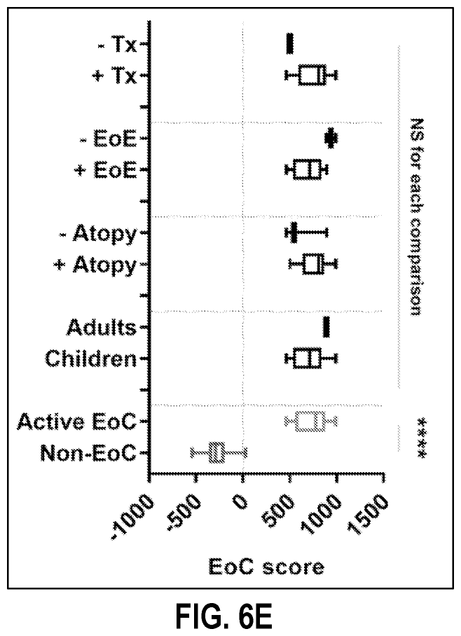

First, Applicant molecularly profiled EoC by using a stringent diagnostic criteria (more than twice the normal number of mucosal eosinophils in colon). To minimize variability and detect meaningful gene dysregulation, Applicant examined the ascending colon, which usually has higher eosinophil counts among colon sites. Applicant generated an RNA sequencing data set from colonic tissue of active EoC (n=6) and Applicant (n=8) and compared gene expression. Applicant identified 987 differentially dysregulated genes in active EoC vs NL biopsies (1.5-fold change, FDR P<0.05) (FIG. 1A). Unsupervised clustering analysis showed separation between active EoC and NL (FIG. 1B). Of these gene signatures (e.g., EoC transcriptome), 577 transcripts were upregulated and 410 were downregulated in active EoC compared with NL (Table 6). Notably, despite clinical heterogeneity, there were no substantial molecular differences in several comparisons, such as EoC with coexisting EoE vs EoC alone, pediatric vs adult patients, atopic vs nonatopic, and treated vs untreated patients (FIG. 6).

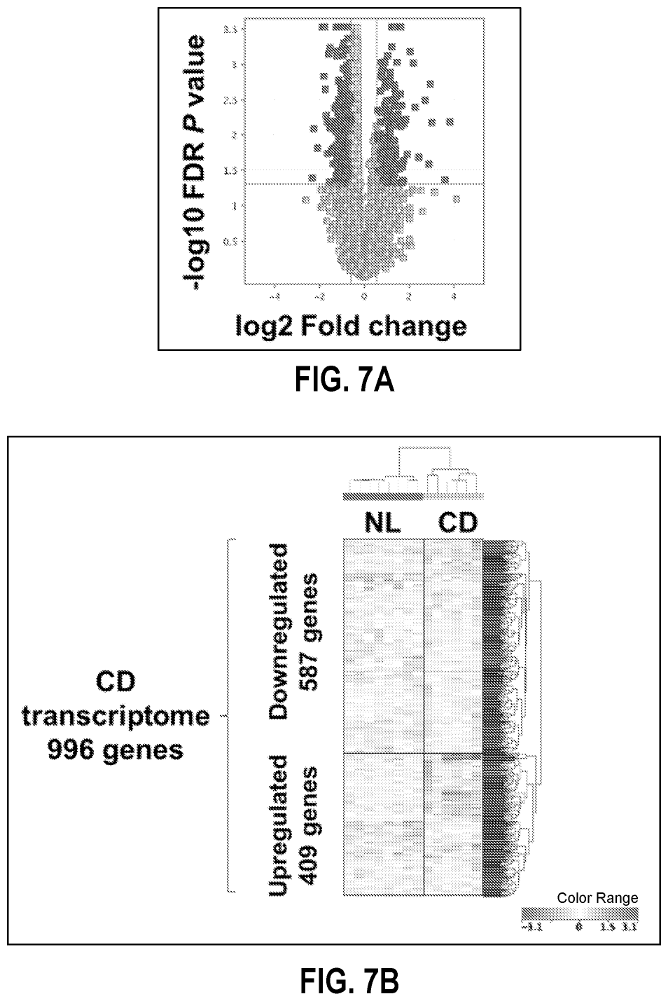

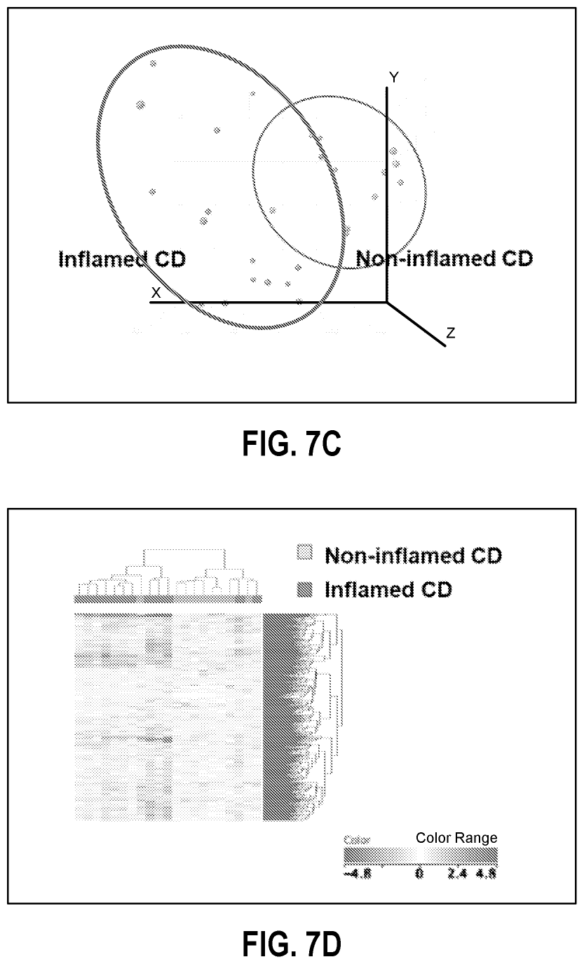

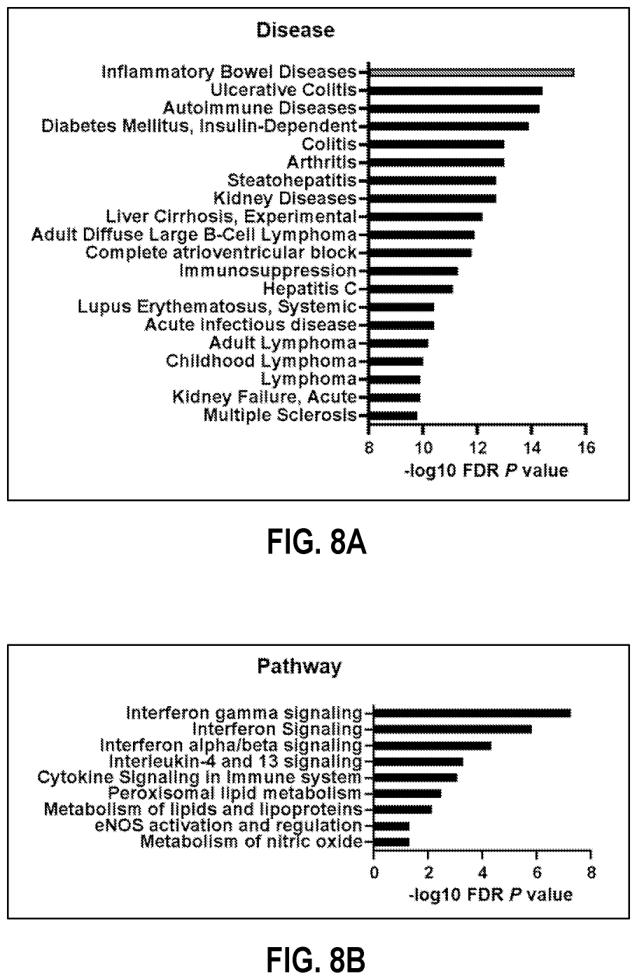

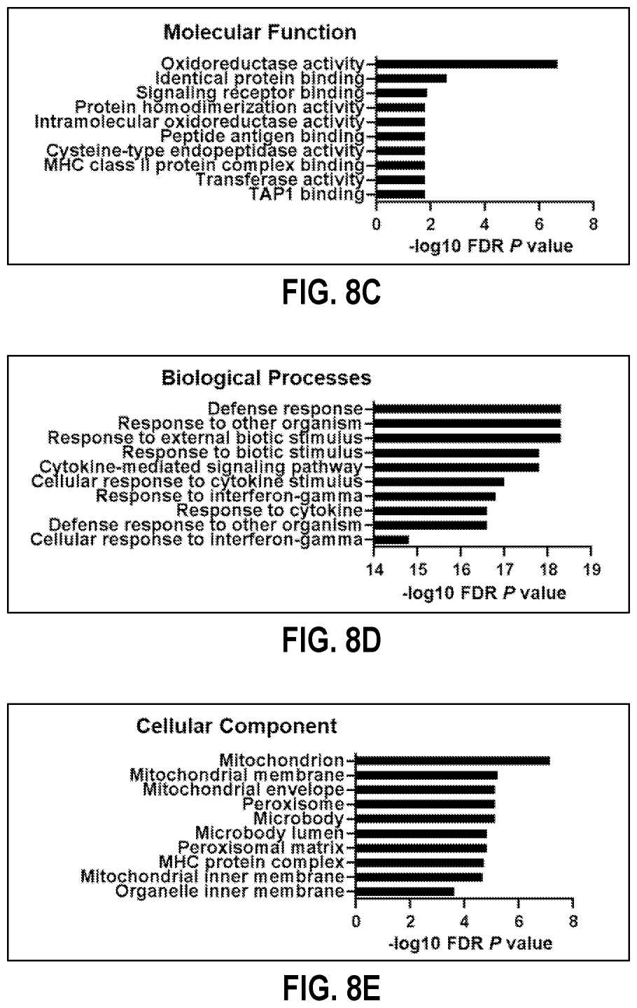

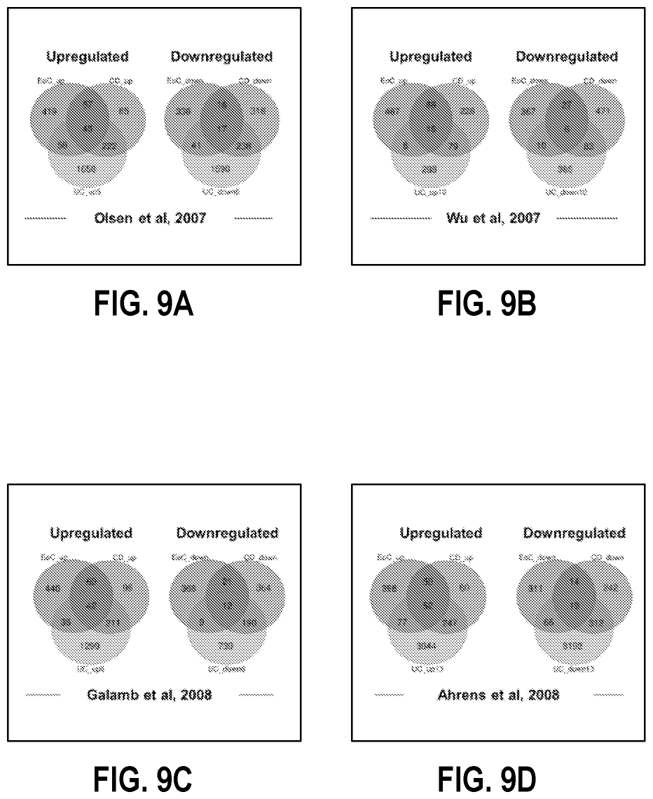

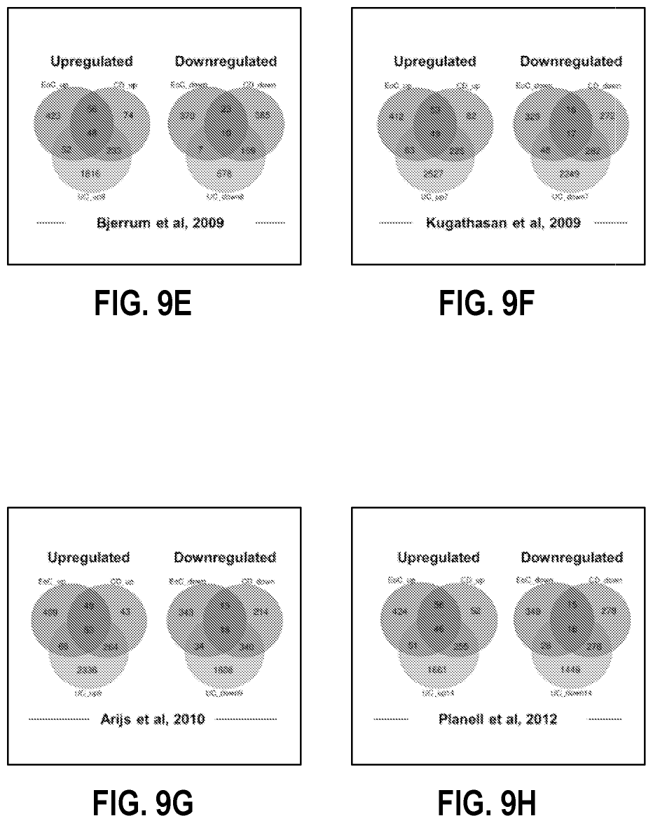

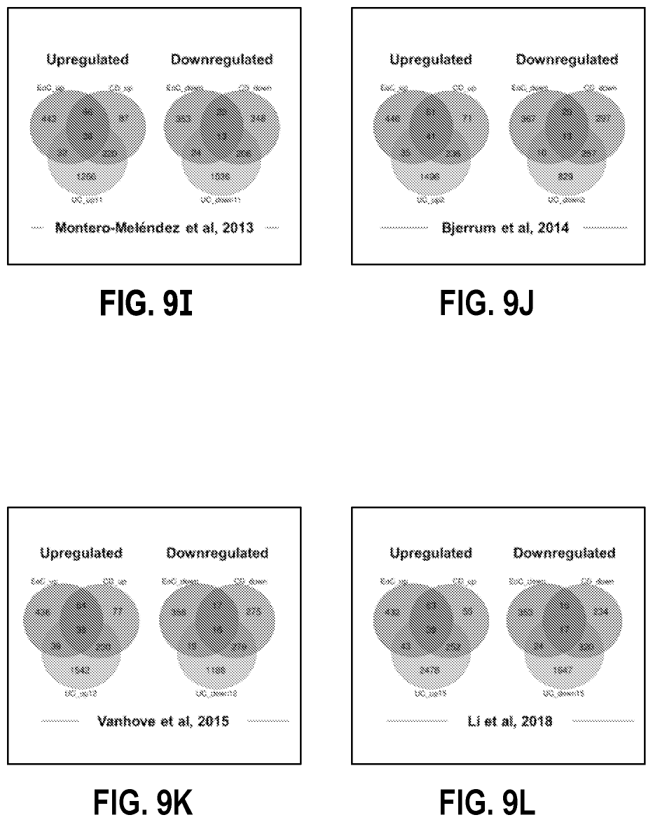

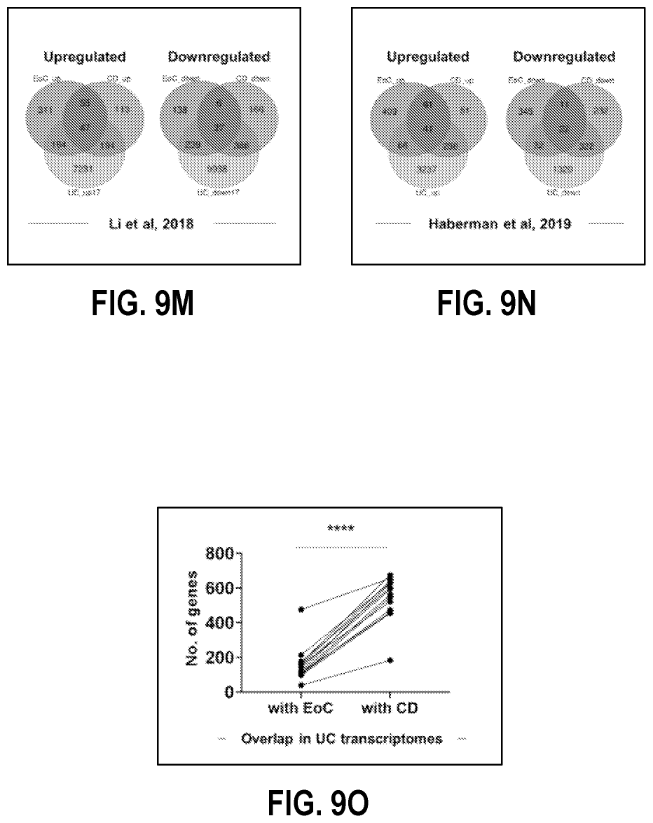

Subsequently, a core gene set was identified for subjects with inflamed CD having active colitis to compare with the EoC transcriptome. Using the same approach as for EoC, a 996-gene CD transcriptome was identified (FIG. 7 and Table 6), which included previously identified genes and pathways associated with inflamed CD (e.g., IBD; C0021390 at DisGeNET) (FIG. 8). Comparing the EoC and IBD (CD, from this study; UC, from 14 published data as summarized in Table 7) transcriptomes demonstrated that EoC was distinct (FIG. 1C and FIG. 9), and unsupervised principal component analysis and hierarchical clustering analysis demonstrated robust separation of active EoC, inflamed CD, and NL groups (FIGS. 1D and E).

EoC Transcriptome Associates with Colonic Eosinophilia and Distinguishes EoC from Other EGIDs

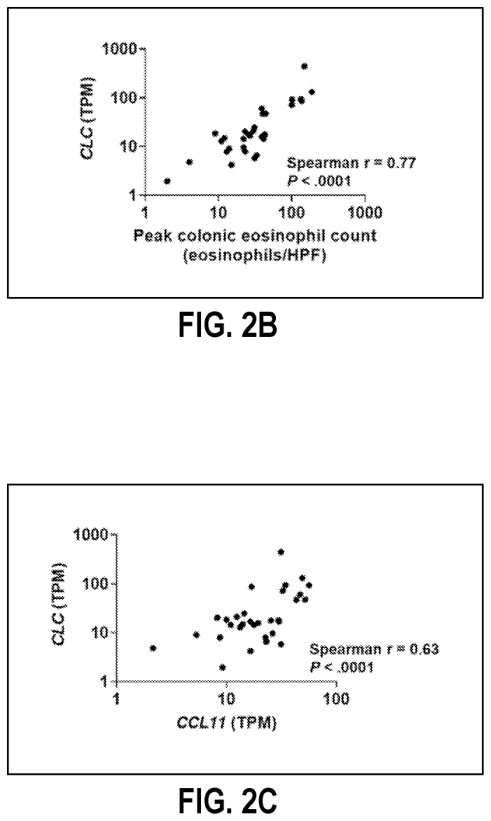

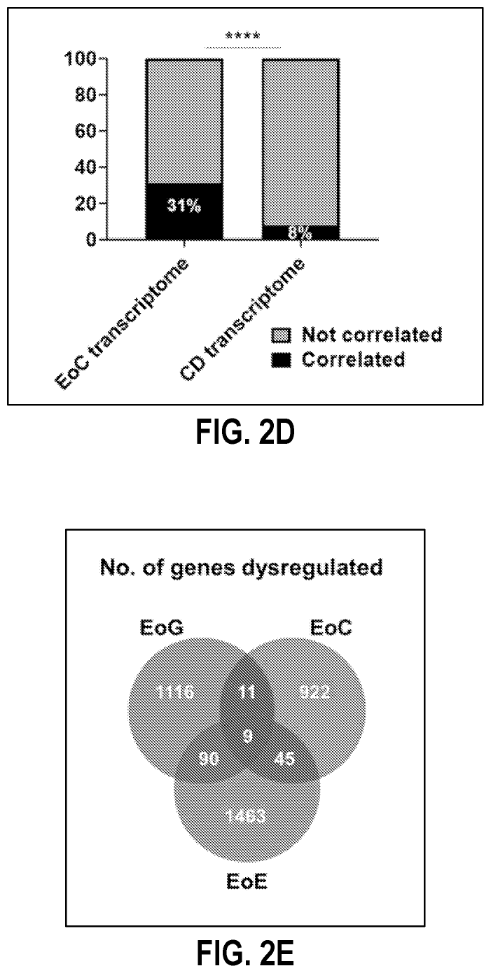

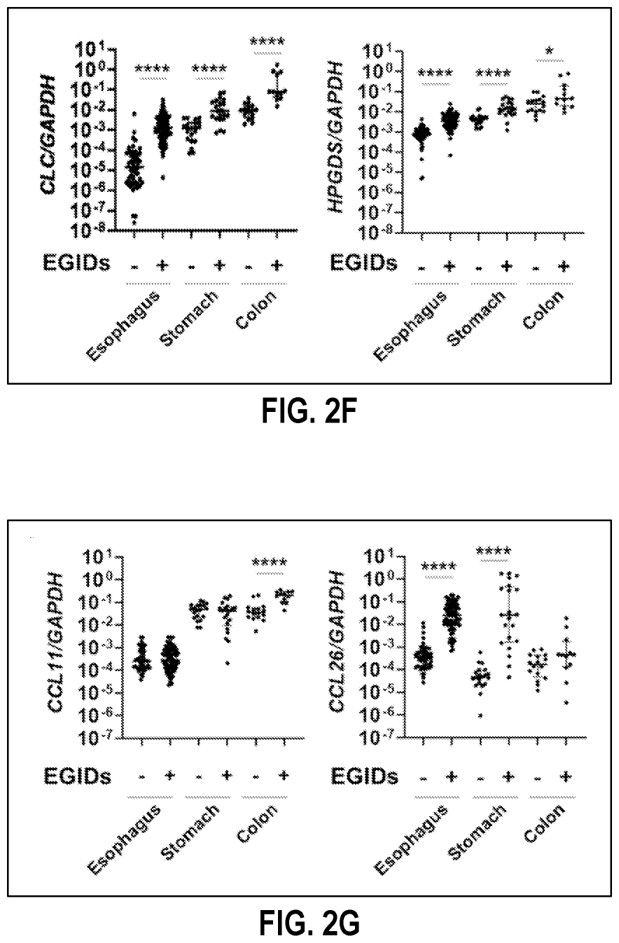

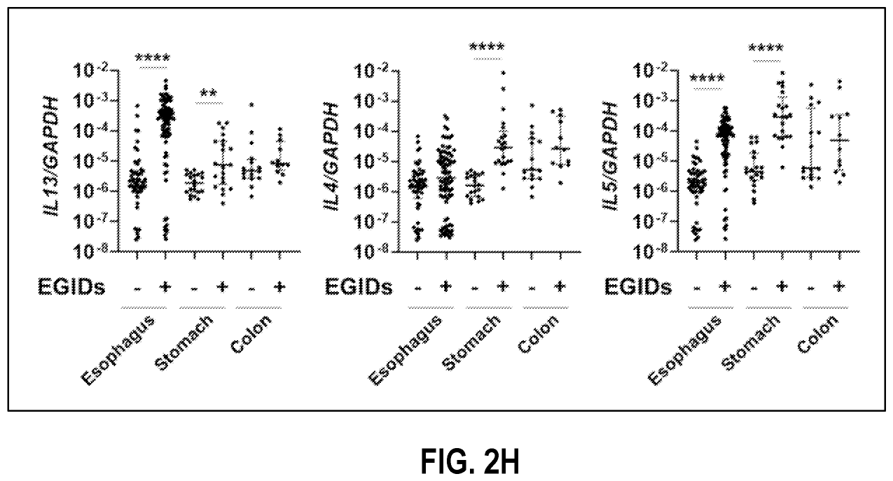

The peak colonic eosinophil count from ascending to sigmoid colon significantly correlated with 31% of the EoC transcriptome (Table 6), most notably with the expression of eosinophil chemoattractant gene C-C motif chemokine ligand 11 (CCL11, r=0.78, P<0.001) and the eosinophil-specific gene Charcot-Leyden crystal (CLC, r=0.77, P<0.001) (FIGS. 2A and B). CCL11 and CLC correlated with each other (r=0.63, P<0.001) (FIG. 2C). Conversely, the peak colonic eosinophil count correlated with 8% of the CD transcriptome (Table 6). The number of genes correlating with the peak colonic eosinophil count significantly differed between the EoC and CD transcriptomes (P<0.0001) (FIG. 2D).

To determine the relationship between EoC and other EGIDs, the EoC colonic transcriptome was compared with the previously published EoE esophageal and EoG gastric transcriptomes. Notably, there was almost no overlap among transcriptomes of these 3 EGIDs (EoE, EoG, EoC) (9 genes; 1% of EoC transcriptome) (FIG. 2E). The common EGID genes, including CLC, which is a hallmark of active eosinophilic inflammation, were regulated in similar manners in subjects with EoE, EoG, and EoC (i.e., upregulated in EoE, EoG, and EoC: ALOX5AP, CD9, CLC, CSF2RB, CXCL1, GAPT, MMP12, NCF2, and SOCS1). For upregulated genes, there were some genes in the EoC transcriptome that modestly overlapped with the EoE and EoG transcriptomes, whereas downregulated genes in the EoC transcriptome did not overlap with other EGIDs (Table 8). Comparing type 2-related gene expression by RT-qPCR (FIG. 2F-H) showed that the main chemotactic factor for EoC (lower GI EGID) was likely CCL11, whereas it was CCL26 for both EoE and EoG (upper GI EGIDs) (FIG. 2G). Expression of type 2 cytokines (e.g. IL13, IL4, and IL5) were increased in patients with EoE and EoG but not in EoC, although there was substantial heterogeneity (FIG. 2H).

### Functions and Cell Types Enriched in the EoC Transcriptome

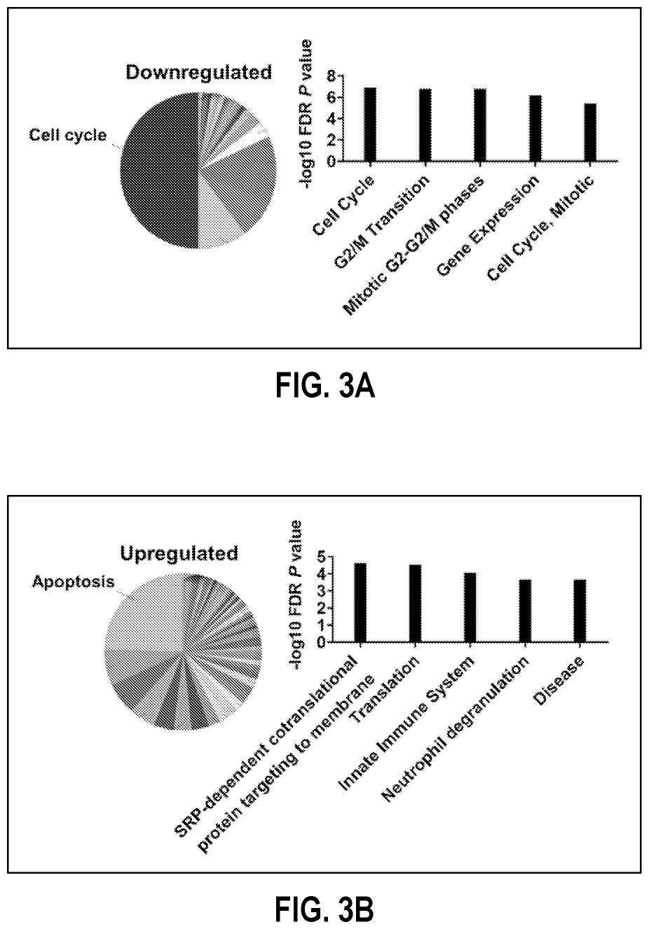

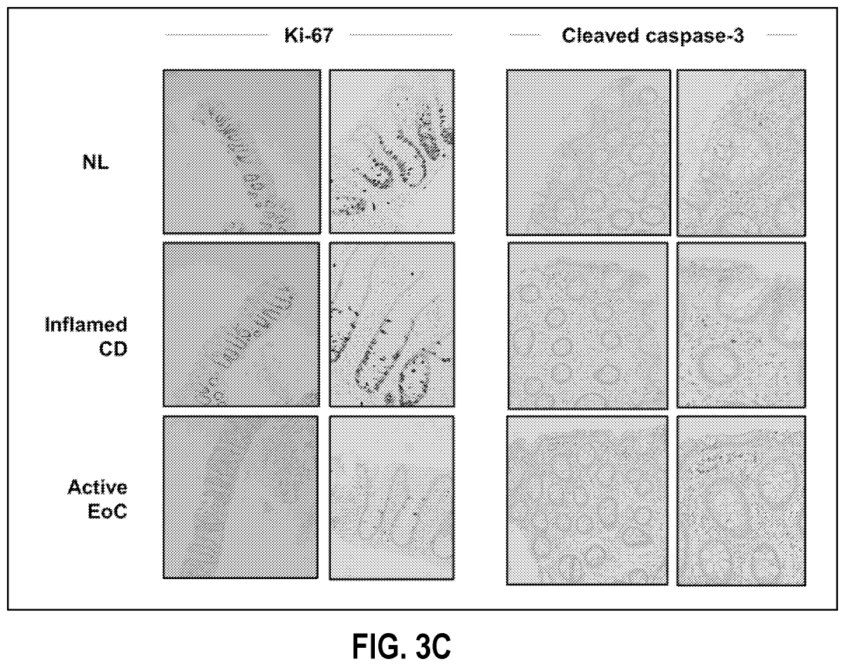

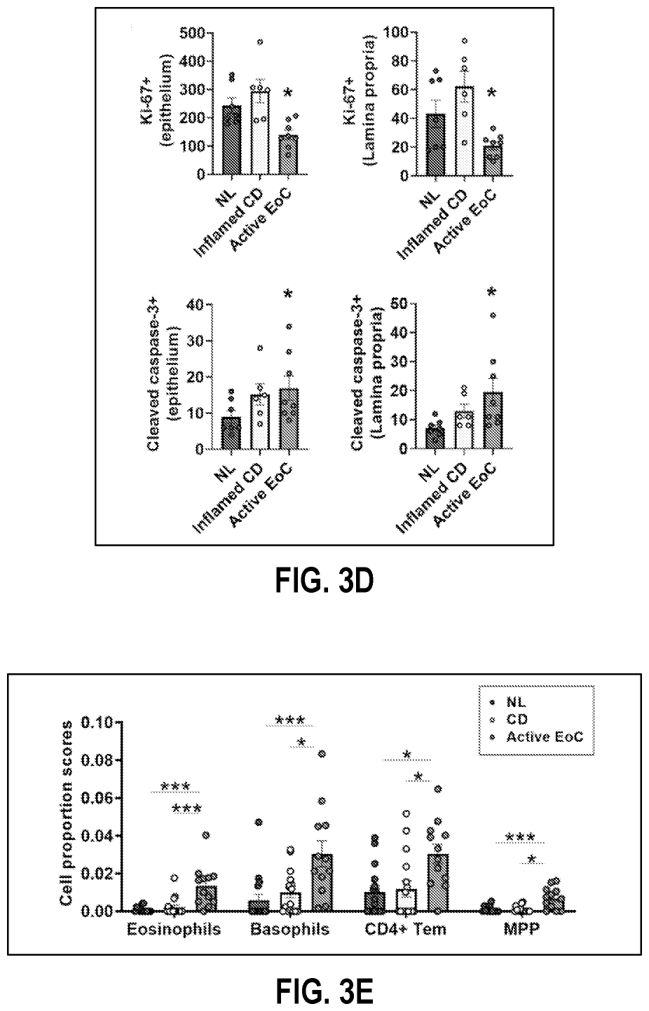

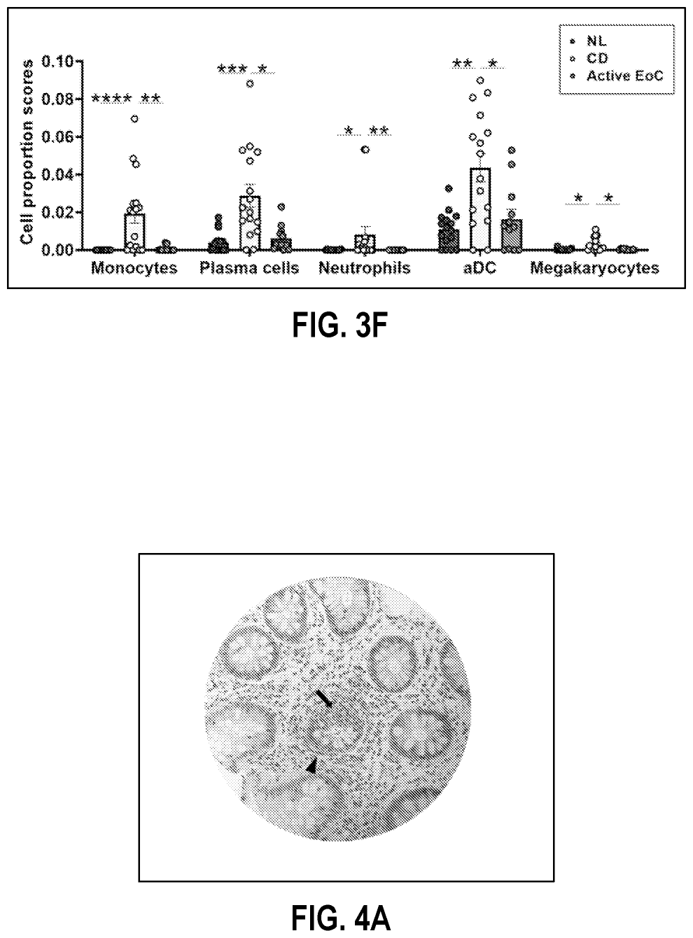

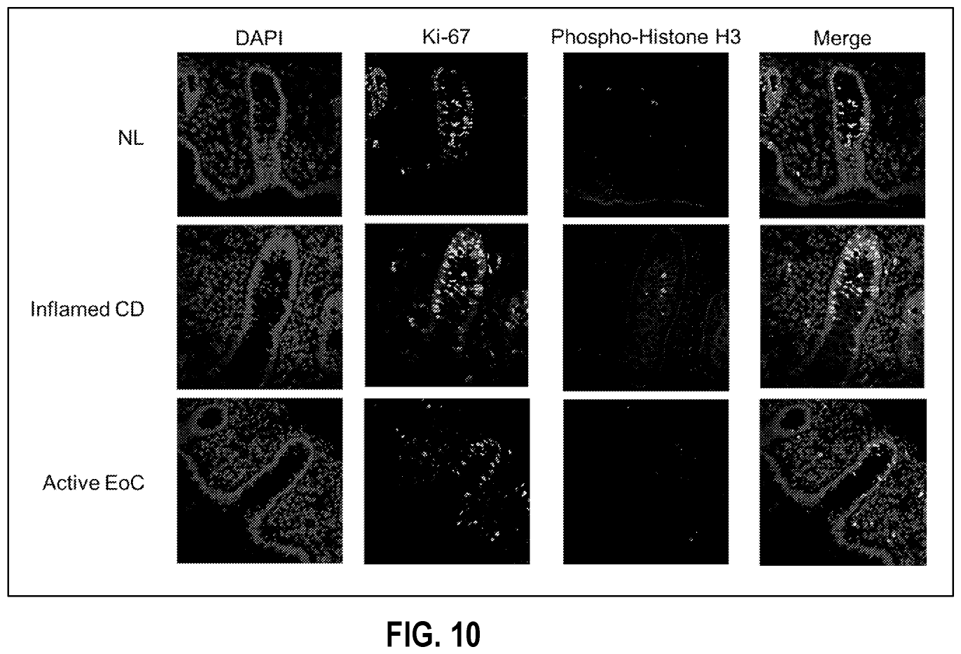

To identify EoC-associated molecular pathways, Applicant performed functional annotation enrichment analyses. The highest enrichments were decreased cell cycle functions and increased apoptosis pathways (FIGS. 3A and B and Table 9). Processes downregulated of the EoC transcriptome showed a decrease in cell cycle transcripts (P=8.6E-7), including proteasome genes. Upregulated processes of the EoC transcriptome were enriched for apoptosis signaling (P=1.9E-4), including ribosomal genes. There was enrichment in granulocyte activation and degranulation and innate immunity (Table 9). Immunohistochemically confirming the pathway analyses showed reduced colonic epithelial and lamina propria cells with positive Ki-67 staining (cell proliferation marker) in biopsy specimens from patients with EoC compared with NL and CD (P<0.05) (FIGS. 3C and D). The number of examined active EoC biopsies was small, but generally Ki-67+ cells appeared reduced in crypts located in areas of dense eosinophilic inflammation. Immunofluorescent staining confirmed reduced Ki-67 staining and also showed decreases in other proliferation markers (phosphohistone H3) (FIG. 10). In contrast, the number of cleaved caspase-3+ (cell apoptosis marker) cells was significantly increased in EoC and CD vs NL biopsy specimens (P<0.05) (FIGS. 3C and D).

Further evaluating the relative composition of immune cell subsets, epithelia, and other stromal cell types in EoC, Applicant applied a computational gene expression deconvolution approach using xCell. Of the 64 cell types represented by gene expression, several immune cells were specifically increased in EoC and CD. Active EoC had increased gene expression associated with eosinophils, basophils, CD4+ effector memory T cells, and multipotent progenitors, whereas inflamed CD had increased gene expression associated with monocytes, plasma cells, neutrophils, activated dendritic cells, and megakaryocytes (FIGS. 3E and F).

Colonic Histologic Features and Transcript Association with Disease

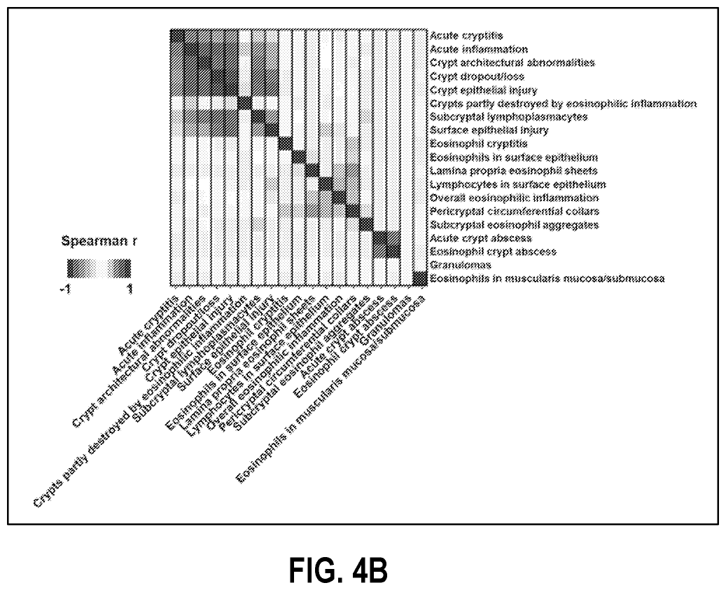

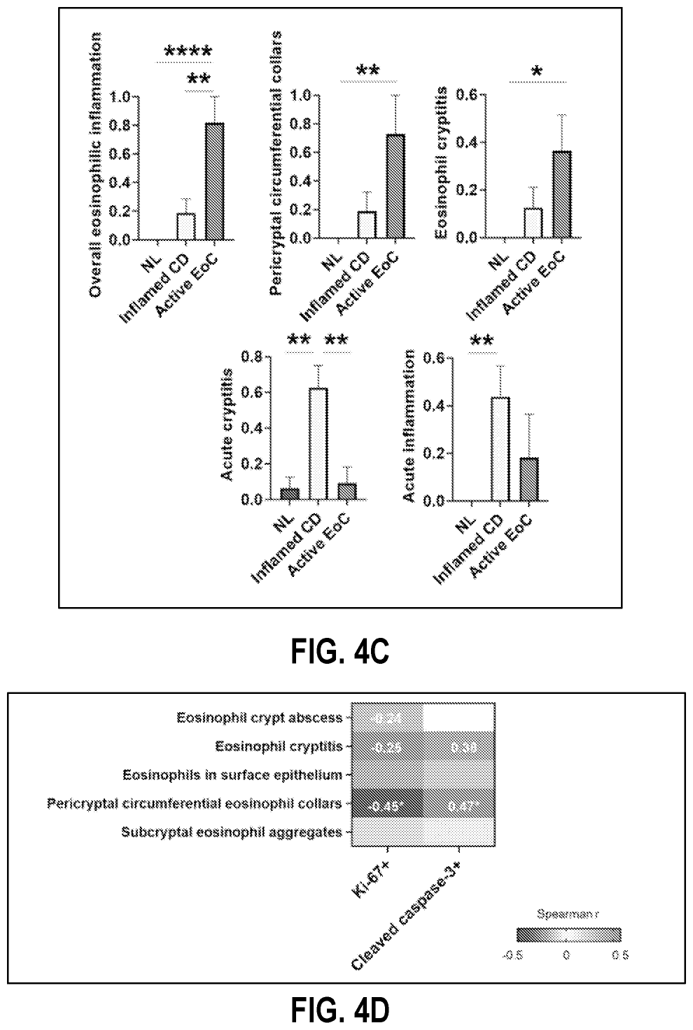

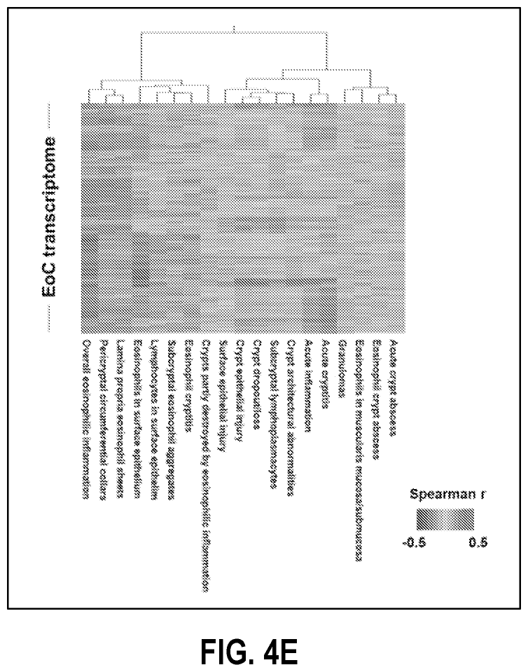

All subjects with active EoC showed marked, though uneven, colonic eosinophilic inflammation (FIG. 4A) even within the same biopsy specimen. In subjects with EoC, the ascending colon had higher peak eosinophil counts than the left colon (mean 96.3 eosinophils/HPF vs. 43.7 eosinophils/HPF, respectively; P=0.007), consistent with the normally higher counts in the right than left colon, whereas histologic features other than eosinophil count were similar regardless of disease activity (active vs. inactive EoC). Notably, tissue eosinophilia with no additional crypt architectural abnormalities was the most common finding (87%) in EoC colonic biopsies.

Assessing the relationships among the EoC colonic histologic features, Applicant generated a correlation plot with clustering arrangement (FIG. 4B). Consistent with features commonly reported by pathologists examining such biopsies, there were strong correlations for inflammatory and structural changes in crypts; the most significant was between crypt epithelial injury and crypt dropout/loss (r=0.80). Also, there were significant correlations within eosinophilic features, the most significant being between pericryptal circumferential eosinophil collars and lamina propria eosinophil sheets (r=0.52). However, possibly due to the low occurrence, features related to eosinophilic and acute crypt abscess had low correlations with other features. Some features were included to distinguish EoC from IBD, including CD, and were not expected to correlate with EoC-related features; for example, sarcoid-like granulomas are a characteristic finding in CD but not EoC and therefore are not expected to correlate with EoC histopathology. Notably, some colonic histologic features specifically associated with diseases, reflecting the intent of the histopathologic examination to distinguish among various colonic diseases. Among the histologic features, 5 features showed differences among the active EoC, inflamed CD, and NL. As expected, overall eosinophilic inflammation, pericryptal circumferential eosinophil collars, and eosinophilic cryptitis were significantly increased in active EoC compared with inflamed CD or NL (FIG. 4C, upper), whereas acute cryptitis and acute inflammation were significantly increased in inflamed CD compared with active EoC or NL (FIG. 4C, lower).

To understand the potential link between eosinophil-associated histologic features and the identified EoC-related functional pathways, correlations were assessed between eosinophilic histologic features and cell proliferation (Ki-67) and apoptosis (cleaved caspase-3) in the epithelium. Pericryptal circumferential eosinophil collars were negatively correlated with cell proliferation (r=−0.45, P<0.05) and positively correlated with apoptosis (r=0.48, P<0.05) (FIG. 4D), suggesting that epithelial-eosinophil cross-talk occurs in areas of eosinophilic collar formation.

Further dissecting the molecular basis for colonic histopathology in EoC, Applicant evaluated associations between the EoC transcriptome and histologic features using Spearman r at the gene level (FIG. 4E). Applicant observed that histologic features commonly observed in EoC biopsies, such as overall eosinophilic inflammation, pericryptal circumferential eosinophil collars, and lamina propria eosinophil sheets, highly correlated with the EoC transcriptome and clustered together. Genes associated with each major histologic feature (r>0.3, P<0.05) showed enrichment in several biological processes: overall eosinophilic inflammation, purine ribonucleotide biosynthetic process (P=2.85E-07); pericryptal circumferential eosinophil collars, mitochondrion organization (P=3.24E-07); and lamina propria eosinophil sheets, protein targeting to endoplasmic reticulum (P=8.63E-06) (Table 10). Overall, Applicant found that EoC had unique pathogenic gene sets and histologic manifestations, suggesting clinical utility of these features because of correlation with pathogenic gene sets.

### EoC Transcriptome as a Function of Differential Diagnosis and Disease Activity

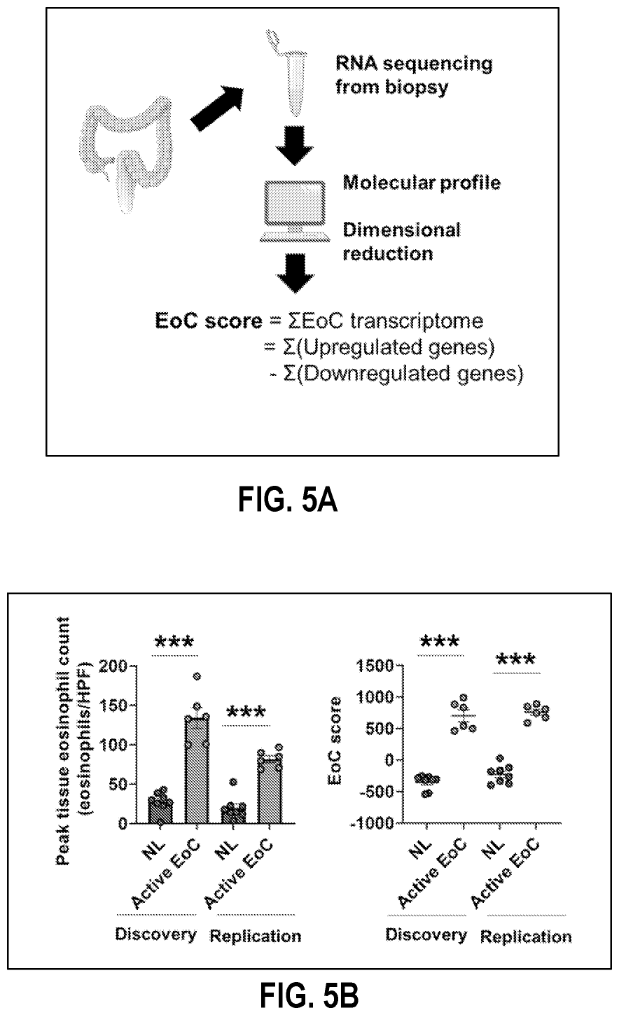

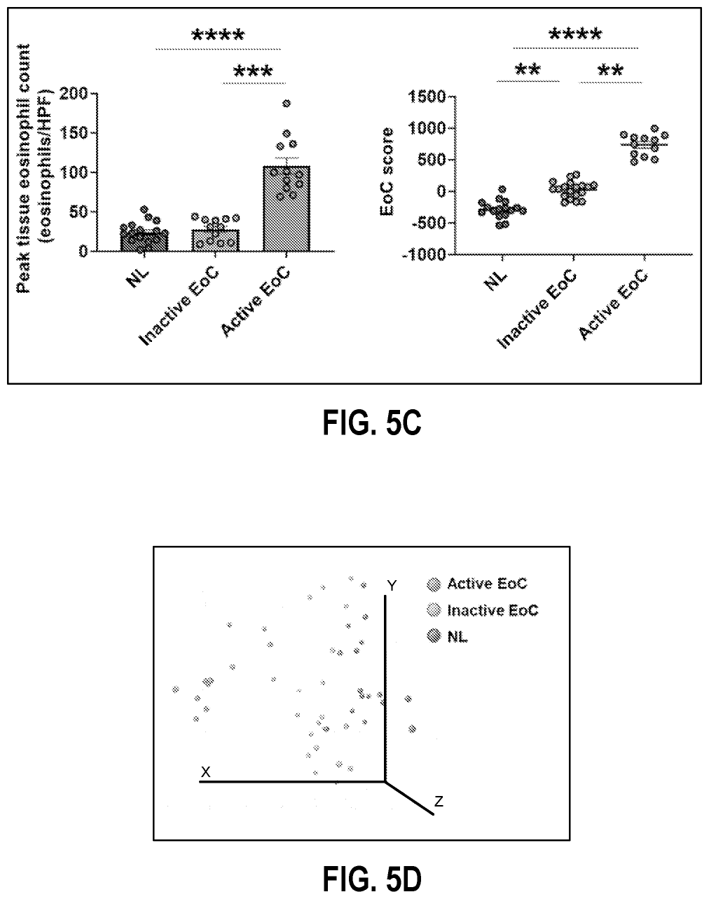

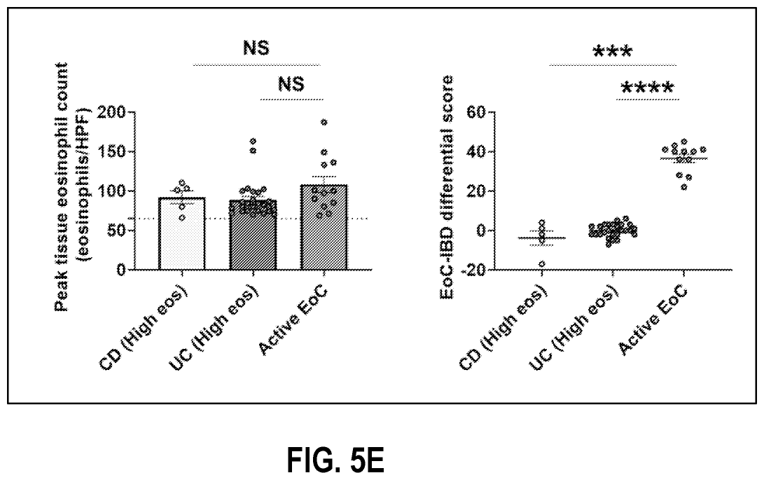

Generating quantitative values to reflect molecular changes, Applicant developed an EoC score by summing the normalized expression values of the dysregulated EoC transcriptome genes (987) (FIG. 5A) to distinguish active EoC from other conditions and quantify EoC severity. The EoC score was increased in patients with active EoC compared with non-EoC (P<0.001) (FIG. 5B, discovery). This finding was replicated in an independent patient cohort, with EoC and non-EoC, regardless of the colon sites (descending and sigmoid colon, P<0.001) (FIG. 5B, replication).

Exploring the potential reversibility of the EoC transcriptome according to disease activity, Applicant compared the EoC score among active EoC, inactive EoC, and NL. Similar to the peak colonic eosinophil count (FIG. 5C, left), the EoC score was specifically increased in patients with active EoC compared with non-EoC patients (P<0.0001) and patients with inactive EoC (P<0.01) (FIG. 5C, right; 5D).

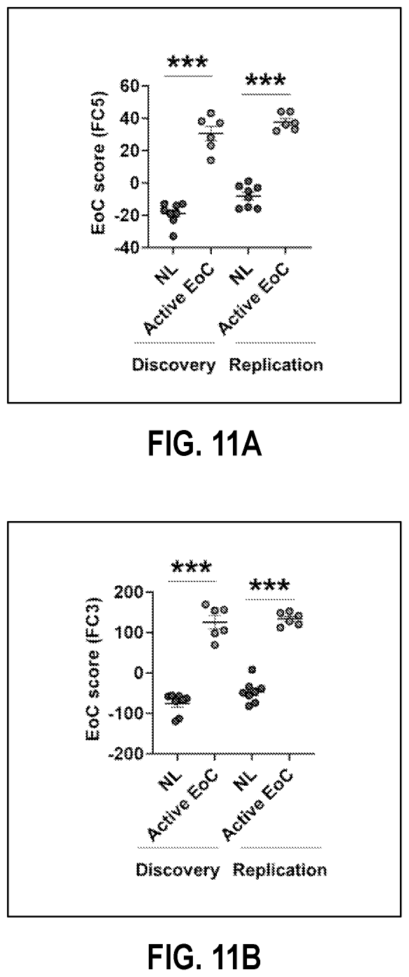

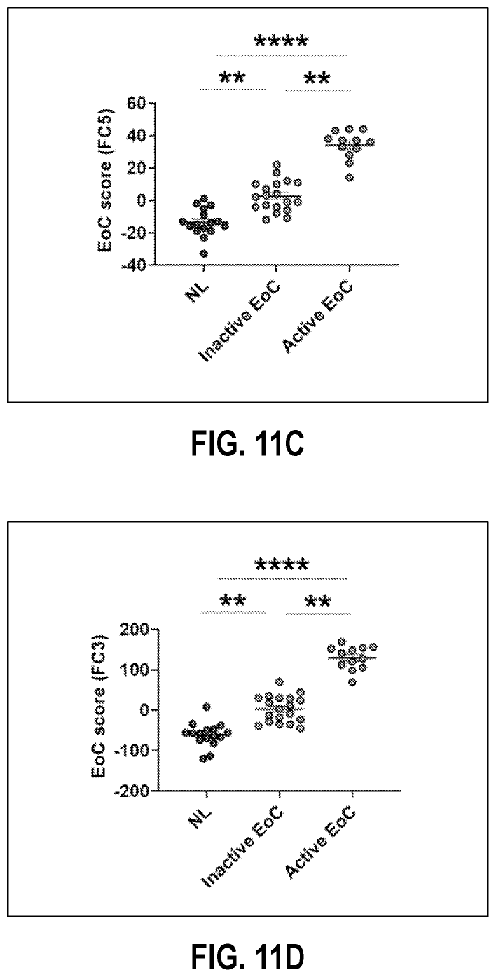

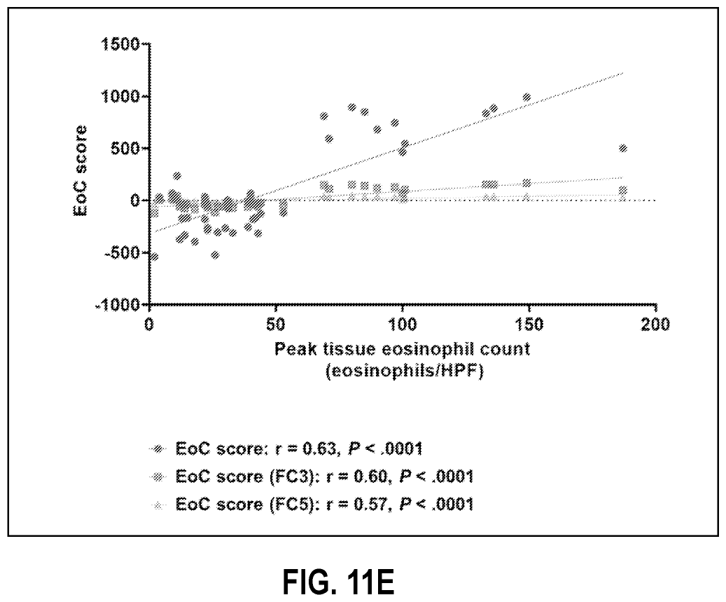

Applicant created a score with the use of a more limited number of genes by using different cutoffs (e.g., 5-fold change, 3-fold change). Although these gene-subset EoC scores (5-fold change or 3-fold change, respectively) showed similar results (FIG. 11), the EoC score based on the entire EoC transcriptome (987 genes) showed a better correlation with peak colonic eosinophil counts (Spearman r=0.63, P<0.0001).

Finally, assessing the potential utility of the EoC transcriptome for definitive diagnosis, Applicant's dataset (EoC and CD) and 1 dataset (UC) having colonic eosinophil counts (GSE109142) were used to generate a modified EoC score, the EoC-IBD differential score, for differential diagnosis against clinically challenging cases. Genes for the modified EoC score were selected from the EoC transcriptome based on the following considerations: dysregulation between EoC and IBD defined by P values and fold changes and bidirectional changes of gene expression. Based on the EoC-IBD differential score derived from 17 genes (Table 11), Applicant compared active EoC and a subset of inflamed IBD (CD and UC) having high colonic eosinophil levels (clinically challenging cases). Although there was no difference in the peak colonic eosinophil count between active EoC and inflamed IBD with high colonic eosinophilia (P=0.211) (FIG. 5E, left), the EoC-IBD differential score separated these groups (P<0.0001) (FIG. 5E, right). A receiver operating characteristic curve analysis demonstrated excellent diagnostic merit for the EoC-IBD differential score (P=0.0001, AUC=1.00) (FIG. 5F).

### DISCUSSION

Molecular and histologic features of EoC are reported herein, as well as the EoC transcriptome, a core gene set believed to be conserved across colon sites in patients with EoC. The EoC transcriptome is associated with tissue eosinophil levels and disease activity and is markedly distinct from upper GI EGID transcriptomes. While type 2 immunity cannot be fully ruled out, there was minimal evidence of strong type 2 allergic inflammation in EoC when compared with the type 2 signature seen in EoE and EoG.

Robust EoC gene expression revealed functional pathways in EoC pathogenesis, including molecular evidence for reduced cell proliferation and increased apoptosis, which were substantiated in biopsies by Ki-67 and cleaved caspase-3 staining. Reduced cell proliferation was unexpected and suggests that distinct cellular mechanisms might be locally operational in EoC. Based on cell deconvolution, Applicant identified the involvement of eosinophils, basophils, CD4+ effector memory T cells, and multipotent progenitors in EoC. The magnitude of molecular changes was linked to histologic changes. Strong correlations with the EoC transcriptome were observed in pericryptal circumferential eosinophil collars, providing a better understanding of histologic features of clinical biopsies.

Applicant further showed that the EoC score, based on the EoC transcriptome, readily assessed disease activity and distinguished EoC from the clinically challenging cases of IBD with high eosinophilia. This collective evidence establishes that EoC is a discrete disease entity involving pathways distinct from those of upper EGIDs and IBD.

CLC is the gene believed to be most highly induced in EoC. Given this gene's specificity to eosinophils and basophils and that eosinophilic inflammation is a hallmark of EoC, this finding substantiates the data's integrity. Indeed, colonic CLC expression levels strongly correlated with eosinophilia-quantified disease severity. CLC protein (i.e., galectin 10) is an eosinophil-specific granule protein that is secreted by activated eosinophils and promotes type 2 immune activity. Antibodies directed against key epitopes of the CLC crystallization interface have been shown to dissolve preexisting CLCs in mucus from patients with asthma and were effective in controlling disease in a humanized mouse model. As CLCs can be found in EoC stool, these antibodies may be beneficial for relieving EoC tissue inflammation.

Although CLC and other eosinophil products likely promote proinflammatory changes in EoC, eosinophil regulation may differ in patients with EoC compared with other EGIDs. Notably, CCL11 (eotaxin-1), but not CCL24 (eotaxin-2) nor CCL26 (eotaxin-3), was highly upregulated in tissue from patients with EoC compared with control tissue and exhibited a significant, positive correlation with colonic CLC expression. This finding is consistent with an essential role for CCL11 (eotaxin-1) in regulating eosinophil-associated GI pathology, from the small intestine to the colon, in a mouse model and humans. CCL11 (eotaxin-1) upregulation is also observed in IBD, suggesting similar colonic eosinophil regulation. Differences in tissue composition (e.g., resident cell types) or distinct disease mechanisms (e.g., differential cell recruitment or altered gene expression programs of resident cells) may account for these findings. The dissimilarities in differentially regulated transcripts, especially CCL26 (eotaxin-3) in patients with upper EGIDs (EoE, EoG) and CCL11 (eotaxin-1) in those with lower EGID (EoC) might arise, at least partially, from the distinct structural cells and immunocytes present in those tissues.

Pathway analysis of the EoC transcriptome identified a robust reduction in cell cycle pathways, which was substantiated by a decreased number of proliferating (Ki-67) cells in EoC colonic biopsies. In contrast, upper GI EGIDs (EoE and EoG) feature expansion of the basal epithelium and increased cell proliferation. A series of downregulated genes, including NADPH oxidase 1 (NOX1), stratifin (SFN), and several 26S proteasome (PSMC1, 3, 6, PSMD4, 7), may relate to the decreased cell proliferation, as decreased NOX1 expression is known to produce a significant decline in reactive oxygen species production and cell cycle arrest. Interestingly, NOX2-deficient mice have interstitial pneumonitis with eosinophilic crystals and granulomas. Inhibiting SFN expression increases apoptosis and cell cycle arrest. In addition, the 26S proteasome is known as the end point of the ubiquitin proteasome pathway that is chiefly required for cell cycle progression. The observed enrichment of decreased expression in 26S proteasome-associated genes might relate to decreased proliferation in patients with EoC. Notably, several cases of colitis were reported after taking bortezomib, an inhibitor of the 26S proteasome. Functional pathway analysis of the EoC transcriptome also showed evidence of increased apoptosis in agreement with an increased number of apoptotic (cleaved caspase-3) cells in EoC colonic biopsies. Positive regulation of apoptosis could slow down epithelial turnover and proliferation in colonic tissue, leading to impaired intestinal barrier function and facilitating inflammatory processes. Relatedly, in colon biopsy specimens from infants with allergic/eosinophilic colitis, high numbers of apoptotic epithelial cells were identified by apoptotic cell-specific histochemical assay. Interestingly, previous microRNA analysis of patients with EoC also suggested this phenomenon. Furthermore, the dominance of caspase 3, in contrast to caspase 8 and the ripoptosome, further contrasts the tissue-specific responses related to EoC and EoE pathogenesis. Applicant's collective data suggest distinct molecular and cellular mechanisms are locally operational in patients with EoC.

Thus, the data highlights colonic histologic changes as having utility in EoC diagnosis. Additional pathologic changes, including the presence of eosinophil sheets, cryptitis or crypt abscesses, and muscular involvement, are also present and may facilitate the diagnosis of EoC. Furthermore, the lack of acute inflammation and cryptitis (features of IBD) should raise suspicion for EoC.

EoC had a distinct molecular profile and correlating histologic features. Of the EoC histologic features, eosinophilic features were highly associated with the EoC transcriptome, with the strongest association being pericryptal circumferential eosinophil collars. As expected, not all histologic features showed strong associations with the EoC transcriptome, possibly because of the low occurrence in patients with EoC of some histologic features that were anticipated to be prominent in CD but not EoC, namely, acute inflammatory cells, surface erosion/ulceration, and lamina propria fibroplasia. Indeed, some colonic histologic features, including pericryptal circumferential eosinophil collars, were specifically associated with the EoC-associated functions (decreased cell proliferation, increased apoptosis).

The imbalance of cell proliferation and cell death, normally maintained in cellular homeostasis, and its correlation with unique histologic features associated with EoC suggest epithelial-eosinophil crosstalk particularly at the interface of eosinophilic collars. The eosinophilic features best reflected the molecular signature changes in EoC, warranting close attention to them when interpreting disease diagnosis and activity.

Applicant notes the small sample size of EoC because of the rarity of the disease may limit the impact of results and heterogeneity in EoC (e.g., comorbid EoC-EoE vs EoC alone) may affect the results, although it may be practical as reflecting the real-world manifestations. The study definition for EoC and its activity was applied for balanced feasibility and accuracy, warranting future analyses with further accurate evaluations (e.g., controlled comorbidity, validated symptom assessment, and standardized endoscopic/histomolecular follow-up). The findings included patients with active EoC and CD who had mixed treatment status (Table 1) and patients who had treatment refractory disease, which might influence the results. However, patients still exhibited signs of active disease clinically, histologically, and molecularly. Therefore, the treatments were not effective in eradicating the disease, and key molecular pathways involved in pathogenesis were likely still active, at least partially. Though unbiased, highly sensitive, genome-wide transcriptome approaches were used to identify key gene signatures, the analyses were performed on whole biopsies, composed of a mixture of cellular components, rather than single cells. Computational deconvolution of cell subset proportions were performed to address this limitation. The data were limited by the cross-sectional approach.

In conclusion, Applicant established EoC as a unique GI disease and identified a conserved colonic transcriptome that associates with colonic eosinophilia, is markedly distinct from that of other GI diseases, and is uniquely associated with distinct histologic features, especially pericryptal circumferential eosinophil collars. Mechanistically, Applicant uncovered that EoC is not related to strong type 2 immunity but rather apoptosis and reduced epithelial cell proliferation.

### Tables

TABLE 1
Demographic and clinical characteristics of study subjects.

| | NL | | CD | | EoC | | P value |
|---|---|---|---|---|---|---|---|
| Subjects (n) | 20 | | 14 | | 27 | | |
| Demographic features | | | | | | | |
| Age at biopsy (min.-max. years) | 15.1 | (4.3-44.9) | 17.3 | (11.7-21.6) | 14.0 | (7.1-64.4) | .26 |
| Gender (% Male) | 8 | (40%) | 7 | (50%) | 14 | (52%) | .71 |
| Race (% White) | 18 | (90%) | 14 | (100%) | 25 | (93%) | .49 |
| Colonic eosinophil counts | | | | | | | |
| Peak (eos/HPF) | 28.5 | (23.8-37.5) | 48.5 | (31.0-68.3) | 55.5 | (23.8-100.8) | .025 |
| Range (min.-max. eos/HPF) | 2-43 | | 16-110 | | 9-187 | | |
| Biopsies (n) | | | | | | | |
| Total | 29 | | 27 | | 31 | | — |
| Active or inflamed | — | | 16 | | 12 | | — |
| Normal | 16 | | — | | — | | — |
| Right colon | 10 | | 14 | | 21 | | — |
| Left colon | 19 | | 13 | | 10 | | — |
| History of EGIDs | | | | | | | |
| EoE | — | | — | | 15 | (56%) | — |
| EoG | — | | — | | 5 | (19%) | — |
| EoC | — | | — | | 27 | (100%) | — |
| History of atopy | | | | | | | |
| Atopy (any) | 15 | (75%) | 8 | (57%) | 15 | (56%) | .36 |
| Asthma | 7 | (35%) | 2 | (14%) | 6 | (22%) | .36 |
| Allergic rhinitis | 13 | (65%) | 5 | (36%) | 11 | (41%) | .16 |
| Eczema | 8 | (40%) | 3 | (21%) | 9 | (33%) | .52 |
| Food allergy | 5 | (25%) | 0 | (0%) | 7 | (26%) | .11 |
| Treatment at biopsy | | | | | | | |
| Ongoing diet therapy | 9 | (45%) | 2 | (14%) | 12 | (44%) | .12 |
| Proton pump inhibitor | 11 | (55%) | 0 | (0%) | 9 | (33%) | .004 |
| Topical steroids | 3 | (15%) | 0 | (0%) | 8 | (30%) | .059 |
| Systemic steroids | 0 | (0%) | 4 | (29%) | 2 | (7%) | .019 |
| Immune modulator | 0 | (0%) | 2 | (14%) | 0 | (0%) | <.001 |
| Biologics | 0 | (0%) | 9 | (64%) | 0 | (0%) | <.001 |

TABLE 2
Number of subjects with EoC from each site

| | Total | Inactive EoC | Active EoC |
|---|---|---|---|
| Number of samples | N = 31 | N = 19 | N = 12 |
| Children's Hospital Colorado | 7 | 6 | 1 |
| Cincinnati Children's Hospital | 20 | 10 | 10 |
| Medical Center | | | |
| National Institutes of Health | 1 | 1 | 0 |
| Northwestern University | 2 | 1 | 1 |
| University of North Carolina | 1 | 1 | 0 |
| School of Medicine | | | |

TABLE 3
Inclusion/exclusion criteria, thresholds of eosinophilia, and definition
of disease activity for EoC. CD, Crohn disease; EoC, eosinophilic colitis; EoG, eosinophilic
gatritis; EGID, eosinophilic gastrointestinal disorder; EoE, eosinophilic esophagitis; GI,
gastrointestinal; HPF, high-power microscopic field; IBD, inflammatory bowel disease; UC,
ulcerative colitis.
Inclusion/Exclusion criteria

| Inclusion | Exclusion |
|---|---|
| Presence of symptoms; symptoms include | History of intestinal surgery other than G tube placement |
| (but are not limited to) hematochezia, | Planned or recent enrollment in blinded investigational |
| bloody/non−bloody diarrhea, tenesmus, and | studies |
| abdominal pain | Other identifiable potential causes for eosinophilia except |
| A history of one of the following at the time | for IBD (CD and UC). In the case of IBD, investigators |
| of biopsy | will review the clinical history, serology, and |
| 1) anemia | accompanying biopsies to determine if the diagnosis |
| 2) peripheral eosinophilia | captures IBD diagnosis in the context of the histologic |
| 3) hemooccult positive stool | finding of mucosal eosinophilia. If the assessment |
| 4) EGID | suggests that the subject clearly has IBD, the subject will |
| 5) allergic diseases (allergic rhinitis, | not be enrolled. If the assessment suggests that the patient |
| asthma, food allergy, eczema or other | may have EoC, then the potential participant could enroll |
| allergic features suggestive of atopic | if all other inclusion exclusion criteria are met. |
| disease) | Any physical, mental, or social condition, history of |
| Mucosal eosinophilia (see below) | concurrent illness or laboratory abnormality that, in the |
| | investigator's judgement, might interfere with study |
| | procedures or the ability of the subject to adhere to and |
| | complete the study |
| | Potential participants will be excluded if the investigator |
| | determines that the potential participant has diminished |
| | capacity and is not cognitively able to participate fully in |
| | the consenting process |

Thresholds of mucosal eosinophilia in the GI tract

| Location | Sites of biopsy | Thresholds | Diagnosis |
|---|---|---|---|
| Esophagus | Distal, proximal | ≥15 eosinophils/HPF | Eosinophilic esophagitis (EoE) |
| Stomach | Antrum, body, fundus | ≥30 eosinophils/HPF in | Eosinophilic gastritis (EoG) |
| | | 5 HPF | |
| Colon | Ascending | ≥100 eosinophils/HPF | Eosinophilic colitis (EoC) |
| | Descending | ≥85 eosinophils/HPF | |
| | Sigmoid | ≥65 eosinophils/HPF | |

Definition of disease activity in EoC

| Location | Sites of biopsy | Active state | Inactive state |
|---|---|---|---|
| Colon | Ascending | ≥100 eosinophils/HPF | <100 eosinophils/HPF |
| | Descending | ≥85 eosinophils/HPF | <85 eosinophils/HPF |
| | Sigmoid. | ≥65 eosinophils/HPF | <65 eosinophils/HPF |

TABLE 4
Definitions of EoC histologic features. EoC, eosinophilic colitis; HPF,
high-power microscopic field; IEL, intraepithelial lymphocytes.

| Features | Definition | Mild/moderate | Marked |
|---|---|---|---|
| Overall eosinophilic | Estimate overall eosinophilic | ≤50% of HPF contain | >50% of HPF |
| inflammation | inflammation | 65 or more eosinophils | contain 65 or more |
| | | | eosinophils |
| Lamina propria | Contiguous eosinophils that | Contiguous eosinophil | Contiguous |
| eosinophil sheets | form masses in the lamina | masses without | eosinophil masses |
| | propria and may increase | increased intercryptal | with increased |
| | intercryptal distance | distance | intercryptal |
| | | | distance |
| Pericryptal | At least one layer of eosinophils | Single layer of | More than 1 layer |
| circumferential | that surround ≥3/4 of | eosinophils forms collar | of eosinophils |
| collars | circumference of crypt cut in | | forms collar |
| | cross section or entire base of a | | |
| | crypt cut lengthwise | | |
| Eosinophils in | Eosinophils in surface | 3-5 eosinophils/100 | >5 eosinophils/100 |
| surface epithelium | epithelium | surface colonocytes | surface colonocytes |
| | | | or any size of group |
| | | | of eosinophils |
| Lymphocytes in | Lymphocytes in surface | <10 IEL/100 surface | >10 IEL/100 |
| surface epithelium | epithelium | colonocytes | surface colonocytes |
| Surface epithelial | Surface colonocytes that do not | Vacuolization, flattening | Detached injured |
| injury | appear homogeneous or intact | or mucin depletion in | epithelial cells |
| | | epithelial cells that are | |
| | | attached to underlying | |
| | | lamina propria | |
| Eosinophil cryptitis | Eosinophils in crypt epithelium ± | <20 eosinophils/crypt | ≥20 |
| | eosinophils in lumen | | eosinophils/crypt or |
| | | | any size of group of |
| | | | eosinophils |
| Eosinophil crypt | Dilated crypt lined by attenuated | ≤10% of crypts | >10% of crypts |
| abscess | epithelium with eosinophils in | | |
| | pericryptal lamina propria, crypt | | |
| | epithelium, and crypt lumen | | |
| Acute cryptitis | Intraepithelial acute | Focal infiltration by | Circumferential |
| | inflammatory cells in any | acute inflammatory cells | infiltration by acute |
| | quantity of crypts | | inflammatory cells |
| Acute crypt abscess | Dilated crypt lined by attenuated | ≤10% of crypts | >10% of crypts |
| | epithelium with acute | | |
| | inflammatory cells in pericryptal | | |
| | lamina propria, crypt epithelium, | | |
| | and crypt lumen | | |
| Subcryptal | Groups of 5 or more contiguous | ≤10% of crypts | >10% of crypts |
| eosinophil | eosinophils in lamina propria | | |
| aggregates | between crypt base and | | |
| | muscularis mucosa | | |
| Subcryptal | Groups or aggregates of plasma | Affects ≤5 crypts | Affects >5 crypts |
| lymphoplasmacytosis | cells or lymphocytes situated | | |
| | between the crypt base and the | | |
| | muscularis mucosa | | |
| Eosinophils in | Eosinophils in muscularis | Found in ≤5 HPF | Found in >5 HPF |
| muscularis | mucosa/submucosa | | |
| mucosa/submucosa | | | |
| Crypt architectural | Elongated crypts, branched | ≤10% of crypts | >10% of crypts |
| abnormalities | crypts, tortuous crypts, | | |
| | atrophic/missing crypts, etc. | | |
| Crypt dropout/loss | Intercryptal distance is at least | ≤5 foci | >5 foci |
| | doubled | | |
| Crypts partly | Any loss of crypt integrity | ≤10% of crypts | >10% of crypts |
| destroyed by | associated with eosinophils, | | |
| eosinophilic | usually part of a crypt wall is | | |
| inflammation | missing and eosinophils are | | |
| | in/near the breach | | |
| Crypt epithelial | Vacuolization, flattening or | Found in ≤5 HPF | Found in >5 HPF |
| injury | mucin depletion in crypt | | |
| | epithelial cells | | |
| Acute inflammation | Acute inflammatory cells in | Found in ≤5 HPF | Found in >5 HPF |
| | lamina propria | | |
| Granulomas | Epithelioid non-necrotizing | Found in ≤5 HPF | Found in >5 HPF |
| | granulomas in any part of biopsy | | |

TABLE 5
Demographic and clinical characteristics of study subjects.

| | | | | | | | | | | CD with | |
|---|---|---|---|---|---|---|---|---|---|---|---|
| | | | | Inactive | | Active | | Inflamed | | high | |
| | | NL | | EoC | | EoC | | CD | | eosinophils | |
| No. of subjects | | 8 | | 16 | | 11 | | 6 | | 5 | |
| Demographics | | | | | | | | | | | |
| Age at biopsy | | 15.1 | (10.4-17.2) | 16.0 | (12.4-23.6) | 11.2 | (7.6-14.2) | 17.7 | (12.8-19.9) | 17.5 | (14.1-20.5) |
| (years) | | | | | | | | | | | |
| Gender | Male | 1 | (12.5%) | 8 | (50.0%) | 6 | (54.5%) | 4 | (66.7%) | 2 | (40.0%) |
| Race | White | 7 | (87.5%) | 15 | (93.8%) | 10 | (90.9%) | 6 | (100%) | 5 | (100%) |
| Colonic | Peak (eos/HPF) | 28.5 | (23.8-37.5) | 22 | (10.5-40) | 100 | (80-136) | 40.5 | (23.3-59.8) | 101 | (73-106.5) |
| eosinophil | | | | | | | | | | | |
| counts | | | | | | | | | | | |
| | Range | 2-43 | | 9-42 | | 69-187 | | 15-62 | | 66-110 | |
| History of | EoE | — | | 6 | (37.5%) | 9 | (81.8%) | — | | — | |
| EGID | EoG | — | | 3 | (18.8%) | 2 | (18.2%) | — | | — | |
| | EoC | — | | 16 | (100%) | 11 | (100%) | — | | — | |
| Atopic status | Atopy (any) | 4 | (50.0%) | 7 | (43.8%) | 8 | (72.7%) | 3 | (50.0%) | 4 | (80.0%) |
| | Asthma | 2 | (25.0%) | 0 | (0%) | 6 | (54.5%) | 1 | (16.7%) | 1 | (20.0%) |
| | Allergic rhinitis | 3 | (37.5%) | 4 | (25.0%) | 7 | (63.6%) | 2 | (33.3%) | 4 | (80.0%) |
| | Eczema | 2 | (25.0%) | 3 | (18.8%) | 6 | (54.5%) | 0 | (0%) | 1 | (20.0%) |
| | Food allergy | 0 | (0%) | 4 | (25.0%) | 3 | (27.3%) | 0 | (0%) | 0 | (0%) |
| Treatment at | Ongoing diet | 1 | (12.5%) | 9 | (56.3%) | 3 | (27.3%) | 1 | (16.7%) | 1 | (20.0%) |
| biopsy | therapy | | | | | | | | | | |
| | Proton pump | 4 | (50.0%) | 2 | (12.5%) | 7 | (63.6%) | 0 | (0%) | 0 | (0%) |
| | inhibitor | | | | | | | | | | |
| | Topical | 0 | (0%) | 4 | (25.0%) | 4 | (36.4%) | 0 | (0%) | 0 | (0%) |
| | steroids | | | | | | | | | | |
| | Systemic | 0 | (0%) | 1 | (6.3%) | 1 | (9.1%) | 2 | (33.3%) | 0 | (0%) |
| | steroids | | | | | | | | | | |
| | Immune | 0 | (0%) | 0 | (0%) | 0 | (0%) | 3 | (50.0%) | 1 | (20.0%) |
| | modulator | | | | | | | | | | |
| | Biologics | 0 | (0%) | 0 | (0%) | 0 | (0%) | 4 | (66.7%) | 4 | (80.0%) |

*Data are n (%) or median (interquartile range [IQR]) unless otherwise stated.
CD, Crohn disease; EoC, eosinophilic colitis; EoG, eosinophilic gastritis; EGID, eosinophilic gastrointestinal disorder; EoE, eosinophilic esophagitis; eos/HPF, eosinophils per high-power microscopic field; NL, normal.

TABLE 6
List of genes of EoC and CD transcriptomes in colon biopsies

| | | | Correlation with | | | | | Correlation with | |
|---|---|---|---|---|---|---|---|---|---|
| EoC transcriptome | | | peak colonic | | CD transcriptome | | | peak colonic | |
| (Active EoC vs NL) | | | eosinophil counts | | (Inflamed CD vs NL) | | | eosinophil counts | |
| Gene | | FDR P | Spearman | FDR P | Gene | | FDR P | Spearman | FDR P |
| Symbol | FC | value | r | value | Symbol | FC | value | r | value |
| CLC | 10.2 | 4.2E−03 | 0.77 | 4.5E−05 | SAA1 | 19.3 | 1.3E−03 | 0.35 | 2.3E−01 |
| GPA33 | 8.8 | 7.0E−03 | 0.47 | 2.9E−02 | DMBT1 | 14.8 | 1.5E−02 | −0.06 | 8.7E−01 |
| IFITM1 | 8.3 | 1.3E−02 | 0.61 | 3.0E−03 | DUOX2 | 14.3 | 1.3E−02 | 0.01 | 9.8E−01 |
| MRNIP | 7.6 | 3.7E−03 | 0.24 | 2.4E−01 | TNFRSF6B | 11.0 | 3.8E−04 | 0.18 | 5.7E−01 |
| MT1H | 7.3 | 2.8E−02 | 0.18 | 3.7E−01 | NOS2 | 10.4 | 1.4E−03 | −0.05 | 8.9E−01 |
| ESRRA | 6.6 | 4.2E−03 | 0.40 | 6.0E−02 | DUOXA2 | 9.9 | 2.9E−02 | 0.09 | 8.0E−01 |
| MT1X | 6.2 | 1.1E−02 | 0.06 | 7.9E−01 | REG1B | 8.9 | 4.6E−02 | 0.11 | 7.5E−01 |
| CDK2AP1 | 6.1 | 3.9E−02 | −0.06 | 7.7E−01 | CXCL3 | 8.6 | 4.6E−04 | 0.36 | 2.0E−01 |
| CDC26 | 6.0 | 2.6E−03 | 0.35 | 9.7E−02 | CXCL9 | 7.7 | 1.4E−02 | 0.02 | 9.7E−01 |
| SLC35B2 | 5.4 | 8.1E−04 | 0.30 | 1.5E−01 | MMP3 | 7.0 | 3.0E−02 | −0.25 | 4.1E−01 |
| TMEM251 | 5.2 | 9.2E−03 | 0.34 | 1.1E−01 | CHI3L1 | 6.9 | 1.2E−02 | 0.04 | 9.1E−01 |
| PPP1R35 | 5.1 | 3.8E−02 | 0.16 | 4.4E−01 | CXCL1 | 6.6 | 2.7E−02 | 0.40 | 1.3E−01 |
| PIM1 | 5.0 | 1.5E−02 | 0.60 | 3.7E−03 | TNIP3 | 6.6 | 6.5E−03 | −0.25 | 4.1E−01 |
| PPP4C | 4.9 | 3.8E−03 | 0.47 | 2.5E−02 | SAA2 | 6.3 | 1.3E−02 | 0.34 | 2.4E−01 |
| NDUFB3 | 4.9 | 3.2E−03 | 0.41 | 5.4E−02 | SOCS3 | 6.2 | 8.6E−04 | −0.04 | 9.2E−01 |
| RAB43 | 4.8 | 1.8E−02 | 0.41 | 5.5E−02 | SAA4 | 5.2 | 1.4E−02 | −0.07 | 8.6E−01 |
| F13A1 | 4.7 | 1.1E−02 | 0.43 | 4.2E−02 | BATF | 5.2 | 1.2E−04 | −0.02 | 9.6E−01 |
| IFNGR1 | 4.6 | 3.5E−02 | 0.36 | 9.0E−02 | G0S2 | 5.1 | 2.2E−02 | −0.12 | 7.4E−01 |
| AC068547.1 | 4.5 | 6.7E−03 | 0.26 | 2.0E−01 | CXCL10 | 5.0 | 4.1E−02 | −0.09 | 8.1E−01 |
| GNB2 | 4.5 | 2.6E−02 | 0.51 | 1.6E−02 | IDO1 | 4.9 | 1.6E−02 | −0.05 | 9.0E−01 |
| CLDN23 | 4.4 | 1.6E−02 | 0.31 | 1.4E−01 | BCL2A1 | 4.8 | 1.5E−02 | 0.31 | 2.9E−01 |
| ZFAND3 | 4.4 | 4.3E−03 | 0.42 | 4.8E−02 | UBD | 4.6 | 3.1E−03 | 0.00 | 9.9E−01 |
| NCSTN | 4.4 | 3.8E−03 | 0.19 | 3.5E−01 | C4BPB | 4.6 | 4.6E−02 | −0.26 | 3.8E−01 |
| PRIMPOL | 4.3 | 3.3E−03 | 0.26 | 2.1E−01 | CALHM6 | 4.3 | 1.2E−03 | 0.34 | 2.3E−01 |
| PTS | 4.2 | 2.4E−03 | 0.22 | 2.7E−01 | GBP5 | 4.3 | 1.4E−03 | 0.13 | 7.1E−01 |
| BCL2L10 | 4.2 | 4.9E−03 | 0.39 | 6.8E−02 | MNDA | 4.1 | 4.8E−03 | 0.50 | 4.5E−02 |
| CTSL | 4.2 | 6.1E−03 | 0.38 | 7.4E−02 | TGM2 | 4.0 | 1.0E−02 | 0.18 | 5.7E−01 |
| LINC00116 | 4.1 | 3.9E−03 | 0.31 | 1.4E−01 | IL1B | 4.0 | 3.9E−02 | 0.14 | 6.8E−01 |
| RPIA | 4.1 | 4.2E−03 | 0.26 | 2.0E−01 | POU2AF1 | 3.9 | 5.2E−05 | 0.05 | 8.9E−01 |
| C5orf15 | 4.1 | 4.9E−03 | 0.46 | 3.2E−02 | IFITM1 | 3.8 | 1.1E−02 | 0.61 | 9.7E−03 |
| JKAMP | 4.1 | 9.1E−03 | 0.23 | 2.6E−01 | CDK2AP1 | 3.8 | 2.2E−02 | −0.06 | 8.7E−01 |
| CLEC10A | 4.0 | 4.9E−03 | 0.60 | 3.6E−03 | GBP1 | 3.7 | 1.1E−02 | 0.42 | 1.2E−01 |
| TXN | 3.9 | 9.1E−03 | 0.19 | 3.5E−01 | CFI | 3.7 | 1.6E−02 | 0.37 | 1.9E−01 |
| MAD2L1BP | 3.9 | 3.7E−03 | 0.48 | 2.3E−02 | GBP4 | 3.7 | 1.9E−03 | 0.27 | 3.7E−01 |
| GAPT | 3.9 | 4.7E−03 | 0.33 | 1.2E−01 | CFB | 3.6 | 2.4E−02 | 0.06 | 8.8E−01 |
| SEC61G | 3.9 | 4.7E−03 | 0.35 | 1.0E−01 | FCGR1A | 3.5 | 1.5E−02 | 0.04 | 9.1E−01 |
| GAS6 | 3.9 | 1.9E−02 | 0.45 | 3.4E−02 | CXCL11 | 3.5 | 2.8E−02 | 0.02 | 9.7E−01 |
| UNC50 | 3.8 | 8.5E−03 | 0.38 | 7.7E−02 | SAMD9L | 3.4 | 1.8E−02 | −0.02 | 9.7E−01 |
| ARL2BP | 3.8 | 3.2E−02 | 0.37 | 8.3E−02 | CCL4 | 3.4 | 1.3E−02 | 0.32 | 2.7E−01 |
| EEF1A1 | 3.8 | 4.6E−03 | 0.34 | 1.0E−01 | MDK | 3.4 | 1.5E−02 | −0.19 | 5.4E−01 |
| CD9 | 3.8 | 3.3E−03 | 0.38 | 7.0E−02 | APOL1 | 3.3 | 3.9E−02 | 0.09 | 8.0E−01 |
| TRPT1 | 3.8 | 3.3E−03 | 0.52 | 1.4E−02 | NNMT | 3.3 | 1.2E−03 | 0.17 | 5.9E−01 |
| MAP2K1 | 3.8 | 8.5E−03 | 0.45 | 3.5E−02 | SNX10 | 3.3 | 7.6E−04 | −0.07 | 8.4E−01 |
| IRF8 | 3.7 | 1.9E−02 | 0.18 | 3.8E−01 | C2 | 3.2 | 1.2E−03 | 0.56 | 1.8E−02 |
| CFAP20 | 3.7 | 4.1E−03 | 0.33 | 1.1E−01 | CD40 | 3.2 | 8.0E−04 | 0.15 | 6.4E−01 |
| FCER1A | 3.7 | 4.9E−03 | 0.27 | 1.8E−01 | TRIB2 | 3.1 | 2.9E−02 | −0.29 | 3.3E−01 |
| CXCL1 | 3.6 | 5.0E−02 | 0.40 | 6.0E−02 | OAS2 | 3.1 | 6.5E−04 | 0.07 | 8.4E−01 |
| TRIM21 | 3.6 | 1.7E−02 | 0.50 | 1.8E−02 | KLF2 | 3.1 | 1.3E−02 | 0.06 | 8.8E−01 |
| RGCC | 3.6 | 2.7E−02 | 0.34 | 1.0E−01 | LYSMD2 | 3.1 | 6.4E−04 | 0.16 | 6.1E−01 |
| IFNLR1 | 3.6 | 6.5E−03 | 0.54 | 1.1E−02 | FGR | 3.1 | 5.6E−05 | 0.05 | 9.0E−01 |
| AKT1 | 3.5 | 2.4E−03 | 0.31 | 1.5E−01 | HLA-F | 3.1 | 1.1E−02 | 0.22 | 4.7E−01 |
| SLC51B | 3.5 | 3.2E−02 | 0.49 | 2.0E−02 | PLA2G16 | 3.1 | 3.2E−03 | 0.29 | 3.2E−01 |
| AC138894.1 | 3.5 | 3.5E−02 | 0.34 | 1.0E−01 | MMP9 | 3.0 | 1.6E−03 | 0.23 | 4.4E−01 |
| PIGF | 3.5 | 1.9E−06 | 0.37 | 8.3E−02 | PDPN | 3.0 | 9.4E−03 | 0.20 | 5.2E−01 |
| CEBPB | 3.4 | 3.7E−03 | 0.47 | 2.5E−02 | KCND3 | 3.0 | 2.6E−02 | 0.45 | 8.2E−02 |
| PDE6D | 3.4 | 9.2E−03 | 0.14 | 5.0E−01 | RPL22L1 | 3.0 | 5.8E−04 | 0.10 | 7.7E−01 |
| TMEM126A | 3.4 | 2.7E−02 | 0.19 | 3.6E−01 | TNFAIP2 | 3.0 | 8.5E−05 | 0.22 | 4.7E−01 |
| CD300A | 3.4 | 4.2E−03 | 0.40 | 5.7E−02 | SOCS1 | 3.0 | 9.3E−03 | 0.29 | 3.1E−01 |
| KCTD14 | 3.4 | 3.3E−02 | 0.27 | 1.9E−01 | TRIM69 | 3.0 | 1.9E−03 | 0.13 | 7.1E−01 |
| MYD88 | 3.4 | 3.3E−03 | 0.27 | 1.9E−01 | GZMB | 3.0 | 2.5E−02 | 0.19 | 5.4E−01 |
| TMEM171 | 3.4 | 1.1E−02 | 0.55 | 1.1E−02 | DOK3 | 3.0 | 3.7E−04 | 0.31 | 3.0E−01 |
| CLIC6 | 3.4 | 2.6E−02 | 0.44 | 4.0E−02 | IFIT3 | 3.0 | 4.8E−02 | 0.12 | 7.4E−01 |
| LRFN4 | 3.3 | 4.3E−02 | 0.24 | 2.4E−01 | DAPP1 | 3.0 | 8.6E−03 | −0.04 | 9.1E−01 |
| TWF2 | 3.3 | 2.9E−02 | 0.26 | 2.0E−01 | RAMP3 | 2.9 | 2.5E−02 | −0.09 | 8.1E−01 |
| PRR14 | 3.2 | 2.6E−02 | 0.20 | 3.3E−01 | CCL11 | 2.9 | 3.1E−02 | 0.78 | 1.0E−04 |
| CHPT1 | 3.2 | 4.2E−03 | 0.28 | 1.7E−01 | TMEM158 | 2.9 | 1.6E−02 | −0.21 | 4.9E−01 |
| NAXD | 3.2 | 1.3E−02 | 0.40 | 5.7E−02 | PIM2 | 2.9 | 1.6E−03 | −0.05 | 9.0E−01 |
| MUS81 | 3.2 | 8.1E−03 | 0.19 | 3.6E−01 | C10orf10 | 2.9 | 2.4E−03 | −0.01 | 9.7E−01 |
| RAB1B | 3.2 | 1.4E−02 | 0.18 | 3.7E−01 | RARRES3 | 2.9 | 9.3E−03 | 0.15 | 6.5E−01 |
| ACKR3 | 3.2 | 9.2E−03 | 0.28 | 1.8E−01 | DUSP2 | 2.9 | 1.7E−03 | 0.24 | 4.2E−01 |
| MRPL32 | 3.2 | 1.9E−02 | 0.36 | 9.1E−02 | CD38 | 2.8 | 5.2E−05 | 0.29 | 3.2E−01 |
| PTGER2 | 3.2 | 4.9E−03 | 0.64 | 2.2E−03 | PTP4A3 | 2.8 | 2.8E−02 | 0.28 | 3.5E−01 |
| MARCKSL1 | 3.2 | 1.9E−03 | 0.25 | 2.2E−01 | PLEK | 2.8 | 9.4E−04 | 0.42 | 1.2E−01 |
| FBXL12 | 3.2 | 3.9E−03 | 0.21 | 3.0E−01 | TIMP1 | 2.8 | 2.2E−02 | −0.39 | 1.5E−01 |
| TTYH3 | 3.2 | 1.0E−02 | 0.44 | 4.0E−02 | REC8 | 2.8 | 3.9E−03 | −0.12 | 7.3E−01 |
| BMF | 3.2 | 1.2E−04 | 0.65 | 1.5E−03 | STAT1 | 2.8 | 6.2E−03 | 0.06 | 8.8E−01 |
| MMP12 | 3.2 | 2.4E−02 | 0.44 | 3.7E−02 | CD53 | 2.8 | 8.9E−03 | 0.27 | 3.6E−01 |
| CYSTM1 | 3.2 | 9.9E−05 | 0.54 | 1.1E−02 | IFNGR1 | 2.8 | 3.3E−02 | 0.36 | 2.0E−01 |
| NCF2 | 3.1 | 1.2E−02 | 0.44 | 4.1E−02 | NMB | 2.7 | 4.3E−02 | −0.11 | 7.4E−01 |
| TMSB4X | 3.1 | 2.7E−02 | 0.33 | 1.1E−01 | IFIT2 | 2.7 | 5.5E−03 | 0.13 | 7.1E−01 |
| LY96 | 3.1 | 9.4E−03 | 0.18 | 3.7E−01 | ARFGAP3 | 2.7 | 1.8E−03 | −0.23 | 4.5E−01 |
| CD53 | 3.1 | 9.5E−03 | 0.27 | 1.8E−01 | CIITA | 2.7 | 6.0E−04 | 0.15 | 6.6E−01 |
| C16orf91 | 3.1 | 2.9E−02 | 0.31 | 1.4E−01 | PARP9 | 2.7 | 1.1E−03 | 0.36 | 2.1E−01 |
| KTI12 | 3.1 | 9.1E−03 | 0.35 | 9.7E−02 | PIK3AP1 | 2.7 | 2.0E−03 | 0.16 | 6.2E−01 |
| NOP10 | 3.1 | 3.1E−04 | 0.37 | 8.2E−02 | SMOC2 | 2.7 | 1.8E−02 | 0.22 | 4.7E−01 |
| PNP | 3.1 | 1.0E−02 | 0.35 | 1.0E−01 | BACE2 | 2.7 | 6.5E−03 | 0.08 | 8.3E−01 |
| MGST1 | 3.1 | 3.8E−03 | 0.48 | 2.4E−02 | SEC14L1 | 2.6 | 3.2E−04 | 0.39 | 1.4E−01 |
| NRAS | 3.1 | 8.4E−04 | 0.45 | 3.4E−02 | TRIM21 | 2.6 | 7.6E−03 | 0.50 | 4.1E−02 |
| DERA | 3.0 | 1.2E−02 | 0.44 | 4.0E−02 | PLAUR | 2.6 | 5.2E−03 | 0.14 | 6.7E−01 |
| SEC14L1 | 3.0 | 4.2E−03 | 0.39 | 6.5E−02 | PNOC | 2.6 | 2.4E−02 | 0.21 | 4.9E−01 |
| CEBPE | 3.0 | 2.7E−02 | 0.53 | 1.3E−02 | FCRL5 | 2.6 | 1.4E−02 | 0.05 | 8.9E−01 |
| CREG1 | 3.0 | 1.4E−02 | 0.33 | 1.2E−01 | HLA-DMA | 2.6 | 6.8E−03 | 0.20 | 5.2E−01 |
| DTD1 | 3.0 | 1.0E−02 | 0.25 | 2.2E−01 | CASP1 | 2.6 | 9.4E−03 | 0.54 | 2.5E−02 |
| MNDA | 3.0 | 2.5E−02 | 0.50 | 2.0E−02 | ARHGAP25 | 2.5 | 4.6E−03 | −0.27 | 3.7E−01 |
| ZNRF2 | 3.0 | 3.7E−02 | 0.46 | 3.3E−02 | RASGRP1 | 2.5 | 3.4E−02 | 0.30 | 3.0E−01 |
| SDHB | 3.0 | 4.2E−03 | 0.35 | 9.3E−02 | LST1 | 2.5 | 6.3E−04 | 0.52 | 3.3E−02 |
| ITGAE | 3.0 | 5.4E−03 | 0.26 | 2.1E−01 | LAX1 | 2.5 | 4.2E−02 | 0.04 | 9.2E−01 |
| C1orf174 | 2.9 | 1.2E−02 | 0.34 | 1.0E−01 | LINC00116 | 2.5 | 4.1E−03 | 0.31 | 2.9E−01 |
| P2RY14 | 2.9 | 9.4E−03 | 0.63 | 2.3E−03 | BIRC3 | 2.5 | 4.3E−03 | 0.01 | 9.7E−01 |
| MGST3 | 2.9 | 2.5E−03 | 0.41 | 5.5E−02 | CARD6 | 2.5 | 3.2E−02 | −0.01 | 9.7E−01 |
| ELK1 | 2.9 | 1.0E−02 | 0.22 | 2.7E−01 | WARS | 2.5 | 7.7E−03 | −0.05 | 8.9E−01 |
| CLDND1 | 2.9 | 1.1E−02 | 0.44 | 3.9E−02 | TTYH3 | 2.5 | 4.5E−02 | 0.44 | 9.1E−02 |
| SLC35A5 | 2.9 | 7.4E−03 | 0.13 | 5.3E−01 | ARID5A | 2.5 | 3.8E−03 | −0.10 | 7.8E−01 |
| TMEM150A | 2.9 | 3.3E−03 | 0.30 | 1.6E−01 | SRGN | 2.5 | 3.5E−03 | 0.31 | 2.8E−01 |
| CHCHD3 | 2.8 | 4.8E−03 | 0.16 | 4.2E−01 | MAP3K8 | 2.5 | 1.9E−03 | −0.32 | 2.8E−01 |
| HLA-DMA | 2.8 | 1.6E−02 | 0.20 | 3.3E−01 | ST8SIA4 | 2.4 | 6.9E−05 | 0.33 | 2.6E−01 |
| RNF146 | 2.8 | 1.0E−02 | 0.25 | 2.1E−01 | C5orf15 | 2.4 | 4.1E−03 | 0.46 | 6.9E−02 |
| CSTB | 2.8 | 1.6E−03 | 0.63 | 2.3E−03 | SERPINB9 | 2.4 | 2.0E−02 | 0.07 | 8.4E−01 |
| HDHD5 | 2.8 | 2.7E−02 | 0.27 | 1.9E−01 | ACSL4 | 2.4 | 5.3E−03 | 0.27 | 3.6E−01 |
| BCL7B | 2.8 | 1.2E−02 | 0.32 | 1.3E−01 | CLEC2B | 2.4 | 1.3E−02 | −0.05 | 8.9E−01 |
| TDRD7 | 2.8 | 8.5E−03 | 0.48 | 2.5E−02 | CSF2RB | 2.4 | 1.8E−04 | 0.28 | 3.4E−01 |
| CXCL3 | 2.8 | 4.6E−02 | 0.36 | 8.7E−02 | RASSF5 | 2.4 | 8.3E−03 | 0.34 | 2.4E−01 |
| HS6ST1 | 2.8 | 2.2E−02 | 0.30 | 1.6E−01 | IRF4 | 2.4 | 1.1E−02 | −0.04 | 9.1E−01 |
| MIPEP | 2.8 | 2.1E−02 | 0.33 | 1.2E−01 | LYN | 2.4 | 3.4E−03 | 0.26 | 3.8E−01 |
| LPCAT3 | 2.8 | 4.7E−02 | 0.38 | 6.9E−02 | MGP | 2.4 | 3.6E−02 | −0.26 | 3.8E−01 |
| PNRC1 | 2.8 | 1.8E−02 | 0.45 | 3.7E−02 | TRIM59 | 2.4 | 6.0E−04 | 0.09 | 8.0E−01 |
| SMOC2 | 2.8 | 4.8E−02 | 0.22 | 2.8E−01 | JAK2 | 2.4 | 3.1E−03 | −0.13 | 7.1E−01 |
| NT5C3B | 2.7 | 3.4E−02 | 0.51 | 1.6E−02 | TAP2 | 2.4 | 3.7E−03 | 0.12 | 7.4E−01 |
| CD4 | 2.7 | 1.2E−02 | 0.35 | 1.0E−01 | HLA-DOB | 2.4 | 4.4E−02 | −0.08 | 8.3E−01 |
| COMMD6 | 2.7 | 2.0E−02 | 0.24 | 2.4E−01 | PRDM1 | 2.4 | 2.3E−02 | 0.40 | 1.3E−01 |
| WRNIP1 | 2.7 | 3.2E−02 | 0.31 | 1.4E−01 | PHLDA1 | 2.4 | 3.2E−02 | −0.19 | 5.3E−01 |
| ALKBH5 | 2.7 | 8.5E−03 | 0.34 | 1.1E−01 | HLA-B | 2.4 | 2.9E−03 | 0.35 | 2.1E−01 |
| SMIM26 | 2.7 | 1.7E−02 | 0.10 | 6.2E−01 | LCP1 | 2.4 | 2.9E−04 | 0.29 | 3.2E−01 |
| CCLAL2 | 2.7 | 2.2E−02 | 0.35 | 9.8E−02 | SCPEP1 | 2.3 | 6.1E−03 | 0.40 | 1.4E−01 |
| TPBG | 2.7 | 6.4E−04 | 0.45 | 3.5E−02 | KLHL6 | 2.3 | 6.6E−03 | 0.16 | 6.2E−01 |
| SERPINF1 | 2.7 | 4.3E−02 | 0.37 | 8.2E−02 | CLEC4E | 2.3 | 2.3E−03 | 0.14 | 6.9E−01 |
| FOXF2 | 2.7 | 1.5E−02 | 0.22 | 2.8E−01 | LRFN4 | 2.3 | 1.1E−02 | 0.24 | 4.2E−01 |
| TMEM230 | 2.7 | 3.7E−03 | 0.35 | 1.0E−01 | ANXA3 | 2.3 | 1.9E−02 | −0.21 | 5.0E−01 |
| PLD3 | 2.7 | 3.3E−03 | 0.37 | 8.3E−02 | RAB8B | 2.3 | 1.8E−03 | 0.37 | 1.9E−01 |
| FOXA1 | 2.7 | 2.7E−02 | 0.51 | 1.7E−02 | LY6E | 2.3 | 4.2E−02 | −0.32 | 2.8E−01 |
| SF3B4 | 2.7 | 2.5E−02 | 0.27 | 1.8E−01 | TSPAN5 | 2.3 | 2.2E−02 | −0.10 | 7.7E−01 |
| TIPARP | 2.7 | 1.6E−02 | 0.67 | 1.1E−03 | CYTIP | 2.3 | 1.5E−02 | −0.09 | 8.1E−01 |
| HLA-DQB2 | 2.7 | 8.5E−03 | 0.30 | 1.5E−01 | NDUFB3 | 2.3 | 2.9E−03 | 0.41 | 1.2E−01 |
| RNF128 | 2.7 | 3.1E−02 | 0.55 | 1.1E−02 | APOBEC3D | 2.3 | 3.7E−02 | −0.31 | 2.9E−01 |
| CTDSP2 | 2.6 | 4.9E−03 | 0.35 | 1.0E−01 | CLEC4A | 2.3 | 4.1E−02 | −0.25 | 4.1E−01 |
| MED20 | 2.6 | 1.8E−02 | 0.40 | 6.2E−02 | UBE2L6 | 2.3 | 6.9E−03 | 0.61 | 9.7E−03 |
| CEBPD | 2.6 | 1.3E−02 | 0.56 | 8.6E−03 | SLC35B2 | 2.3 | 1.9E−02 | 0.30 | 3.0E−01 |
| GCH1 | 2.6 | 8.1E−03 | 0.43 | 4.2E−02 | PNO1 | 2.3 | 1.1E−03 | 0.01 | 9.8E−01 |
| ZNF706 | 2.6 | 4.2E−03 | 0.14 | 5.0E−01 | ITGAX | 2.3 | 1.0E−02 | 0.30 | 3.0E−01 |
| NDUFC1 | 2.6 | 1.2E−02 | 0.09 | 6.7E−01 | NLRC5 | 2.3 | 4.0E−03 | −0.48 | 5.6E−02 |
| YAP1 | 2.6 | 4.0E−03 | 0.56 | 9.5E−03 | KLHL5 | 2.3 | 5.7E−03 | 0.20 | 5.2E−01 |
| ITM2C | 2.6 | 3.1E−02 | 0.11 | 5.9E−01 | MZB1 | 2.3 | 1.8E−02 | 0.20 | 5.2E−01 |
| BRPF3 | 2.6 | 1.6E−02 | 0.07 | 7.3E−01 | PDP1 | 2.2 | 3.1E−03 | −0.14 | 6.7E−01 |
| ACVR1B | 2.6 | 1.9E−02 | 0.36 | 8.7E−02 | LILRB3 | 2.2 | 4.9E−02 | 0.19 | 5.4E−01 |
| OST4 | 2.6 | 4.1E−03 | 0.20 | 3.2E−01 | COL1A2 | 2.2 | 3.7E−02 | 0.64 | 6.2E−03 |
| SDHAF3 | 2.6 | 1.1E−02 | 0.05 | 8.3E−01 | FAM129A | 2.2 | 7.6E−03 | 0.13 | 6.9E−01 |
| ZNF513 | 2.6 | 1.1E−02 | 0.35 | 9.7E−02 | IRF8 | 2.2 | 4.7E−02 | 0.18 | 5.7E−01 |
| RPL30 | 2.6 | 3.9E−03 | 0.29 | 1.6E−01 | GPX2 | 2.2 | 8.3E−03 | −0.43 | 9.7E−02 |
| DNAJB9 | 2.6 | 3.1E−02 | 0.33 | 1.2E−01 | CCL4L2 | 2.2 | 2.0E−02 | 0.35 | 2.2E−01 |
| TCEAL7 | 2.6 | 2.6E−02 | 0.40 | 6.0E−02 | CYSLTR1 | 2.2 | 2.8E−02 | 0.29 | 3.2E−01 |
| FAM214B | 2.6 | 4.1E−02 | 0.55 | 1.1E−02 | ITGB2 | 2.2 | 1.9E−03 | 0.10 | 7.8E−01 |
| CDK8 | 2.6 | 4.4E−02 | 0.48 | 2.3E−02 | SKAP1 | 2.2 | 3.2E−02 | 0.08 | 8.3E−01 |
| CYP2C9 | 2.6 | 2.0E−02 | 0.66 | 1.4E−03 | PPP1R18 | 2.2 | 3.8E−02 | 0.26 | 3.8E−01 |
| WDR6 | 2.6 | 1.8E−02 | 0.34 | 1.1E−01 | CTSK | 2.2 | 3.5E−02 | 0.19 | 5.2E−01 |
| LHFPL6 | 2.6 | 3.1E−02 | 0.25 | 2.3E−01 | PFKFB3 | 2.2 | 4.2E−02 | −0.20 | 5.2E−01 |
| CTGF | 2.5 | 6.8E−03 | 0.34 | 1.1E−01 | PLEKHO1 | 2.2 | 1.1E−03 | 0.10 | 7.9E−01 |
| EIF2D | 2.5 | 1.3E−02 | 0.35 | 9.8E−02 | SLAMF7 | 2.2 | 1.8E−02 | 0.44 | 9.4E−02 |
| EIF1AD | 2.5 | 1.2E−03 | 0.48 | 2.5E−02 | IFI27L2 | 2.2 | 2.4E−02 | −0.26 | 3.8E−01 |
| DCTN6 | 2.5 | 1.8E−02 | 0.15 | 4.6E−01 | TMEM126A | 2.2 | 4.1E−03 | 0.19 | 5.4E−01 |
| KLF4 | 2.5 | 2.9E−02 | 0.55 | 9.8E−03 | CD300A | 2.2 | 3.6E−03 | 0.40 | 1.3E−01 |
| MRPL34 | 2.5 | 3.0E−03 | 0.35 | 9.6E−02 | MCUB | 2.2 | 1.4E−03 | 0.06 | 8.8E−01 |
| MFSD1 | 2.5 | 5.7E−03 | 0.35 | 9.9E−02 | LPGAT1 | 2.2 | 1.9E−02 | 0.49 | 4.6E−02 |
| GLRX | 2.5 | 2.1E−02 | 0.54 | 1.1E−02 | PTPRC | 2.2 | 2.8E−02 | 0.03 | 9.4E−01 |
| ACAT1 | 2.5 | 3.1E−02 | 0.44 | 3.8E−02 | LYZ | 2.2 | 1.4E−02 | −0.11 | 7.4E−01 |
| DDHD2 | 2.5 | 3.3E−03 | 0.29 | 1.6E−01 | SLFN5 | 2.2 | 3.7E−02 | 0.12 | 7.4E−01 |
| ELOC | 2.5 | 1.4E−02 | 0.28 | 1.8E−01 | SCRN1 | 2.2 | 1.2E−02 | 0.08 | 8.2E−01 |
| B4GALT4 | 2.5 | 1.7E−02 | 0.19 | 3.5E−01 | MSN | 2.2 | 3.5E−04 | 0.13 | 7.1E−01 |
| TMEM14C | 2.5 | 1.4E−02 | 0.37 | 7.9E−02 | SERPING1 | 2.2 | 2.2E−02 | −0.21 | 5.0E−01 |
| SH3BP4 | 2.5 | 4.9E−03 | 0.08 | 7.2E−01 | SAMSN1 | 2.2 | 1.8E−02 | 0.29 | 3.3E−01 |
| EMC7 | 2.5 | 2.5E−02 | 0.10 | 6.4E−01 | DNAJB9 | 2.2 | 2.0E−02 | 0.33 | 2.6E−01 |
| TAF5L | 2.5 | 7.6E−03 | 0.14 | 5.0E−01 | CD274 | 2.2 | 1.8E−02 | 0.03 | 9.5E−01 |
| C2orf76 | 2.5 | 5.7E−03 | 0.42 | 5.0E−02 | PLEKHA4 | 2.2 | 4.7E−02 | −0.16 | 6.2E−01 |
| TMEM35B | 2.5 | 1.5E−02 | 0.43 | 4.2E−02 | HLA-DRA | 2.1 | 3.0E−03 | 0.33 | 2.4E−01 |
| PHLPP2 | 2.5 | 1.8E−02 | 0.68 | 1.0E−03 | PCDH7 | 2.1 | 4.7E−02 | 0.34 | 2.3E−01 |
| RPL22L1 | 2.5 | 1.4E−02 | 0.10 | 6.1E−01 | TMEM251 | 2.1 | 1.8E−02 | 0.34 | 2.4E−01 |
| ABCB10 | 2.5 | 3.7E−03 | 0.47 | 2.6E−02 | IGFBP7 | 2.1 | 3.4E−02 | 0.42 | 1.2E−01 |
| HOXB2 | 2.5 | 4.4E−02 | 0.41 | 5.2E−02 | DRAM1 | 2.1 | 3.2E−02 | 0.23 | 4.6E−01 |
| GLIPR1 | 2.5 | 4.0E−02 | 0.18 | 3.7E−0 | JKAMP | 2.1 | 5.5E−03 | 0.23 | 4.5E−01 |
| HLA-F | 2.5 | 1.7E−02 | 0.22 | 2.7E−01 | TEAD3 | 2.1 | 1.9E−03 | 0.03 | 9.5E−01 |
| IGIP | 2.5 | 1.3E−02 | 0.25 | 2.1E−01 | EVI2B | 2.1 | 6.5E−03 | 0.18 | 5.6E−01 |
| GRAMD4 | 2.5 | 1.9E−02 | 0.58 | 5.6E−03 | SLA | 2.1 | 4.7E−02 | 0.31 | 2.9E−01 |
| RAP2A | 2.5 | 4.5E−02 | 0.11 | 5.9E−01 | CHST11 | 2.1 | 3.6E−03 | 0.26 | 3.9E−01 |
| RPS25 | 2.5 | 3.7E−03 | 0.24 | 2.3E−01 | GIMAP5 | 2.1 | 3.6E−02 | −0.02 | 9.6E−01 |
| SPHK2 | 2.5 | 4.3E−02 | 0.56 | 8.7E−03 | BCL6 | 2.1 | 2.7E−02 | −0.02 | 9.7E−01 |
| LIPA | 2.4 | 5.0E−03 | 0.45 | 3.7E−02 | LPIN1 | 2.1 | 2.3E−03 | −0.19 | 5.4E−01 |
| RNF5 | 2.4 | 3.8E−03 | 0.47 | 2.6E−02 | GIMAP1 | 2.1 | 1.1E−02 | 0.12 | 7.3E−01 |
| PHC2 | 2.4 | 1.3E−02 | 0.37 | 8.3E−02 | PDE6D | 2.1 | 1.9E−02 | 0.14 | 6.7E−01 |
| SDHC | 2.4 | 3.7E−03 | 0.34 | 1.1E−01 | CLEC7A | 2.1 | 9.6E−03 | 0.13 | 7.1E−01 |
| PCDH18 | 2.4 | 1.8E−02 | 0.49 | 2.2E−02 | TRIM22 | 2.1 | 2.3E−02 | 0.26 | 3.9E−01 |
| SEC61B | 2.4 | 3.5E−02 | 0.30 | 1.5E−01 | ASPHD2 | 2.1 | 1.6E−02 | 0.15 | 6.6E−01 |
| LYSMD3 | 2.4 | 1.8E−03 | 0.43 | 4.5E−02 | FBXO5 | 2.1 | 3.9E−02 | −0.14 | 6.6E−01 |
| TMEM242 | 2.4 | 3.8E−03 | 0.24 | 2.4E−01 | RIOX2 | 2.1 | 8.3E−04 | 0.20 | 5.2E−01 |
| LGMN | 2.4 | 1.7E−02 | 0.53 | 1.2E−02 | CD82 | 2.1 | 2.9E−02 | 0.12 | 7.3E−01 |
| CKLF | 2.4 | 2.4E−02 | 0.40 | 5.7E−02 | PSMB9 | 2.1 | 1.8E−02 | 0.31 | 2.9E−01 |
| LSM6 | 2.4 | 3.0E−02 | 0.41 | 5.4E−02 | TAP1 | 2.1 | 1.4E−02 | 0.15 | 6.6E−01 |
| MAGEFI | 2.4 | 6.3E−03 | 0.01 | 9.5E−01 | LAP3 | 2.1 | 5.2E−03 | 0.35 | 2.2E−01 |
| PRIM2 | 2.4 | 1.3E−02 | 0.20 | 3.3E−01 | CEBPB | 2.1 | 2.3E−02 | 0.47 | 5.7E−02 |
| ZNF319 | 2.4 | 4.3E−02 | 0.18 | 3.7E−01 | PLCG2 | 2.1 | 1.7E−02 | 0.13 | 7.1E−01 |
| TMTC2 | 2.4 | 1.1E−02 | 0.28 | 1.7E−01 | ISG20 | 2.1 | 3.7E−02 | 0.17 | 5.8E−01 |
| SF3B6 | 2.4 | 9.1E−03 | 0.45 | 3.7E−02 | RNF24 | 2.1 | 3.4E−04 | 0.63 | 7.2E−03 |
| TCF21 | 2.4 | 2.8E−02 | 0.52 | 1.5E−02 | SIRPA | 2.1 | 7.6E−03 | 0.43 | 9.7E−02 |
| KLRB1 | 2.4 | 4.0E−02 | 0.20 | 3.4E−01 | ANGPT2 | 2.1 | 8.2E−03 | −0.20 | 5.1E−01 |
| TFB2M | 2.4 | 1.2E−02 | 0.45 | 3.4E−02 | TCEAL3 | 2.1 | 2.8E−02 | −0.06 | 8.8E−01 |
| WRAP73 | 2.4 | 1.0E−02 | 0.32 | 1.3E−01 | IFI16 | 2.0 | 3.9E−03 | −0.07 | 8.5E−01 |
| GATM | 2.4 | 2.2E−02 | 0.38 | 7.4E−02 | FAM117A | 2.0 | 1.4E−02 | 0.04 | 9.1E−01 |
| HSPA1A | 2.4 | 4.9E−02 | 0.62 | 2.5E−03 | XBP1 | 2.0 | 2.9E−02 | −0.01 | 9.7E−01 |
| YRDC | 2.4 | 2.4E−02 | 0.16 | 4.3E−01 | CCDC71L | 2.0 | 7.6E−03 | 0.31 | 3.0E−01 |
| IGFBP7 | 2.4 | 4.3E−02 | 0.42 | 5.1E−02 | IFITM3 | 2.0 | 7.8E−03 | 0.25 | 4.0E−01 |
| ZC3H7B | 2.4 | 4.7E−02 | 0.26 | 2.0E−01 | CDC26 | 2.0 | 1.6E−02 | 0.35 | 2.2E−01 |
| CYBRD1 | 2.4 | 9.1E−03 | 0.45 | 3.4E−02 | VAMP5 | 2.0 | 1.9E−02 | 0.23 | 4.4E−01 |
| SGSH | 2.4 | 3.7E−03 | 0.32 | 1.3E−01 | PMEPA1 | 2.0 | 2.0E−02 | 0.11 | 7.6E−01 |
| MAPK7 | 2.3 | 9.9E−05 | 0.37 | 8.1E−02 | PTAFR | 2.0 | 1.1E−02 | 0.49 | 4.9E−02 |
| MYC | 2.3 | 3.0E−02 | 0.34 | 1.1E−01 | SOD2 | 2.0 | 9.6E−04 | 0.35 | 2.2E−01 |
| HIKESH1 | 2.3 | 2.8E−02 | 0.25 | 2.2E−01 | STX2 | 2.0 | 1.3E−02 | 0.12 | 7.1E−01 |
| SRP9 | 2.3 | 4.2E−02 | 0.34 | 1.1E−01 | PTS | 2.0 | 7.6E−03 | 0.22 | 4.6E−01 |
| OXER1 | 2.3 | 2.7E−02 | 0.17 | 4.0E−01 | MRPL32 | 2.0 | 1.3E−02 | 0.36 | 2.1E−01 |
| B3GALT1 | 2.3 | 4.0E−02 | 0.03 | 8.7E−01 | IL3RA | 2.0 | 3.2E−02 | −0.24 | 4.2E−01 |
| TBC1D22B | 2.3 | 2.3E−02 | 0.15 | 4.6E−01 | BASP1 | 2.0 | 3.1E−02 | 0.32 | 2.7E−01 |
| SLBP | 2.3 | 1.8E−02 | 0.33 | 1.1E−01 | TBXAS1 | 2.0 | 4.7E−02 | 0.21 | 4.9E−01 |
| LUM | 2.3 | 1.0E−02 | 0.49 | 2.2E−02 | IRF9 | 2.0 | 3.5E−02 | 0.22 | 4.7E−01 |
| OGT | 2.3 | 1.9E−02 | 0.36 | 9.1E−02 | VCAM1 | 2.0 | 3.2E−02 | 0.30 | 3.0E−01 |
| POLR2K | 2.3 | 1.9E−02 | 0.02 | 9.4E−01 | CTSL | 2.0 | 4.4E−02 | 0.38 | 1.7E−01 |
| B2M | 2.3 | 1.0E−02 | 0.34 | 1.1E−01 | CASP8 | 2.0 | 2.8E−02 | −0.05 | 9.0E−01 |
| DUSP2 | 2.3 | 8.5E−03 | 0.24 | 2.4E−01 | MAP2K1 | 2.0 | 2.5E−02 | 0.45 | 8.0E−02 |
| CPSF7 | 2.3 | 5.7E−03 | 0.41 | 5.2E−02 | PECAM1 | 2.0 | 2.7E−02 | −0.21 | 5.0E−01 |
| NBPF11 | 2.3 | 1.9E−02 | 0.17 | 3.9E−01 | DCK | 2.0 | 1.3E−02 | −0.34 | 2.3E−01 |
| RHPN2 | 2.3 | 3.5E−02 | 0.25 | 2.2E−01 | MUS81 | 2.0 | 7.7E−03 | 0.19 | 5.4E−01 |
| SPINT2 | 2.3 | 4.7E−03 | 0.22 | 2.8E−01 | CD74 | 2.0 | 8.3E−03 | 0.11 | 7.4E−01 |
| TM9SF2 | 2.3 | 6.2E−03 | 0.42 | 4.7E−02 | IRF1 | 1.9 | 3.8E−02 | 0.26 | 3.8E−01 |
| HLA-B | 2.3 | 6.8E−03 | 0.35 | 9.3E−02 | HLA-DMB | 1.9 | 6.2E−03 | 0.25 | 4.0E−01 |
| SNX27 | 2.3 | 7.6E−03 | 0.44 | 3.7E−02 | DPYD | 1.9 | 3.0E−02 | 0.23 | 4.6E−01 |
| PFN2 | 2.3 | 1.5E−02 | 0.54 | 1.1E−02 | IFITM2 | 1.9 | 7.4E−03 | 0.12 | 7.3E−01 |
| COL4A1 | 2.3 | 3.1E−02 | 0.53 | 1.2E−02 | ASRGL1 | 1.9 | 3.1E−02 | −0.60 | 9.9E−03 |
| PPP1R14B | 2.3 | 8.5E−03 | 0.48 | 2.5E−02 | CHST15 | 1.9 | 1.2E−02 | 0.37 | 1.8E−01 |
| SLC16A5 | 2.3 | 4.3E−02 | 0.59 | 4.7E−03 | ARHGDIB | 1.9 | 4.6E−02 | 0.01 | 9.8E−01 |
| IL15RA | 2.3 | 1.8E−02 | 0.55 | 9.7E−03 | RAB31 | 1.9 | 1.2E−02 | 0.17 | 5.8E−01 |
| RGP1 | 2.3 | 3.1E−02 | 0.48 | 2.5E−02 | ZYX | 1.9 | 4.4E−02 | 0.30 | 3.0E−01 |
| DYNLT1 | 2.3 | 2.4E−03 | 0.32 | 1.2E−01 | BLVRA | 1.9 | 3.4E−02 | 0.05 | 9.0E−01 |
| C19orf38 | 2.3 | 1.6E−02 | 0.25 | 2.2E−01 | USP18 | 1.9 | 3.7E−02 | 0.02 | 9.7E−01 |
| SRSF2 | 2.3 | 1.9E−02 | 0.07 | 7.5E−01 | TAF5L | 1.9 | 8.3E−04 | 0.14 | 6.8E−01 |
| RB1 | 2.3 | 4.9E−03 | 0.25 | 2.2E−01 | TMEM165 | 1.9 | 2.6E−03 | 0.20 | 5.2E−01 |
| SIAH2 | 2.3 | 1.4E−02 | 0.27 | 1.9E−01 | ADGRE2 | 1.9 | 2.1E−02 | 0.36 | 1.9E−01 |
| PCNP | 2.3 | 6.3E−03 | 0.23 | 2.5E−01 | TXN | 1.9 | 2.8E−02 | 0.19 | 5.4E−01 |
| NUP37 | 2.3 | 2.8E−02 | 0.42 | 4.8E−02 | B2M | 1.9 | 1.4E−02 | 0.34 | 2.4E−01 |
| PIK3AP1 | 2.3 | 2.7E−02 | 0.16 | 4.3E−01 | PARP14 | 1.9 | 1.6E−02 | 0.07 | 8.4E−01 |
| CSF2RB | 2.3 | 2.6E−02 | 0.28 | 1.7E−01 | ST6GAL1 | 1.9 | 2.0E−02 | −0.05 | 8.9E−01 |
| REPIN1 | 2.2 | 3.7E−03 | 0.28 | 1.8E−01 | IFI30 | 1.9 | 1.6E−02 | −0.07 | 8.4E−01 |
| OCRL | 2.2 | 3.7E−03 | 0.43 | 4.2E−02 | MTHFD2 | 1.9 | 2.5E−04 | −0.16 | 6.2E−01 |
| RPS15A | 2.2 | 3.4E−03 | 0.12 | 5.5E−01 | HERPUD1 | 1.9 | 1.1E−02 | 0.23 | 4.5E−01 |
| NECTIN1 | 2.2 | 3.9E−03 | 0.21 | 3.0E−01 | SGMS1 | 1.9 | 3.9E−02 | 0.28 | 3.5E−01 |
| BCL2L11 | 2.2 | 2.9E−02 | 0.39 | 6.3E−02 | COL6A3 | 1.9 | 4.4E−02 | 0.49 | 4.9E−02 |
| SDF2 | 2.2 | 1.5E−02 | 0.49 | 2.2E−02 | TDRD7 | 1.9 | 3.2E−02 | 0.48 | 5.4E−02 |
| SDCCAG3 | 2.2 | 1.4E−02 | 0.28 | 1.7E−01 | PDE4B | 1.9 | 1.6E−02 | 0.07 | 8.6E−01 |
| RDH10 | 2.2 | 3.1E−02 | 0.48 | 2.3E−02 | ANXA5 | 1.9 | 2.1E−02 | 0.40 | 1.4E−01 |
| WLS | 2.2 | 1.7E−02 | 0.39 | 6.3E−02 | SETD7 | 1.9 | 1.3E−02 | −0.33 | 2.6E−01 |
| MCM2 | 2.2 | 4.4E−02 | 0.03 | 9.0E−01 | HIKESHI | 1.9 | 1.3E−02 | 0.25 | 4.1E−01 |
| OAT | 2.2 | 2.7E−02 | 0.50 | 1.9E−02 | TNFAIP8 | 1.9 | 1.3E−02 | −0.03 | 9.3E−01 |
| GTF2A1 | 2.2 | 3.7E−03 | 0.39 | 6.5E−02 | ADA2 | 1.9 | 6.5E−03 | 0.46 | 6.5E−02 |
| AMY2B | 2.2 | 2.0E−02 | 0.25 | 2.2E−01 | ORC6 | 1.9 | 2.6E−02 | −0.35 | 2.2E−01 |
| DUS3L | 2.2 | 1.9E−02 | 0.02 | 9.4E−01 | GLRX | 1.9 | 3.3E−02 | 0.54 | 2.6E−02 |
| TMEM206 | 2.2 | 2.4E−02 | 0.29 | 1.6E−01 | CARD8 | 1.9 | 7.0E−03 | 0.18 | 5.7E−01 |
| ZBTB5 | 2.2 | 2.5E−02 | 0.31 | 1.4E−01 | ORAI2 | 1.9 | 3.4E−02 | 0.12 | 7.4E−01 |
| PAN2 | 2.2 | 2.5E−02 | 0.46 | 3.1E−02 | RAP2A | 1.9 | 2.5E−02 | 0.11 | 7.6E−01 |
| SIRPA | 2.2 | 1.8E−02 | 0.43 | 4.2E−02 | GIMAP8 | 1.9 | 4.0E−02 | 0.21 | 5.1E−01 |
| CLDN7 | 2.2 | 9.1E−03 | 0.60 | 3.7E−03 | LRP8 | 1.9 | 2.0E−02 | −0.53 | 2.8E−02 |
| SGCE | 2.2 | 4.2E−02 | 0.27 | 1.9E−01 | HAPLN3 | 1.9 | 2.4E−02 | 0.03 | 9.3E−01 |
| TMIGD3 | 2.2 | 3.7E−02 | 0.35 | 9.3E−02 | ICAM1 | 1.9 | 2.6E−02 | −0.18 | 5.7E−01 |
| DTX3 | 2.2 | 2.7E−02 | 0.24 | 2.4E−01 | PI4K2B | 1.9 | 4.3E−02 | 0.12 | 7.3E−01 |
| C15orf48 | 2.2 | 1.1E−02 | 0.30 | 1.5E−01 | TIFA | 1.8 | 4.5E−02 | 0.46 | 6.5E−02 |
| TNS3 | 2.2 | 2.2E−02 | 0.62 | 2.5E−03 | FSTL1 | 1.8 | 2.9E−02 | 0.19 | 5.3E−01 |
| CD276 | 2.2 | 1.0E−02 | 0.29 | 1.7E−01 | ARHGAP15 | 1.8 | 4.3E−02 | 0.09 | 8.0E−01 |
| ISCA2 | 2.2 | 6.2E−03 | 0.36 | 9.1E−02 | GCH1 | 1.8 | 3.5E−03 | 0.43 | 9.7E−02 |
| GADD45B | 2.2 | 4.0E−02 | 0.62 | 2.5E−03 | SYT11 | 1.8 | 2.7E−02 | 0.14 | 6.7E−01 |
| TICAM1 | 2.2 | 3.3E−02 | 0.30 | 1.5E−01 | FKBP5 | 1.8 | 1.3E−02 | −0.02 | 9.6E−01 |
| ZBED6CL | 2.2 | 4.7E−02 | 0.14 | 5.0E−01 | DBF4 | 1.8 | 3.9E−02 | −0.07 | 8.4E−01 |
| TEAD3 | 2.2 | 2.1E−02 | 0.03 | 9.0E−01 | FAM46C | 1.8 | 4.3E−02 | −0.02 | 9.7E−01 |
| MS4A4A | 2.2 | 3.8E−02 | 0.41 | 5.5E−02 | LILRB4 | 1.8 | 3.3E−02 | 0.05 | 8.9E−01 |
| LST1 | 2.2 | 4.5E−02 | 0.52 | 1.5E−02 | CKLF | 1.8 | 1.9E−02 | 0.40 | 1.3E−01 |
| F2RL1 | 2.1 | 2.2E−02 | 0.51 | 1.6E−02 | TVP23B | 1.8 | 2.7E−02 | 0.00 | 1.0E+00 |
| ZFAND1 | 2.1 | 1.9E−02 | 0.20 | 3.2E−01 | MALT1 | 1.8 | 2.5E−03 | −0.25 | 4.0E−01 |
| FAM117A | 2.1 | 8.8E−03 | 0.04 | 8.4E−01 | MANF | 1.8 | 7.7E−03 | 0.27 | 3.6E−01 |
| VIM | 2.1 | 2.2E−02 | 0.50 | 1.9E−02 | PELO | 1.8 | 1.4E−02 | −0.20 | 5.2E−01 |
| PATL1 | 2.1 | 2.2E−02 | 0.28 | 1.7E−01 | MYD88 | 1.8 | 6.3E−03 | 0.27 | 3.6E−01 |
| CCL11 | 2.1 | 5.0E−02 | 0.78 | 4.5E−05 | ELOVL5 | 1.8 | 1.4E−02 | 0.18 | 5.7E−01 |
| CNKSR1 | 2.1 | 3.7E−02 | 0.30 | 1.5E−01 | RBPMS | 1.8 | 3.2E−02 | 0.19 | 5.4E−01 |
| MXD3 | 2.1 | 4.3E−02 | 0.19 | 3.5E−01 | CCDC102B | 1.8 | 3.2E−02 | −0.02 | 9.7E−01 |
| RNPEP | 2.1 | 5.7E−03 | 0.34 | 1.1E−01 | SYVN1 | 1.8 | 2.1E−02 | 0.21 | 5.0E−01 |
| DNAJA3 | 2.1 | 3.2E−02 | 0.26 | 2.1E−01 | TRAM1 | 1.8 | 3.8E−02 | 0.47 | 5.9E−02 |
| PODXL | 2.1 | 1.1E−02 | 0.25 | 2.1E−01 | QKI | 1.8 | 6.9E−03 | 0.42 | 1.1E−01 |
| ZDHHC5 | 2.1 | 6.7E−03 | 0.55 | 1.1E−02 | MYC | 1.8 | 1.6E−02 | 0.34 | 2.4E−01 |
| NR112 | 2.1 | 1.6E−02 | 0.44 | 4.0E−02 | HPS5 | 1.8 | 3.4E−02 | 0.06 | 8.7E−01 |
| ADAMDEC1 | 2.1 | 1.6E−03 | 0.51 | 1.6E−02 | C1R | 1.8 | 1.2E−02 | 0.24 | 4.2E−01 |
| MDH1 | 2.1 | 4.6E−03 | 0.33 | 1.2E−01 | WRAP73 | 1.8 | 1.3E−03 | 0.32 | 2.7E−01 |
| NUDT21 | 2.1 | 2.0E−02 | 0.29 | 1.6E−01 | PIM3 | 1.8 | 2.1E−03 | −0.41 | 1.2E−01 |
| UBL5 | 2.1 | 3.0E−02 | 0.05 | 8.2E−01 | MCFD2 | 1.8 | 1.9E−02 | −0.27 | 3.7E−01 |
| NECAP2 | 2.1 | 1.8E−03 | 0.45 | 3.4E−02 | IRF7 | 1.8 | 1.4E−03 | 0.25 | 4.0E−01 |
| PLCG2 | 2.1 | 2.1E−02 | 0.13 | 5.3E−01 | GLCCI1 | 1.8 | 2.8E−02 | 0.04 | 9.1E−01 |
| COL3A1 | 2.1 | 4.4E−02 | 0.60 | 3.7E−03 | NCOA7 | 1.8 | 1.5E−02 | 0.18 | 5.5E−01 |
| QSOX2 | 2.1 | 2.8E−02 | 0.45 | 3.4E−02 | C1orf174 | 1.8 | 1.4E−02 | 0.34 | 2.3E−01 |
| PLAU | 2.1 | 4.8E−02 | 0.42 | 5.0E−02 | MCM2 | 1.8 | 4.3E−02 | 0.03 | 9.5E−01 |
| CHEK2 | 2.1 | 1.9E−02 | 0.25 | 2.2E−01 | CTSC | 1.8 | 1.8E−04 | 0.61 | 9.9E−03 |
| ADD1 | 2.1 | 5.7E−03 | 0.53 | 1.1E−02 | RBMS1 | 1.8 | 3.8E−02 | 0.30 | 3.0E−01 |
| JOSD1 | 2.1 | 3.3E−03 | 0.70 | 5.5E−04 | ABCB10 | 1.8 | 1.0E−02 | 0.47 | 5.8E−02 |
| ZFP36L2 | 2.1 | 1.3E−02 | 0.33 | 1.1E−01 | AKAP2 | 1.8 | 1.4E−02 | 0.00 | 1.0E+00 |
| ETNK1 | 2.1 | 1.4E−02 | 0.70 | 5.5E−04 | MTMR6 | 1.8 | 2.8E−04 | 0.08 | 8.4E−01 |
| LZTS2 | 2.1 | 2.3E−02 | 0.48 | 2.5E−02 | PIP4K2A | 1.8 | 1.1E−02 | 0.38 | 1.6E−01 |
| C2 | 2.1 | 3.8E−02 | 0.56 | 8.5E−03 | FAM49A | 1.7 | 3.4E−03 | 0.23 | 4.6E−01 |
| ZNF816 | 2.1 | 4.6E−02 | 0.52 | 1.5E−02 | ZC3H7B | 1.7 | 2.0E−02 | 0.26 | 3.8E−01 |
| MRPS14 | 2.1 | 2.8E−02 | 0.24 | 2.3E−01 | LMO4 | 1.7 | 2.3E−02 | 0.22 | 4.7E−01 |
| DYRK4 | 2.1 | 4.9E−02 | 0.26 | 2.0E−01 | TRAFD1 | 1.7 | 4.0E−02 | 0.29 | 3.3E−01 |
| ADA2 | 2.1 | 1.3E−02 | 0.46 | 3.0E−02 | SLC25A28 | 1.7 | 7.3E−03 | −0.09 | 8.0E−01 |
| HBP1 | 2.1 | 1.9E−02 | 0.41 | 5.5E−02 | H2AFX | 1.7 | 1.1E−02 | 0.12 | 7.1E−01 |
| RCC1 | 2.1 | 5.1E−03 | 0.40 | 6.0E−02 | AMPD2 | 1.7 | 2.4E−02 | 0.20 | 5.2E−01 |
| MAT2B | 2.1 | 7.4E−03 | 0.36 | 8.7E−02 | CYTH4 | 1.7 | 5.5E−03 | −0.15 | 6.6E−01 |
| WDR11 | 2.1 | 2.1E−02 | 0.19 | 3.5E−01 | TFEC | 1.7 | 1.2E−02 | 0.28 | 3.4E−01 |
| CCNG1 | 2.0 | 4.6E−03 | 0.25 | 2.1E−01 | ALKBH5 | 1.7 | 1.2E−02 | 0.34 | 2.4E−01 |
| SLC38A9 | 2.0 | 3.4E−02 | 0.32 | 1.3E−01 | CHPT1 | 1.7 | 1.5E−02 | 0.28 | 3.3E−01 |
| GTF3C2 | 2.0 | 3.8E−02 | 0.23 | 2.7E−01 | ST3GAL1 | 1.7 | 3.6E−02 | −0.04 | 9.2E−01 |
| ANXA5 | 2.0 | 4.1E−02 | 0.40 | 6.2E−02 | SP110 | 1.7 | 5.3E−03 | −0.05 | 8.9E−01 |
| KBTBD3 | 2.0 | 9.5E−03 | 0.12 | 5.7E−01 | PSMB8 | 1.7 | 3.3E−02 | 0.31 | 2.8E−01 |
| SNRPF | 2.0 | 3.5E−02 | −0.06 | 7.7E−01 | RNF145 | 1.7 | 2.8E−03 | 0.18 | 5.7E−01 |
| LTA4H | 2.0 | 3.1E−03 | 0.54 | 1.1E−02 | LINS1 | 1.7 | 2.6E−02 | 0.20 | 5.2E−01 |
| TRAM1 | 2.0 | 4.4E−02 | 0.47 | 2.6E−02 | ARID3A | 1.7 | 2.8E−02 | 0.48 | 5.1E−02 |
| ZNF598 | 2.0 | 4.1E−02 | 0.22 | 2.7E−01 | TCIRG1 | 1.7 | 2.7E−02 | −0.16 | 6.1E−01 |
| XYLT1 | 2.0 | 1.8E−02 | 0.54 | 1.1E−02 | CREB3L2 | 1.7 | 4.7E−03 | 0.41 | 1.2E−01 |
| AMPD2 | 2.0 | 5.0E−02 | 0.20 | 3.3E−01 | PPT1 | 1.7 | 3.6E−03 | 0.25 | 4.1E−01 |
| ARMC8 | 2.0 | 1.8E−02 | 0.21 | 3.0E−01 | IL15RA | 1.7 | 4.2E−02 | 0.55 | 2.1E−02 |
| FERMT2 | 2.0 | 2.7E−02 | 0.51 | 1.6E−02 | FAM241B | 1.7 | 1.6E−02 | 0.13 | 7.1E−01 |
| PHF1 | 2.0 | 4.3E−03 | 0.10 | 6.2E−01 | MAFB | 1.7 | 2.9E−03 | 0.42 | 1.1E−01 |
| LYPLA1 | 2.0 | 4.2E−02 | 0.45 | 3.6E−02 | HIF1A | 1.7 | 7.3E−03 | 0.21 | 4.9E−01 |
| TADA2B | 2.0 | 9.2E−03 | 0.33 | 1.2E−01 | CDK17 | 1.7 | 5.1E−03 | 0.33 | 2.6E−01 |
| WDR48 | 2.0 | 1.3E−02 | 0.52 | 1.4E−02 | FAM3C | 1.7 | 4.9E−03 | −0.20 | 5.2E−01 |
| TSEN54 | 2.0 | 2.6E−02 | 0.36 | 8.7E−02 | RNF213 | 1.7 | 1.3E−02 | 0.32 | 2.8E−01 |
| C1QBP | 2.0 | 3.1E−02 | 0.06 | 7.9E−01 | ELOC | 1.7 | 1.0E−02 | 0.28 | 3.5E−01 |
| CTSC | 2.0 | 8.5E−03 | 0.61 | 3.5E−03 | COTL1 | 1.7 | 4.2E−02 | 0.12 | 7.1E−01 |
| WASHC5 | 2.0 | 4.3E−02 | 0.19 | 3.4E−01 | KLHL2 | 1.7 | 4.0E−02 | −0.01 | 9.7E−01 |
| SIK3 | 2.0 | 3.1E−02 | 0.41 | 5.5E−02 | ZBTB17 | 1.7 | 3.1E−02 | 0.23 | 4.5E−01 |
| GON7 | 2.0 | 8.1E−03 | 0.32 | 1.3E−01 | WDR11 | 1.7 | 5.7E−03 | 0.19 | 5.3E−01 |
| RBM27 | 2.0 | 2.0E−02 | 0.19 | 3.6E−01 | PIEZO1 | 1.7 | 3.5E−02 | 0.25 | 4.0E−01 |
| LEFTY1 | 2.0 | 4.6E−02 | 0.05 | 8.1E−01 | MAGEF1 | 1.7 | 1.0E−02 | 0.01 | 9.7E−01 |
| PYCARD | 2.0 | 1.3E−02 | 0.28 | 1.7E−01 | MMP25 | 1.7 | 1.8E−02 | 0.43 | 9.7E−02 |
| LSM14B | 2.0 | 1.6E−02 | 0.28 | 1.7E−01 | ODC1 | 1.7 | 3.6E−02 | −0.16 | 6.1E−01 |
| RPS18 | 2.0 | 1.2E−02 | 0.07 | 7.3E−01 | DCTN6 | 1.7 | 4.2E−02 | 0.15 | 6.4E−01 |
| KCTD5 | 2.0 | 3.8E−02 | 0.51 | 1.6E−02 | ASAP1 | 1.7 | 1.2E−02 | −0.03 | 9.5E−01 |
| PYURF | 2.0 | 3.5E−02 | 0.45 | 3.4E−02 | HRH2 | 1.7 | 1.3E−02 | 0.38 | 1.6E−01 |
| GCHFR | 2.0 | 3.1E−02 | 0.16 | 4.2E−01 | RHOQ | 1.7 | 6.7E−03 | 0.10 | 7.8E−01 |
| MANSC1 | 2.0 | 3.2E−02 | 0.41 | 5.5E−02 | RRP15 | 1.7 | 8.1E−04 | −0.43 | 9.7E−02 |
| EFNB1 | 2.0 | 3.8E−02 | 0.40 | 6.2E−02 | DSE | 1.7 | 2.5E−02 | 0.19 | 5.3E−01 |
| DENND6A | 2.0 | 5.0E−02 | 0.57 | 6.9E−03 | ELK1 | 1.6 | 4.2E−02 | 0.22 | 4.6E−01 |
| SLC44A3 | 2.0 | 2.5E−02 | 0.37 | 8.0E−02 | BTN3A1 | 1.6 | 1.5E−02 | −0.13 | 7.1E−01 |
| KDM6B | 2.0 | 2.5E−02 | 0.44 | 4.0E−02 | PAPSS1 | 1.6 | 3.7E−02 | 0.24 | 4.2E−01 |
| PIP4P1 | 2.0 | 4.0E−02 | 0.13 | 5.4E−01 | SH3BP4 | 1.6 | 4.6E−02 | 0.08 | 8.4E−01 |
| KDM4C | 2.0 | 1.2E−02 | 0.57 | 7.6E−03 | SNRPF | 1.6 | 7.6E−03 | −0.06 | 8.7E−01 |
| RPS27 | 2.0 | 3.7E−02 | 0.06 | 7.7E−01 | HELZ2 | 1.6 | 4.5E−02 | 0.36 | 2.1E−01 |
| FAM122A | 1.9 | 3.7E−02 | 0.18 | 3.7E−01 | C1GALT1 | 1.6 | 4.1E−03 | 0.39 | 1.4E−01 |
| GPR82 | 1.9 | 5.0E−02 | 0.55 | 9.7E−03 | IGIF | 1.6 | 4.5E−02 | 0.25 | 4.0E−01 |
| MINOS1 | 1.9 | 4.0E−02 | 0.30 | 1.5E−01 | MTMR14 | 1.6 | 1.5E−02 | 0.20 | 5.2E−01 |
| AP3M1 | 1.9 | 4.3E−03 | 0.31 | 1.4E−01 | CASP4 | 1.6 | 3.6E−02 | −0.19 | 5.4E−01 |
| RFT1 | 1.9 | 4.4E−02 | 0.21 | 3.0E−01 | HLA-DPA1 | 1.6 | 1.2E−02 | 0.17 | 5.9E−01 |
| HSPA8 | 1.9 | 4.7E−02 | 0.08 | 7.2E−01 | RASGEF1B | 1.6 | 4.8E−02 | 0.01 | 9.7E−01 |
| FASTKD3 | 1.9 | 4.4E−02 | 0.55 | 1.1E−02 | FBXW11 | 1.6 | 1.3E−02 | 0.46 | 6.8E−02 |
| LIX1L | 1.9 | 4.8E−02 | 0.26 | 2.0E−01 | SESTD1 | 1.6 | 1.5E−02 | −0.54 | 2.5E−02 |
| UMAD1 | 1.9 | 3.2E−02 | 0.23 | 2.5E−01 | CARD16 | 1.6 | 3.7E−02 | 0.31 | 3.0E−01 |
| NCAPG2 | 1.9 | 1.1E−02 | −0.01 | 9.6E−01 | TRAF1 | 1.6 | 2.6E−02 | −0.09 | 8.1E−01 |
| ATP6V0D2 | 1.9 | 4.4E−02 | 0.32 | 1.3E−01 | NUCB2 | 1.6 | 2.3E−02 | −0.57 | 1.4E−02 |
| DENND3 | 1.9 | 2.1E−02 | 0.38 | 7.4E−02 | TGFBR2 | 1.6 | 1.7E−02 | 0.27 | 3.7E−01 |
| SPATA33 | 1.9 | 4.7E−02 | −0.06 | 7.7E−01 | STAT5A | 1.6 | 3.5E−02 | 0.22 | 4.6E−01 |
| HIGD2A | 1.9 | 1.7E−02 | −0.01 | 9.6E−01 | MARCKSL1 | 1.6 | 4.7E−02 | 0.25 | 4.1E−01 |
| PCMTD2 | 1.9 | 3.5E−02 | 0.48 | 2.5E−02 | TC2N | 1.6 | 9.2E−03 | −0.24 | 4.2E−01 |
| PNMA1 | 1.9 | 1.3E−02 | 0.27 | 1.9E−01 | PPA1 | 1.6 | 9.4E−03 | 0.22 | 4.8E−01 |
| QPCT | 1.9 | 4.3E−02 | 0.63 | 2.3E−03 | CCDC69 | 1.6 | 2.0E−02 | 0.11 | 7.6E−01 |
| PPP1R14C | 1.9 | 9.1E−03 | 0.49 | 2.2E−02 | RB1 | 1.6 | 4.9E−03 | 0.25 | 4.0E−01 |
| USP32 | 1.9 | 2.8E−02 | 0.48 | 2.4E−02 | PEA15 | 1.6 | 1.9E−02 | −0.29 | 3.3E−01 |
| ANXA4 | 1.9 | 4.2E−03 | 0.41 | 5.5E−02 | PRKAR2B | 1.6 | 3.8E−02 | −0.33 | 2.4E−01 |
| SMIM20 | 1.9 | 4.2E−02 | 0.34 | 1.0E−01 | PHC2 | 1.6 | 3.9E−02 | 0.37 | 1.9E−01 |
| NUS1 | 1.9 | 8.5E−03 | 0.45 | 3.7E−02 | PPP1R2 | 1.6 | 3.9E−03 | 0.16 | 6.2E−01 |
| ARPC1A | 1.9 | 1.5E−02 | 0.08 | 7.0E−01 | RGL1 | 1.6 | 1.4E−02 | 0.49 | 4.9E−02 |
| RASSF5 | 1.9 | 1.2E−02 | 0.34 | 1.1E−01 | M6PR | 1.6 | 4.9E−03 | 0.30 | 3.0E−01 |
| PEX5 | 1.9 | 2.1E−02 | 0.22 | 2.8E−01 | C19orf66 | 1.6 | 4.0E−02 | 0.38 | 1.6E−01 |
| COG3 | 1.9 | 4.3E−02 | 0.22 | 2.8E−01 | AIF1 | 1.6 | 1.4E−02 | −0.01 | 9.7E−01 |
| DERL1 | 1.9 | 1.6E−02 | 0.49 | 2.2E−02 | CD44 | 1.6 | 1.2E−02 | 0.21 | 4.9E−01 |
| PLK2 | 1.9 | 5.0E−02 | 0.02 | 9.4E−01 | CREM | 1.6 | 2.7E−02 | 0.09 | 8.0E−01 |
| NDUFC2 | 1.9 | 4.7E−02 | 0.06 | 7.8E−01 | SEMA4A | 1.6 | 1.4E−02 | 0.08 | 8.4E−01 |
| DACH1 | 1.9 | 2.5E−02 | 0.40 | 5.9E−02 | ZNF618 | 1.6 | 4.6E−02 | −0.14 | 6.8E−01 |
| WDR26 | 1.9 | 1.9E−03 | 0.41 | 5.2E−02 | MARS | 1.6 | 2.3E−02 | −0.49 | 4.8E−02 |
| THAP11 | 1.9 | 8.5E−03 | 0.36 | 9.0E−02 | LMAN1 | 1.6 | 4.3E−03 | −0.23 | 4.5E−01 |
| CDAN1 | 1.9 | 3.0E−02 | 0.21 | 3.0E−01 | TNFRSF1B | 1.6 | 1.9E−02 | 0.09 | 8.1E−01 |
| ABHD17B | 1.9 | 2.3E−02 | 0.00 | 9.9E−01 | BICDL1 | 1.6 | 1.6E−02 | 0.04 | 9.2E−01 |
| KLF7 | 1.9 | 1.9E−02 | 0.33 | 1.2E−01 | SLC1A4 | 1.6 | 2.6E−03 | −0.14 | 6.7E−01 |
| ERLIN1 | 1.9 | 1.8E−02 | 0.15 | 4.6E−01 | STAT2 | 1.6 | 1.9E−02 | 0.24 | 4.2E−01 |
| INTS4 | 1.9 | 2.7E−02 | 0.33 | 1.2E−01 | JADE1 | 1.6 | 2.5E−02 | −0.10 | 8.0E−01 |
| PRDX3 | 1.9 | 1.3E−02 | 0.02 | 9.4E−01 | MLKL | 1.6 | 1.3E−02 | −0.20 | 5.2E−01 |
| AIDA | 1.9 | 4.1E−02 | 0.37 | 7.7E−02 | GART | 1.6 | 1.9E−03 | 0.39 | 1.4E−01 |
| MYL12B | 1.9 | 1.8E−02 | 0.48 | 2.3E−02 | GTF2A1 | 1.6 | 4.7E−03 | 0.39 | 1.4E−01 |
| SLC35B3 | 1.9 | 2.2E−02 | 0.42 | 4.7E−02 | MX1 | 1.5 | 1.2E−02 | −0.04 | 9.2E−01 |
| FPR1 | 1.9 | 4.9E−02 | 0.48 | 2.5E−02 | IL6ST | 1.5 | 1.5E−02 | 0.40 | 1.3E−01 |
| MT-CO2 | 1.9 | 5.7E−03 | 0.36 | 8.9E−02 | MYO1F | 1.5 | 2.1E−02 | 0.33 | 2.4E−01 |
| ERBB2 | 1.9 | 2.5E−02 | 0.62 | 2.9E−03 | IL1R1 | 1.5 | 4.7E−02 | 0.40 | 1.3E−01 |
| CAMTA1 | 1.9 | 4.2E−02 | 0.44 | 3.9E−02 | CXXC5 | 1.5 | 3.3E−02 | −0.09 | 8.1E−01 |
| GATA2 | 1.9 | 2.1E−02 | 0.27 | 1.9E−01 | FCER1G | 1.5 | 3.3E−02 | 0.08 | 8.4E−01 |
| MAFG | 1.9 | 1.9E−02 | 0.35 | 1.0E−01 | NUS1 | 1.5 | 4.3E−03 | 0.45 | 8.2E−02 |
| CASP9 | 1.9 | 5.0E−02 | 0.12 | 5.6E−01 | LIPA | 1.5 | 4.4E−02 | 0.45 | 8.2E−02 |
| SERTAD3 | 1.9 | 3.4E−02 | 0.25 | 2.2E−01 | GLS | 1.5 | 9.3E−03 | 0.38 | 1.7E−01 |
| HTT | 1.9 | 7.6E−03 | 0.53 | 1.2E−02 | OST4 | 1.5 | 3.9E−02 | 0.20 | 5.2E−01 |
| PSMB8 | 1.9 | 3.5E−02 | 0.31 | 1.3E−01 | MYCBP2 | 1.5 | 2.7E−02 | 0.30 | 3.0E−01 |
| FUCA2 | 1.9 | 1.9E−02 | 0.24 | 2.4E−01 | UBE2J1 | 1.5 | 1.2E−02 | 0.19 | 5.4E−01 |
| TAF4 | 1.9 | 4.0E−03 | 0.47 | 2.7E−02 | ZNF267 | 1.5 | 4.0E−02 | −0.09 | 8.1E−01 |
| RPL17 | 1.9 | 9.3E−03 | 0.20 | 3.3E−01 | PATL1 | 1.5 | 3.8E−02 | 0.28 | 3.4E−01 |
| S100A6 | 1.9 | 8.4E−03 | 0.28 | 1.7E−01 | IRAK4 | 1.5 | 2.6E−02 | 0.24 | 4.2E−01 |
| TMEM70 | 1.9 | 9.2E−03 | 0.13 | 5.4E−01 | PHACTR4 | −1.5 | 1.1E−02 | −0.25 | 4.1E−01 |
| CCT4 | 1.9 | 8.2E−03 | 0.16 | 4.2E−01 | SLC25A39 | −1.5 | 2.2E−02 | 0.13 | 7.1E−01 |
| HLA-DPA1 | 1.9 | 1.1E−02 | 0.17 | 4.1E−01 | COX10 | −1.5 | 1.1E−02 | −0.17 | 5.9E−01 |
| HERC5 | 1.9 | 4.8E−02 | 0.25 | 2.2E−01 | PFKL | −1.5 | 1.5E−02 | −0.06 | 8.7E−01 |
| COMT | 1.9 | 1.2E−02 | 0.22 | 2.8E−01 | ZNF20 | −1.5 | 2.8E−02 | −0.07 | 8.4E−01 |
| EIF3H | 1.9 | 1.1E−02 | 0.11 | 5.9E−01 | GATB | −1.5 | 4.6E−02 | −0.02 | 9.7E−01 |
| SOX13 | 1.8 | 2.4E−02 | 0.29 | 1.6E−01 | SPRY1 | −1.5 | 1.2E−02 | 0.32 | 2.7E−01 |
| ATP6V1H | 1.8 | 3.3E−03 | 0.41 | 5.4E−02 | HCRT | −1.5 | 4.8E−02 | −0.04 | 9.2E−01 |
| SLC25A5 | 1.8 | 1.8E−02 | 0.53 | 1.2E−02 | ISCA1 | −1.5 | 6.1E−03 | 0.29 | 3.2E−01 |
| WIPI1 | 1.8 | 3.3E−02 | 0.32 | 1.2E−01 | HADHA | −1.5 | 1.4E−02 | 0.21 | 5.0E−01 |
| POLR1D | 1.8 | 3.8E−03 | 0.30 | 1.5E−01 | ITPR3 | −1.5 | 4.1E−02 | 0.35 | 2.2E−01 |
| LPGAT1 | 1.8 | 4.8E−02 | 0.49 | 2.0E−02 | PWWP2B | −1.5 | 2.4E−02 | −0.01 | 9.8E−01 |
| KIAA1147 | 1.8 | 1.6E−02 | 0.26 | 2.0E−01 | ADCK2 | −1.5 | 1.6E−02 | −0.20 | 5.2E−01 |
| PLXND1 | 1.8 | 1.6E−02 | 0.42 | 4.6E−02 | LEXM | −1.5 | 2.7E−02 | −0.11 | 7.6E−01 |
| PEF1 | 1.8 | 1.6E−02 | 0.26 | 2.0E−01 | NBPF1 | −1.5 | 3.9E−02 | −0.08 | 8.2E−01 |
| COX7A2L | 1.8 | 4.2E−03 | 0.37 | 7.7E−02 | EXOC3 | −1.5 | 3.1E−02 | 0.67 | 3.4E−03 |
| PGLS | 1.8 | 2.6E−02 | 0.41 | 5.3E−02 | URGCP | −1.5 | 2.8E−02 | −0.29 | 3.3E−01 |
| DYNLT3 | 1.8 | 1.9E−03 | 0.28 | 1.8E−01 | MRPS18B | −1.5 | 2.0E−02 | −0.32 | 2.7E−01 |
| RPL27 | 1.8 | 1.1E−02 | 0.27 | 1.9E−01 | PEBP1 | −1.5 | 2.7E−02 | 0.30 | 3.0E−01 |
| LAMP2 | 1.8 | 5.7E−03 | 0.61 | 3.5E−03 | TSTD1 | −1.5 | 3.3E−03 | −0.30 | 3.0E−01 |
| SOD2 | 1.8 | 6.5E−03 | 0.35 | 9.6E−02 | IL10RB | −1.5 | 1.0E−02 | −0.19 | 5.4E−01 |
| ARF4 | 1.8 | 4.3E−02 | 0.06 | 7.7E−01 | DDO | −1.5 | 4.0E−03 | −0.26 | 3.8E−01 |
| HERPUD1 | 1.8 | 4.1E−02 | 0.23 | 2.6E−01 | SPR | −1.5 | 4.5E−02 | −0.16 | 6.0E−01 |
| ARHGAP27 | 1.8 | 2.9E−02 | 0.14 | 4.8E−01 | C11orf71 | −1.5 | 3.2E−02 | −0.15 | 6.4E−01 |
| LAMTOR3 | 1.8 | 5.4E−03 | 0.36 | 8.5E−02 | MBOAT7 | −1.5 | 4.7E−03 | −0.03 | 9.4E−01 |
| TOR1A | 1.8 | 2.5E−02 | 0.09 | 6.6E−01 | SLC25A2 | −1.5 | 3.6E−02 | 0.01 | 9.8E−01 |
| COTL1 | 1.8 | 1.1E−02 | 0.12 | 5.4E−01 | ZDHHC19 | −1.5 | 3.1E−02 | 0.12 | 7.1E−01 |
| LLGL2 | 1.8 | 1.5E−02 | 0.51 | 1.6E−02 | PCDH1 | −1.5 | 4.2E−02 | −0.31 | 3.0E−01 |
| PIGP | 1.8 | 4.4E−02 | 0.12 | 5.6E−01 | MAL2 | −1.5 | 4.3E−02 | −0.16 | 6.2E−01 |
| SARAF | 1.8 | 1.4E−02 | 0.31 | 1.4E−01 | SCG3 | −1.5 | 2.7E−02 | −0.26 | 3.8E−01 |
| MPZL1 | 1.8 | 1.0E−02 | 0.36 | 9.0E−02 | APLP2 | −1.5 | 2.8E−03 | 0.10 | 7.8E−01 |
| PRKAA1 | 1.8 | 4.6E−02 | 0.33 | 1.2E−01 | PGRMC2 | −1.5 | 2.6E−04 | −0.28 | 3.4E−01 |
| DSCR3 | 1.8 | 2.5E−02 | 0.30 | 1.5E−01 | ABHD14B | −1.5 | 2.0E−02 | −0.07 | 8.4E−01 |
| CCNY | 1.8 | 7.2E−03 | 0.37 | 8.2E−02 | MRPL41 | −1.5 | 3.5E−02 | −0.49 | 4.6E−02 |
| ZSWIM8 | 1.8 | 1.9E−02 | 0.16 | 4.3E−01 | OTUD7B | −1.5 | 2.3E−02 | 0.02 | 9.7E−01 |
| CAPN12 | 1.8 | 1.5E−02 | 0.33 | 1.2E−01 | KDM4A | −1.5 | 4.1E−03 | 0.14 | 6.8E−01 |
| GTF2H1 | 1.8 | 1.0E−02 | 0.32 | 1.3E−01 | IL17C | −1.5 | 3.9E−02 | 0.17 | 5.9E−01 |
| SELENOM | 1.8 | 4.0E−02 | 0.02 | 9.3E−01 | NDUFS1 | −1.5 | 1.9E−02 | 0.07 | 8.4E−01 |
| CGGBP1 | 1.8 | 2.2E−02 | 0.31 | 1.5E−01 | ANKDD1B | −1.5 | 4.2E−02 | 0.20 | 5.2E−01 |
| SLC35A2 | 1.8 | 9.9E−03 | 0.15 | 4.6E−01 | PUF60 | −1.5 | 2.5E−02 | −0.17 | 5.9E−01 |
| CLPTM1L | 1.8 | 9.7E−03 | 0.24 | 2.4E−01 | DLST | −1.5 | 1.0E−02 | 0.02 | 9.7E−01 |
| BDH2 | 1.8 | 3.2E−02 | 0.43 | 4.6E−02 | TCEAL5 | −1.5 | 3.5E−02 | 0.01 | 9.7E−01 |
| DPY30 | 1.8 | 2.6E−02 | 0.27 | 1.8E−01 | GPRASP2 | −1.5 | 2.5E−02 | −0.19 | 5.3E−01 |
| SRPRA | 1.8 | 3.0E−02 | 0.32 | 1.3E−01 | FBXW5 | −1.5 | 9.2E−03 | −0.29 | 3.3E−01 |
| CHKA | 1.8 | 1.9E−02 | 0.62 | 2.9E−03 | STAB1 | −1.5 | 3.1E−02 | −0.15 | 6.6E−01 |
| ABCG1 | 1.8 | 3.1E−02 | 0.32 | 1.3E−01 | SCAF1 | −1.5 | 1.8E−03 | 0.08 | 8.2E−01 |
| LASP1 | 1.8 | 1.0E−02 | 0.61 | 3.0E−03 | AFG3L2 | −1.5 | 5.9E−03 | 0.14 | 6.8E−01 |
| EIF4E3 | 1.8 | 3.0E−02 | 0.67 | 1.1E−03 | IQCC | −1.5 | 2.5E−02 | −0.18 | 5.7E−01 |
| SEC16B | 1.8 | 2.7E−02 | 0.22 | 2.7E−01 | HSDL2 | −1.5 | 1.0E−02 | 0.06 | 8.8E−01 |
| RBPMS | 1.8 | 3.1E−02 | 0.19 | 3.6E−01 | MFSD4B | −1.5 | 3.5E−02 | −0.07 | 8.4E−01 |
| NAIP | 1.8 | 1.8E−02 | 0.31 | 1.3E−01 | SNX7 | −1.5 | 1.2E−02 | −0.31 | 2.8E−01 |
| RIN3 | 1.8 | 5.1E−03 | 0.39 | 6.8E−02 | GAREM1 | −1.5 | 3.6E−02 | 0.49 | 4.9E−02 |
| HNRNPH3 | 1.8 | 4.7E−02 | 0.28 | 1.8E−01 | CLDN8 | −1.5 | 3.0E−02 | −0.64 | 6.6E−03 |
| NOTCH1 | 1.8 | 2.3E−02 | 0.38 | 7.4E−02 | LIAS | −1.5 | 1.6E−02 | 0.01 | 9.7E−01 |
| RNPS1 | 1.8 | 3.2E−02 | 0.28 | 1.7E−01 | GSTO2 | −1.5 | 1.6E−02 | −0.30 | 3.0E−01 |
| C9orf152 | 1.8 | 2.3E−02 | 0.04 | 8.7E−01 | NFKBIL1 | −1.5 | 5.0E−04 | 0.20 | 5.2E−01 |
| EIF4A3 | 1.8 | 8.3E−03 | 0.29 | 1.6E−01 | AC002310.4 | −1.5 | 3.8E−02 | 0.06 | 8.8E−01 |
| SLC35D2 | 1.8 | 1.7E−02 | 0.42 | 4.9E−02 | IMMT | −1.5 | 1.1E−03 | 0.14 | 6.6E−01 |
| CD164 | 1.8 | 2.2E−02 | 0.27 | 1.9E−01 | TIMM22 | −1.5 | 3.2E−02 | −0.01 | 9.8E−01 |
| MS4A6A | 1.8 | 3.7E−03 | 0.39 | 6.8E−02 | CDS1 | −1.5 | 3.4E−02 | 0.15 | 6.4E−01 |
| PSMA2 | 1.8 | 1.9E−02 | 0.19 | 3.6E−01 | PGPEP1 | −1.5 | 1.5E−02 | −0.09 | 8.1E−01 |
| ABAT | 1.8 | 4.6E−02 | 0.72 | 4.3E−04 | BRF1 | −1.5 | 1.7E−03 | −0.11 | 7.5E−01 |
| TWSG1 | 1.8 | 4.3E−02 | 0.07 | 7.4E−01 | COX4I1 | −1.5 | 3.7E−03 | −0.54 | 2.5E−02 |
| MGST2 | 1.7 | 2.3E−02 | 0.03 | 9.0E−01 | RXRA | −1.5 | 1.5E−03 | 0.24 | 4.2E−01 |
| CEACAM1 | 1.7 | 9.8E−03 | 0.65 | 1.5E−03 | DNASE1L2 | −1.5 | 1.2E−02 | 0.17 | 5.9E−01 |
| MTX3 | 1.7 | 1.2E−02 | 0.28 | 1.8E−01 | AL591806.3 | −1.5 | 4.7E−02 | 0.17 | 5.9E−01 |
| SOCS1 | 1.7 | 3.4E−02 | 0.29 | 1.6E−01 | HSD11B1L | −1.5 | 1.6E−02 | 0.09 | 8.1E−01 |
| COMMD2 | 1.7 | 5.0E−02 | 0.30 | 1.5E−01 | NPIPB12 | −1.5 | 2.9E−02 | 0.09 | 8.0E−01 |
| B3GALNT2 | 1.7 | 4.1E−02 | 0.18 | 3.6E−01 | GPR37L1 | −1.5 | 3.7E−02 | 0.25 | 4.1E−01 |
| GABARAPL2 | 1.7 | 2.8E−02 | 0.29 | 1.6E−01 | CNPPD1 | −1.5 | 1.1E−02 | 0.01 | 9.8E−01 |
| PSMD9 | 1.7 | 3.8E−02 | 0.28 | 1.8E−01 | AMELX | −1.5 | 4.8E−02 | −0.04 | 9.1E−01 |
| PDZRN3 | 1.7 | 3.3E−02 | 0.06 | 7.7E−01 | PYGO2 | −1.5 | 2.7E−02 | −0.15 | 6.5E−01 |
| CNOT8 | 1.7 | 3.5E−02 | 0.33 | 1.2E−01 | UQCRC2 | −1.5 | 7.0E−03 | −0.36 | 2.0E−01 |
| M6PR | 1.7 | 2.4E−02 | 0.30 | 1.5E−01 | PDE5A | −1.5 | 3.9E−02 | 0.46 | 6.9E−02 |
| USMG5 | 1.7 | 4.2E−02 | 0.14 | 5.0E−01 | TJP3 | −1.5 | 3.8E−02 | 0.08 | 8.4E−01 |
| GOLM1 | 1.7 | 1.3E−02 | 0.49 | 2.2E−02 | MINPP1 | −1.5 | 2.0E−02 | 0.32 | 2.8E−01 |
| HLA-DPB1 | 1.7 | 1.7E−02 | 0.20 | 3.2E−01 | TMEM8B | −1.5 | 2.5E−03 | −0.04 | 9.3E−01 |
| ALOX5AP | 1.7 | 3.4E−02 | 0.49 | 2.0E−02 | UEVLD | −1.5 | 4.4E−02 | 0.17 | 5.8E−01 |
| PHF10 | 1.7 | 1.5E−02 | 0.39 | 6.4E−02 | PHLDB1 | −1.5 | 7.9E−03 | 0.30 | 3.0E−01 |
| ATP5C1 | 1.7 | 4.4E−02 | 0.22 | 2.7E−01 | DLL4 | −1.5 | 1.6E−02 | 0.00 | 9.9E−01 |
| RPS29 | 1.7 | 1.9E−02 | 0.03 | 8.7E−01 | MEGF8 | −1.5 | 1.6E−03 | 0.46 | 6.6E−02 |
| MCL1 | 1.7 | 3.8E−03 | 0.42 | 4.8E−02 | PLEKHA6 | −1.5 | 4.0E−02 | 0.06 | 8.7E−01 |
| EXOC3L2 | 1.7 | 2.8E−02 | 0.54 | 1.1E−02 | RILP | −1.5 | 4.0E−02 | −0.07 | 8.4E−01 |
| SMAD4 | 1.7 | 4.1E−02 | 0.27 | 1.8E−01 | OR6F1 | −1.6 | 1.3E−02 | −0.03 | 9.5E−01 |
| TMEM59 | 1.7 | 3.2E−02 | 0.26 | 2.0E−01 | GFER | −1.6 | 1.7E−02 | −0.22 | 4.9E−01 |
| RRNAD1 | 1.7 | 1.9E−02 | 0.42 | 4.8E−02 | ANKRD40 | −1.6 | 4.1E−02 | 0.21 | 4.9E−01 |
| CCNG2 | 1.7 | 4.3E−02 | 0.32 | 1.3E−01 | FGF9 | −1.6 | 3.1E−02 | 0.00 | 1.0E+00 |
| IL1R1 | 1.7 | 3.1E−02 | 0.40 | 5.7E−02 | PDLIM1 | −1.6 | 4.0E−03 | −0.01 | 9.7E−01 |
| NOA1 | 1.7 | 3.8E−02 | 0.43 | 4.3E−02 | ECH1 | −1.6 | 1.0E−03 | −0.28 | 3.5E−01 |
| PIGG | 1.7 | 4.1E−02 | 0.44 | 4.1E−02 | SLC22A23 | −1.6 | 3.4E−02 | −0.26 | 3.8E−01 |
| MEF2D | 1.7 | 3.5E−02 | 0.20 | 3.3E−01 | ECI1 | −1.6 | 5.3E−03 | −0.04 | 9.1E−01 |
| RPS7 | 1.7 | 2.3E−02 | 0.03 | 8.8E−01 | NOL4L | −1.6 | 5.5E−04 | 0.17 | 5.9E−01 |
| ATRAID | 1.7 | 2.7E−02 | 0.28 | 1.7E−01 | CMTM4 | −1.6 | 1.9E−02 | −0.22 | 4.7E−01 |
| ATP2C1 | 1.7 | 2.4E−02 | 0.06 | 7.7E−01 | RETREG2 | −1.6 | 8.9E−04 | −0.05 | 9.0E−01 |
| PTPN9 | 1.7 | 2.7E−02 | 0.46 | 3.1E−02 | MOB2 | −1.6 | 2.2E−02 | −0.07 | 8.5E−01 |
| CRLS1 | 1.7 | 1.5E−02 | 0.24 | 2.3E−01 | C10orf143 | −1.6 | 3.8E−02 | 0.19 | 5.3E−01 |
| SAR1B | 1.7 | 1.7E−02 | 0.36 | 9.1E−02 | TMEM9B | −1.6 | 3.0E−02 | −0.61 | 9.9E−03 |
| AXIN1 | 1.7 | 4.6E−02 | 0.24 | 2.4E−01 | ZNF354B | −1.6 | 4.9E−02 | 0.26 | 3.8E−01 |
| RPL19 | 1.7 | 8.5E−03 | 0.03 | 9.0E−01 | HECTD3 | −1.6 | 9.8E−03 | −0.29 | 3.2E−01 |
| PGM5 | 1.7 | 4.7E−02 | 0.47 | 2.7E−02 | 43526 | −1.6 | 3.3E−02 | −0.04 | 9.2E−01 |
| MYL12A | 1.7 | 2.7E−02 | 0.36 | 9.1E−02 | UNC5B | −1.6 | 3.4E−02 | 0.21 | 4.9E−01 |
| SLCO2B1 | 1.7 | 2.2E−02 | 0.45 | 3.5E−02 | GNG12 | −1.6 | 1.3E−02 | 0.52 | 3.2E−02 |
| CMC2 | 1.7 | 3.3E−02 | 0.51 | 1.5E−02 | C19orf33 | −1.6 | 3.3E−02 | −0.33 | 2.4E−01 |
| MRPL11 | 1.7 | 3.1E−02 | 0.06 | 7.8E−01 | COX5B | −1.6 | 1.9E−02 | 0.01 | 9.8E−01 |
| ARRDC2 | 1.7 | 3.1E−02 | 0.31 | 1.5E−01 | SLC35F2 | −1.6 | 1.9E−02 | −0.08 | 8.3E−01 |
| RPL7 | 1.7 | 1.0E−02 | 0.15 | 4.7E−01 | ACBD4 | −1.6 | 2.7E−02 | 0.21 | 4.9E−01 |
| HLA-DRA | 1.7 | 7.1E−03 | 0.33 | 1.2E−01 | ZNF850 | −1.6 | 5.5E−04 | 0.12 | 7.3E−01 |
| EFTUD2 | 1.7 | 1.9E−02 | 0.31 | 1.4E−01 | FBXO48 | −1.6 | 3.9E−02 | 0.09 | 8.0E−01 |
| NSMF | 1.6 | 2.7E−02 | 0.32 | 1.3E−01 | SPSB3 | −1.6 | 3.3E−03 | −0.13 | 6.9E−01 |
| NCKAP1 | 1.6 | 2.7E−02 | 0.32 | 1.3E−01 | GBA2 | −1.6 | 2.9E−02 | −0.41 | 1.2E−01 |
| PPP6R2 | 1.6 | 1.7E−02 | −0.17 | 3.9E−01 | FAM218A | −1.6 | 3.5E−02 | 0.08 | 8.3E−01 |
| SLC44A1 | 1.6 | 2.2E−02 | 0.28 | 1.8E−01 | SYNPO | −1.6 | 3.0E−03 | 0.34 | 2.4E−01 |
| HDAC5 | 1.6 | 3.3E−02 | 0.22 | 2.7E−01 | MAPK13 | −1.6 | 4.7E−02 | −0.43 | 9.7E−02 |
| ZNF227 | 1.6 | 3.7E−02 | 0.28 | 1.8E−01 | IMPA2 | −1.6 | 1.7E−02 | 0.16 | 6.2E−01 |
| UBE2Q1 | 1.6 | 2.0E−02 | 0.35 | 9.6E−02 | ETFB | −1.6 | 3.1E−02 | −0.27 | 3.6E−01 |
| RNF138 | 1.6 | 3.8E−02 | −0.12 | 5.7E−01 | RTL8A | −1.6 | 1.7E−02 | −0.14 | 6.7E−01 |
| CD209 | 1.6 | 3.3E−02 | 0.77 | 4.5E−05 | FAM71E1 | −1.6 | 2.6E−02 | −0.08 | 8.3E−01 |
| SCARB2 | 1.6 | 2.7E−02 | 0.52 | 1.5E−02 | TPRG1L | −1.6 | 4.5E−04 | −0.16 | 6.2E−01 |
| UBE2L6 | 1.6 | 2.3E−02 | 0.61 | 3.1E−03 | DHRS1 | −1.6 | 6.8E−03 | −0.22 | 4.9E−01 |
| TRIOBP | 1.6 | 3.3E−02 | 0.38 | 7.7E−02 | ZKSCAN1 | −1.6 | 1.1E−02 | −0.20 | 5.2E−01 |
| 43710 | 1.6 | 2.8E−02 | 0.42 | 4.8E−02 | VSIG10 | −1.6 | 3.4E−02 | 0.20 | 5.2E−01 |
| APPBP2 | 1.6 | 3.8E−02 | 0.29 | 1.6E−01 | LUZP2 | −1.6 | 7.8E−04 | 0.02 | 9.6E−01 |
| ASH2L | 1.6 | 4.6E−02 | 0.45 | 3.4E−02 | PMPCA | −1.6 | 5.6E−03 | 0.30 | 3.0E−01 |
| BICRA | 1.6 | 2.8E−02 | 0.55 | 1.1E−02 | FBXL15 | −1.6 | 2.7E−02 | 0.10 | 7.8E−01 |
| FYTTD1 | 1.6 | 2.7E−02 | 0.38 | 7.0E−02 | PSKH1 | −1.6 | 2.8E−02 | −0.40 | 1.3E−01 |
| TMEM41B | 1.6 | 1.9E−02 | 0.39 | 6.5E−02 | LRRC57 | −1.6 | 2.6E−02 | −0.04 | 9.2E−01 |
| CDC42EP4 | 1.6 | 4.7E−02 | 0.41 | 5.4E−02 | ZNF768 | −1.6 | 1.9E−02 | 0.00 | 9.9E−01 |
| SPARC | 1.6 | 3.2E−02 | 0.49 | 2.2E−02 | SLC10A5 | −1.6 | 2.1E−02 | −0.26 | 3.9E−01 |
| LARP4B | 1.6 | 2.5E−02 | 0.28 | 1.8E−01 | LANCL3 | −1.6 | 5.2E−03 | 0.09 | 8.1E−01 |
| THBD | 1.6 | 2.2E−02 | 0.43 | 4.2E−02 | USH1C | −1.6 | 3.3E−02 | −0.03 | 9.3E−01 |
| LRRC41 | 1.6 | 2.8E−02 | 0.40 | 6.2E−02 | SPON1 | −1.6 | 4.6E−02 | −0.43 | 1.0E−01 |
| C1GALT1 | 1.6 | 2.4E−02 | 0.39 | 6.3E−02 | TRIM45 | −1.6 | 3.8E−02 | −0.16 | 6.2E−01 |
| FGGY | 1.6 | 2.7E−02 | 0.24 | 2.3E−01 | SCAP | −1.6 | 1.1E−02 | −0.14 | 6.8E−01 |
| ESS2 | 1.6 | 3.8E−02 | 0.22 | 2.8E−01 | ABHD17C | −1.6 | 2.1E−02 | 0.06 | 8.7E−01 |
| BAHD1 | 1.6 | 3.7E−02 | 0.54 | 1.1E−02 | KCNE2 | −1.6 | 6.6E−03 | −0.08 | 8.4E−01 |
| AP3S1 | 1.6 | 4.6E−02 | 0.59 | 4.1E−03 | CAT | −1.6 | 3.9E−04 | 0.48 | 5.3E−02 |
| KHDC4 | 1.6 | 3.8E−02 | 0.49 | 2.1E−02 | KIAA1671 | −1.6 | 3.3E−03 | 0.27 | 3.6E−01 |
| PPP1R2 | 1.6 | 5.0E−02 | 0.16 | 4.3E−01 | SRGAP1 | −1.6 | 9.2E−03 | −0.40 | 1.4E−01 |
| ARRDC1 | 1.6 | 3.1E−02 | 0.31 | 1.4E−01 | HSD17B11 | −1.6 | 4.2E−03 | −0.08 | 8.3E−01 |
| MAPRE1 | 1.6 | 3.6E−02 | 0.16 | 4.2E−01 | PGM1 | −1.6 | 1.3E−02 | 0.19 | 5.4E−01 |
| EIF4A2 | 1.6 | 2.7E−02 | 0.15 | 4.7E−01 | PANK3 | −1.6 | 3.3E−02 | 0.54 | 2.5E−02 |
| FTL | 1.6 | 3.1E−02 | 0.50 | 2.0E−02 | NR1H3 | −1.6 | 3.5E−02 | 0.20 | 5.2E−01 |
| INF2 | 1.6 | 1.0E−02 | 0.48 | 2.3E−02 | TFRC | −1.6 | 4.6E−02 | 0.29 | 3.2E−01 |
| ADGRE5 | 1.6 | 4.0E−02 | 0.14 | 5.1E−01 | ZBED1 | −1.6 | 2.6E−03 | 0.26 | 3.9E−01 |
| KDM1A | 1.6 | 4.9E−02 | 0.35 | 9.6E−02 | PET117 | −1.6 | 1.1E−02 | 0.09 | 8.1E−01 |
| AZIN1 | 1.6 | 4.4E−02 | 0.39 | 6.6E−02 | NME7 | −1.6 | 2.2E−02 | −0.27 | 3.6E−01 |
| UQCRH | 1.6 | 4.8E−02 | −0.01 | 9.7E−01 | FOXN3 | −1.6 | 9.5E−03 | 0.11 | 7.4E−01 |
| ARNT | 1.6 | 5.0E−02 | 0.34 | 1.1E−01 | NDUFA10 | −1.6 | 2.1E−02 | 0.16 | 6.1E−01 |
| ABR | 1.5 | 2.9E−02 | 0.25 | 2.1E−01 | MUL1 | −1.6 | 2.7E−02 | −0.10 | 7.8E−01 |
| BCAR1 | 1.5 | 4.3E−02 | −0.04 | 8.6E−01 | TRAK2 | −1.6 | 2.1E−03 | −0.01 | 9.8E−01 |
| GHITM | 1.5 | 3.7E−02 | 0.43 | 4.4E−02 | MAPK3 | −1.6 | 3.5E−03 | 0.09 | 8.1E−01 |
| NAGA | 1.5 | 3.1E−02 | 0.06 | 7.9E−01 | SSUH2 | −1.6 | 4.7E−02 | −0.24 | 4.2E−01 |
| TM7SF3 | 1.5 | 1.5E−02 | 0.14 | 5.0E−01 | MT-CYB | −1.6 | 2.4E−02 | −0.41 | 1.2E−01 |
| HNRNPA3 | 1.5 | 4.2E−02 | 0.30 | 1.6E−01 | PLPP1 | −1.6 | 3.3E−02 | 0.53 | 2.7E−02 |
| RPL38 | 1.5 | 4.3E−03 | 0.23 | 2.6E−01 | DAB2IP | −1.6 | 2.1E−02 | 0.41 | 1.2E−01 |
| HMGN1 | 1.5 | 1.6E−02 | 0.05 | 8.0E−01 | RAD52 | −1.6 | 4.0E−02 | −0.07 | 8.4E−01 |
| PSMA1 | 1.5 | 7.0E−03 | 0.29 | 1.6E−01 | TDRD10 | −1.6 | 2.2E−02 | −0.10 | 7.9E−01 |
| RNF13 | 1.5 | 1.9E−02 | 0.23 | 2.6E−01 | THRA | −1.6 | 9.0E−04 | 0.31 | 3.0E−01 |
| VCPIP1 | 1.5 | 1.4E−02 | 0.21 | 3.1E−01 | PRSS36 | −1.6 | 1.3E−02 | −0.13 | 6.9E−01 |
| MYRF | 1.5 | 8.0E−03 | 0.41 | 5.2E−02 | DNAJC27 | −1.6 | 1.8E−02 | −0.17 | 5.9E−01 |
| YTHDF1 | 1.5 | 4.9E−02 | 0.05 | 8.2E−01 | GP2 | −1.6 | 1.8E−03 | −0.28 | 3.3E−01 |
| SPINT1 | 1.5 | 2.3E−02 | 0.26 | 2.0E−01 | GALNT12 | −1.6 | 6.5E−03 | 0.05 | 9.0E−01 |
| SPTSSA | 1.5 | 4.0E−02 | 0.18 | 3.8E−01 | GNMT | −1.6 | 3.8E−02 | 0.01 | 9.8E−01 |
| SRSF7 | 1.5 | 5.0E−02 | 0.00 | 9.8E−01 | RAD9A | −1.6 | 4.8E−03 | −0.05 | 9.0E−01 |
| PFDN2 | 1.5 | 4.4E−02 | 0.17 | 4.1E−01 | GRB7 | −1.6 | 2.9E−02 | 0.26 | 3.8E−01 |
| GLS | 1.5 | 3.2E−02 | 0.38 | 7.4E−02 | AAMDC | −1.6 | 4.6E−02 | −0.22 | 4.6E−01 |
| ERGIC1 | 1.5 | 3.9E−02 | 0.32 | 1.3E−01 | LNPK | −1.6 | 8.8E−03 | 0.18 | 5.7E−01 |
| SLC35A1 | 1.5 | 3.1E−02 | 0.09 | 6.5E−01 | SLC25A20 | −1.6 | 3.5E−02 | 0.22 | 4.8E−01 |
| KRT18 | −1.5 | 3.2E−02 | −0.51 | 1.7E−02 | SCNN1A | −1.6 | 3.8E−02 | −0.06 | 8.8E−01 |
| NAP1L4 | −1.5 | 4.9E−03 | −0.49 | 2.1E−02 | IQSEC2 | −1.6 | 1.2E−02 | −0.15 | 6.4E−01 |
| NEDD4L | −1.5 | 3.8E−02 | −0.67 | 1.0E−03 | CLCN2 | −1.6 | 3.8E−02 | −0.01 | 9.8E−01 |
| AFDN | −1.5 | 1.9E−02 | −0.25 | 2.2E−01 | RITA1 | −1.6 | 3.4E−02 | 0.03 | 9.4E−01 |
| MARVELD2 | −1.5 | 3.0E−02 | 0.12 | 5.6E−01 | ABCD3 | −1.6 | 3.7E−02 | 0.53 | 2.8E−02 |
| DCAF8 | −1.5 | 8.4E−03 | −0.26 | 2.0E−01 | SDCBP2 | −1.6 | 3.3E−02 | −0.10 | 7.8E−01 |
| DENND4C | −1.5 | 4.3E−02 | −0.05 | 8.0E−01 | NUBPL | −1.6 | 1.6E−02 | −0.36 | 2.1E−01 |
| SRP68 | −1.5 | 4.1E−02 | −0.22 | 2.8E−01 | ATP51 | −1.7 | 3.8E−02 | −0.32 | 2.8E−01 |
| FUBP3 | −1.5 | 3.1E−02 | −0.36 | 9.1E−02 | DDR1 | −1.7 | 9.5E−03 | −0.07 | 8.4E−01 |
| STT3B | −1.5 | 2.8E−02 | −0.05 | 8.3E−01 | MLYCD | −1.7 | 4.0E−04 | −0.08 | 8.3E−01 |
| USP42 | −1.5 | 2.6E−02 | −0.09 | 6.6E−01 | TBC1D3 | −1.7 | 3.9E−02 | 0.00 | 9.9E−01 |
| CHMP3 | −1.5 | 5.4E−03 | −0.41 | 5.5E−02 | CHP1 | −1.7 | 3.2E−03 | 0.53 | 2.8E−02 |
| SCO1 | −1.5 | 2.1E−02 | 0.28 | 1.7E−01 | TIMM13 | −1.7 | 2.6E−02 | −0.29 | 3.2E−01 |
| PPHLN1 | −1.5 | 1.2E−02 | −0.08 | 6.9E−01 | SLC1A7 | −1.7 | 1.6E−04 | 0.41 | 1.2E−01 |
| BRD9 | −1.5 | 2.2E−02 | −0.46 | 3.2E−02 | AUTS2 | −1.7 | 5.1E−03 | −0.09 | 8.1E−01 |
| DYNC1L12 | −1.5 | 2.7E−02 | −0.16 | 4.4E−01 | MSI1 | −1.7 | 7.4E−03 | 0.18 | 5.7E−01 |
| ZNF318 | −1.5 | 4.3E−02 | −0.29 | 1.7E−01 | ANKRD62 | −1.7 | 1.6E−02 | −0.26 | 3.9E−01 |
| SLTM | −1.5 | 3.8E−02 | −0.40 | 5.7E−02 | GLYCTK | −1.7 | 4.1E−02 | 0.25 | 4.1E−01 |
| PEAK1 | −1.5 | 2.1E−02 | −0.15 | 4.7E−01 | AC058822.1 | −1.7 | 3.2E−02 | −0.02 | 9.7E−01 |
| BTBD7 | −1.5 | 2.9E−02 | −0.31 | 1.4E−01 | MAEL | −1.7 | 2.8E−02 | 0.25 | 4.0E−01 |
| RAB8A | −1.5 | 2.3E−02 | −0.28 | 1.8E−01 | COLEC11 | −1.7 | 1.7E−02 | −0.06 | 8.8E−01 |
| OLA1 | −1.5 | 3.3E−02 | −0.37 | 8.3E−02 | UQCRC1 | −1.7 | 5.0E−02 | −0.19 | 5.3E−01 |
| SUGT1 | −1.5 | 3.3E−02 | −0.27 | 1.8E−01 | IQGAP2 | −1.7 | 1.7E−02 | 0.34 | 2.4E−01 |
| SAP30BP | −1.5 | 1.2E−02 | −0.40 | 5.8E−02 | MYRIP | −1.7 | 2.4E−02 | −0.19 | 5.4E−01 |
| UBN1 | −1.5 | 8.8E−03 | −0.33 | 1.2E−01 | IDH3G | −1.7 | 2.1E−02 | −0.25 | 4.0E−01 |
| TLK2 | −1.5 | 8.5E−03 | −0.23 | 2.7E−01 | TMCC3 | −1.7 | 2.2E−02 | 0.26 | 3.9E−01 |
| SRSF11 | −1.5 | 1.3E−02 | −0.55 | 1.1E−02 | HMGCL | −1.7 | 2.4E−02 | 0.41 | 1.2E−01 |
| PSMC1 | −1.5 | 4.0E−02 | −0.54 | 1.1E−02 | AP3S2 | −1.7 | 1.2E−02 | −0.08 | 8.3E−01 |
| BRCC3 | −1.5 | 3.2E−02 | −0.28 | 1.8E−01 | KCNQ1 | −1.7 | 8.8E−03 | −0.30 | 3.0E−01 |
| ETFB | −1.5 | 4.7E−02 | −0.27 | 1.9E−01 | TMEM120A | −1.7 | 4.2E−03 | −0.31 | 2.8E−01 |
| STK39 | −1.5 | 3.9E−02 | 0.18 | 3.8E−01 | RNF223 | −1.7 | 1.9E−02 | 0.03 | 9.3E−01 |
| CALM1 | −1.5 | 1.3E−02 | −0.26 | 2.1E−01 | MAGI1 | −1.7 | 5.2E−05 | 0.39 | 1.4E−01 |
| POLR2J3 | −1.5 | 2.2E−02 | −0.10 | 6.1E−01 | NRGN | −1.7 | 1.9E−02 | 0.27 | 3.7E−01 |
| ZC3H18 | −1.5 | 3.5E−02 | −0.18 | 3.7E−01 | HIGD1A | −1.7 | 3.1E−02 | 0.00 | 9.9E−01 |
| ZNF326 | −1.6 | 2.6E−02 | −0.29 | 1.6E−01 | CRYBB2 | −1.7 | 2.9E−02 | −0.05 | 8.9E−01 |
| GOLGA2 | −1.6 | 4.1E−02 | −0.32 | 1.3E−01 | C1orf115 | −1.7 | 1.5E−02 | 0.62 | 8.9E−03 |
| CHCHD10 | −1.6 | 2.4E−02 | −0.37 | 8.3E−02 | TMEM82 | −1.7 | 4.1E−02 | −0.13 | 7.1E−01 |
| EIF4E2 | −1.6 | 2.3E−02 | −0.39 | 6.3E−02 | AP1M2 | −1.7 | 4.3E−02 | 0.18 | 5.6E−01 |
| TLN1 | −1.6 | 3.2E−02 | −0.14 | 4.8E−01 | ACKR2 | −1.7 | 2.6E−03 | 0.05 | 9.0E−01 |
| TAF15 | −1.6 | 2.6E−02 | −0.50 | 1.8E−02 | ERBB3 | −1.7 | 2.6E−02 | 0.25 | 3.9E−01 |
| BRD2 | −1.6 | 2.8E−02 | −0.65 | 1.7E−03 | RAB40C | −1.7 | 2.9E−02 | 0.07 | 8.4E−01 |
| KRIT1 | −1.6 | 5.7E−03 | −0.35 | 1.0E−01 | TMEM63C | −1.7 | 9.5E−03 | −0.17 | 5.9E−01 |
| TFCP2L1 | −1.6 | 1.5E−02 | −0.67 | 1.0E−03 | DENND4C | −1.7 | 1.5E−03 | −0.05 | 8.9E−01 |
| ACOT11 | −1.6 | 3.9E−02 | −0.32 | 1.3E−01 | DDT | −1.7 | 2.5E−04 | −0.20 | 5.2E−01 |
| CHCHD2 | −1.6 | 2.3E−02 | −0.49 | 2.0E−02 | ZDHHC23 | −1.7 | 3.7E−02 | −0.14 | 6.6E−01 |
| ABCF1 | −1.6 | 1.4E−02 | −0.28 | 1.8E−01 | FAM229A | −1.7 | 2.1E−02 | 0.17 | 5.8E−01 |
| SH3GLB2 | −1.6 | 3.8E−02 | −0.38 | 7.4E−02 | C4orf19 | −1.7 | 3.2E−02 | −0.05 | 9.0E−01 |
| KIAA0141 | −1.6 | 4.2E−02 | −0.54 | 1.1E−02 | GKN2 | −1.7 | 4.9E−03 | −0.35 | 2.1E−01 |
| EPM2AIP1 | −1.6 | 3.9E−02 | −0.26 | 2.0E−01 | EIF4EBP2 | −1.7 | 2.4E−04 | 0.55 | 2.1E−02 |
| SRRM1 | −1.6 | 5.0E−02 | −0.26 | 2.1E−01 | VDR | −1.7 | 1.5E−02 | −0.69 | 2.9E−03 |
| RNF168 | −1.6 | 4.6E−02 | −0.06 | 7.8E−01 | ENDOD1 | −1.7 | 4.1E−02 | −0.62 | 8.4E−03 |
| CPSF2 | −1.6 | 3.2E−02 | −0.13 | 5.2E−01 | LAMTOR4 | −1.7 | 1.9E−02 | −0.60 | 1.1E−02 |
| SARS | −1.6 | 3.5E−02 | −0.46 | 3.2E−02 | SERF1B | −1.7 | 5.6E−03 | −0.02 | 9.7E−01 |
| NOP56 | −1.6 | 2.3E−02 | −0.41 | 5.2E−02 | PRADC1 | −1.7 | 3.3E−02 | −0.19 | 5.3E−01 |
| FBXW7 | −1.6 | 1.2E−02 | −0.30 | 1.6E−01 | ZNF677 | −1.7 | 4.7E−02 | −0.23 | 4.4E−01 |
| NCOR1 | −1.6 | 3.7E−03 | −0.19 | 3.5E−01 | GDE1 | −1.7 | 3.9E−03 | 0.28 | 3.4E−01 |
| ENAH | −1.6 | 2.0E−02 | −0.48 | 2.3E−02 | FITM1 | −1.7 | 3.3E−02 | −0.29 | 3.2E−01 |
| OS9 | −1.6 | 2.4E−02 | −0.27 | 1.8E−01 | CCDC15 | −1.7 | 3.7E−02 | 0.06 | 8.8E−01 |
| ZCRB1 | −1.6 | 8.1E−03 | −0.05 | 8.0E−01 | COMMD9 | −1.7 | 1.0E−03 | −0.17 | 5.9E−01 |
| TTC1 | −1.6 | 2.2E−02 | −0.35 | 1.0E−01 | SFXN5 | −1.7 | 3.2E−02 | −0.15 | 6.4E−01 |
| CMTM6 | −1.6 | 3.5E−02 | −0.27 | 1.8E−01 | CXorf56 | −1.7 | 2.4E−02 | 0.11 | 7.6E−01 |
| PPIG | −1.6 | 2.2E−02 | −0.54 | 1.1E−02 | ZNF525 | −1.7 | 1.0E−02 | −0.22 | 4.7E−01 |
| NEMF | −1.6 | 2.4E−02 | −0.27 | 1.9E−01 | SLC19A1 | −1.7 | 2.5E−03 | −0.21 | 5.0E−01 |
| GGNBP2 | −1.6 | 1.9E−02 | −0.47 | 2.6E−02 | ECHS1 | −1.7 | 6.9E−03 | 0.24 | 4.2E−01 |
| CTR9 | −1.6 | 1.2E−02 | −0.31 | 1.3E−01 | EXD3 | −1.7 | 4.1E−02 | −0.21 | 5.0E−01 |
| FIP1L1 | −1.6 | 2.4E−02 | −0.55 | 9.7E−03 | BMP1 | −1.7 | 2.8E−02 | −0.13 | 7.1E−01 |
| SPOP | −1.6 | 1.7E−02 | −0.39 | 6.6E−02 | MXI1 | −1.7 | 4.3E−02 | 0.12 | 7.2E−01 |
| BAZ2A | −1.6 | 1.0E−02 | −0.05 | 8.2E−01 | HDHD3 | −1.7 | 4.0E−02 | 0.47 | 5.8E−02 |
| MCU | −1.6 | 4.3E−02 | −0.60 | 3.7E−03 | ACO2 | −1.7 | 2.5E−03 | 0.14 | 6.8E−01 |
| TPRKB | −1.6 | 4.2E−02 | −0.29 | 1.6E−01 | DDAH1 | −1.7 | 6.4E−03 | −0.49 | 5.1E−02 |
| COMMD1 | −1.6 | 2.7E−02 | −0.21 | 2.9E−01 | THG1L | −1.7 | 8.1E−03 | 0.04 | 9.1E−01 |
| SLU7 | −1.6 | 1.0E−02 | −0.40 | 6.0E−02 | ARL6IP6 | −1.7 | 1.5E−02 | 0.20 | 5.2E−01 |
| SSRP1 | −1.6 | 7.2E−03 | −0.40 | 6.0E−02 | ATP9A | −1.7 | 5.2E−05 | 0.20 | 5.2E−01 |
| EZR | −1.6 | 1.9E−02 | 0.20 | 3.3E−01 | PCK2 | −1.7 | 2.0E−02 | 0.11 | 7.6E−01 |
| UBE2E1 | −1.6 | 2.3E−02 | −0.51 | 1.6E−02 | CBSL | −1.7 | 2.9E−02 | 0.09 | 8.0E−01 |
| CLIP1 | −1.6 | 4.2E−02 | −0.31 | 1.4E−01 | PSMD3 | −1.7 | 2.9E−02 | −0.21 | 5.0E−01 |
| CALCOCO2 | −1.6 | 2.4E−02 | −0.32 | 1.2E−01 | NOMO3 | −1.7 | 3.6E−02 | −0.11 | 7.5E−01 |
| NUPR1 | −1.6 | 2.1E−02 | −0.62 | 2.9E−03 | CACFD1 | −1.7 | 1.7E−02 | −0.17 | 5.9E−01 |
| ABHD15 | −1.6 | 9.5E−03 | 0.21 | 3.0E−01 | SUCLG1 | −1.7 | 1.5E−02 | −0.22 | 4.7E−01 |
| RPN2 | −1.6 | 3.5E−02 | −0.38 | 7.6E−02 | SORBS3 | −1.7 | 4.4E−02 | 0.15 | 6.4E−01 |
| FRG1 | −1.6 | 2.0E−02 | −0.30 | 1.5E−01 | PLXNA2 | −1.7 | 2.3E−02 | 0.12 | 7.3E−01 |
| ZC3H13 | −1.6 | 4.0E−02 | −0.28 | 1.7E−01 | GTPBP6 | −1.7 | 3.6E−02 | −0.09 | 8.1E−01 |
| YKT6 | −1.6 | 2.2E−02 | −0.50 | 1.9E−02 | AVPI1 | −1.7 | 9.7E−03 | 0.16 | 6.1E−01 |
| ESF1 | −1.6 | 9.4E−03 | −0.53 | 1.2E−02 | AK3 | −1.7 | 5.5E−04 | 0.24 | 4.2E−01 |
| SRPK2 | −1.6 | 2.4E−02 | −0.15 | 4.5E−01 | CYTL1 | −1.7 | 1.1E−02 | 0.04 | 9.2E−01 |
| CHD3 | −1.6 | 8.5E−03 | −0.29 | 1.6E−01 | PEX26 | −1.7 | 1.1E−02 | −0.22 | 4.7E−01 |
| ANKS4B | −1.6 | 3.0E−02 | −0.30 | 1.5E−01 | PIGB | −1.7 | 3.0E−02 | 0.12 | 7.1E−01 |
| TRPM7 | −1.6 | 1.4E−02 | −0.02 | 9.4E−01 | SFXN1 | −1.7 | 8.2E−03 | 0.51 | 3.8E−02 |
| CTBP2 | −1.6 | 1.2E−02 | −0.32 | 1.3E−01 | NDUFV1 | −1.7 | 2.8E−02 | −0.33 | 2.5E−01 |
| DYNC1I2 | −1.6 | 1.5E−02 | −0.53 | 1.2E−02 | HNF1B | −1.7 | 3.2E−02 | −0.11 | 7.6E−01 |
| RWDD2B | −1.6 | 2.8E−02 | −0.12 | 5.7E−01 | LRRN2 | −1.7 | 4.3E−03 | 0.07 | 8.5E−01 |
| RPS12 | −1.6 | 4.2E−02 | −0.69 | 7.3E−04 | METTL7A | −1.7 | 1.9E−02 | 0.22 | 4.7E−01 |
| NMT1 | −1.6 | 1.9E−02 | −0.44 | 3.8E−02 | RAP1GAP | −1.7 | 4.7E−02 | −0.17 | 5.9E−01 |
| SLC12A2 | −1.6 | 2.7E−02 | −0.23 | 2.6E−01 | AMN | −1.7 | 4.7E−02 | 0.02 | 9.6E−01 |
| BRD4 | −1.6 | 1.0E−02 | −0.12 | 5.5E−01 | BDH1 | −1.7 | 4.9E−03 | 0.08 | 8.3E−01 |
| SFMBT1 | −1.6 | 3.1E−02 | −0.29 | 1.6E−01 | MT-CO1 | −1.7 | 5.2E−05 | 0.36 | 2.0E−01 |
| SRRT | −1.6 | 3.8E−02 | −0.71 | 5.0E−04 | EPB41L1 | −1.7 | 1.6E−02 | −0.07 | 8.6E−01 |
| CEBPZOS | −1.6 | 8.5E−03 | −0.56 | 8.3E−03 | MICAL3 | −1.7 | 2.5E−02 | 0.23 | 4.4E−01 |
| RETSAT | −1.6 | 2.2E−02 | −0.39 | 6.5E−02 | MRPS33 | −1.8 | 1.3E−04 | −0.07 | 8.4E−01 |
| DDX46 | −1.6 | 1.1E−02 | −0.56 | 8.5E−03 | GNG4 | −1.8 | 2.6E−02 | −0.28 | 3.5E−01 |
| WDR3 | −1.6 | 7.0E−03 | −0.29 | 1.6E−01 | SLC25A10 | −1.8 | 4.3E−02 | 0.01 | 9.7E−01 |
| ROCK2 | −1.6 | 1.9E−02 | −0.13 | 5.3E−01 | ZNF91 | −1.8 | 1.5E−02 | −0.20 | 5.2E−01 |
| DENND5B | −1.6 | 3.1E−02 | −0.02 | 9.4E−01 | TMEM125 | −1.8 | 2.0E−02 | −0.43 | 1.0E−01 |
| CTNNBL1 | −1.6 | 1.4E−02 | −0.29 | 1.6E−01 | PRDX6 | −1.8 | 4.6E−02 | −0.57 | 1.6E−02 |
| HCFC2 | −1.6 | 2.1E−02 | −0.33 | 1.2E−01 | ADGRA3 | −1.8 | 2.9E−02 | −0.06 | 8.7E−01 |
| UQCRB | −1.6 | 8.5E−03 | −0.64 | 2.2E−03 | ANKMY2 | −1.8 | 9.8E−03 | 0.02 | 9.7E−01 |
| NUBPL | −1.6 | 3.7E−03 | −0.36 | 9.1E−02 | SPATA18 | −1.8 | 2.5E−03 | −0.05 | 8.9E−01 |
| ZRSR2 | −1.6 | 3.3E−02 | −0.48 | 2.3E−02 | GPX3 | −1.8 | 2.8E−02 | 0.16 | 6.1E−01 |
| PUS7L | −1.6 | 2.9E−02 | −0.27 | 1.8E−01 | LGALS4 | −1.8 | 1.1E−02 | −0.40 | 1.3E−01 |
| DDX21 | −1.6 | 1.1E−02 | −0.43 | 4.4E−02 | FAM13A | −1.8 | 5.8E−04 | 0.58 | 1.2E−02 |
| SMARCA4 | −1.6 | 4.2E−02 | −0.42 | 4.7E−02 | ANO9 | −1.8 | 4.4E−02 | −0.06 | 8.6E−01 |
| DDAH1 | −1.7 | 3.6E−02 | −0.49 | 2.3E−02 | KDF1 | −1.8 | 8.3E−03 | −0.05 | 8.9E−01 |
| PARD3 | −1.7 | 1.3E−02 | −0.11 | 6.0E−01 | SRI | −1.8 | 5.7E−03 | 0.48 | 5.3E−02 |
| NDUFA5 | −1.7 | 9.2E−03 | −0.67 | 1.1E−03 | ABHD11 | −1.8 | 1.1E−02 | −0.33 | 2.4E−01 |
| SETBP1 | −1.7 | 4.3E−02 | −0.05 | 8.1E−01 | LIPC | −1.8 | 1.1E−03 | −0.14 | 6.8E−01 |
| IGF2BP2 | −1.7 | 1.5E−02 | −0.09 | 6.7E−01 | TMC4 | −1.8 | 1.1E−02 | 0.25 | 4.1E−01 |
| DCAF7 | −1.7 | 3.9E−03 | −0.21 | 3.0E−01 | NMT2 | −1.8 | 2.0E−02 | −0.06 | 8.7E−01 |
| C1orf131 | −1.7 | 2.3E−02 | −0.38 | 7.7E−02 | PYGB | −1.8 | 1.9E−02 | 0.05 | 9.0E−01 |
| ZMAT2 | −1.7 | 1.8E−02 | −0.20 | 3.1E−01 | SPIRE2 | −1.8 | 2.3E−02 | 0.13 | 7.1E−01 |
| URGCP | −1.7 | 1.2E−02 | −0.29 | 1.7E−01 | TMEM98 | −1.8 | 2.6E−03 | −0.53 | 2.8E−02 |
| PSMD1 | −1.7 | 4.0E−02 | −0.21 | 3.0E−01 | SLC22A18 | −1.8 | 2.2E−02 | −0.24 | 4.2E−01 |
| MON1B | −1.7 | 4.9E−03 | −0.24 | 2.3E−01 | LRP12 | −1.8 | 3.2E−02 | 0.48 | 5.1E−02 |
| NDUFB9 | −1.7 | 6.2E−03 | −0.63 | 2.3E−03 | DTX4 | −1.8 | 1.2E−02 | 0.03 | 9.5E−01 |
| TFG | −1.7 | 2.1E−03 | −0.37 | 8.3E−02 | MYOT | −1.8 | 1.3E−02 | −0.35 | 2.2E−01 |
| TPM4 | −1.7 | 1.9E−02 | −0.24 | 2.4E−01 | CPNE2 | −1.8 | 4.0E−03 | 0.41 | 1.2E−01 |
| CHD2 | −1.7 | 3.7E−02 | −0.18 | 3.8E−01 | CHCHD10 | −1.8 | 1.3E−03 | −0.37 | 1.9E−01 |
| TAF11 | −1.7 | 9.0E−03 | −0.51 | 1.7E−02 | GAL3ST4 | −1.8 | 4.0E−02 | −0.53 | 2.9E−02 |
| DR1 | −1.7 | 4.9E−03 | −0.54 | 1.1E−02 | NR2F6 | −1.8 | 3.4E−03 | −0.29 | 3.2E−01 |
| FAM114A1 | −1.7 | 1.9E−02 | −0.16 | 4.2E−01 | PTGR2 | −1.8 | 2.2E−02 | −0.27 | 3.6E−01 |
| PNISR | −1.7 | 2.9E−02 | −0.46 | 3.2E−02 | GIPC2 | −1.8 | 3.9E−02 | 0.24 | 4.2E−01 |
| DDX24 | −1.7 | 4.2E−03 | −0.15 | 4.6E−01 | ACSS2 | −1.8 | 3.1E−02 | −0.02 | 9.7E−01 |
| CEP41 | −1.7 | 4.9E−02 | −0.31 | 1.4E−01 | PGAP3 | −1.8 | 1.2E−02 | −0.09 | 8.1E−01 |
| ZCCHC6 | −1.7 | 2.4E−02 | −0.18 | 3.9E−01 | EVI5L | −1.8 | 4.7E−02 | 0.08 | 8.3E−01 |
| RRM1 | −1.7 | 4.2E−02 | −0.39 | 6.5E−02 | SREBF2 | −1.8 | 3.4E−03 | −0.19 | 5.4E−01 |
| HSP90AB1 | −1.7 | 1.4E−02 | −0.26 | 2.0E−01 | RRAS2 | −1.8 | 7.2E−03 | 0.33 | 2.4E−01 |
| TSC22D2 | −1.7 | 1.8E−02 | −0.37 | 7.9E−02 | SUCLG2 | −1.8 | 4.5E−02 | 0.38 | 1.6E−01 |
| PRPF6 | −1.7 | 1.8E−02 | −0.53 | 1.2E−02 | SFN | −1.8 | 4.1E−02 | −0.11 | 7.6E−01 |
| YLPM1 | −1.7 | 1.2E−02 | −0.35 | 9.8E−02 | CLYBL | −1.8 | 6.2E−03 | 0.04 | 9.2E−01 |
| PHACTR4 | −1.7 | 1.0E−02 | −0.25 | 2.2E−01 | BAX | −1.8 | 1.8E−03 | 0.02 | 9.7E−01 |
| VTI1A | −1.7 | 1.6E−02 | −0.39 | 6.5E−02 | CPE | −1.8 | 1.9E−02 | −0.20 | 5.2E−01 |
| RTFDC1 | −1.7 | 1.8E−02 | −0.53 | 1.2E−02 | ZNF441 | −1.8 | 2.8E−03 | −0.20 | 5.2E−01 |
| LRRFIP1 | −1.7 | 1.6E−02 | −0.29 | 1.6E−01 | GTF2H2C | −1.8 | 2.5E−02 | −0.03 | 9.4E−01 |
| SMARCA2 | −1.7 | 2.4E−02 | −0.23 | 2.7E−01 | AGPAT2 | −1.8 | 3.5E−02 | 0.32 | 2.7E−01 |
| LYRM2 | −1.7 | 1.7E−02 | −0.40 | 6.2E−02 | SMPDL3B | −1.8 | 3.5E−04 | −0.53 | 2.9E−02 |
| HYI | −1.7 | 3.0E−02 | −0.26 | 2.0E−01 | PTK6 | −1.8 | 3.2E−02 | −0.02 | 9.7E−01 |
| DNASE1 | −1.7 | 4.5E−02 | −0.48 | 2.5E−02 | LMBR1L | −1.8 | 3.2E−02 | 0.23 | 4.5E−01 |
| PARD3B | −1.7 | 4.1E−02 | −0.15 | 4.6E−01 | MT-ND1 | −1.8 | 2.5E−03 | −0.13 | 7.1E−01 |
| CHD6 | −1.7 | 4.2E−02 | −0.25 | 2.1E−01 | MIGA2 | −1.8 | 5.0E−04 | 0.26 | 3.8E−01 |
| CNOT11 | −1.7 | 1.7E−02 | −0.59 | 4.5E−03 | MT-ATP6 | −1.8 | 6.9E−03 | −0.50 | 4.5E−02 |
| C5orf63 | −1.7 | 1.9E−02 | −0.10 | 6.1E−01 | EHHADH | −1.8 | 5.3E−03 | 0.67 | 3.4E−03 |
| DNAJC5 | −1.7 | 8.5E−03 | −0.62 | 2.7E−03 | ARF5 | −1.8 | 4.0E−02 | 0.35 | 2.2E−01 |
| CCDC43 | −1.7 | 4.1E−02 | −0.39 | 6.5E−02 | TRAF3IP2 | −1.8 | 9.0E−03 | 0.13 | 7.1E−01 |
| RPL26 | −1.7 | 1.6E−02 | −0.70 | 5.5E−04 | EVPL | −1.8 | 7.3E−03 | −0.16 | 6.2E−01 |
| POF1B | −1.7 | 4.2E−02 | −0.43 | 4.3E−02 | SLC25A35 | −1.8 | 4.5E−03 | −0.15 | 6.6E−01 |
| PA2G4 | −1.7 | 1.8E−02 | −0.39 | 6.4E−02 | ZNF219 | −1.8 | 3.8E−04 | −0.41 | 1.2E−01 |
| RBM33 | −1.7 | 3.1E−03 | −0.36 | 9.1E−02 | SERINC2 | −1.8 | 7.3E−03 | −0.10 | 7.7E−01 |
| INO80 | −1.7 | 4.2E−03 | −0.30 | 1.5E−01 | C4orf48 | −1.8 | 9.7E−03 | 0.04 | 9.1E−01 |
| MAN1A2 | −1.7 | 3.0E−02 | −0.44 | 3.9E−02 | PLCE1 | −1.8 | 1.9E−02 | −0.07 | 8.5E−01 |
| CEP63 | −1.7 | 3.6E−02 | −0.29 | 1.6E−01 | ANKRD9 | −1.8 | 1.8E−02 | −0.11 | 7.7E−01 |
| STT3A | −1.7 | 3.4E−02 | −0.29 | 1.7E−01 | ADGRL3 | −1.8 | 7.9E−05 | 0.16 | 6.2E−01 |
| ARHGAP11A | −1.7 | 3.2E−02 | −0.29 | 1.6E−01 | ANO10 | −1.8 | 2.2E−03 | 0.08 | 8.4E−01 |
| KIAA0368 | −1.7 | 4.3E−02 | −0.25 | 2.1E−01 | RAPGEFL1 | −1.8 | 1.3E−02 | 0.23 | 4.5E−01 |
| TMEM253 | −1.7 | 2.0E−02 | −0.35 | 9.6E−02 | XK | −1.8 | 4.0E−02 | 0.52 | 3.3E−02 |
| PSMD7 | −1.7 | 9.8E−03 | −0.46 | 3.0E−02 | FAM234A | −1.8 | 1.4E−03 | 0.44 | 8.4E−02 |
| LARP7 | −1.7 | 2.7E−02 | −0.44 | 4.1E−02 | MGAT4B | −1.8 | 1.6E−03 | −0.45 | 8.1E−02 |
| ZNFX1 | −1.7 | 3.2E−02 | −0.43 | 4.2E−02 | CES1 | −1.8 | 4.3E−02 | 0.37 | 1.9E−01 |
| RBBP6 | −1.7 | 1.4E−02 | −0.55 | 9.7E−03 | CYP27A1 | −1.8 | 1.8E−02 | −0.09 | 8.1E−01 |
| HNRNPH1 | −1.7 | 3.3E−02 | −0.33 | 1.2E−01 | TLN2 | −1.8 | 6.8E−04 | −0.32 | 2.8E−01 |
| SCAPER | −1.7 | 3.4E−02 | −0.53 | 1.3E−02 | SLC10A1 | −1.8 | 5.1E−03 | −0.01 | 9.8E−01 |
| DSP | −1.7 | 4.1E−02 | −0.30 | 1.5E−01 | BBIP1 | −1.8 | 2.9E−02 | −0.03 | 9.4E−01 |
| SLC30A9 | −1.7 | 1.4E−02 | −0.52 | 1.5E−02 | MT-ND3 | −1.9 | 2.5E−04 | −0.20 | 5.2E−01 |
| PPCS | −1.7 | 8.6E−03 | −0.27 | 1.8E−01 | ABCA8 | −1.9 | 1.4E−02 | 0.17 | 5.9E−01 |
| SUPT16H | −1.7 | 6.2E−03 | −0.37 | 8.4E−02 | SF3A2 | −1.9 | 3.8E−02 | −0.10 | 8.0E−01 |
| NIPBL | −1.7 | 3.3E−03 | −0.27 | 1.9E−01 | MT-CO3 | −1.9 | 7.6E−03 | 0.02 | 9.6E−01 |
| SLC37A4 | −1.7 | 6.5E−03 | −0.46 | 3.2E−02 | POU5F1B | −1.9 | 1.2E−02 | 0.10 | 7.9E−01 |
| POLE3 | −1.7 | 3.2E−02 | −0.70 | 5.5E−04 | MLX1P | −1.9 | 7.3E−04 | 0.25 | 4.1E−01 |
| C22orf46 | −1.7 | 1.6E−02 | −0.25 | 2.1E−01 | ABHD5 | −1.9 | 2.0E−02 | 0.32 | 2.8E−01 |
| CEP78 | −1.7 | 3.9E−02 | −0.34 | 1.1E−01 | TFCP2L1 | −1.9 | 8.8E−03 | −0.67 | 3.4E−03 |
| NASP | −1.7 | 7.6E−03 | −0.48 | 2.5E−02 | RAVER2 | −1.9 | 1.3E−02 | 0.16 | 6.2E−01 |
| EIF5B | −1.7 | 4.9E−03 | −0.49 | 2.1E−02 | GSTZ1 | −1.9 | 4.0E−02 | −0.14 | 6.6E−01 |
| KMT2B | −1.7 | 4.2E−02 | −0.32 | 1.3E−01 | EMILIN3 | −1.9 | 2.0E−02 | −0.20 | 5.2E−01 |
| CCDC82 | −1.7 | 2.9E−02 | −0.32 | 1.3E−01 | PHYH | −1.9 | 4.3E−03 | 0.24 | 4.2E−01 |
| ARL11 | −1.7 | 3.2E−02 | −0.13 | 5.3E−01 | TMEM100 | −1.9 | 1.2E−02 | 0.38 | 1.7E−01 |
| ARHGEF3 | −1.7 | 1.1E−02 | −0.51 | 1.5E−02 | FAM183A | −1.9 | 4.0E−02 | −0.06 | 8.7E−01 |
| ACADS | −1.8 | 3.9E−03 | −0.12 | 5.5E−01 | THAP3 | −1.9 | 2.5E−02 | −0.09 | 8.0E−01 |
| PPP2R2A | −1.8 | 5.0E−03 | −0.32 | 1.3E−01 | SH2D6 | −1.9 | 4.1E−02 | −0.18 | 5.7E−01 |
| RSRC1 | −1.8 | 9.3E−03 | −0.59 | 4.6E−03 | ACOT11 | −1.9 | 4.7E−04 | −0.32 | 2.7E−01 |
| MYLK | −1.8 | 2.1E−02 | −0.65 | 1.7E−03 | BSG | −1.9 | 4.9E−02 | −0.14 | 6.7E−01 |
| MFHAS1 | −1.8 | 1.1E−02 | −0.48 | 2.3E−02 | HOXB5 | −1.9 | 3.9E−02 | 0.56 | 1.9E−02 |
| NRP1 | −1.8 | 4.3E−02 | −0.10 | 6.3E−01 | MT-ND4L | −1.9 | 1.7E−02 | −0.31 | 2.8E−01 |
| MRE11 | −1.8 | 2.5E−02 | −0.31 | 1.4E−01 | FMO5 | −1.9 | 3.4E−02 | −0.04 | 9.2E−01 |
| METAP2 | −1.8 | 4.8E−03 | −0.32 | 1.3E−01 | GNA11 | −1.9 | 1.5E−02 | 0.34 | 2.4E−01 |
| PDE9A | −1.8 | 4.9E−02 | −0.10 | 6.4E−01 | HSPB1 | −1.9 | 3.6E−02 | 0.59 | 1.2E−02 |
| CTCF | −1.8 | 1.5E−02 | −0.31 | 1.4E−01 | BCAS1 | −1.9 | 1.9E−02 | −0.55 | 2.3E−02 |
| CDC37 | −1.8 | 1.6E−02 | −0.50 | 2.0E−02 | ODF3L1 | −1.9 | 1.1E−02 | 0.06 | 8.8E−01 |
| AUP1 | −1.8 | 3.5E−02 | −0.73 | 2.8E−04 | ETHE1 | −1.9 | 8.3E−03 | −0.41 | 1.2E−01 |
| PSMD4 | −1.8 | 2.5E−02 | −0.53 | 1.1E−02 | MAST2 | −1.9 | 8.4E−05 | 0.28 | 3.3E−01 |
| DSN1 | −1.8 | 1.8E−02 | −0.26 | 2.0E−01 | PLIN2 | −1.9 | 2.6E−02 | 0.30 | 3.0E−01 |
| CHD7 | −1.8 | 1.6E−02 | −0.22 | 2.9E−01 | NR5A2 | −1.9 | 3.7E−02 | 0.03 | 9.5E−01 |
| LTV1 | −1.8 | 2.7E−02 | −0.33 | 1.2E−01 | CRNN | −1.9 | 4.2E−02 | 0.29 | 3.2E−01 |
| DGKA | −1.8 | 4.4E−02 | −0.39 | 6.4E−02 | TTR | −1.9 | 2.5E−03 | −0.32 | 2.7E−01 |
| PRRC2C | −1.8 | 2.4E−02 | −0.26 | 2.0E−01 | ABHD15 | −1.9 | 6.2E−04 | 0.21 | 5.0E−01 |
| PNPO | −1.8 | 1.5E−02 | −0.44 | 4.1E−02 | RAB17 | −1.9 | 2.0E−03 | −0.17 | 5.8E−01 |
| CCNA2 | −1.8 | 1.7E−02 | −0.42 | 4.8E−02 | CAMK2N1 | −1.9 | 3.0E−02 | −0.14 | 6.7E−01 |
| ZNF219 | −1.8 | 1.8E−02 | −0.41 | 5.5E−02 | ZNF701 | −1.9 | 1.3E−02 | 0.00 | 1.0E+00 |
| FAM49B | −1.8 | 3.8E−02 | −0.28 | 1.7E−01 | PRR26 | −1.9 | 3.2E−04 | −0.23 | 4.5E−01 |
| MFAP1 | −1.8 | 3.9E−03 | −0.33 | 1.1E−01 | ANKS4B | −1.9 | 2.2E−02 | −0.30 | 3.0E−01 |
| CEP170B | −1.8 | 3.1E−02 | −0.60 | 3.6E−03 | TTC38 | −1.9 | 4.0E−02 | −0.25 | 4.0E−01 |
| FAAP20 | −1.8 | 4.0E−02 | −0.28 | 1.8E−01 | ESPN | −1.9 | 4.0E−03 | 0.18 | 5.5E−01 |
| EIF6 | −1.8 | 3.3E−02 | −0.44 | 4.0E−02 | CPEB3 | −1.9 | 2.1E−03 | −0.07 | 8.5E−01 |
| PSME2 | −1.8 | 2.2E−02 | −0.35 | 9.3E−02 | FAH | −1.9 | 1.8E−03 | −0.20 | 5.2E−01 |
| OPA3 | −1.8 | 3.1E−02 | 0.01 | 9.6E−01 | OPLAH | −1.9 | 2.0E−02 | 0.11 | 7.4E−01 |
| ANKRD36 | −1.8 | 4.0E−02 | −0.28 | 1.8E−01 | SLC9A3R1 | −1.9 | 7.5E−04 | 0.24 | 4.3E−01 |
| PSMC6 | −1.8 | 8.1E−03 | −0.14 | 4.8E−01 | MYO1D | −1.9 | 9.8E−03 | 0.24 | 4.2E−01 |
| TCF25 | −1.8 | 3.0E−03 | −0.32 | 1.2E−01 | DIO3 | −1.9 | 1.2E−02 | −0.58 | 1.3E−02 |
| TNPO1 | −1.8 | 3.3E−03 | −0.34 | 1.0E−01 | CLDN3 | −1.9 | 2.8E−02 | −0.15 | 6.6E−01 |
| MPHOSPH9 | −1.8 | 2.4E−02 | −0.42 | 4.8E−02 | EFNA1 | −1.9 | 2.4E−02 | 0.31 | 2.9E−01 |
| DDX27 | −1.8 | 4.2E−03 | −0.40 | 6.0E−02 | AOC1 | −1.9 | 1.4E−02 | 0.01 | 9.8E−01 |
| RAB11FIP2 | −1.8 | 1.9E−02 | −0.26 | 2.0E−01 | MT-ND4 | −1.9 | 3.6E−04 | −0.52 | 3.1E−02 |
| CDK16 | −1.8 | 3.8E−02 | −0.02 | 9.4E−01 | CHDH | −1.9 | 2.9E−03 | −0.30 | 3.1E−01 |
| DROSHA | −1.8 | 4.2E−03 | −0.55 | 1.1E−02 | PTGR1 | −1.9 | 1.2E−03 | 0.40 | 1.3E−01 |
| NEU3 | −1.8 | 5.0E−02 | −0.03 | 9.0E−01 | PNPLA2 | −1.9 | 9.4E−04 | −0.08 | 8.3E−01 |
| PPFIBP2 | −1.8 | 1.3E−02 | −0.31 | 1.4E−01 | GPRC5C | −1.9 | 1.9E−03 | 0.08 | 8.2E−01 |
| HMGB2 | −1.8 | 3.3E−02 | −0.28 | 1.8E−01 | OSBPL1A | −2.0 | 2.5E−03 | 0.24 | 4.2E−01 |
| KCNQ1 | −1.8 | 1.5E−02 | −0.30 | 1.5E−01 | TSPAN7 | −2.0 | 3.4E−03 | 0.05 | 8.9E−01 |
| PGRMC2 | −1.8 | 6.4E−04 | −0.28 | 1.7E−01 | MARVELD3 | −2.0 | 2.4E−03 | 0.39 | 1.5E−01 |
| WSB2 | −1.8 | 2.7E−02 | −0.19 | 3.6E−01 | MGLL | −2.0 | 2.9E−03 | 0.49 | 4.9E−02 |
| NOP14 | −1.8 | 1.4E−02 | −0.30 | 1.6E−01 | HHLA2 | −2.0 | 2.6E−02 | −0.63 | 7.8E−03 |
| GON4L | −1.8 | 5.0E−03 | −0.29 | 1.6E−01 | EPHX2 | −2.0 | 6.1E−03 | −0.09 | 8.1E−01 |
| ZNF141 | −1.8 | 3.4E−02 | 0.13 | 5.2E−01 | VIL1 | −2.0 | 2.5E−02 | −0.37 | 1.8E−01 |
| SORD | −1.8 | 2.6E−02 | −0.32 | 1.3E−01 | CXorf40A | −2.0 | 1.7E−02 | −0.27 | 3.6E−01 |
| WDR60 | −1.8 | 3.0E−02 | −0.42 | 4.8E−02 | TGFA | −2.0 | 1.5E−03 | 0.06 | 8.7E−01 |
| WDR92 | −1.8 | 2.7E−02 | −0.36 | 8.7E−02 | PDCD4 | −2.0 | 1.4E−02 | −0.05 | 8.9E−01 |
| MEA1 | −1.8 | 2.1E−02 | −0.57 | 6.8E−03 | CPT1A | −2.0 | 1.3E−03 | 0.36 | 2.1E−01 |
| SPTLC2 | −1.8 | 2.3E−03 | −0.12 | 5.5E−01 | ZNF341 | −2.0 | 7.4E−03 | 0.15 | 6.4E−01 |
| MYO1A | −1.8 | 4.5E−02 | −0.42 | 4.9E−02 | ARHGAP44 | −2.0 | 3.7E−02 | 0.43 | 9.7E−02 |
| RBM25 | −1.8 | 1.7E−02 | −0.43 | 4.4E−02 | ASAP3 | −2.0 | 4.0E−02 | 0.65 | 5.6E−03 |
| PRPF38B | −1.8 | 1.9E−02 | −0.54 | 1.1E−02 | TSEN2 | −2.0 | 9.1E−05 | −0.01 | 9.8E−01 |
| DDX10 | −1.8 | 4.1E−02 | −0.37 | 8.2E−02 | CDIP1 | −2.0 | 2.6E−02 | 0.24 | 4.2E−01 |
| RPS27A | −1.8 | 1.7E−02 | −0.72 | 4.3E−04 | ETNK1 | −2.0 | 4.0E−02 | 0.70 | 2.9E−03 |
| LRRC47 | −1.8 | 2.7E−02 | −0.64 | 2.0E−03 | HES5 | −2.0 | 4.5E−02 | −0.18 | 5.7E−01 |
| MMS19 | −1.8 | 4.6E−02 | −0.58 | 5.7E−03 | PXMP2 | −2.0 | 7.4E−03 | 0.19 | 5.3E−01 |
| WWP2 | −1.8 | 4.9E−03 | −0.43 | 4.3E−02 | USP30 | −2.0 | 6.0E−03 | 0.38 | 1.6E−01 |
| SREK1 | −1.8 | 9.2E−03 | −0.45 | 3.6E−02 | UGDH | −2.0 | 1.9E−02 | 0.10 | 7.7E−01 |
| HSPA5 | −1.8 | 2.7E−02 | −0.28 | 1.7E−01 | PLCD3 | −2.0 | 3.5E−02 | −0.40 | 1.3E−01 |
| DEK | −1.8 | 8.5E−03 | −0.36 | 8.5E−02 | GRAMD2A | −2.0 | 4.0E−02 | 0.24 | 4.2E−01 |
| HYPK | −1.8 | 2.7E−02 | −0.27 | 1.9E−01 | LETM1 | −2.0 | 3.4E−03 | 0.07 | 8.4E−01 |
| 43719 | −1.8 | 7.2E−03 | −0.16 | 4.4E−01 | SLC25A42 | −2.0 | 4.8E−03 | 0.27 | 3.6E−01 |
| DNASE2 | −1.8 | 3.9E−02 | −0.10 | 6.4E−01 | RNASE1 | −2.0 | 3.2E−02 | −0.29 | 3.2E−01 |
| SNRPD1 | −1.8 | 1.9E−02 | −0.66 | 1.3E−03 | CPT2 | −2.0 | 9.8E−03 | 0.46 | 6.8E−02 |
| JAGN1 | −1.8 | 3.7E−03 | −0.35 | 1.0E−01 | RNF152 | −2.0 | 4.1E−02 | −0.53 | 2.9E−02 |
| CKMT1A | −1.9 | 1.2E−02 | −0.40 | 5.7E−02 | CDX2 | −2.0 | 3.2E−02 | 0.30 | 3.0E−01 |
| B3GNT2 | −1.9 | 5.0E−02 | −0.39 | 6.4E−02 | SLC22A5 | −2.0 | 3.8E−02 | 0.26 | 3.9E−01 |
| RPS24 | −1.9 | 2.4E−02 | −0.66 | 1.3E−03 | LONRF3 | −2.0 | 2.9E−03 | 0.08 | 8.2E−01 |
| BCAS1 | −1.9 | 1.3E−02 | −0.55 | 1.0E−02 | MPST | −2.0 | 1.4E−02 | 0.42 | 1.2E−01 |
| USP48 | −1.9 | 3.7E−03 | −0.37 | 8.2E−02 | RCN3 | −2.1 | 4.3E−02 | 0.15 | 6.6E−01 |
| TOP2A | −1.9 | 2.8E−02 | −0.29 | 1.6E−01 | NCKAP5 | −2.1 | 1.5E−02 | 0.46 | 7.0E−02 |
| SDAD1 | −1.9 | 9.9E−03 | −0.43 | 4.4E−02 | SH3BGRL2 | −2.1 | 8.4E−03 | 0.14 | 6.7E−01 |
| TLCD2 | −1.9 | 1.6E−02 | −0.33 | 1.2E−01 | SERPINA6 | −2.1 | 3.5E−02 | −0.23 | 4.5E−01 |
| RPP21 | −1.9 | 4.4E−02 | −0.59 | 4.1E−03 | EFCAB5 | −2.1 | 2.0E−03 | −0.09 | 8.1E−01 |
| NUCKS1 | −1.9 | 5.7E−03 | −0.43 | 4.2E−02 | OAF | −2.1 | 5.2E−05 | −0.30 | 3.1E−01 |
| RBM34 | −1.9 | 4.7E−02 | −0.37 | 7.7E−02 | IL37 | −2.1 | 8.6E−03 | −0.10 | 7.9E−01 |
| DDAH2 | −1.9 | 4.4E−02 | −0.12 | 5.7E−01 | ADIRF | −2.1 | 4.9E−02 | 0.27 | 3.6E−01 |
| PUM3 | −1.9 | 3.5E−02 | −0.46 | 3.0E−02 | TPRN | −2.1 | 1.5E−02 | −0.09 | 8.1E−01 |
| BDP1 | −1.9 | 3.1E−02 | −0.45 | 3.4E−02 | MYZAP | −2.1 | 1.4E−04 | 0.26 | 3.9E−01 |
| PRR11 | −1.9 | 4.4E−02 | −0.10 | 6.4E−01 | PLA2R1 | −2.1 | 3.8E−02 | 0.26 | 3.8E−01 |
| MYADM | −1.9 | 2.4E−02 | −0.43 | 4.4E−02 | GJB1 | −2.1 | 3.1E−03 | −0.39 | 1.4E−01 |
| IL32 | −1.9 | 2.2E−02 | −0.22 | 2.7E−01 | TCTA | −2.1 | 1.5E−02 | −0.26 | 3.9E−01 |
| CKAP4 | −1.9 | 7.0E−03 | −0.69 | 7.3E−04 | CLDN15 | −2.1 | 1.1E−03 | −0.03 | 9.3E−01 |
| ATF6B | −1.9 | 3.8E−02 | −0.41 | 5.5E−02 | LCE2A | −2.1 | 1.8E−02 | −0.11 | 7.6E−01 |
| MEPCE | −1.9 | 1.8E−02 | −0.49 | 2.3E−02 | PRR15 | −2.1 | 4.9E−03 | −0.11 | 7.6E−01 |
| C6orf132 | −1.9 | 3.1E−02 | −0.39 | 6.4E−02 | CYP4F12 | −2.1 | 6.5E−03 | 0.00 | 1.0E+00 |
| ZNF69 | −1.9 | 4.5E−02 | −0.24 | 2.3E−01 | P2RY1 | −2.1 | 4.4E−02 | −0.58 | 1.3E−02 |
| CCDC112 | −1.9 | 1.6E−02 | −0.32 | 1.3E−01 | MOGAT2 | −2.1 | 1.5E−03 | −0.34 | 2.3E−01 |
| CWC15 | −1.9 | 3.3E−03 | −0.60 | 3.8E−03 | PCOTH | −2.1 | 4.8E−02 | −0.32 | 2.8E−01 |
| MRPL21 | −1.9 | 3.5E−02 | −0.43 | 4.2E−02 | GLOD5 | −2.1 | 3.1E−02 | −0.19 | 5.3E−01 |
| NDUFV3 | −1.9 | 3.0E−02 | −0.45 | 3.4E−02 | GLB1L2 | −2.1 | 2.1E−02 | −0.21 | 4.9E−01 |
| WDR43 | −1.9 | 7.3E−03 | −0.48 | 2.4E−02 | TM4SF5 | −2.1 | 4.0E−02 | −0.10 | 7.8E−01 |
| LGALS9 | −1.9 | 3.2E−02 | −0.14 | 4.8E−01 | TMEM177 | −2.1 | 1.5E−02 | −0.40 | 1.3E−01 |
| TNPO2 | −1.9 | 2.7E−02 | −0.22 | 2.7E−01 | KLF8 | −2.1 | 1.4E−02 | 0.30 | 3.0E−01 |
| EIF3CL | −1.9 | 3.8E−02 | −0.37 | 7.7E−02 | FLVCR1 | −2.1 | 4.4E−03 | 0.46 | 6.8E−02 |
| NDRG2 | −1.9 | 3.4E−02 | −0.57 | 6.7E−03 | TMEM37 | −2.1 | 3.1E−02 | 0.14 | 6.7E−01 |
| COX16 | −1.9 | 1.8E−02 | −0.35 | 9.3E−02 | NETO2 | −2.1 | 7.5E−03 | 0.42 | 1.2E−01 |
| CWF19L2 | −1.9 | 1.6E−02 | −0.54 | 1.1E−02 | A1CF | −2.1 | 2.5E−02 | 0.11 | 7.7E−01 |
| TAPBPL | −1.9 | 2.6E−02 | −0.36 | 8.7E−02 | TMEM253 | −2.2 | 2.0E−02 | −0.35 | 2.2E−01 |
| EPB41L1 | −1.9 | 3.8E−02 | −0.07 | 7.6E−01 | EPB41L4B | −2.2 | 8.8E−03 | 0.35 | 2.2E−01 |
| BRCA1 | −1.9 | 2.6E−02 | −0.27 | 1.8E−01 | SLITRK6 | −2.2 | 4.7E−02 | −0.08 | 8.3E−01 |
| ANP32B | −1.9 | 4.0E−03 | −0.27 | 1.9E−01 | ZBTB7C | −2.2 | 3.3E−02 | −0.20 | 5.2E−01 |
| ZC3H15 | −1.9 | 3.3E−03 | −0.43 | 4.3E−02 | TRPM4 | −2.2 | 5.0E−03 | −0.23 | 4.5E−01 |
| PIK3R3 | −1.9 | 2.1E−02 | −0.28 | 1.7E−01 | CES2 | −2.2 | 3.7E−03 | 0.02 | 9.6E−01 |
| WDR76 | −1.9 | 2.7E−02 | −0.20 | 3.3E−01 | NDRG2 | −2.2 | 2.8E−02 | −0.57 | 1.4E−02 |
| SPAG7 | −2.0 | 2.7E−02 | −0.71 | 5.0E−04 | ACAA1 | −2.2 | 6.2E−05 | −0.30 | 3.0E−01 |
| AP2B1 | −2.0 | 3.1E−02 | −0.64 | 2.2E−03 | MAOA | −2.2 | 1.6E−02 | −0.24 | 4.2E−01 |
| ATAD2 | −2.0 | 1.7E−02 | −0.51 | 1.7E−02 | FXYD3 | −2.2 | 5.3E−05 | −0.16 | 6.0E−01 |
| EBP | −2.0 | 2.6E−02 | −0.49 | 2.0E−02 | SEMA5A | −2.2 | 2.3E−02 | 0.10 | 7.9E−01 |
| MRPS18B | −2.0 | 3.7E−03 | −0.32 | 1.3E−01 | CDKN2B | −2.2 | 6.6E−03 | 0.06 | 8.8E−01 |
| FAM193A | −2.0 | 3.0E−02 | −0.16 | 4.3E−01 | PDK4 | −2.2 | 2.3E−02 | 0.20 | 5.2E−01 |
| FAM208B | −2.0 | 4.0E−02 | −0.24 | 2.4E−01 | NICN1 | −2.2 | 1.7E−03 | 0.13 | 7.1E−01 |
| THOC5 | −2.0 | 2.7E−02 | −0.30 | 1.5E−01 | CMBL | −2.2 | 2.7E−03 | 0.59 | 1.1E−02 |
| PKP4 | −2.0 | 1.2E−03 | −0.39 | 6.8E−02 | UFSP1 | −2.3 | 3.2E−03 | 0.04 | 9.0E−01 |
| CEP104 | −2.0 | 9.2E−03 | −0.46 | 3.3E−02 | PDSS1 | −2.3 | 4.4E−02 | 0.12 | 7.3E−01 |
| APOBEC3C | −2.0 | 1.7E−02 | −0.63 | 2.3E−03 | HOXB8 | −2.3 | 2.6E−02 | 0.34 | 2.4E−01 |
| GNL2 | −2.0 | 4.2E−02 | −0.57 | 7.0E−03 | AGFG2 | −2.3 | 1.9E−03 | 0.34 | 2.4E−01 |
| PPP1R10 | −2.0 | 1.5E−02 | −0.26 | 2.0E−01 | DDAH2 | −2.3 | 5.2E−05 | −0.12 | 7.4E−01 |
| CEP152 | −2.0 | 1.1E−02 | −0.11 | 5.7E−01 | TMEM56 | −2.3 | 1.1E−02 | 0.46 | 6.7E−02 |
| NOMO1 | −2.0 | 1.7E−02 | −0.29 | 1.7E−01 | ALKAL2 | −2.3 | 5.5E−04 | 0.07 | 8.4E−01 |
| PSMG3 | −2.0 | 3.3E−02 | −0.56 | 8.5E−03 | ARSE | −2.3 | 6.2E−03 | −0.24 | 4.3E−01 |
| ENDOD1 | −2.0 | 1.2E−02 | −0.62 | 2.5E−03 | AHCYL2 | −2.3 | 1.5E−03 | −0.13 | 7.1E−01 |
| TRA2A | −2.0 | 1.1E−02 | −0.36 | 9.1E−02 | RHOU | −2.3 | 1.7E−03 | 0.04 | 9.2E−01 |
| KMT5A | −2.0 | 1.4E−02 | −0.25 | 2.2E−01 | FAM213A | −2.3 | 1.2E−03 | 0.18 | 5.7E−01 |
| LYAR | −2.0 | 4.2E−02 | −0.42 | 4.8E−02 | TINAG | −2.3 | 3.8E−02 | −0.47 | 6.2E−02 |
| ZFP62 | −2.0 | 2.1E−02 | −0.46 | 3.2E−02 | MAP7D2 | −2.3 | 2.9E−02 | −0.58 | 1.3E−02 |
| TRAPPC6A | −2.0 | 2.4E−02 | −0.24 | 2.4E−01 | SLC19A3 | −2.3 | 1.0E−02 | 0.21 | 4.9E−01 |
| MYH14 | −2.0 | 4.5E−02 | −0.01 | 9.4E−01 | TBX10 | −2.3 | 3.5E−02 | −0.23 | 4.4E−01 |
| TMEM125 | −2.0 | 1.3E−02 | −0.43 | 4.5E−02 | BMP5 | −2.3 | 6.9E−03 | 0.60 | 1.1E−02 |
| HAUS6 | −2.0 | 7.0E−03 | −0.31 | 1.4E−01 | FOXO4 | −2.3 | 2.9E−02 | 0.06 | 8.8E−01 |
| DHRS7B | −2.0 | 1.9E−02 | −0.44 | 4.1E−02 | SLC4A10 | −2.3 | 1.5E−02 | 0.22 | 4.6E−01 |
| CCDC47 | −2.1 | 4.2E−03 | −0.18 | 3.9E−01 | MACROD1 | −2.3 | 2.3E−02 | 0.17 | 5.8E−01 |
| HSF2 | −2.1 | 3.5E−02 | −0.32 | 1.3E−01 | CA12 | −2.4 | 2.4E−03 | 0.33 | 2.4E−01 |
| CDT1 | −2.1 | 3.9E−02 | −0.30 | 1.5E−01 | AC092587.1 | −2.4 | 5.0E−02 | −0.10 | 7.8E−01 |
| CPSF3 | −2.1 | 3.7E−03 | −0.42 | 4.8E−02 | SLC27A4 | −2.4 | 2.5E−02 | 0.28 | 3.5E−01 |
| FAM32A | −2.1 | 7.6E−03 | −0.61 | 3.1E−03 | NUDT4P1 | −2.4 | 8.6E−03 | 0.05 | 8.9E−01 |
| SYTL4 | −2.1 | 3.1E−02 | −0.60 | 3.8E−03 | TST | −2.4 | 1.5E−03 | −0.02 | 9.6E−01 |
| SRFBP1 | −2.1 | 4.8E−02 | −0.29 | 1.6E−01 | CCDC153 | −2.4 | 1.0E−02 | −0.36 | 2.1E−01 |
| DTX3L | −2.1 | 3.4E−02 | −0.05 | 8.2E−01 | PMM1 | −2.4 | 1.3E−03 | 0.10 | 7.8E−01 |
| GRIPAP1 | −2.1 | 6.2E−03 | −0.21 | 3.0E−01 | TMUB1 | −2.4 | 8.5E−03 | 0.18 | 5.7E−01 |
| GTSE1 | −2.1 | 2.4E−02 | −0.32 | 1.3E−01 | SLC13A2 | −2.4 | 2.6E−02 | −0.60 | 1.1E−02 |
| AURKA | −2.1 | 3.7E−03 | −0.45 | 3.6E−02 | PPP2R5D | −2.4 | 2.5E−02 | 0.01 | 9.8E−01 |
| TANGO2 | −2.1 | 3.5E−02 | −0.28 | 1.8E−01 | NAT8 | −2.4 | 1.7E−02 | −0.08 | 8.3E−01 |
| GPI | −2.1 | 1.0E−02 | −0.47 | 2.9E−02 | AMACR | −2.4 | 1.5E−02 | −0.34 | 2.3E−01 |
| HDAC7 | −2.1 | 2.3E−03 | −0.42 | 4.7E−02 | DHRS11 | −2.4 | 3.2E−03 | 0.53 | 2.7E−02 |
| ADRM1 | −2.1 | 4.8E−02 | −0.54 | 1.1E−02 | METTL7B | −2.4 | 4.2E−02 | 0.44 | 9.0E−02 |
| NDST1 | −2.1 | 4.3E−02 | −0.38 | 7.7E−02 | PLA2G12B | −2.4 | 2.2E−02 | −0.34 | 2.4E−01 |
| UPF3B | −2.1 | 9.0E−03 | −0.46 | 3.2E−02 | ACOT4 | −2.4 | 1.3E−02 | 0.15 | 6.6E−01 |
| STAU2 | −2.1 | 6.2E−03 | −0.24 | 2.3E−01 | INPP5J | −2.5 | 5.8E−03 | 0.07 | 8.4E−01 |
| CENPL | −2.1 | 1.4E−02 | −0.48 | 2.4E−02 | MEP1A | −2.5 | 2.2E−02 | 0.40 | 1.3E−01 |
| PPDPF | −2.1 | 4.2E−03 | −0.19 | 3.5E−01 | AC007906.2 | −2.5 | 1.8E−02 | −0.07 | 8.4E−01 |
| EEF1D | −2.1 | 1.5E−02 | −0.61 | 3.1E−03 | ACADS | −2.5 | 1.7E−03 | −0.12 | 7.2E−01 |
| GJB1 | −2.1 | 2.5E−02 | −0.39 | 6.5E−02 | ADH1C | −2.5 | 3.9E−02 | 0.40 | 1.3E−01 |
| SLC39A11 | −2.1 | 2.1E−02 | −0.59 | 4.3E−03 | HR | −2.5 | 1.7E−03 | −0.48 | 5.2E−02 |
| USP28 | −2.1 | 2.4E−02 | −0.20 | 3.3E−01 | PDXP | −2.5 | 4.2E−02 | −0.22 | 4.7E−01 |
| GPR89B | −2.1 | 3.5E−02 | −0.44 | 4.1E−02 | PDK2 | −2.5 | 2.3E−03 | −0.02 | 9.7E−01 |
| RNF214 | −2.1 | 3.7E−02 | −0.40 | 5.7E−02 | IQCM | −2.5 | 3.6E−02 | −0.19 | 5.4E−01 |
| KIF15 | −2.1 | 4.6E−02 | −0.42 | 4.8E−02 | MUC20 | −2.5 | 1.3E−02 | −0.09 | 8.1E−01 |
| PES1 | −2.1 | 3.1E−02 | −0.14 | 4.8E−01 | SYCP3 | −2.5 | 3.4E−04 | −0.35 | 2.3E−01 |
| SAAL1 | −2.1 | 6.2E−03 | −0.28 | 1.8E−01 | MTRNR2L12 | −2.5 | 3.2E−02 | −0.15 | 6.5E−01 |
| MKI67 | −2.1 | 1.7E−02 | −0.37 | 8.2E−02 | FCGRT | −2.5 | 2.4E−04 | 0.10 | 7.8E−01 |
| TNFAIP8L1 | −2.2 | 3.7E−03 | −0.23 | 2.7E−01 | LTK | −2.5 | 4.2E−03 | 0.21 | 5.0E−01 |
| CMIP | −2.2 | 1.3E−02 | −0.26 | 2.0E−01 | SGK2 | −2.5 | 3.7E−03 | 0.07 | 8.4E−01 |
| SYCP3 | −2.2 | 8.5E−03 | −0.35 | 1.0E−01 | SLC16A1 | −2.5 | 1.7E−03 | 0.31 | 2.9E−01 |
| KHSRP | −2.2 | 1.8E−02 | −0.17 | 4.0E−01 | CDK3 | −2.5 | 1.5E−03 | −0.11 | 7.6E−01 |
| DYNC1H1 | −2.2 | 3.2E−03 | −0.44 | 3.7E−02 | NDRG1 | −2.6 | 6.7E−05 | 0.41 | 1.3E−01 |
| TRIAP1 | −2.2 | 1.3E−02 | −0.52 | 1.5E−02 | NUDT14 | −2.6 | 5.9E−03 | 0.11 | 7.6E−01 |
| RAD9A | −2.2 | 8.5E−03 | −0.05 | 8.3E−01 | ANK3 | −2.6 | 9.1E−04 | 0.11 | 7.5E−01 |
| SIRT6 | −2.2 | 8.4E−03 | −0.39 | 6.3E−02 | WSCD1 | −2.6 | 2.8E−02 | −0.02 | 9.7E−01 |
| TTK | −2.2 | 2.2E−02 | −0.37 | 7.7E−02 | PDE6A | −2.6 | 2.8E−02 | 0.20 | 5.2E−01 |
| SLC39A7 | −2.2 | 1.2E−02 | −0.49 | 2.0E−02 | RETSAT | −2.6 | 1.5E−04 | −0.39 | 1.4E−01 |
| AQP1 | −2.2 | 3.3E−02 | −0.54 | 1.1E−02 | SCIN | −2.6 | 2.5E−02 | 0.19 | 5.4E−01 |
| SDHAF2 | −2.2 | 1.0E−02 | −0.46 | 3.2E−02 | RAB6B | −2.6 | 5.2E−03 | 0.47 | 6.0E−02 |
| NCL | −2.3 | 4.9E−03 | −0.51 | 1.6E−02 | FABP1 | −2.6 | 6.8E−03 | 0.19 | 5.4E−01 |
| PCDH1 | −2.3 | 3.2E−02 | −0.31 | 1.5E−01 | NEURL1B | −2.6 | 3.1E−03 | −0.21 | 5.1E−01 |
| STRN3 | −2.3 | 1.1E−02 | −0.39 | 6.6E−02 | SAMD13 | −2.6 | 8.7E−04 | −0.13 | 7.1E−01 |
| MPHOSPH6 | −2.3 | 3.3E−03 | −0.45 | 3.4E−02 | HSD17B2 | −2.6 | 9.2E−03 | 0.66 | 4.8E−03 |
| ZFC3H1 | −2.3 | 1.5E−02 | −0.26 | 2.0E−01 | SLC22A18AS | −2.6 | 1.2E−03 | −0.20 | 5.2E−01 |
| SMC1A | −2.3 | 3.3E−03 | −0.42 | 4.8E−02 | LDHD | −2.7 | 1.8E−02 | −0.07 | 8.5E−01 |
| FUBP1 | −2.3 | 6.3E−03 | −0.38 | 7.7E−02 | WDR78 | −2.7 | 1.8E−02 | 0.30 | 3.1E−01 |
| PPP3R1 | −2.3 | 6.2E−03 | −0.39 | 6.5E−02 | ACSF2 | −2.7 | 3.1E−02 | 0.29 | 3.3E−01 |
| CKAP2L | −2.3 | 3.3E−02 | −0.51 | 1.6E−02 | ACOX1 | −2.7 | 4.2E−04 | 0.27 | 3.6E−01 |
| MUC3A | −2.3 | 3.1E−02 | −0.19 | 3.5E−01 | SELENBP1 | −2.7 | 4.4E−02 | −0.41 | 1.2E−01 |
| TTI1 | −2.3 | 1.1E−02 | −0.44 | 3.7E−02 | MYLPF | −2.7 | 5.0E−03 | −0.14 | 6.7E−01 |
| DCAKD | −2.3 | 4.0E−02 | −0.42 | 4.6E−02 | NEURL3 | −2.7 | 2.0E−02 | −0.04 | 9.1E−01 |
| GPT2 | −2.4 | 2.0E−02 | −0.05 | 8.0E−01 | KRT12 | −2.7 | 3.0E−02 | 0.30 | 3.0E−01 |
| THOP1 | −2.4 | 1.8E−02 | −0.54 | 1.1E−02 | SOWAHA | −2.7 | 6.9E−03 | 0.43 | 9.7E−02 |
| CNTLN | −2.4 | 4.5E−02 | −0.30 | 1.5E−01 | C2orf54 | −2.8 | 3.7E−03 | −0.08 | 8.3E−01 |
| PTMA | −2.4 | 4.3E−03 | −0.39 | 6.4E−02 | SLC38A4 | −2.8 | 2.0E−02 | 0.58 | 1.3E−02 |
| TUBA1C | −2.4 | 2.2E−02 | −0.54 | 1.1E−02 | NHEJ1 | −2.8 | 6.1E−03 | −0.50 | 4.0E−02 |
| HYOU1 | −2.4 | 1.1E−02 | 0.00 | 9.9E−01 | PHGR1 | −2.8 | 4.1E−03 | −0.30 | 3.0E−01 |
| ARHGEF11 | −2.4 | 3.1E−02 | −0.09 | 6.5E−01 | IGSF9 | −2.8 | 2.3E−02 | −0.35 | 2.2E−01 |
| NCAPG | −2.4 | 3.8E−02 | −0.48 | 2.3E−02 | GXYLT2 | −2.9 | 3.5E−02 | 0.48 | 5.2E−02 |
| NDUFS2 | −2.4 | 1.5E−02 | −0.53 | 1.3E−02 | ENTPD5 | −2.9 | 1.0E−03 | 0.29 | 3.2E−01 |
| ATP5EP2 | −2.4 | 8.1E−03 | −0.44 | 4.0E−02 | VIPR1 | −2.9 | 9.5E−05 | −0.24 | 4.2E−01 |
| FER1L6 | −2.5 | 4.1E−02 | −0.27 | 1.8E−01 | BMP3 | −2.9 | 2.0E−02 | −0.06 | 8.8E−01 |
| HHLA2 | −2.5 | 2.6E−02 | −0.63 | 2.4E−03 | SLC20A1 | −3.0 | 3.3E−03 | 0.59 | 1.2E−02 |
| HMGN5 | −2.5 | 1.2E−02 | −0.39 | 6.5E−02 | PLCD1 | −3.0 | 4.0E−03 | 0.42 | 1.1E−01 |
| PADI2 | −2.5 | 9.2E−03 | −0.22 | 2.7E−01 | PIGZ | −3.0 | 1.1E−02 | −0.38 | 1.7E−01 |
| PSMC3 | −2.5 | 4.1E−03 | −0.50 | 1.9E−02 | SLC39A5 | −3.0 | 4.2E−02 | 0.13 | 6.9E−01 |
| IL2RA | −2.5 | 2.2E−02 | −0.17 | 4.1E−01 | CCNJL | −3.1 | 4.1E−03 | 0.31 | 3.0E−01 |
| SMARCB1 | −2.5 | 1.1E−02 | −0.60 | 3.8E−03 | CYP2B6 | −3.1 | 1.3E−02 | 0.50 | 4.3E−02 |
| S100A13 | −2.6 | 1.3E−02 | −0.52 | 1.5E−02 | TIGD6 | −3.1 | 8.8E−04 | 0.04 | 9.2E−01 |
| TRMT11 | −2.6 | 1.1E−02 | −0.19 | 3.5E−01 | CHP2 | −3.2 | 1.6E−02 | −0.52 | 3.0E−02 |
| MND1 | −2.6 | 4.4E−02 | −0.35 | 1.0E−01 | SLC23A3 | −3.2 | 4.2E−03 | 0.61 | 9.9E−03 |
| SWI5 | −2.6 | 4.2E−02 | −0.35 | 9.4E−02 | DISP2 | −3.3 | 9.2E−05 | −0.02 | 9.7E−01 |
| ZNF625 | −2.6 | 4.2E−02 | −0.30 | 1.6E−01 | SULT1A2 | −3.4 | 1.1E−02 | 0.02 | 9.7E−01 |
| B3GNT7 | −2.6 | 9.0E−03 | −0.66 | 1.3E−03 | BEST2 | −3.4 | 4.2E−02 | −0.50 | 4.5E−02 |
| DONSON | −2.7 | 2.4E−02 | −0.32 | 1.3E−01 | PRKG2 | −3.4 | 3.3E−03 | −0.33 | 2.5E−01 |
| NCDN | −2.7 | 2.7E−02 | −0.45 | 3.5E−02 | ABCG2 | −3.5 | 3.4E−02 | 0.25 | 4.0E−01 |
| CETN3 | −2.8 | 3.7E−03 | −0.41 | 5.4E−02 | SLC17A4 | −3.5 | 3.0E−03 | 0.29 | 3.3E−01 |
| VNN2 | −2.8 | 2.9E−02 | −0.40 | 6.2E−02 | SLC16A9 | −3.5 | 4.3E−02 | −0.12 | 7.4E−01 |
| IL11RA | −2.8 | 9.9E−03 | −0.40 | 5.7E−02 | OTOP2 | −3.6 | 2.7E−02 | −0.32 | 2.7E−01 |
| PYCR1 | −2.9 | 4.6E−02 | −0.29 | 1.6E−01 | TMIGD1 | −3.6 | 4.6E−02 | −0.58 | 1.2E−02 |
| POC1A | −2.9 | 3.7E−03 | −0.29 | 1.6E−01 | CKB | −3.7 | 1.2E−02 | −0.54 | 2.6E−02 |
| SFN | −2.9 | 9.2E−03 | −0.11 | 5.9E−01 | DEFB1 | −3.7 | 9.8E−05 | 0.49 | 4.6E−02 |
| PAK1IP1 | −3.8 | 2.0E−02 | −0.52 | 1.5E−02 | LYPD8 | −3.7 | 4.2E−02 | 0.27 | 3.7E−01 |
| PLTP | −3.9 | 3.1E−02 | −0.35 | 9.8E−02 | MEP1B | −3.9 | 8.5E−03 | 0.46 | 6.8E−02 |
| NOX1 | −4.3 | 2.3E−02 | −0.63 | 2.3E−03 | PITX2 | −3.9 | 4.2E−02 | 0.60 | 1.0E−02 |
| EME1 | −6.2 | 1.1E−02 | −0.28 | 1.7E−01 | AQP7 | −4.0 | 1.2E−04 | −0.02 | 9.7E−01 |
| AC073610.2 | −8.4 | 1.1E−02 | −0.07 | 7.4E−01 | FRMD1 | −4.1 | 2.0E−03 | −0.19 | 5.4E−01 |
| NME7 | −9.5 | 1.8E−03 | −0.27 | 1.8E−01 | SLC51A | −4.1 | 2.4E−04 | 0.54 | 2.5E−02 |
| DND1 | −13.6 | 3.2E−03 | −0.36 | 8.7E−02 | B4GALNT2 | −4.3 | 3.5E−02 | 0.54 | 2.5E−02 |
| SULT1A4 | −13.9 | 3.1E−04 | −0.51 | 1.6E−02 | PADI2 | −4.3 | 9.3E−04 | −0.22 | 4.7E−01 |
| | | | | | ZG16 | −4.4 | 4.9E−02 | −0.24 | 4.3E−01 |
| | | | | | PCK1 | −4.6 | 1.9E−02 | 0.03 | 9.5E−01 |
| | | | | | GSTA1 | −4.7 | 2.6E−02 | −0.20 | 5.2E−01 |
| | | | | | TNNC2 | −4.8 | 2.7E−03 | −0.04 | 9.2E−01 |
| | | | | | SLC26A2 | −4.8 | 3.3E−02 | −0.05 | 8.9E−01 |
| | | | | | SULT1A4 | −4.8 | 1.3E−02 | −0.51 | 3.7E−02 |
| | | | | | GUCA2B | −5.5 | 3.8E−02 | −0.52 | 3.1E−02 |
| | | | | | HMGCS2 | −7.5 | 9.1E−03 | 0.52 | 3.0E−02 |
| | | | | | AQP8 | −13.8 | 2.2E−02 | 0.26 | 3.9E−01 |

TABLE 7
Summary of UC transcriptomic datasets. UC, ulcerative colitis; GSE, genomic spatial event.

| | | | | | Sites of | Age | No. of | No. of | References |
|---|---|---|---|---|---|---|---|---|---|
| No | Study | Year | Accession | Tissue | biopsies | group | UC cases | controls | (PMID) |
| 1 | Olsen et | 2007 | GSE9452 | Colon | Descending | Adults | 8 | 5 | 19177426 |
| | al. | | | | or sigmoid | | | | |
| 2 | Wu et al. | 2007 | GSE6731 | Colon | Affected | Adults | 5 | 4 | 17262812 |
| | | | | | area or | | | | |
| | | | | | sigmoid | | | | |
| 3 | Galamb et | 2008 | GSE10714 | Colon | Affected | Adults | 3 | 3 | 18843029 |
| | al. | | | | area | | | | |
| 4 | Ahrens et | 2008 | GSE10191 | Colon | Affected | Children | 8 | 11 | 18981162 |
| | al. | | | | area | | | | |
| 5 | Bjerrum et | 2009 | GSE13367 | Colon | Descending | Adults | 8 | 10 | 19834973 |
| | al. | | | | | | | | |
| 6 | Kugathasa | 2009 | GSE10616 | Colon | Ascending | Children | 10 | 11 | 18758464 |
| | n et al. | | | | | | | | |
| 7 | Arijs et al. | 2010 | GSE14580 | Colon | Rectum | Adults | 24 | 6 | 19700435 |
| 8 | Planell et | 2012 | GSE38713 | Colon | Sigmoid or | Adults | 15 | 13 | 23135761 |
| | al. | | | | rectum | | | | |
| 9 | Montero- | 2013 | GSE36807 | Colon | Ascending | Adults | 15 | 7 | 24155895 |
| | Meléndez | | | | | | | | |
| | et al. | | | | | | | | |
| 10 | Bjerrum et | 2014 | GSE47908 | Colon | Descending | Adults | 20 | 15 | 25358065 |
| | al. | | | | | | | | |
| 11 | Vanhove | 2015 | GSE59071 | Colon | Sigmoid or | Adults | 74 | 11 | 26313692 |
| | et al. | | | | rectum | | | | |
| 12 | Li et al. | 2018 | GSE87473 | Colon | Sigmoid | Adults | 27 | 21 | 29401083 |
| 13 | Li et al. | 2018 | GSE87473 | Colon | Ascending | Children | 13 | 21 | 29401083 |
| 14 | Haberman | 2019 | GSE109142 | Colon | Rectum | Children | 206 | 20 | 30604764 |
| | et al. | | | | | | | | |

TABLE 8
List of genes in the EoC transcriptome overlapped with EoE and EoG transcriptomes. EoC, eosinophilic
colitis; EoE, eosinophilic esophagitis; EoG, eosinophilic gastritis; FC, fold change; NL, normal.

| Upregulated genes in the EoC transcriptome | | | | Downregulated genes in the EoC transcriptome | | | |
|---|---|---|---|---|---|---|---|
| Overlapped with EoE | | Overlapped with EoG | | Overlapped with EoE | | Overlapped with EoG | |
| upregulated genes | | upregulated genes | | downregulated genes | | downregulated genes | |
| Gene | FC (Active | Gene | FC (Active | Gene | FC (Active | Gene | FC (Active |
| Symbol | EoC vs NL) | Symbol | EoC vs NL) | Symbol | EoC vs NL) | Symbol | EoC vs NL) |
| CLC | 10.2 | CLC | 10.2 | PPDPF | −2.1 | NUPR1 | −1.6 |
| IFITM1 | 8.3 | GAPT | 3.9 | C6orf132 | −1.9 | | |
| F13A1 | 4.7 | CD9 | 3.8 | TSC22D2 | −1.7 | | |
| IFNGR1 | 4.6 | CXCL1 | 3.6 | DDAH1 | −1.7 | | |
| CLDN23 | 4.4 | MMP12 | 3.2 | ACOT11 | −1.6 | | |
| GAPT | 3.9 | NCF2 | 3.1 | | | | |
| CD9 | 3.8 | CEBPE | 3.0 | | | | |
| CXCL1 | 3.6 | CSF2RB | 2.3 | | | | |
| TRIM21 | 3.6 | CLDN7 | 2.2 | | | | |
| CD300A | 3.4 | TMIGD3 | 2.2 | | | | |
| MMP12 | 3.2 | C15orf48 | 2.2 | | | | |
| NCF2 | 3.1 | LST1 | 2.2 | | | | |
| LY96 | 3.1 | CCL11 | 2.1 | | | | |
| CD53 | 3.1 | ADAMDEC1 | 2.1 | | | | |
| SERPINF1 | 2.7 | PLCG2 | 2.1 | | | | |
| GCH1 | 2.6 | FPR1 | 1.9 | | | | |
| ITM2C | 2.6 | SOCS1 | 1.7 | | | | |
| GLRX | 2.5 | ALOX5AP | 1.7 | | | | |
| HLA-F | 2.5 | CD209 | 1.6 | | | | |
| LIPA | 2.4 | | | | | | |
| PRIM2 | 2.4 | | | | | | |
| KLRB1 | 2.4 | | | | | | |
| LUM | 2.3 | | | | | | |
| B2M | 2.3 | | | | | | |
| HLA-B | 2.3 | | | | | | |
| IL15RA | 2.3 | | | | | | |
| CSF2RB | 2.3 | | | | | | |
| RDH10 | 2.2 | | | | | | |
| AMY2B | 2.2 | | | | | | |
| SIRPA | 2.2 | | | | | | |
| VIM | 2.1 | | | | | | |
| COL3A1 | 2.1 | | | | | | |
| PLAU | 2.1 | | | | | | |
| CHEK2 | 2.1 | | | | | | |
| C2 | 2.1 | | | | | | |
| SLC38A9 | 2.0 | | | | | | |
| LTA4H | 2.0 | | | | | | |
| CTSC | 2.0 | | | | | | |
| HIGD2A | 1.9 | | | | | | |
| ANXA4 | 1.9 | | | | | | |
| GATA2 | 1.9 | | | | | | |
| PSMB8 | 1.9 | | | | | | |
| COTL1 | 1.8 | | | | | | |
| LASPI | 1.8 | | | | | | |
| SOCS1 | 1.7 | | | | | | |
| ALOX5AP | 1.7 | | | | | | |
| PTPN9 | 1.7 | | | | | | |
| UBE2L6 | 1.6 | | | | | | |
| THBD | 1.6 | | | | | | |

TABLE 9
Functional annotation enrichment of the EoC transcriptome

| | | | | | | |
|---|---|---|---|---|---|---|
| | | | | | Genes | |
| | | | P | FDR | from | Genes in |
| ID | Name | Source | value | B&H | Input | Annotation |

327 Upregulated genes
GO: Molecular Function

| | | | | | | | |
|---|---|---|---|---|---|---|---|
| 1 | GO: 0015932 | nucleobase-containing | GO: Molecular | 4.3E−05 | 3.4E−02 | 8 | 45 |
| | | compound transmembrane | Function | | | | |
| | | transporter activity | | | | | |
| 2 | GO: 0008234 | cysteine-type peptidase | GO: Molecular | 6.6E−05 | 3.4E−02 | 40 | 711 |
| | | activity | Function | | | | |
| 3 | GO: 0004464 | leukotriene-C4 synthase | GO: Molecular | 9.8E−05 | 3.4E−02 | 3 | 4 |
| | | activity | Function | | | | |
| 4 | GO: 0003735 | structural constituent of | GO: Molecular | 1.2E−04 | 3.4E−02 | 17 | 205 |
| | | ribosome | Function | | | | |
| 5 | GO: 0003723 | RNA binding | GO: Molecular | 1.8E−04 | 4.1E−02 | 85 | 1973 |
| | | | Function | | | | |
| 6 | GO: 0015165 | pyrimidine nucleotide- | GO: Molecular | 3.0E−04 | 4.9E−02 | 4 | 12 |
| | | sugar transmembrane | Function | | | | |
| | | transporter activity | | | | | |
| 7 | GO: 0004865 | protein serine/threonine | GO: Molecular | 3.0E−04 | 4.9E−02 | 4 | 12 |
| | | phosphatase inhibitor | Function | | | | |
| | | activity | | | | | |

GO: Biological Process

| | | | | | | | |
|---|---|---|---|---|---|---|---|
| 1 | GO: 0044265 | cellular macromolecule | GO: Biological | 2.0E−09 | 1.2E−05 | 71 | 1201 |
| | | catabolic process | Process | | | | |
| 2 | GO: 0043299 | leukocyte degranulation | GO: Biological | 3.8E−09 | 1.2E−05 | 42 | 546 |
| | | | Process | | | | |
| 3 | GO: 0002275 | myeloid cell activation | GO: Biological | 6.8E−09 | 1.4E−05 | 42 | 557 |
| | | involved in immune | Process | | | | |
| | | response | | | | | |
| 4 | GO: 0009057 | macromolecule catabolic | GO: Biological | 9.8E−09 | 1.5E−05 | 79 | 1453 |
| | | process | Process | | | | |
| 5 | GO: 0002444 | myeloid leukocyte | GO: Biological | 1.2E−08 | 1.5E−05 | 42 | 568 |
| | | mediated immunity | Process | | | | |
| 6 | GO: 0042119 | neutrophil activation | GO: Biological | 3.3E−08 | 2.7E−05 | 38 | 502 |
| | | | Process | | | | |
| 7 | GO: 0006614 | SRP-dependent | GO: Biological | 3.7E−08 | 2.7E−05 | 16 | 105 |
| | | cotranslational protein | Process | | | | |
| | | targeting to membrane | | | | | |
| 8 | GO: 0002446 | neutrophil mediated | GO: Biological | 4.1E−08 | 2.7E−05 | 38 | 506 |
| | | immunity | Process | | | | |
| 9 | GO: 0045047 | protein targeting to ER | GO: Biological | 4.2E−08 | 2.7E−05 | 17 | 120 |
| | | | Process | | | | |
| 10 | GO: 0036230 | granulocyte activation | GO: Biological | 4.5E−08 | 2.7E−05 | 38 | 508 |
| | | | Process | | | | |
| 11 | GO: 0043312 | neutrophil degranulation | GO: Biological | 4.8E−08 | 2.7E−05 | 37 | 488 |
| | | | Process | | | | |
| 12 | GO: 0002283 | neutrophil activation | GO: Biological | 5.6E−08 | 2.9E−05 | 37 | 491 |
| | | involved in immune | Process | | | | |
| | | response | | | | | |
| 13 | GO: 0006613 | cotranslational protein | GO: Biological | 6.3E−08 | 3.0E−05 | 16 | 109 |
| | | targeting to membrane | Process | | | | |
| 14 | GO: 0072599 | establishment of protein | GO: Biological | 6.9E−08 | 3.0E−05 | 17 | 124 |
| | | localization to | Process | | | | |
| | | endoplasmic reticulum | | | | | |
| 15 | GO: 0006402 | mRNA catabolic process | GO: Biological | 1.0E−07 | 4.1E−05 | 31 | 377 |
| | | | Process | | | | |
| 16 | GO: 0016032 | viral process | GO: Biological | 1.3E−07 | 5.1E−05 | 52 | 853 |
| | | | Process | | | | |
| 17 | GO: 0045055 | regulated exocytosis | GO: Biological | 1.8E−07 | 6.3E−05 | 51 | 837 |
| | | | Process | | | | |
| 18 | GO: 0044403 | symbiotic process | GO: Biological | 1.8E−07 | 6.3E−05 | 54 | 911 |
| | | | Process | | | | |
| 19 | GO: 0070972 | protein localization to | GO: Biological | 2.0E−07 | 6.4E−05 | 18 | 149 |
| | | endoplasmic reticulum | Process | | | | |
| 20 | GO: 0080134 | regulation of response to | GO: Biological | 2.1E−07 | 6.4E−05 | 83 | 1671 |
| | | stress | Process | | | | |
| 21 | GO: 0016071 | mRNA metabolic process | GO: Biological | 7.1E−07 | 1.7E−04 | 52 | 901 |
| | | | Process | | | | |
| 22 | GO: 0002274 | myeloid leukocyte | GO: Biological | 7.3E−07 | 1.7E−04 | 43 | 683 |
| | | activation | Process | | | | |
| 23 | GO: 0002237 | response to molecule of | GO: Biological | 7.5E−07 | 1.7E−04 | 29 | 371 |
| | | bacterial origin | Process | | | | |
| 24 | GO: 0001775 | cell activation | GO: Biological | 7.5E−07 | 1.7E−04 | 77 | 1558 |
| | | | Process | | | | |
| 25 | GO: 0006887 | exocytosis | GO: Biological | 7.6E−07 | 1.7E−04 | 54 | 953 |
| | | | Process | | | | |

GO: Cellular Component

| | | | | | | | |
|---|---|---|---|---|---|---|---|
| 1 | GO: 0005764 | lysosome | GO: Cellular | 1.3E−09 | 5.2E−07 | 51 | 735 |
| | | | Component | | | | |
| 2 | GO: 0000323 | lytic vacuole | GO: Cellular | 1.4E−09 | 5.2E−07 | 51 | 736 |
| | | | Component | | | | |
| 3 | GO: 0005774 | vacuolar membrane | GO: Cellular | 5.2E−09 | 9.1E−07 | 35 | 417 |
| | | | Component | | | | |
| 4 | GO: 0005765 | lysosomal membrane | GO: Cellular | 5.6E−09 | 9.1E−07 | 32 | 359 |
| | | | Component | | | | |
| 5 | GO: 0098852 | lytic vacuole membrane | GO: Cellular | 6.0E−09 | 9.1E−07 | 32 | 360 |
| | | | Component | | | | |
| 1 | GO: 0005739 | mitochondrion | GO: Cellular | 8.5E−08 | 1.1E−05 | 90 | 1866 |
| 6 | | | Component | | | | |
| 7 | GO: 0005789 | endoplasmic reticulum | GO: Cellular | 1.1E−07 | 1.2E−05 | 61 | 1093 |
| | | membrane | Component | | | | |
| 8 | GO: 0042175 | nuclear outer membrane- | GO: Cellular | 2.3E−07 | 2.2E−05 | 61 | 1116 |
| | | endoplasmic reticulum | Component | | | | |
| | | membrane network | | | | | |
| 9 | GO: 0042611 | MHC protein complex | GO: Cellular | 3.1E−07 | 2.6E−05 | 8 | 26 |
| | | | Component | | | | |
| 10 | GO: 0005925 | focal adhesion | GO: Cellular | 4.3E−07 | 3.2E−05 | 31 | 411 |
| | | | Component | | | | |
| 11 | GO: 0030055 | cell-substrate junction | GO: Cellular | 7.2E−07 | 4.9E−05 | 31 | 421 |
| | | | Component | | | | |
| 12 | GO: 0022626 | cytosolic ribosome | GO: Cellular | 8.6E−07 | 5.4E−05 | 16 | 134 |
| | | | Component | | | | |
| 13 | GO: 0005768 | endosome | GO: Cellular | 9.9E−07 | 5.7E−05 | 53 | 956 |
| | | | Component | | | | |
| 14 | GO: 0031967 | organelle envelope | GO: Cellular | 1.2E−06 | 6.0E−05 | 65 | 1279 |
| | | | Component | | | | |
| 15 | GO: 0031975 | envelope | GO: Cellular | 1.2E−06 | 6.0E−05 | 65 | 1280 |
| | | | Component | | | | |
| 16 | GO: 0005794 | Golgi apparatus | GO: Cellular | 1.3E−06 | 6.2E−05 | 78 | 1643 |
| | | | Component | | | | |
| 17 | GO: 0000139 | Golgi membrane | GO: Cellular | 1.7E−06 | 7.3E−05 | 45 | 770 |
| | | | Component | | | | |
| 18 | GO: 1990234 | transferase complex | GO: Cellular | 1.8E−06 | 7.3E−05 | 47 | 821 |
| | | | Component | | | | |
| 19 | GO: 1902494 | catalytic complex | GO: Cellular | 2.9E−06 | 1.1E−04 | 73 | 1536 |
| | | | Component | | | | |
| 20 | GO: 0012506 | vesicle membrane | GO: Cellular | 4.1E−06 | 1.6E−04 | 46 | 822 |
| | | | Component | | | | |
| 21 | GO: 0005740 | mitochondrial envelope | GO: Cellular | 4.6E−06 | 1.6E−04 | 45 | 800 |
| | | | Component | | | | |
| 22 | GO: 0030659 | cytoplasmic vesicle | GO: Cellular | 4.6E−06 | 1.6E−04 | 45 | 800 |
| | | membrane | Component | | | | |
| 23 | GO: 0030141 | secretory granule | GO: Cellular | 5.7E−06 | 1.9E−04 | 48 | 884 |
| | | | Component | | | | |
| 24 | GO: 0005912 | ladherens junction | GO: Cellular | 5.9E−06 | 1.9E−04 | 35 | 560 |
| | | | Component | | | | |
| 25 | GO: 0012507 | ER to Golgi transport | GO: Cellular | 6.7E−06 | 2.0E−04 | 10 | 62 |
| | | vesicle membrane | Component | | | | |

Pathway

| | | | | | | | |
|---|---|---|---|---|---|---|---|
| 1 | 1268689 | SRP-dependent | BioSystems: | 2.4E−08 | 2.4E−05 | 18 | 116 |
| | | cotranslational protein | REACTOME | | | | |
| | | targeting to membrane | | | | | |
| 2 | 1268678 | Translation | BioSystems: | 5.8E−08 | 2.9E−05 | 21 | 165 |
| | | | REACTOME | | | | |
| 3 | 1269203 | Innate Immune System | BioSystems: | 2.5E−07 | 8.6E−05 | 75 | 1312 |
| | | | REACTOME | | | | |
| 4 | 1457780 | Neutrophil degranulation | BioSystems: | 9.7E−07 | 2.1E−04 | 37 | 492 |
| | | | REACTOME | | | | |
| 5 | 1268854 | Disease | BioSystems: | 1.3E−06 | 2.1E−04 | 54 | 867 |
| | | | REACTOME | | | | |
| 6 | 1268691 | Peptide chain elongation | BioSystems: | 1.3E−06 | 2.1E−04 | 14 | 93 |
| | | | REACTOME | | | | |
| 7 | 1268690 | Eukaryotic Translation | BioSystems: | 2.4E−06 | 3.5E−04 | 14 | 98 |
| | | Elongation | REACTOME | | | | |
| 8 | 1269056 | Infectious disease | BioSystems: | 2.9E−06 | 3.7E−04 | 31 | 393 |
| | | | REACTOME | | | | |
| 9 | 1268686 | GTP hydrolysis and | BioSystems: | 5.2E−06 | 4.8E−04 | 15 | 119 |
| | | joining of the 60S | REACTOME | | | | |
| | | ribosomal subunit | | | | | |
| 10 | 1268688 | L13a-mediated | BioSystems: | 5.2E−06 | 4.8E−04 | 15 | 119 |
| | | translational silencing of | REACTOME | | | | |
| | | Ceruloplasmin expression | | | | | |
| 11 | 1269716 | Nonsense-Mediated Decay | BioSystems: | 6.4E−06 | 4.8E−04 | 15 | 121 |
| | | (NMD) | REACTOME | | | | |
| 12 | 1269717 | Nonsense Mediated Decay | BioSystems: | 6.4E−06 | 4.8E−04 | 15 | 121 |
| | | (NMD) enhanced by the | REACTOME | | | | |
| | | Exon Junction Complex | | | | | |
| | | (EJC) | | | | | |
| 13 | 1268681 | Formation of a pool of free | BioSystems: | 6.9E−06 | 4.8E−04 | 14 | 107 |
| | | 40S subunits | REACTOME | | | | |
| 14 | 1269941 | Transport of nucleotide | BioSystems: | 6.9E−06 | 4.8E−04 | 5 | 10 |
| | | sugars | REACTOME | | | | |
| 15 | 1269120 | Viral mRNA Translation | BioSystems: | 7.1E−06 | 4.8E−04 | 13 | 93 |
| | | | REACTOME | | | | |
| 16 | 1339156 | Selenocysteine synthesis | BioSystems: | 1.0E−05 | 6.3E−04 | 13 | 96 |
| | | | REACTOME | | | | |
| 17 | 1268692 | Eukaryotic Translation | BioSystems: | 1.1E−05 | 6.3E−04 | 13 | 97 |
| | | Termination | REACTOME | | | | |
| 18 | 1268680 | Cap-dependent Translation | BioSystems: | 1.2E−05 | 6.3E−04 | 15 | 127 |
| | | Initiation | REACTOME | | | | |
| 19 | 1268679 | Eukaryotic Translation | BioSystems: | 1.2E−05 | 6.3E−04 | 15 | 127 |
| | | Initiation | REACTOME | | | | |
| 20 | 1269718 | Nonsense Mediated Decay | BioSystems: | 1.6E−05 | 8.0E−04 | 13 | 100 |
| | | (NMD) independent of the | REACTOME | | | | |
| | | Exon Junction Complex | | | | | |
| | | (EJC) | | | | | |
| 21 | 1269109 | Influenza Life Cycle | BioSystems: | 1.7E−05 | 8.3E−04 | 16 | 147 |
| | | | REACTOME | | | | |
| 22 | 1269195 | Antigen processing-Cross | BioSystems: | 2.2E−05 | 1.0E−03 | 13 | 103 |
| | | presentation | REACTOME | | | | |
| 23 | 1269311 | Interferon Signaling | BioSystems: | 2.3E−05 | 1.0E−03 | 19 | 202 |
| | | | REACTOME | | | | |
| 24 | 1269115 | Influenza Viral RNA | BioSystems: | 3.2E−05 | 1.3E−03 | 15 | 138 |
| | | Transcription and | REACTOME | | | | |
| | | Replication | | | | | |
| 25 | 1269310 | Cytokine Signaling in | BioSystems: | 4.1E−05 | 1.6E−03 | 45 | 763 |
| | | Immune system | REACTOME | | | | |

310 Downregulated genes
GO: Molecular Function

| | | | | | | | |
|---|---|---|---|---|---|---|---|
| 1 | GO: 0003723 | RNA binding | GO: Molecular | 3.40E−26 | 2.99E−23 | 117 | 1973 |
| | | | Function | | | | |
| 2 | GO: 0042393 | histone binding | GO: Molecular | 6.28E−08 | 2.76E−05 | 19 | 203 |
| | | | Function | | | | |
| 3 | GO: 0008301 | DNA binding, bending | GO: Molecular | 1.13E−05 | 3.32E−03 | 17 | 236 |
| | | | Function | | | | |
| 4 | GO: 0070577 | lysine-acetylated histone | GO: Molecular | 3.67E−05 | 6.45E−03 | 5 | 19 |
| | | binding | Function | | | | |
| 5 | GO: 0140033 | acetylation-dependent | GO: Molecular | 3.67E−05 | 6.45E−03 | 5 | 19 |
| | | protein binding | Function | | | | |
| 6 | GO: 0016887 | ATPase activity | GO: Molecular | 6.91E−05 | 1.01E−02 | 28 | 596 |
| | | | Function | | | | |
| 7 | GO: 0004386 | helicase activity | GO: Molecular | 9.93E−05 | 1.25E−02 | 13 | 177 |
| | | | Function | | | | |
| 8 | GO: 0003682 | chromatin binding | GO: Molecular | 1.71E−04 | 1.88E−02 | 28 | 629 |
| | | | Function | | | | |
| 9 | GO: 0004843 | thiol-dependent ubiquitin- | GO: Molecular | 4.08E−04 | 2.81E−02 | 16 | 287 |
| | | specific protease activity | Function | | | | |
| 10 | GO: 0043022 | ribosome binding | GO: Molecular | 4.22E−04 | 2.81E−02 | 7 | 65 |
| | | | Function | | | | |
| 11 | GO: 0101005 | ubiquitinyl hydrolase | GO: Molecular | 4.40E−04 | 2.81E−02 | 16 | 289 |
| | | activity | Function | | | | |
| 12 | GO: 0016403 | dimethylargininase | GO: Molecular | 14.51E−04 | 2.81E−02 | 2 | 2 |
| | | activity | Function | | | | |
| 13 | GO: 0043021 | ribonucleoprotein complex | GO: Molecular | 4.76E−04 | 2.81E−02 | 11 | 156 |
| | | binding | Function | | | | |
| 14 | GO: 0001164 | RNA polymerase I core | GO: Molecular | 4.79E−04 | 2.81E−02 | 3 | 8 |
| | | promoter sequence- | Function | | | | |
| | | specific DNA binding | | | | | |
| 15 | GO: 0001163 | RNA polymerase I | GO: Molecular | 4.79E−04 | 2.81E−02 | 3 | 8 |
| | | transcription regulatory | Function | | | | |
| | | region sequence-specific | | | | | |
| | | DNA binding | | | | | |
| 16 | GO: 0045296 | cadherin binding | GO: Molecular | 8.71E−04 | 4.79E−02 | 17 | 338 |
| | | | Function | | | | |

GO: Biological Process

| | | | | | | | |
|---|---|---|---|---|---|---|---|
| 1 | GO: 0007049 | cell cycle | GO: Biological | 1.04E−12 | 4.98E−09 | 87 | 1971 |
| | | | Process | | | | |
| 2 | GO: 0016071 | mRNA metabolic process | GO: Biological | 6.78E−12 | 1.63E−08 | 52 | 901 |
| | | | Process | | | | |
| 3 | GO: 0006325 | chromatin organization | GO: Biological | 16.96E−11 | 5.67E−08 | 48 | 841 |
| | | | Process | | | | |
| 4 | GO: 0000278 | mitotic cell cycle | GO: Biological | 7.08E−11 | 5.67E−08 | 56 | 1083 |
| | | | Process | | | | |
| 5 | GO: 0140014 | mitotic nuclear division | GO: Biological | 7.08E−11 | 5.67E−08 | 56 | 1083 |
| | | | Process | | | | |
| 6 | GO: 1903047 | mitotic cell cycle process | GO: Biological | 7.08E−11 | 5.67E−08 | 56 | 1083 |
| | | | Process | | | | |
| 7 | GO: 0051276 | chromosome organization | GO: Biological | 1.09E−10 | 7.50E−08 | 62 | 1287 |
| | | | Process | | | | |
| 8 | GO: 0031570 | DNA integrity checkpoint | GO: Biological | 8.50E−10 | 5.03E−07 | 19 | 164 |
| | | | Process | | | | |
| 9 | GO: 0022402 | cell cycle process | GO: Biological | 9.43E−10 | 5.03E−07 | 70 | 1632 |
| | | | Process | | | | |
| 10 | GO: 0010564 | regulation of cell cycle | GO: Biological | 2.05E−09 | 9.36E−07 | 45 | 839 |
| | | process | Process | | | | |
| 11 | GO: 0000280 | nuclear division | GO: Biological | 2.31E−09 | 9.36E−07 | 58 | 1256 |
| | | | Process | | | | |
| 12 | GO: 0044839 | cell cycle G2/M phase | GO: Biological | 2.43E−09 | 9.36E−07 | 24 | 279 |
| | | transition | Process | | | | |
| 13 | GO: 0042254 | ribosome biogenesis | GO: Biological | 2.65E−09 | 9.36E−07 | 25 | 303 |
| | | | Process | | | | |
| 14 | GO: 0051726 | regulation of cell cycle | GO: Biological | 2.73E−09 | 9.36E−07 | 59 | 1295 |
| | | | Process | | | | |
| 15 | GO: 1901987 | regulation of cell cycle | GO: Biological | 3.47E−09 | 1.11E−06 | 33 | 508 |
| | | phase transition | Process | | | | |
| 16 | GO: 0022613 | ribonucleoprotein complex | GO: Biological | 4.21E−09 | 1.26E−06 | 33 | 512 |
| | | biogenesis | Process | | | | |
| 17 | GO: 1902749 | regulation of cell cycle | GO: Biological | 4.96E−09 | 1.40E−06 | 21 | 223 |
| | | G2/M phase transition | Process | | | | |
| 18 | GO: 0071824 | protein-DNA complex | GO: Biological | 7.27E−09 | 1.87E−06 | 27 | 367 |
| | | subunit organization | Process | | | | |
| 19 | GO: 1901990 | regulation of | GO: Biological | 7.38E−09 | 1.87E−06 | 31 | 470 |
| | | mitotic cell | Process | | | | |
| | | cycle phase transition | | | | | |
| 20 | GO: 0048285 | organelle fission | GO: Biological | 8.32E−09 | 1.93E−06 | 58 | 1301 |
| | | | Process | | | | |
| 21 | GO: 0044770 | cell cycle phase transition | GO: Biological | 8.42E−09 | 1.93E−06 | 38 | 668 |
| | | | Process | | | | |
| 22 | GO: 0008380 | RNA splicing | GO: Biological | 9.93E−09 | 2.17E−06 | 31 | 476 |
| | | | Process | | | | |
| 23 | GO: 0000077 | DNA damage checkpoint | GO: Biological | 1.14E−08 | 2.37E−06 | 17 | 152 |
| | | | Process | | | | |
| 24 | GO: 0044772 | mitotic cell cycle phase | GO: Biological | 1.37E−08 | 2.73E−06 | 36 | 622 |
| | | transition | Process | | | | |
| 25 | GO: 0000086 | G2/M transition of mitotic | GO: Biological | 1.53E−08 | 2.86E−06 | 22 | 260 |
| | | cell cycle | Process | | | | |

GO: Cellular Component

| | | | | | | | |
|---|---|---|---|---|---|---|---|
| 1 | GO: 1902494 | catalytic complex | GO: Cellular | 1.80E−14 | 1.29E−11 | 77 | 1536 |
| | | | Component | | | | |
| 2 | GO: 0005730 | nucleolus | GO: Cellular | 1.17E−09 | 4.17E−07 | 65 | 1495 |
| | | | Component | | | | |
| 3 | GO: 0022624 | proteasome accessory | GO: Cellular | 1.69E−08 | 4.04E−06 | 8 | 25 |
| | | complex | Component | | | | |
| 4 | GO: 0005838 | proteasome regulatory | GO: Cellular | 1.43E−07 | 2.56E−05 | 7 | 22 |
| | | particle | Component | | | | |
| 5 | GO: 0016604 | nuclear body | GO: Cellular | 5.26E−07 | 7.53E−05 | 38 | 799 |
| | | | Component | | | | |
| 6 | GO: 0008540 | proteasome regulatory | GO: Cellular | 2.03E−06 | 2.43E−04 | 5 | 12 |
| | | particle, base subcomplex | Component | | | | |
| 7 | GO: 0005681 | spliceosomal complex | GO: Cellular | 4.40E−06 | 4.50E−04 | 15 | 186 |
| | | | Component | | | | |
| 8 | GO: 0005814 | centriole | GO: Cellular | 5.23E−06 | 4.68E−04 | 29 | 583 |
| | | | Component | | | | |
| 9 | GO: 0000502 | proteasome complex | GO: Cellular | 6.31E−06 | 5.02E−04 | 9 | 67 |
| | | | Component | | | | |
| 10 | GO: 1905369 | endopeptidase complex | GO: Cellular | 7.15E−06 | 5.12E−04 | 9 | 68 |
| | | | Component | | | | |
| 11 | GO: 0005815 | microtubule organizing | GO: Cellular | 1.80E−05 | 1.17E−03 | 33 | 756 |
| | | center | Component | | | | |
| 12 | GO: 0016607 | nuclear speck | GO: Cellular | 2.01E−05 | 1.20E−03 | 22 | 406 |
| | | | Component | | | | |
| 13 | GO: 0005813 | centrosome | GO: Cellular | 2.19E−05 | 1.21E−03 | 26 | 532 |
| | | | Component | | | | |
| 14 | GO: 0000922 | spindle pole | GO: Cellular | 2.95E−05 | 1.51E−03 | 13 | 168 |
| | | | Component | | | | |
| 15 | GO: 0005819 | spindle | GO: Cellular | 3.24E−05 | 1.55E−03 | 20 | 359 |
| | | | Component | | | | |
| 16 | GO: 0015630 | microtubule cytoskeleton | GO: Cellular | 3.57E−05 | 1.60E−03 | 49 | 1368 |
| | | | Component | | | | |
| 17 | GO: 0000775 | Ichromosome, centromeric | GO: Cellular | 4.98E−05 | 2.10E−03 | 14 | 202 |
| | | region | Component | | | | |
| 18 | GO: 0000793 | condensed chromosome | GO: Cellular | 7.29E−05 | 2.90E−03 | 15 | 236 |
| | | | Component | | | | |
| 19 | GO: 1905368 | peptidase complex | GO: Cellular | 9.05E−05 | 3.41E−03 | 9 | 93 |
| | | | Component | | | | |
| 20 | GO: 1990234 | transferase complex | GO: Cellular | 2.00E−04 | 7.15E−03 | 32 | 821 |
| | | | Component | | | | |
| 21 | GO: 0098687 | chromosomal region | GO: Cellular | 3.25E−04 | 1.11E−02 | 18 | 362 |
| | | | Component | | | | |
| 22 | GO: 0005682 | U5 snRNP | GO: Cellular | 3.88E−04 | 1.23E−02 | 10 | 137 |
| | | | Component | | | | |
| 23 | GO: 1990565 | HSP90-CDC37 chaperone | GO: Cellular | 3.94E−04 | 1.23E−02 | 2 | 2 |
| | | complex | Component | | | | |
| 24 | GO: 0016514 | SWI/SNF complex | GO: Cellular | 4.53E−04 | 1.35E−02 | 4 | 19 |
| | | | Component | | | | |
| 25 | GO: 0000974 | Prp19 complex | GO: Cellular | 5.34E−04 | 1.53E−02 | 8 | 94 |
| | | | Component | | | | |

Pathway

| | | | | | | | |
|---|---|---|---|---|---|---|---|
| 1 | 1269741 | Cell Cycle | BioSystems: | 1.44E−10 | 1.21E−07 | 41 | 624 |
| | | | REACTOME | | | | |
| 2 | 1269799 | G2/M Transition | BioSystems: | 4.47E−10 | 1.53E−07 | 21 | 184 |
| | | | REACTOME | | | | |
| 3 | 1269797 | Mitotic G2-G2/M phases | BioSystems: | 5.47E−10 | 1.53E−07 | 21 | 186 |
| | | | REACTOME | | | | |
| 4 | 1269649 | Gene Expression | BioSystems: | 2.99E−09 | 6.26E−07 | 77 | 1844 |
| | | | REACTOME | | | | |
| 5 | 1269763 | Cell Cycle, Mitotic | BioSystems: | 2.21E−08 | 3.69E−06 | 33 | 517 |
| | | | REACTOME | | | | |
| 6 | 1383017 | The role of GTSE1 in | BioSystems: | 8.66E−07 | 1.21E−04 | 10 | 63 |
| | | G2/M progression after G2 | REACTOME | | | | |
| | | checkpoint | | | | | |
| 7 | 1269742 | Cell Cycle Checkpoints | BioSystems: | 2.03E−06 | 2.34E−04 | 17 | 204 |
| | | | REACTOME | | | | |
| 8 | 1269837 | Regulation of mitotic cell | BioSystems: | 3.18E−06 | 2.34E−04 | 11 | 89 |
| | | cycle | REACTOME | | | | |
| 9 | 1269838 | APC/C-mediated | BioSystems: | 3.18E−06 | 2.34E−04 | 11 | 89 |
| | | degradation of cell cycle | REACTOME | | | | |
| | | proteins | | | | | |
| 10 | 1457807 | FBXL7 down-regulates | BioSystems: | 3.75E−06 | 2.34E−04 | 9 | 58 |
| | | AURKA during mitotic | REACTOME | | | | |
| | | entry and in early mitosis | | | | | |
| 11 | 1269781 | Switching of origins to a | BioSystems: | 3.95E−06 | 2.34E−04 | 10 | 74 |
| | | post-replicative state | REACTOME | | | | |
| 12 | 1269782 | Orc1 removal from | BioSystems: | 3.95E−06 | 2.34E−04 | 10 | 74 |
| | | chromatin | REACTOME | | | | |
| 13 | 1269851 | APC/C: Cdh1 mediated | BioSystems: | 4.47E−06 | 2.34E−04 | 10 | 75 |
| | | degradation of Cdc20 and | REACTOME | | | | |
| | | other APC/C: Cdh1 | | | | | |
| | | targeted proteins in late | | | | | |
| | | mitosis/early G1 | | | | | |
| 14 | 1269846 | Cdc20: Phospho-APC/C | BioSystems: | 4.47E−06 | 2.34E−04 | 10 | 75 |
| | | mediated degradation of | REACTOME | | | | |
| | | Cyclin A | | | | | |
| 15 | 1269753 | G2/M Checkpoints | BioSystems: | 4.68E−06 | 2.34E−04 | 15 | 172 |
| | | | REACTOME | | | | |
| 16 | 1268928 | Hh mutants that don't | BioSystems: | 5.02E−06 | 2.34E−04 | 9 | 60 |
| | | undergo autocatalytic | REACTOME | | | | |
| | | processing are degraded | | | | | |
| | | by ERAD | | | | | |
| 17 | 1269796 | Removal of licensing | BioSystems: | 15.05E−06 | 2.34E−04 | 10 | 76 |
| | | factors from origins | REACTOME | | | | |
| 18 | 1269845 | APC: Cdc20 mediated | BioSystems: | 5.05E−06 | 2.34E−04 | 10 | 76 |
| | | degradation of cell cycle | REACTOME | | | | |
| | | proteins prior to satisfation | | | | | |
| | | of the cell cycle | | | | | |
| | | checkpoint | | | | | |
| 19 | 1269689 | mRNA Splicing | BioSystems: | 5.31E−06 | 2.34E−04 | 16 | 196 |
| | | | REACTOME | | | | |
| 20 | 1269844 | APC/C: Cdc20 mediated | BioSystems: | 6.41E−06 | 2.51E−04 | 10 | 78 |
| | | degradation of mitotic | REACTOME | | | | |
| | | proteins | | | | | |
| 21 | 1268927 | Hh mutants abrogate | BioSystems: | 6.63E−06 | 2.51E−04 | 9 | 62 |
| | | ligand secretion | REACTOME | | | | |
| 22 | 1269836 | CDT1 association with the | BioSystems: | 6.63E−06 | 2.51E−04 | 9 | 62 |
| | | CDC6: ORC: origin | REACTOME | | | | |
| | | complex | | | | | |
| 23 | 1269794 | Regulation of DNA | BioSystems: | 7.20E−06 | 2.51E−04 | 10 | 79 |
| | | replication | REACTOME | | | | |
| 24 | 1269842 | Activation of APC/C and | BioSystems: | 7.20E−06 | 2.51E−04 | 10 | 79 |
| | | APC/C: Cdc20 mediated | REACTOME | | | | |
| | | degradation of mitotic | | | | | |
| | | proteins | | | | | |
| 25 | 1269770 | SCF(Skp2)-mediated | BioSystems: | 7.59E−06 | 2.54E−04 | 9 | 63 |
| | | degradation of p27/p21 | REACTOME | | | | |

TABLE 10
Biological processes enriched in histo-molecular correlation for EoC. B&H, Benjamini and
Hochberg; EoC, eosinophilic colitis; FDR, false discovery rate; GO, gene ontology.

| | | | | | |
|---|---|---|---|---|---|
| | | | FDR | Genes from | Genes in |
| ID | Name | P value | B&H | Input | Annotation |

Overall eosinophilic inflammation

| | | | | | | |
|---|---|---|---|---|---|---|
| 1 | GO: 0009206 | purine ribonucleoside | 2.8E−07 | 5.3E−04 | 13 | 204 |
| | | triphosphate biosynthetic | | | | |
| | | process | | | | |
| 2 | GO: 0009145 | purine nucleoside triphosphate | 3.0E−07 | 5.3E−04 | 13 | 205 |
| | | biosynthetic process | | | | |
| 3 | GO: 0009201 | ribonucleoside triphosphate | 4.0E−07 | 5.3E−04 | 13 | 210 |
| | | biosynthetic process | | | | |
| 4 | GO: 0009205 | purine ribonucleoside | 6.1E−07 | 5.3E−04 | 13 | 218 |
| | | triphosphate metabolic process | | | | |
| 5 | GO: 0009142 | nucleoside triphosphate | 6.7E−07 | 5.3E−04 | 13 | 220 |
| | | biosynthetic process | | | | |
| 6 | GO: 0009199 | ribonucleoside triphosphate | 8.7E−07 | 5.3E−04 | 13 | 225 |
| | | metabolic process | | | | |
| 7 | GO: 0009144 | purine nucleoside triphosphate | 9.6E−07 | 5.3E−04 | 13 | 227 |
| | | metabolic process | | | | |
| 8 | GO: 0006754 | ATP biosynthetic process | 1.0E−06 | 5.3E−04 | 12 | 192 |
| 9 | GO: 0009152 | purine ribonucleotide | 1.4E−06 | 5.7E−04 | 15 | 314 |
| | | biosynthetic process | | | | |
| 10 | GO: 0042775 | mitochondrial ATP synthesis | 1.4E−06 | 5.7E−04 | 9 | 102 |
| | | coupled electron transport | | | | |

Pericryptal circumferential eosinophil collars

| | | | | | | |
|---|---|---|---|---|---|---|
| 1 | GO: 0007005 | mitochondrion organization | 3.2E−07 | 9.7E−04 | 15 | 599 |

Lamina propria eosinophil sheets

| | | | | | | |
|---|---|---|---|---|---|---|
| 1 | GO: 0045047 | protein targeting to ER | 8.6E−06 | 3.0E−03 | 5 | 123 |
| 2 | GO: 0043604 | amide biosynthetic process | 6.4E−06 | 3.0E−03 | 11 | 943 |
| 3 | GO: 0006518 | peptide metabolic process | 8.8E−06 | 3.0E−03 | 11 | 975 |
| 4 | GO: 0043043 | peptide biosynthetic process | 9.9E−06 | 3.0E−03 | 10 | 799 |
| 5 | GO: 0072599 | establishment of protein | 1.0E−05 | 3.0E−03 | 5 | 127 |
| | | localization to endoplasmic | | | | |
| | | reticulum | | | | |
| 6 | GO: 0043603 | cellular amide metabolic | 2.0E−05 | 4.9E−03 | 12 | 1276 |
| | | process | | | | |
| 7 | GO: 0070972 | protein localization to | 2.6E−05 | 5.6E−03 | 5 | 155 |
| | | endoplasmic reticulum | | | | |
| 8 | GO: 0006414 | translational elongation | 5.0E−05 | 8.2E−03 | 9 | 770 |
| 9 | GO: 0006412 | translation | 5.0E−05 | 8.2E−03 | 9 | 770 |
| 10 | GO: 0006413 | translational initiation | 8.7E−05 | 1.3E−02 | 5 | 199 |

TABLE 11
Gene list for EoC-IBD differential score. EoC, eosinophilic colitis;
IBD, inflammatory bowel disease; UC, ulcerative colitis; CD, Crohn
disease. Gene accession numbers for each of the genes listed below
are provided in Table 12, as indicated by an asterisk.

| | P value | | | P value | | |
|---|---|---|---|---|---|---|
| | Active | | | Active | | |
| Gene | EoC vs UC | Fold | | EoC vs CD | Fold | |
| symbol | (High eos) | Change | Regulation | (High eos) | Change | Regulation |
| CAPN12 | 1.0E−20 | 46.6 | up | 8.6E−04 | 1.9 | up |
| CD9 | 9.8E−11 | 2.1 | up | 2.0E−07 | 5.0 | up |
| COMMD6 | 3.2E−07 | 2.9 | up | 1.8E−04 | 5.5 | up |
| F13A1 | 2.1E−04 | 2.4 | up | 1.5E−04 | 5.5 | up |
| IFITM1 | 4.5E−02 | 1.4 | up | 1.9E−03 | 5.3 | up |
| MGST3 | 8.6E−05 | 1.6 | up | 8.1E−06 | 5.1 | up |
| MT1H | 2.3E−14 | 59.3 | up | 2.7E−04 | 15.8 | up |
| MT1X | 3.6E−03 | 2.1 | up | 1.4E−04 | 10.9 | up |
| PHLPP2 | 3.6E−12 | 3.9 | up | 2.9E−06 | 6.9 | up |
| SLC51B | 7.5E−06 | 2.3 | up | 1.1E−04 | 7.2 | up |
| TMEM171 | 1.2E−12 | 4.4 | up | 1.0E−04 | 8.3 | up |
| TMEM251 | 4.4E−04 | 1.5 | up | 1.2E−05 | 5.2 | up |
| ZFAND3 | 2.7E−05 | 1.9 | up | 9.8E−06 | 5.0 | up |
| EEF1D | 3.9E−21 | −10.6 | down | 1.6E−04 | −2.6 | down |
| HYOUI | 2.0E−28 | −20.5 | down | 3.0E−04 | −2.5 | down |
| STT3A | 2.8E−27 | −12.6 | down | 4.3E−04 | −2.4 | down |
| VNN2 | 5.8E−07 | −3.5 | down | 7.2E−05 | −5.3 | down |

TABLE 12
EoC Transcriptome Gene Set. Accession numbers, regulation and fold change
(FC) of Active EC compared to Non-EC is shown. p(corr) = FDR-correct
p value; EC = eosinophilic colitis.

| | | p (Corr) | Regulation | FC |
|---|---|---|---|---|
| Accession | | ([Active EC] | ([Active EC] | ([Active EC] |
| Number | Gene | Vs [Non-EC]) | Vs [Non-EC]) | Vs [Non-EC]) |
| NM_001828 | CLC | 0.004210975 | up | 10.220028 |
| NM_005814 | GPA33 | 0.007037839 | up | 8.783629 |
| NM_003641 | IFITM1* | 0.013335236 | up | 8.335931 |
| NM_016175.3 | MRNIP | 0.003710429 | up | 7.609262 |
| NM_005951 | MT1H* | 0.0284492 | up | 7.2740874 |
| NM_004451 | ESRRA | 0.004210975 | up | 6.5544634 |
| NM_005952 | MT1X* | 0.011370473 | up | 6.2094765 |
| NM_004642 | CDK2AP1 | 0.039349694 | up | 6.1043262 |
| NM_139286 | CDC26 | 0.002556037 | up | 6.0323796 |
| NM_178148 | SLC35B2 | 8.10E−04 | up | 5.3754053 |
| NM_001098621 | TMEM251* | 0.009178896 | up | 5.2104416 |
| NM_145030 | PPP1R35 | 0.037678093 | up | 5.1193066 |
| NM_001243186 | PIM1 | 0.015453761 | up | 4.9580317 |
| NM_002720 | PPP4C | 0.003819645 | up | 4.9246044 |
| NM_001257102 | NDUFB3 | 0.003177543 | up | 4.8545833 |
| NM_001204883 | RAB43 | 0.017527176 | up | 4.82019 |
| NM_000129 | F13A1* | 0.011290508 | up | 4.6969686 |
| NM_000416 | IFNGR1 | 0.03471796 | up | 4.600993 |
| NM_000086.2 | AC068547.1 | 0.00669022 | up | 4.5352635 |
| NM_005273 | GNB2 | 0.02634847 | up | 4.500225 |
| NM 194284 | CLDN23 | 0.016122842 | up | 4.429366 |
| NM_021943 | ZFAND3* | 0.004334522 | up | 4.407066 |
| NM_015331 | NCSTN | 0.003787587 | up | 4.399303 |
| NM_001300767.2 | PRIMPOL | 0.003266768 | up | 4.322756 |
| NM_000317 | PTS | 0.002368791 | up | 4.239456 |
| NM_020396 | BCL2L10 | 0.004932866 | up | 4.223554 |
| NM_001257972.1 | CTSL | 0.006060636 | up | 4.1825037 |
| NR_027063 | LINC00116 | 0.003895655 | up | 4.1254683 |
| NM_144563 | RPIA | 0.004210975 | up | 4.1144743 |
| NM_020199 | C5orf15 | 0.004932866 | up | 4.096953 |
| NM_001098625 | JKAMP | 0.009069772 | up | 4.0812693 |
| NM_006344 | CLEC10A | 0.004932866 | up | 4.0085154 |
| NM_001244938 | TXN | 0.009069772 | up | 3.9422529 |
| NM_014628 | MAD2L1BP | 0.003742307 | up | 3.936985 |
| NM_152687 | GAPT | 0.004701612 | up | 3.9280708 |
| NM_001012456 | SEC61G | 0.004701612 | up | 3.9224207 |
| NM_000820 | GAS6 | 0.018768316 | up | 3.9012094 |
| NM_014044 | UNC50 | 0.008473022 | up | 3.8271036 |
| NM_012106 | ARL2BP | 0.031907003 | up | 3.8024054 |
| NM_001402 | EEF1A1 | 0.004611528 | up | 3.788493 |
| NM_001769 | CD9* | 0.003315543 | up | 3.7676835 |
| NM_001033678 | TRPT1 | 0.003315543 | up | 3.7603955 |
| NM_002755 | MAP2K1 | 0.008473022 | up | 3.7590864 |
| NM_002163 | IRF8 | 0.019370817 | up | 3.7400599 |
| NM_013242.3 | CFAP20 | 0.004091352 | up | 3.7187612 |
| NM_002001 | FCER1A | 0.004864265 | up | 3.6668355 |
| NM_001511 | CXCL1 | 0.04953627 | up | 3.6247535 |
| NM_003141 | TRIM21 | 0.01673271 | up | 3.6184297 |
| NM_014059 | RGCC | 0.027377954 | up | 3.609193 |
| NM_170743.4 | IFNLR1 | 0.006502778 | up | 3.557911 |
| NM_005163 | AKT1 | 0.002368791 | up | 3.5343723 |
| NM_178859.4 | SLC51B* | 0.032116402 | up | 3.501255 |
| NM_000086.2 | AC138894.1 | 0.034710333 | up | 3.4897807 |
| NM_002643 | PIGF | 1.88E−06 | up | 3.4716992 |
| NM_005194 | CEBPB | 0.003742307 | up | 3.4352903 |
| NM_002601 | PDE6D | 0.009178896 | up | 3.412739 |
| NM_001244735 | TMEM126A | 0.027227553 | up | 3.4100373 |
| NM_001256841 | CD300A | 0.004210975 | up | 3.4098573 |
| NM_023930 | KCTD14 | 0.033025254 | up | 3.4074836 |
| NM_001172566 | MYD88 | 0.00325619 | up | 3.3665164 |
| NM_001161342 | TMEM171* | 0.010966295 | up | 3.3599892 |
| NM_053277 | CLIC6 | 0.02606485 | up | 3.3593204 |
| NM_024036 | LRFN4 | 0.042864818 | up | 3.2991536 |
| NM_007284 | TWF2 | 0.029363045 | up | 3.2885072 |
| NM_024031 | PRR14 | 0.025869396 | up | 3.2493029 |
| NM_020244 | CHPT1 | 0.004210975 | up | 3.2483656 |
| NM_001242881.2 | NAXD | 0.012854803 | up | 3.2339764 |
| NM_025128 | MUS81 | 0.008065605 | up | 3.2319486 |
| NM_030981 | RAB1B | 0.014264353 | up | 3.2235627 |
| NM_020311.2 | ACKR3 | 0.009178896 | up | 3.220978 |
| NM_031903 | MRPL32 | 0.018768316 | up | 3.2099476 |
| NM_000956 | PTGER2 | 0.004932866 | up | 3.2017634 |
| NM_023009 | MARCKSL1 | 0.001947274 | up | 3.1897786 |
| NM_017703 | FBXL12 | 0.003895655 | up | 3.1894152 |
| NM_025250 | TTYH3 | 0.010112403 | up | 3.1866546 |
| NM_033503 | BMF | 1.23E−04 | up | 3.1690712 |
| NM_002426 | MMP12 | 0.024362616 | up | 3.1652362 |
| NM_032412 | CYSTM1 | 9.89E−05 | up | 3.153946 |
| NM_000433 | NCF2 | 0.011713471 | up | 3.1344638 |
| NM_021109 | TMSB4X | 0.02740049 | up | 3.1306262 |
| NM_001195797 | LY96 | 0.009350898 | up | 3.1302702 |
| NM_000560 | CD53 | 0.009496425 | up | 3.1297143 |
| NM_001010878 | C16orf91 | 0.029150745 | up | 3.1199102 |
| NM_138417 | KTI12 | 0.009069772 | up | 3.1029558 |
| NM_018648 | NOP10 | 3.08E−04 | up | 3.0817778 |
| NM_000270 | PNP | 0.010243632 | up | 3.0780032 |
| NM_001260511 | MGST1 | 0.003803315 | up | 3.0516744 |
| NM_002524 | NRAS | 8.40E−04 | up | 3.050425 |
| NM_015954 | DERA | 0.012443559 | up | 3.0410771 |
| NM_001039573 | SEC14L1 | 0.004210975 | up | 3.0064592 |
| NM_001805 | CEBPE | 0.027201273 | up | 2.9989872 |
| NM_003851 | CREG1 | 0.014249554 | up | 2.9986837 |
| NM_080820 | DTD1 | 0.010097842 | up | 2.992293 |
| NM_002432 | MNDA | 0.025059672 | up | 2.9783196 |
| NM_147128 | ZNRF2 | 0.036740094 | up | 2.9681437 |
| NM_003000 | SDHB | 0.004210975 | up | 2.9579275 |
| NM_002208 | ITGAE | 0.005419708 | up | 2.9517078 |
| NM_207356 | C1orf174 | 0.01179124 | up | 2.9473066 |
| NM_014879 | P2RY14 | 0.00942147 | up | 2.9469385 |
| NM_004528 | MGST3* | 0.002452131 | up | 2.9308705 |
| NM_001114123 | ELK1 | 0.009983414 | up | 2.9176705 |
| NM_001040181 | CLDND1 | 0.011266685 | up | 2.9148898 |
| NM_017945 | SLC35A5 | 0.007374769 | up | 2.8919435 |
| NM_001031738 | TMEM150A | 0.00325619 | up | 2.858618 |
| NM_017812 | CHCHD3 | 0.004823584 | up | 2.8417788 |
| NM_006120 | HLA-DMA | 0.01600121 | up | 2.835315 |
| NM_001242844 | RNF146 | 0.010243632 | up | 2.8348286 |
| NM_000100 | CSTB | 0.001643641 | up | 2.8280902 |
| NM_017829.6 | HDHD5 | 0.026882868 | up | 2.807696 |
| NM_001197244 | BCL7B | 0.012293227 | up | 2.8070135 |
| NM_014290 | TDRD7 | 0.008473022 | up | 2.8058555 |
| NM_002090 | CXCL3 | 0.045844223 | up | 2.7902927 |
| NM_004807 | HS6ST1 | 0.021827964 | up | 2.7690513 |
| NM_005932 | MIPEP | 0.020793192 | up | 2.7673297 |
| NM_005768 | LPCAT3 | 0.047335915 | up | 2.7625701 |
| NM_006813 | PNRC1 | 0.0182891 | up | 2.7600238 |
| NM_001166412 | SMOC2 | 0.047551215 | up | 2.755008 |
| NM_052935.4 | NT5C3B | 0.03372394 | up | 2.7498856 |
| NM_000616 | CD4 | 0.012087019 | up | 2.747555 |
| NM_203495 | COMMD6* | 0.019934328 | up | 2.7435725 |
| NM_020135 | WRNIP1 | 0.032116402 | up | 2.7414675 |
| NM_017758 | ALKBH5 | 0.008473022 | up | 2.7401352 |
| NM_001348957.2 | SMIM26 | 0.01691245 | up | 2.73992 |
| NM_001291468.2 | CCL4L2 | 0.021686953 | up | 2.7327225 |
| NM_001166392 | TPBG | 6.40E−04 | up | 2.7311025 |
| NM_002615 | SERPINF1 | 0.042856753 | up | 2.7023246 |
| NM_001452 | FOXF2 | 0.014787058 | up | 2.702212 |
| NM_001009923 | TMEM230 | 0.003742307 | up | 2.6992357 |
| NM_001031696 | PLD3 | 0.003315543 | up | 2.6810699 |
| NM_004496 | FOXA1 | 0.026586808 | up | 2.66697 |
| NM_005850 | SF3B4 | 0.025265217 | up | 2.6622038 |
| NM_015508 | TIPARP | 0.016062709 | up | 2.6589332 |
| NM_001198858 | HLA-DQB2 | 0.008473022 | up | 2.6579337 |
| NM_194463 | RNF128 | 0.030561026 | up | 2.6565154 |
| NM_005730 | CTDSP2 | 0.004932866 | up | 2.649168 |
| NM_004275 | MED20 | 0.018459477 | up | 2.6469913 |
| NM_005195 | CEBPD | 0.013335236 | up | 2.6414704 |
| NM_000161 | GCH1 | 0.008065605 | up | 2.6382978 |
| NM_001042510 | ZNF706 | 0.004210975 | up | 2.6377375 |
| NM_001184987 | NDUFC1 | 0.011692687 | up | 2.6377 |
| NM_001130145 | YAP1 | 0.003957412 | up | 2.6362863 |
| NM_001012514 | ITM2C | 0.030820062 | up | 2.6218712 |
| NM_015695 | BRPF3 | 0.015984528 | up | 2.6148024 |
| NM_004302 | ACVR1B | 0.018768316 | up | 2.6125748 |
| NM_001134693 | OST4 | 0.004091352 | up | 2.6061177 |
| NM_020186.3 | SDHAF3 | 0.011151491 | up | 2.6007366 |
| NM_144631 | ZNF513 | 0.01146189 | up | 2.600469 |
| NM_000989 | RPL30 | 0.003895655 | up | 2.5997205 |
| NM_012328 | DNAJB9 | 0.030544128 | up | 2.5949523 |
| NM_152278 | TCEAL7 | 0.025571894 | up | 2.5920582 |
| NM_025182 | FAM214B | 0.040935516 | up | 2.5778558 |
| NM_001260 | CDK8 | 0.04428628 | up | 2.5748737 |
| NM_000771 | CYP2C9 | 0.01953677 | up | 2.5726807 |
| NM_018031 | WDR6 | 0.018125646 | up | 2.5623796 |
| NM_005780.3 | LHFPL6 | 0.031174282 | up | 2.559627 |
| NM_001901 | CTGF | 0.00679769 | up | 2.549982 |
| NM_001201478 | EIF2D | 0.012876909 | up | 2.5484118 |
| NM_001242481 | EIF1AD | 0.001198882 | up | 2.54735 |
| NM_006571 | DCTN6 | 0.018235652 | up | 2.5454326 |
| NM_004235 | KLF4 | 0.028559262 | up | 2.540729 |
| NM_023937 | MRPL34 | 0.003021401 | up | 2.5359607 |
| NM_001167903 | MFSD1 | 0.005718236 | up | 2.5223217 |
| NM_001118890 | GLRX | 0.02101984 | up | 2.5194862 |
| NM_000019 | ACAT1 | 0.031489152 | up | 2.5093243 |
| NM_001164234 | DDHD2 | 0.00325619 | up | 2.5000305 |
| NM_001204857.2 | ELOC | 0.014097312 | up | 2.4980526 |
| NM_003778 | B4GALT4 | 0.016616996 | up | 2.4933841 |
| NM_001165258 | TMEM14C | 0.013733716 | up | 2.4900644 |
| NM_014521 | SH3BP4 | 0.004932866 | up | 2.4887266 |
| NM_020154 | EMC7 | 0.025059672 | up | 2.4866495 |
| NM_001025247 | TAF5L | 0.007555221 | up | 2.4858346 |
| NM_001017927 | C2orf76 | 0.005718236 | up | 2.4857183 |
| NM_001195156.2 | TMEM35B | 0.014787058 | up | 2.4773622 |
| NM_015020 | PHLPP2* | 0.017705536 | up | 2.4733045 |
| NM_001099645 | RPL22L1 | 0.013780518 | up | 2.4730387 |
| NM_012089 | ABCB10 | 0.003713761 | up | 2.4722536 |
| NM_002145 | HOXB2 | 0.04397283 | up | 2.470447 |
| NM_006851 | GLIPR1 | 0.03963494 | up | 2.4679654 |
| NM_001098478 | HLA-F | 0.017128667 | up | 2.4593215 |
| NM_001007189 | IGIP | 0.013335236 | up | 2.4562597 |
| NM_015124 | GRAMD4 | 0.019133938 | up | 2.4555018 |
| NM_021033 | RAP2A | 0.04522721 | up | 2.4554043 |
| NM_001028 | RPS25 | 0.003713761 | up | 2.4539628 |
| NM_001204159 | SPHK2 | 0.043186285 | up | 2.4512846 |
| NM_000235 | LIPA | 0.004950345 | up | 2.4481926 |
| NM_006913 | RNF5 | 0.003803315 | up | 2.4452991 |
| NM_004427 | PHC2 | 0.012795187 | up | 2.4403849 |
| NM_001035511 | SDHC | 0.003742307 | up | 2.4362912 |
| NM_019035 | PCDH18 | 0.0182891 | up | 2.4246528 |
| NM_006808 | SEC61B | 0.034948073 | up | 2.4239697 |
| NM_198273 | LYSMD3 | 0.001782755 | up | 2.4198818 |
| NM_018452 | TMEM242 | 0.003819645 | up | 2.4190977 |
| NM_001008530 | LGMN | 0.016616996 | up | 2.3973942 |
| NM_001040138.3 | CKLF | 0.024068533 | up | 2.3926225 |
| NM_007080 | LSM6 | 0.029535152 | up | 2.3865223 |
| NM_022149 | MAGEF1 | 0.006318725 | up | 2.3834023 |
| NM_000947 | PRIM2 | 0.012809332 | up | 2.3812392 |
| NM_020807 | ZNF319 | 0.043165725 | up | 2.380961 |
| NM_152588 | TMTC2 | 0.011266685 | up | 2.3781412 |
| NM_016047.4 | SF3B6 | 0.009091702 | up | 2.3770714 |
| NM_198392 | TCF21 | 0.0284492 | up | 2.375863 |
| NM_002258 | KLRB1 | 0.040346943 | up | 2.3743818 |
| NM_022366 | TFB2M | 0.011713471 | up | 2.3694317 |
| NM_017818 | WRAP73 | 0.010399488 | up | 2.3621185 |
| NM_001482 | GATM | 0.02179076 | up | 2.3603234 |
| NM_005345 | HSPA1A | 0.048875146 | up | 2.3581834 |
| NM_024640 | YRDC | 0.02389813 | up | 2.3580894 |
| NM_001253835 | IGFBP7 | 0.043144166 | up | 2.3579726 |
| NM_017590 | ZC3H7B | 0.047335915 | up | 2.3574162 |
| NM_001127383 | CYBRD1 | 0.009069772 | up | 2.3534691 |
| NM_000199 | SGSH | 0.003710429 | up | 2.3520844 |
| NM_139032 | MAPK7 | 9.89E−05 | up | 2.3479128 |
| NM_002467 | MYC | 0.030030547 | up | 2.342226 |
| NM_016401.3 | HIKESHI | 0.027685571 | up | 2.335233 |
| NM_001130440 | SRP9 | 0.04217389 | up | 2.334562 |
| NM_148962 | OXER1 | 0.026632665 | up | 2.3341165 |
| NM_020981 | B3GALT1 | 0.03996587 | up | 2.3265893 |
| NM_017772 | TBC1D22B | 0.02300072 | up | 2.3220754 |
| NM_006527 | SLBP | 0.017848339 | up | 2.3175437 |
| NM_002345 | LUM | 0.010243632 | up | 2.317502 |
| NM_181672 | OGT | 0.019370817 | up | 2.3091493 |
| NM_005034 | POLR2K | 0.019244099 | up | 2.306297 |
| NM_004048 | B2M | 0.010464292 | up | 2.3046784 |
| NM_004418 | DUSP2 | 0.008473022 | up | 2.3044846 |
| NM_024811 | CPSF7 | 0.005718236 | up | 2.301918 |
| NM_183372 | NBPF11 | 0.019370817 | up | 2.3019075 |
| NM_033103 | RHPN2 | 0.03519735 | up | 2.2998178 |
| NM_001166103 | SPINT2 | 0.004701612 | up | 2.297813 |
| NM_004800 | TM9SF2 | 0.006167668 | up | 2.2976265 |
| NM_005514 | HLA-B | 0.00679769 | up | 2.2967684 |
| NM_030918 | SNX27 | 0.007595959 | up | 2.2963238 |
| NM_002628 | PFN2 | 0.014599939 | up | 2.29605 |
| NM_001845 | COL4A1 | 0.031356245 | up | 2.2960026 |
| NM_138689 | PPP1R14B | 0.008473022 | up | 2.2907493 |
| NM_004695 | SLC16A5 | 0.043076806 | up | 2.28569 |
| NM_001256765 | IL15RA | 0.018125646 | up | 2.283115 |
| NM_001080496 | RGP1 | 0.030987326 | up | 2.2827764 |
| NM_006519 | DYNLT1 | 0.002368791 | up | 2.2822328 |
| NM_001136482 | C19orf38 | 0.015564358 | up | 2.2797635 |
| NM_001195427 | SRSF2 | 0.01850465 | up | 2.2679112 |
| NM_000321 | RB1 | 0.004932866 | up | 2.265351 |
| NM_005067 | SIAH2 | 0.014112802 | up | 2.2650049 |
| NM_020357 | PCNP | 0.006318725 | up | 2.262048 |
| NM_024057 | NUP37 | 0.027877325 | up | 2.2546895 |
| NM_152309 | PIK3AP1 | 0.027220141 | up | 2.25288 |
| NM_000395 | CSF2RB | 0.025642958 | up | 2.251584 |
| NM_001099695 | REPIN1 | 0.003713761 | up | 2.2443175 |
| NM_000276 | OCRL | 0.003742307 | up | 2.2396593 |
| NM_001019 | RPS15A | 0.003365616 | up | 2.2376504 |
| NM_002855.4 | NECTIN1 | 0.003895655 | up | 2.232188 |
| NM_001204106.2 | BCL2L11 | 0.028695412 | up | 2.2321122 |
| NM_006923 | SDF2 | 0.014867771 | up | 2.230162 |
| NM_001039707 | SDCCAG3 | 0.013785731 | up | 2.2289605 |
| NM_172037 | RDH10 | 0.031356245 | up | 2.2260385 |
| NM_001002292 | WLS | 0.017128667 | up | 2.222883 |
| NM_004526 | MCM2 | 0.044155836 | up | 2.2175345 |
| NM_000274 | OAT | 0.027260883 | up | 2.2163508 |
| NM_015859 | GTF2A1 | 0.003713761 | up | 2.2119656 |
| NM_020978 | AMY2B | 0.020120177 | up | 2.199955 |
| NM_001161619 | DUS3L | 0.018502776 | up | 2.1985006 |
| NM_018252 | TMEM206 | 0.0239216 | up | 2.198085 |
| NM_014872 | ZBTB5 | 0.025059672 | up | 2.1948957 |
| NM_001166279 | PAN2 | 0.024991969 | up | 2.1925285 |
| NM_001040022 | SIRPA | 0.018235652 | up | 2.1901789 |
| NM_001185022 | CLDN7 | 0.009069772 | up | 2.190091 |
| NM_001099400 | SGCE | 0.041577 | up | 2.1869023 |
| NM_001081976.3 | TMIGD3 | 0.037290797 | up | 2.1774695 |
| NM_178502 | DTX3 | 0.027260883 | up | 2.1745684 |
| NM_032413 | C15orf48 | 0.01095495 | up | 2.1699302 |
| NM_022748 | TNS3 | 0.02179076 | up | 2.1672914 |
| NM_001024736 | CD276 | 0.010464292 | up | 2.1667056 |
| NM_194279 | ISCA2 | 0.006167668 | up | 2.1626048 |
| NM_015675 | GADD45B | 0.03963494 | up | 2.1623101 |
| NM_182919 | TICAM1 | 0.033178218 | up | 2.1620193 |
| NM_138434.2 | ZBED6CL | 0.04740986 | up | 2.1611328 |
| NM_003214 | TEAD3 | 0.021303346 | up | 2.1564555 |
| NM_001243266 | MS4A4A | 0.0380645 | up | 2.1521432 |
| NM_205838 | LST1 | 0.044616915 | up | 2.1511352 |
| NM_005242 | F2RL1 | 0.021827964 | up | 2.1472404 |
| NM_001170796 | ZFAND1 | 0.019370817 | up | 2.1464903 |
| NM_030802 | FAM117A | 0.008764876 | up | 2.1414773 |
| NM_003380 | VIM | 0.022493577 | up | 2.141324 |
| NM_152716 | PATL1 | 0.021675533 | up | 2.1402793 |
| NM_002986 | CCL11 | 0.049590055 | up | 2.1394277 |
| NR_023345 | CNKSR1 | 0.036685612 | up | 2.1376991 |
| NM_001142935 | MXD3 | 0.043200973 | up | 2.1365986 |
| NM_020216 | RNPEP | 0.005730095 | up | 2.13276 |
| NM_001135110 | DNAJA3 | 0.03243209 | up | 2.1311846 |
| NM_001018111 | PODXL | 0.011066498 | up | 2.1306689 |
| NM_015457 | ZDHHC5 | 0.006710657 | up | 2.1300106 |
| NM_003889 | NR1I2 | 0.015616336 | up | 2.1270947 |
| NM_001145271 | ADAMDEC1 | 0.001643641 | up | 2.1214368 |
| NM_001199111 | MDH1 | 0.004563964 | up | 2.1190352 |
| NM_007006 | NUDT21 | 0.02035276 | up | 2.1154838 |
| NM_001048241 | UBL5 | 0.029528696 | up | 2.111334 |
| NM_001145277 | NECAP2 | 0.001782755 | up | 2.1055353 |
| NM_002661 | PLCG2 | 0.020793192 | up | 2.097583 |
| NM_000090 | COL3A1 | 0.044370178 | up | 2.097479 |
| NM_181701 | QSOX2 | 0.027765444 | up | 2.0949786 |
| NM_001145031 | PLAU | 0.047718264 | up | 2.0903327 |
| NM_001005735 | CHEK2 | 0.018768316 | up | 2.0875335 |
| NM_001119 | ADD1 | 0.00572592 | up | 2.08361 |
| NM_014876 | JOSD1 | 0.00325619 | up | 2.0834026 |
| NM_006887 | ZFP36L2 | 0.012825956 | up | 2.081311 |
| NM_001039481 | ETNK1 | 0.013672209 | up | 2.073722 |
| NM_032429 | LZTS2 | 0.02300072 | up | 2.0737216 |
| NM_000063 | C2 | 0.037678093 | up | 2.0706122 |
| NM_001202457.1 | ZNF816 | 0.046243005 | up | 2.069782 |
| NM_022100 | MRPS14 | 0.028109793 | up | 2.0689495 |
| NM_003845 | DYRK4 | 0.04922643 | up | 2.0611157 |
| NM_001282225.2 | ADA2 | 0.012825956 | up | 2.0594652 |
| NM_012257 | HBP1 | 0.01864376 | up | 2.0590646 |
| NM_001048194 | RCC1 | 0.005092292 | up | 2.054866 |
| NM_182796 | MAT2B | 0.007400775 | up | 2.0536606 |
| NM_018117 | WDR11 | 0.020929493 | up | 2.0504227 |
| NM_004060 | CCNG1 | 0.004611528 | up | 2.0482893 |
| NM_173514 | SLC38A9 | 0.033511817 | up | 2.045902 |
| NM_001035521 | GTF3C2 | 0.0380645 | up | 2.045272 |
| NM_001154 | ANXA5 | 0.041447155 | up | 2.0416849 |
| NM_152433 | KBTBD3 | 0.009464388 | up | 2.0408256 |
| NM_003095 | SNRPF | 0.034715828 | up | 2.0351217 |
| NM_001256643 | LTA4H | 0.003111369 | up | 2.0320163 |
| NM_014294 | TRAM1 | 0.04397283 | up | 2.0319018 |
| NM_178167 | ZNF598 | 0.041447155 | up | 2.028497 |
| NM_022166 | XYLT1 | 0.0182891 | up | 2.0262322 |
| NM_203404 | AMPD2 | 0.049806733 | up | 2.023703 |
| NM_001267041 | ARMC8 | 0.017881768 | up | 2.0210407 |
| NM_001134999 | FERMT2 | 0.027377954 | up | 2.020345 |
| NM_002636 | PHF1 | 0.004278281 | up | 2.019123 |
| NM_006330 | LYPLA1 | 0.041725997 | up | 2.0137284 |
| NM_152293 | TADA2B | 0.009178896 | up | 2.007766 |
| NM_020839 | WDR48 | 0.013031696 | up | 2.0052443 |
| NM_207346 | TSEN54 | 0.025832525 | up | 2.0033913 |
| NM_001212 | C1QBP | 0.031237299 | up | 1.9974942 |
| NM_001114173 | CTSC | 0.008473022 | up | 1.9962889 |
| NM_001330609.1 | WASHC5 | 0.043072492 | up | 1.9928637 |
| NM_025164 | SIK3 | 0.030544128 | up | 1.9920492 |
| NM_032490.5 | GON7 | 0.008055674 | up | 1.9894632 |
| NM_018989 | RBM27 | 0.01953677 | up | 1.9874715 |
| NM_020997 | LEFTY1 | 0.045543827 | up | 1.9794596 |
| NM_013258 | PYCARD | 0.013335236 | up | 1.9780332 |
| NM_144703 | LSM14B | 0.015564358 | up | 1.9778225 |
| NM_022551 | RPS18 | 0.011682549 | up | 1.9771968 |
| NM_018992 | KCTD5 | 0.037556242 | up | 1.975232 |
| NM_032906.5 | PYURF | 0.034710333 | up | 1.9706327 |
| NM_005258 | GCHFR | 0.03083604 | up | 1.9703721 |
| NM_018050 | MANSC1 | 0.031724196 | up | 1.9692444 |
| NM_004429 | EFNB1 | 0.03772432 | up | 1.9689035 |
| NM_152678.2 | DENND6A | 0.04956045 | up | 1.9645382 |
| NM_001114106 | SLC44A3 | 0.025329078 | up | 1.9635475 |
| NM_001080424 | KDM6B | 0.024991969 | up | 1.9594885 |
| NM_001100814.3 | PIP4P1 | 0.040346943 | up | 1.956642 |
| NM_001146694 | KDM4C | 0.011682549 | up | 1.9555211 |
| NM_001030 | RPS27 | 0.03696922 | up | 1.9549375 |
| NM_138333 | FAM122A | 0.03696922 | up | 1.9447224 |
| NM_080817 | GPR82 | 0.049590055 | up | 1.943233 |
| NM_001032363.4 | MINOS1 | 0.04020767 | up | 1.9421092 |
| NM_012095 | AP3M1 | 0.004334522 | up | 1.9383994 |
| NM_052859 | RFT1 | 0.044337697 | up | 1.938069 |
| NM_006597 | HSPA8 | 0.04707265 | up | 1.9364115 |
| NM_024091 | FASTKD3 | 0.04409394 | up | 1.936206 |
| NM_153713 | LIX1L | 0.047972605 | up | 1.9340376 |
| NM_001302348.1 | UMAD1 | 0.032116402 | up | 1.9332341 |
| NM_017760 | NCAPG2 | 0.011290508 | up | 1.9322106 |
| NM_152565 | ATP6VOD2 | 0.044337697 | up | 1.9301066 |
| NM_014957 | DENND3 | 0.020785723 | up | 1.929668 |
| NM_001271907.2 | SPATA33 | 0.04677974 | up | 1.9254403 |
| NM_138820 | HIGD2A | 0.01673271 | up | 1.9236933 |
| NM_001104925 | PCMTD2 | 0.034516975 | up | 1.9211159 |
| NM_006029 | PNMA1 | 0.012795187 | up | 1.9193023 |
| NM_012413 | QPCT | 0.042856753 | up | 1.9167643 |
| NM_030949 | PPP1R14C | 0.009069772 | up | 1.9162388 |
| NM_032582 | USP32 | 0.027702793 | up | 1.9142467 |
| NM_001153 | ANXA4 | 0.004210975 | up | 1.9141276 |
| NM_001145432.3 | SMIM20 | 0.041725997 | up | 1.9110974 |
| NM_138459 | NUS1 | 0.008473022 | up | 1.9104067 |
| NM_001190996 | ARPC1A | 0.015474429 | up | 1.9089541 |
| NM_182663 | RASSF5 | 0.012087019 | up | 1.9086809 |
| NM_000319 | PEX5 | 0.02076771 | up | 1.9086287 |
| NM_031431 | COG3 | 0.04298059 | up | 1.9084483 |
| NM_001134671 | DERL1 | 0.016338587 | up | 1.9075325 |
| NM_001252226 | PLK2 | 0.049744274 | up | 1.9065976 |
| NM_001204054.3 | NDUFC2 | 0.047335915 | up | 1.9020344 |
| NM_004392 | DACH1 | 0.025265217 | up | 1.901771 |
| NM_001115113 | WDR26 | 0.001916015 | up | 1.90118 |
| NM_020457 | THAP11 | 0.008473022 | up | 1.8965292 |
| NM_138477 | CDAN1 | 0.030030653 | up | 1.8948582 |
| NM_001025780.3 | ABHD17B | 0.022944553 | up | 1.8937802 |
| NM_003709 | KLF7 | 0.019316047 | up | 1.893558 |
| NM_001100626 | ERLIN1 | 0.018169425 | up | 1.8889014 |
| NM_033547 | INTS4 | 0.026783794 | up | 1.8842746 |
| NM_006793 | PRDX3 | 0.012825956 | up | 1.8841565 |
| NM_022831 | AIDA | 0.04117375 | up | 1.8837267 |
| NM_033546 | MYL12B | 0.018125646 | up | 1.8791443 |
| NM_001142540 | SLC35B3 | 0.02179076 | up | 1.87894 |
| NM_001193306 | FPR1 | 0.04920006 | up | 1.8777233 |
| NM_001286104.2 | MT-CO2 | 0.005718236 | up | 1.8748386 |
| NM_004448 | ERBB2 | 0.025059672 | up | 1.8714395 |
| NM_001195563 | CAMTA1 | 0.041869946 | up | 1.8660821 |
| NM_032638 | GATA2 | 0.021133333 | up | 1.8653115 |
| NM_032711 | MAFG | 0.019244099 | up | 1.8644695 |
| NM_032996 | CASP9 | 0.049646936 | up | 1.8620793 |
| NM_203344 | SERTAD3 | 0.034128226 | up | 1.8615158 |
| NM_002111 | HTT | 0.007625648 | up | 1.8609084 |
| NM_148919 | PSMB8 | 0.034710333 | up | 1.8599638 |
| NM_032020 | FUCA2 | 0.019244099 | up | 1.8597256 |
| NM_003185 | TAF4 | 0.003964963 | up | 1.8586906 |
| NM_001199341 | RPL17 | 0.009259853 | up | 1.8583919 |
| NM_014624 | S100A6 | 0.008405923 | up | 1.8559287 |
| NM_001040613 | TMEM70 | 0.009178896 | up | 1.8557858 |
| NM_001256721 | CCT4 | 0.008193326 | up | 1.8544706 |
| NM_001242524 | HLA-DPA1 | 0.011266685 | up | 1.8524837 |
| NM_016323 | HERC5 | 0.047610644 | up | 1.8524679 |
| NM_007310 | COMT | 0.011856554 | up | 1.85151 |
| NM_003756 | EIF3H | 0.011370473 | up | 1.8505871 |
| NM_005686 | SOX13 | 0.023652306 | up | 1.8482711 |
| NM_015941 | ATP6V1H | 0.003315543 | up | 1.8430299 |
| NM_001152 | SLC25A5 | 0.018235652 | up | 1.8426461 |
| NM_017983 | WIPI1 | 0.032509267 | up | 1.8379121 |
| NM_015972 | POLR1D | 0.003803315 | up | 1.8365537 |
| NM_014873 | LPGAT1 | 0.047972605 | up | 1.835749 |
| NM_001080392 | KIAA1147 | 0.01600121 | up | 1.8337963 |
| NM_015103 | PLXND1 | 0.01600121 | up | 1.8315651 |
| NR_033688 | PEF1 | 0.015671546 | up | 1.8260796 |
| NM_004718 | COX7A2L | 0.004210975 | up | 1.8253112 |
| NM_012088 | PGLS | 0.026344195 | up | 1.8200532 |
| NM_006520 | DYNLT3 | 0.001916015 | up | 1.8165487 |
| NM_000988 | RPL27 | 0.011290508 | up | 1.8155575 |
| NM_001122606 | LAMP2 | 0.005718236 | up | 1.8154001 |
| NM_000636 | SOD2 | 0.006486211 | up | 1.8135957 |
| NM_001660 | ARF4 | 0.042687792 | up | 1.812726 |
| NM_001010989 | HERPUD1 | 0.040550947 | up | 1.8123755 |
| NM_174919 | ARHGAP27 | 0.028559262 | up | 1.8121307 |
| NM_001243736 | LAMTOR3 | 0.005358789 | up | 1.807467 |
| NM_000113 | TOR1A | 0.025401045 | up | 1.8032511 |
| NM_021149 | COTL1 | 0.011290508 | up | 1.8020258 |
| NM_001015002 | LLGL2 | 0.015105499 | up | 1.8003055 |
| NM_153682 | PIGP | 0.044286482 | up | 1.7975069 |
| NM_001284239.1 | SARAF | 0.014235281 | up | 1.7967473 |
| NM_001146191 | MPZL1 | 0.010197458 | up | 1.796615 |
| NM_006251 | PRKAA1 | 0.04606175 | up | 1.7950981 |
| NM_006052 | DSCR3 | 0.025338167 | up | 1.7888299 |
| NM_145012 | CCNY | 0.007196175 | up | 1.7888186 |
| NM_001242488.1 | ZSWIM8 | 0.019370817 | up | 1.7854128 |
| NM_144691 | CAPN12* | 0.015474429 | up | 1.7814658 |
| NM_001142307 | GTF2H1 | 0.010243632 | up | 1.7813134 |
| NM_080430.3 | SELENOM | 0.04020767 | up | 1.7808527 |
| NM_001008390 | CGGBP1 | 0.021827964 | up | 1.7806388 |
| NM_005660 | SLC35A2 | 0.009914366 | up | 1.7781903 |
| NM_030782 | CLPTM1L | 0.009679377 | up | 1.7765193 |
| NM_020139 | BDH2 | 0.032097448 | up | 1.7748661 |
| NM_032574 | DPY30 | 0.02606485 | up | 1.7735896 |
| NM_001177842.1 | SRPRA | 0.030008137 | up | 1.7712345 |
| NM_001277 | CHKA | 0.018865926 | up | 1.7708563 |
| NM_207174 | ABCG1 | 0.030542143 | up | 1.7687229 |
| NM_006148 | LASP1 | 0.010243632 | up | 1.7678987 |
| NM_001134651 | EIF4E3 | 0.03005725 | up | 1.7674637 |
| NM_033127 | SEC16B | 0.027220141 | up | 1.7648323 |
| NM_001008710 | RBPMS | 0.030542143 | up | 1.7642797 |
| NM_004536 | NAIP | 0.0182891 | up | 1.763956 |
| NM_024832 | RIN3 | 0.005136897 | up | 1.7613589 |
| NM_012207 | HNRNPH3 | 0.04725462 | up | 1.7613375 |
| NM_017617 | NOTCH1 | 0.02300072 | up | 1.7594174 |
| NM_080594 | RNPS1 | 0.032116402 | up | 1.7579039 |
| NM_001012993 | C9orf152 | 0.022833334 | up | 1.7571924 |
| NM_014740 | EIF4A3 | 0.008289631 | up | 1.7563244 |
| NM_007001 | SLC35D2 | 0.017406993 | up | 1.7554071 |
| NM_001142401 | CD164 | 0.021686953 | up | 1.7542875 |
| NM_022349 | MS4A6A | 0.003742307 | up | 1.7531717 |
| NM_002787 | PSMA2 | 0.018768316 | up | 1.7530059 |
| NM_001127448 | ABAT | 0.046352275 | up | 1.7521049 |
| NM_020648 | TWSG1 | 0.04256384 | up | 1.7502661 |
| NM_001204366 | MGST2 | 0.0232449 | up | 1.7486887 |
| NM_001024912 | CEACAM1 | 0.00981432 | up | 1.7486451 |
| NM_001010891 | MTX3 | 0.011787499 | up | 1.7466294 |
| NM_003745 | SOCS1 | 0.033589147 | up | 1.746323 |
| NM_016094 | COMMD2 | 0.049590055 | up | 1.7422259 |
| NM_152490 | B3GALNT2 | 0.04143761 | up | 1.7378538 |
| NM_007285 | GABARAPL2 | 0.028367871 | up | 1.7300761 |
| NM_001261400 | PSMD9 | 0.037816215 | up | 1.7261047 |
| NM_015009 | PDZRN3 | 0.033025254 | up | 1.7245244 |
| NM_004779 | CNOT8 | 0.035266526 | up | 1.7241116 |
| NM_001207024 | M6PR | 0.024362616 | up | 1.7207357 |
| NM_001206426 | USMG5 | 0.04217389 | up | 1.7186455 |
| NM_177937 | GOLM1 | 0.013066018 | up | 1.7157797 |
| NM_002121 | HLA-DPB1 | 0.017128667 | up | 1.715746 |
| NM_001629 | ALOX5AP | 0.03354008 | up | 1.7151344 |
| NM_018288 | PHF10 | 0.015297736 | up | 1.7101696 |
| NM_001001973 | ATP5C1 | 0.04413763 | up | 1.7075061 |
| NM_001030001 | RPS29 | 0.018768316 | up | 1.7068626 |
| NM_001197320 | MCL1 | 0.003819645 | up | 1.7068541 |
| NM_138568 | EXOC3L2 | 0.02849948 | up | 1.703277 |
| NM_005359 | SMAD4 | 0.04108565 | up | 1.7018416 |
| NM_004872 | TMEM59 | 0.032116644 | up | 1.700819 |
| NM_001142560 | RRNAD1 | 0.019239979 | up | 1.6994234 |
| NM_004354 | CCNG2 | 0.04256384 | up | 1.6992953 |
| NM_000877 | IL1R1 | 0.03114437 | up | 1.6990693 |
| NM_032313 | NOA1 | 0.037556242 | up | 1.6966022 |
| NM_001127178 | PIGG | 0.041096877 | up | 1.6959955 |
| NM_005920 | MEF2D | 0.034763977 | up | 1.6951872 |
| NM_001011 | RPS7 | 0.02300072 | up | 1.6951805 |
| NM_080592.3 | ATRAID | 0.027220141 | up | 1.6881133 |
| NM_001199180 | ATP2C1 | 0.024245992 | up | 1.6852529 |
| NM_002833 | PTPN9 | 0.027154233 | up | 1.6836917 |
| NM_019095 | CRLS1 | 0.014572199 | up | 1.6810064 |
| NM_001033503 | SAR1B | 0.017128667 | up | 1.6802101 |
| NM_003502 | AXIN1 | 0.04570906 | up | 1.6782374 |
| NM_000981 | RPL19 | 0.008547212 | up | 1.6763917 |
| NM_021965 | PGM5 | 0.04707265 | up | 1.6763104 |
| NM_006471 | MYL12A | 0.027220141 | up | 1.6758157 |
| NM_001145212 | SLCO2B1 | 0.02179076 | up | 1.6732384 |
| NM_020188 | CMC2 | 0.03292249 | up | 1.6669636 |
| NM_016050 | MRPL11 | 0.030987326 | up | 1.6649948 |
| NM_015683 | ARRDC2 | 0.031356245 | up | 1.6642159 |
| NM_000971 | RPL7 | 0.010243632 | up | 1.6603949 |
| NM_019111 | HLA-DRA | 0.007096998 | up | 1.6563185 |
| NM_001142605 | EFTUD2 | 0.018561976 | up | 1.6521358 |
| NM_001130969.3 | NSMF | 0.026788713 | up | 1.6471772 |
| NM_013436 | NCKAP1 | 0.02740049 | up | 1.6436874 |
| NM_001242898 | PPP6R2 | 0.017128667 | up | 1.6436238 |
| NM_080546 | SLC44A1 | 0.021675533 | up | 1.6423388 |
| NM_001015053 | HDAC5 | 0.033025254 | up | 1.6374153 |
| NM_182490 | ZNF227 | 0.03696922 | up | 1.6268303 |
| NM_017582 | UBE2Q1 | 0.01986324 | up | 1.6254896 |
| NM_016271 | RNF138 | 0.038096014 | up | 1.6152276 |
| NM_001144893 | CD209 | 0.03292249 | up | 1.6145098 |
| NM_001204255 | SCARB2 | 0.026836647 | up | 1.614064 |
| NM_004223 | UBE2L6 | 0.022763751 | up | 1.613715 |
| NM_007032 | TRIOBP | 0.033331167 | up | 1.6134425 |
| NM_001317338.1 | MARC2 | 0.028147453 | up | 1.613052 |
| NM_006380 | APPBP2 | 0.037678093 | up | 1.6127737 |
| NM_004674 | ASH2L | 0.046459515 | up | 1.611441 |
| NM_001394372.1 | BICRA | 0.0284492 | up | 1.6084399 |
| NM_032288 | FYTTD1 | 0.027236586 | up | 1.6079959 |
| NM_001165030 | TMEM41B | 0.019244099 | up | 1.6059387 |
| NM_012121 | CDC42EP4 | 0.047389053 | up | 1.6033587 |
| NM_003118 | SPARC | 0.032116644 | up | 1.6009698 |
| NM_015155 | LARP4B | 0.025329078 | up | 1.6006632 |
| NM_000361 | THBD | 0.021827964 | up | 1.5995328 |
| NM_006369 | LRRC41 | 0.02822031 | up | 1.5974282 |
| NM_020156 | C1GALT1 | 0.02436869 | up | 1.5970287 |
| NM_001113411 | FGGY | 0.026625704 | up | 1.5957259 |
| NM_022719.3 | ESS2 | 0.03788989 | up | 1.5884773 |
| NM_014952 | BAHD1 | 0.036685612 | up | 1.5877318 |
| NM_001284 | AP3S1 | 0.045609288 | up | 1.5874997 |
| NM_014949.4 | KHDC4 | 0.03772432 | up | 1.5804299 |
| NM_006241 | PPP1R2 | 0.049590055 | up | 1.5760974 |
| NM_152285 | ARRDC1 | 0.03143654 | up | 1.5726885 |
| NM_012325 | MAPRE1 | 0.036275133 | up | 1.5724943 |
| NM_001967 | EIF4A2 | 0.027494337 | up | 1.5669676 |
| NM_000146 | FTL | 0.030561026 | up | 1.5651076 |
| NM_001031714 | INF2 | 0.010243632 | up | 1.558921 |
| NM_001784.4 | ADGRE5 | 0.039582595 | up | 1.5581626 |
| NM_001009999 | KDM1A | 0.04918285 | up | 1.5579001 |
| NM_015878 | AZIN1 | 0.04413763 | up | 1.5574664 |
| NM_006004 | UQCRH | 0.047551215 | up | 1.5535363 |
| NM_001197325 | ARNT | 0.04956045 | up | 1.5521286 |
| NM_001092 | ABR | 0.02892616 | up | 1.5465313 |
| NM_001170714 | BCAR1 | 0.043186344 | up | 1.5455999 |
| NM_014394 | GHITM | 0.03696922 | up | 1.5386801 |
| NM_000262 | NAGA | 0.031356245 | up | 1.5353056 |
| NM_016551 | TM7SF3 | 0.014852795 | up | 1.5332787 |
| NM_194247 | HNRNPA3 | 0.041825365 | up | 1.5326526 |
| NM_000999 | RPL38 | 0.004334522 | up | 1.5323576 |
| NM_004965 | HMGN1 | 0.016062709 | up | 1.5319546 |
| NM_001143937 | PSMA1 | 0.007036462 | up | 1.5289323 |
| NM_007282 | RNF13 | 0.019370817 | up | 1.5287242 |
| NM_025054 | VCPIP1 | 0.013851468 | up | 1.5258412 |
| NM_001127392.2 | MYRF | 0.007959749 | up | 1.5255142 |
| NM_017798 | YTHDF1 | 0.049041808 | up | 1.5242223 |
| NM_003710 | SPINT1 | 0.023354473 | up | 1.5201715 |
| NM_138288 | SPTSSA | 0.03996587 | up | 1.5149455 |
| NM_001031684 | SRSF7 | 0.049744274 | up | 1.5122627 |
| NM_012394 | PFDN2 | 0.044370178 | up | 1.5097313 |
| NM_001256310 | GLS | 0.032408174 | up | 1.5095975 |
| NM_001031711 | ERGIC1 | 0.039047424 | up | 1.5020016 |
| NM_001168398 | SLC35A1 | 0.031058153 | up | 1.5001494 |
| NM_000224 | KRT18 | 0.031784367 | down | −1.5013014 |
| NM_005969 | NAP1L4 | 0.004919563 | down | −1.5032731 |
| NM_001144966 | NEDD4L | 0.03788989 | down | −1.5052266 |
| NM_001040000.2 | AFDN | 0.01850465 | down | −1.5055146 |
| NM_001038603 | MARVELD2 | 0.030128995 | down | −1.5055859 |
| NR_028106 | DCAF8 | 0.008405923 | down | −1.5073216 |
| NM_017925 | DENND4C | 0.043078408 | down | −1.5092858 |
| NM_001260503 | SRP68 | 0.04133102 | down | −1.5093642 |
| NM_003934 | FUBP3 | 0.030687189 | down | −1.5105941 |
| NM_178862 | STT3B | 0.027600804 | down | 1.5115358 |
| NM_032172 | USP42 | 0.026030412 | down | −1.5132701 |
| NM_001005753 | CHMP3 | 0.005358789 | down | −1.5155338 |
| NM_004589 | SCO1 | 0.020793192 | down | −1.5164549 |
| NM_001143787 | PPHLN1 | 0.011682549 | down | −1.5172632 |
| NM_023924 | BRD9 | 0.021784456 | down | −1.51805 |
| NM_006141 | DYNC1LI2 | 0.027220141 | down | −1.5194842 |
| NM_014345 | ZNF318 | 0.04286494 | down | −1.5202403 |
| NM_001013843 | SLTM | 0.03772432 | down | −1.52055 |
| NM_024776 | PEAK1 | 0.02141228 | down | −1.5206296 |
| NM_001002860 | BTBD7 | 0.029456656 | down | −1.5206753 |
| NM_005370 | RAB8A | 0.022679541 | down | −1.5213159 |
| NM_001011708 | OLA1 | 0.033178218 | down | −1.5227772 |
| NM_001130912 | SUGT1 | 0.03302928 | down | −1.5231005 |
| NM_013260 | SAP30BP | 0.011817915 | down | −1.5264447 |
| NM_001079514 | UBN1 | 0.008764876 | down | −1.5319088 |
| NM_001112707 | TLK2 | 0.008473022 | down | −1.5325893 |
| NM_001190987 | SRSF11 | 0.013081002 | down | −1.5326271 |
| NM_002802 | PSMC1 | 0.040346943 | down | −1.534994 |
| NM_001018055 | BRCC3 | 0.032441206 | down | −1.5351803 |
| NM_001985 | ETFB | 0.047005836 | down | −1.5373194 |
| NM_013233 | STK39 | 0.039021995 | down | −1.537665 |
| NM_006888 | CALM1 | 0.013458865 | down | −1.5379046 |
| NM_001097615 | POLR2J3 | 0.021812491 | down | −1.5423274 |
| NM_144604 | ZC3H18 | 0.035454735 | down | −1.5436989 |
| NM_182975 | ZNF326 | 0.02640228 | down | −1.5523969 |
| NM_004486 | GOLGA2 | 0.04108565 | down | −1.5528092 |
| NM_213720 | CHCHD10 | 0.023851642 | down | 1.5532156 |
| NM_004846 | EIF4E2 | 0.02300072 | down | −1.5535591 |
| NM_006289 | TLN1 | 0.03243209 | down | −1.5571034 |
| NM_003487 | TAF15 | 0.025869396 | down | −1.5571127 |
| NM_005104 | BRD2 | 0.027557794 | down | −1.5575494 |
| NM_001013406 | KRIT1 | 0.005730095 | down | −1.560392 |
| NM_014553 | TFCP2L1 | 0.01516808 | down | −1.5620695 |
| NM_015547 | ACOT11 | 0.039047424 | down | −1.564651 |
| NM_016139 | CHCHD2 | 0.0232449 | down | −1.5656625 |
| NM_001025091 | ABCF1 | 0.013733716 | down | −1.5672146 |
| NM_020145 | SH3GLB2 | 0.038126666 | down | −1.5681896 |
| NM_001142603 | KIAA0141 | 0.042247687 | down | −1.5693303 |
| NM_014805 | EPM2AIP1 | 0.039047424 | down | −1.5708998 |
| NM_005839 | SRRM1 | 0.04956045 | down | −1.5710309 |
| NM_152617 | RNF168 | 0.046243005 | down | −1.5725105 |
| NM_017437 | CPSF2 | 0.032116402 | down | −1.575654 |
| NM_006513 | SARS | 0.035339225 | down | −1.5758668 |
| NM_006392 | NOP56 | 0.023181079 | down | −1.5786008 |
| NM_033632 | FBXW7 | 0.012443559 | down | −1.5790751 |
| NM_001190438 | NCOR1 | 0.003742307 | down | −1.5793679 |
| NM_001008493 | ENAH | 0.01953677 | down | −1.5854431 |
| NM_001017956 | OS9 | 0.02436869 | down | −1.5856779 |
| NM_033114 | ZCRB1 | 0.00805831 | down | −1.5873162 |
| NM_003314 | TTC1 | 0.021686953 | down | −1.5897663 |
| NM_017801 | CMTM6 | 0.035377268 | down | −1.5918846 |
| NM_004792 | PPIG | 0.02199174 | down | −1.5921834 |
| NM_004713 | NEMF | 0.023515671 | down | −1.5936347 |
| NM_024835 | GGNBP2 | 0.018952131 | down | −1.5936925 |
| NM_014633 | CTR9 | 0.011682549 | down | −1.5978363 |
| NM_001134937 | FIP1L1 | 0.023815794 | down | −1.5982077 |
| NM_001007226 | SPOP | 0.017128667 | down | −1.5984762 |
| NM_013449 | BAZ2A | 0.010243632 | down | −1.5989635 |
| NM_138357 | MCU | 0.04329822 | down | −1.6004424 |
| NM_016058 | TPRKB | 0.041577 | down | −1.6005198 |
| NM_152516 | COMMD1 | 0.027220141 | down | −1.6039861 |
| NM_006425 | SLU7 | 0.010399488 | down | −1.6069827 |
| NM_003146 | SSRP1 | 0.007196175 | down | −1.6070321 |
| NM_001111077 | EZR | 0.018768316 | down | −1.6085258 |
| NM_003341 | UBE2E1 | 0.022833334 | down | −1.6092811 |
| NM_001247997 | CLIP1 | 0.041577 | down | −1.6109707 |
| NM_001261390 | CALCOCO2 | 0.02436869 | down | −1.6123476 |
| NM_001042483 | NUPR1 | 0.020793192 | down | −1.6128305 |
| NM_198147 | ABHD15 | 0.009547523 | down | −1.6137214 |
| NM_001135771 | RPN2 | 0.03514353 | down | −1.6146907 |
| NM_004477 | FRG1 | 0.020215632 | down | −1.6199828 |
| NM_015070 | ZC3H13 | 0.040169016 | down | −1.6207484 |
| NM_006555 | YKT6 | 0.021827964 | down | −1.6213622 |
| NM_016649 | ESF1 | 0.00939711 | down | −1.6216259 |
| NM_182691 | SRPK2 | 0.024362616 | down | −1.6229502 |
| NM_001005271 | CHD3 | 0.008473022 | down | −1.6232407 |
| NM_145865 | ANKS4B | 0.02975445 | down | −1.6238415 |
| NM_017672 | TRPM7 | 0.013851468 | down | −1.6252431 |
| NM_001329 | CTBP2 | 0.012045463 | down | −1.6256835 |
| NM_001378 | DYNC1I2 | 0.014574828 | down | −1.6263639 |
| NM_016940 | RWDD2B | 0.028367871 | down | −1.6289558 |
| NM_001016 | RPS12 | 0.041725997 | down | −1.6289958 |
| NM_021079 | NMT1 | 0.018912492 | down | −1.6302067 |
| NM_001046 | SLC12A2 | 0.02740049 | down | −1.6304427 |
| NM_014299 | BRD4 | 0.009983414 | down | −1.6307768 |
| NM_016329 | SFMBT1 | 0.030943058 | down | −1.6319441 |
| NM_001128852 | SRRT | 0.03788989 | down | −1.633981 |
| NM_001322373.2 | CEBPZOS | 0.008473022 | down | −1.6345502 |
| NM_017750 | RETSAT | 0.02179076 | down | −1.6353341 |
| NM_014829 | DDX46 | 0.010501327 | down | −1.6361353 |
| NM_006784 | WDR3 | 0.006968922 | down | −1.636463 |
| NM_004850 | ROCK2 | 0.019239979 | down | −1.6391218 |
| NM_144973 | DENND5B | 0.031397145 | down | −1.6399112 |
| NM_030877 | CTNNBL1 | 0.014075262 | down | −1.6411155 |
| NM_013320 | HCFC2 | 0.02101984 | down | −1.6429707 |
| NM_001199975 | UQCRB | 0.008473022 | down | −1.6435834 |
| NM_025152 | NUBPL | 0.003713761 | down | −1.6438527 |
| NM_005089 | ZRSR2 | 0.03306411 | down | −1.6441697 |
| NM_001098614 | PUS7L | 0.028559262 | down | −1.6451559 |
| NM_001256910 | DDX21 | 0.011421336 | down | −1.645982 |
| NM_001128845 | SMARCA4 | 0.041939165 | down | −1.6460872 |
| NM_012137 | DDAH1 | 0.036449004 | down | −1.6574352 |
| NM_001184790.1 | PARD3 | 0.013335236 | down | −1.65801 |
| NM_005000 | NDUFA5 | 0.009178896 | down | −1.65843 |
| NM_001130110 | SETBP1 | 0.043186344 | down | −1.6585159 |
| NM_001007225 | IGF2BP2 | 0.014852795 | down | −1.6594272 |
| NM_005828 | DCAF7 | 0.003895655 | down | −1.6596383 |
| NM_152379 | C1orf131 | 0.022763751 | down | −1.6620939 |
| NM_144723 | ZMAT2 | 0.0182891 | down | −1.6634482 |
| NM_001077664 | URGCP | 0.011933004 | down | −1.664606 |
| NM_001191037 | PSMD1 | 0.04031514 | down | −1.6652397 |
| NM_014940 | MON1B | 0.004932866 | down | −1.667509 |
| NM_005005 | NDUFB9 | 0.006167668 | down | −1.668117 |
| NM_001195478 | TFG | 0.002076559 | down | −1.6685567 |
| NM_003290 | TPM4 | 0.01873239 | down | −1.6689639 |
| NM_001042572 | CHD2 | 0.03696922 | down | −1.6696458 |
| NM_001270488 | TAF11 | 0.009009345 | down | −1.6716096 |
| NM_001938 | DR1 | 0.004889318 | down | −1.6722345 |
| NM_138389 | FAM114A1 | 0.018865926 | down | 1.6747183 |
| NM_015491 | PNISR | 0.029122181 | down | −1.6792839 |
| NM_020414 | DDX24 | 0.004210975 | down | −1.6807153 |
| NM_001257158 | CEP41 | 0.04922643 | down | −1.6820585 |
| NM_001185059 | ZCCHC6 | 0.02389813 | down | −1.6826327 |
| NM_001033 | RRM1 | 0.041577 | down | −1.6826409 |
| NM_007355 | HSP90AB1 | 0.013851468 | down | −1.6830696 |
| NM_014779 | TSC22D2 | 0.018430533 | down | −1.6869287 |
| NM_012469 | PRPF6 | 0.018209273 | down | −1.6881567 |
| NM_019589 | YLPM1 | 0.011856554 | down | −1.6883955 |
| NM_023923 | PHACTR4 | 0.010243632 | down | −1.6886494 |
| NM_145206 | VTI1A | 0.01600121 | down | −1.6895213 |
| NM_001283035.2 | RTFDC1 | 0.018125646 | down | −1.6897551 |
| NM_001137551 | LRRFIP1 | 0.01600121 | down | −1.6899005 |
| NM_003070 | SMARCA2 | 0.023851642 | down | −1.692152 |
| NM_020466 | LYRM2 | 0.017139304 | down | −1.6948234 |
| NM_001190880 | HYI | 0.030463208 | down | −1.6950924 |
| NM_005223 | DNASE1 | 0.04509934 | down | −1.6953535 |
| NM_057177 | PARD3B | 0.040927615 | down | −1.699122 |
| NM_032221 | CHD6 | 0.04241438 | down | −1.6992472 |
| NM_017546.4 | CNOT11 | 0.017277125 | down | 1.7005603 |
| NM_001164478 | C5orf63 | 0.019244099 | down | 1.7008582 |
| NM_025219 | DNAJC5 | 0.008473022 | down | 1.7013147 |
| NM_001099225 | CCDC43 | 0.04108565 | down | −1.703165 |
| NM_000987 | RPL26 | 0.016432088 | down | −1.7031944 |
| NM_024921 | POF1B | 0.042209648 | down | −1.7044595 |
| NM_006191 | PA2G4 | 0.017730126 | down | −1.7064443 |
| NM_053043 | RBM33 | 0.003062089 | down | −1.7070541 |
| NM_017553 | INO80 | 0.004210975 | down | −1.7077302 |
| NM_006699 | MAN1A2 | 0.030008137 | down | −1.7130605 |
| NM_025180 | CEP63 | 0.036284976 | down | −1.7141076 |
| NM_152713 | STT3A* | 0.03372394 | down | −1.7180325 |
| NM_014783 | ARHGAP11A | 0.03243209 | down | −1.7181059 |
| NM_001080398 | KIAA0368 | 0.043072492 | down | −1.7184577 |
| NM_001146683.1 | TMEM253 | 0.020186352 | down | −1.7188228 |
| NM_002811 | PSMD7 | 0.009763492 | down | −1.7193028 |
| NM_001267039 | LARP7 | 0.026882868 | down | −1.7219539 |
| NM_021035 | ZNFX1 | 0.031506922 | down | −1.7223219 |
| NM_006910 | RBBP6 | 0.013863794 | down | −1.7239275 |
| NM_005520 | HNRNPH1 | 0.033254527 | down | −1.7266439 |
| NM_001145923 | SCAPER | 0.033589147 | down | −1.7269214 |
| NM_001008844 | DSP | 0.040738646 | down | −1.7275516 |
| NM_006345 | SLC30A9 | 0.013979633 | down | −1.7279623 |
| NM_001077447 | PPCS | 0.008627984 | down | −1.7285151 |
| NM_007192 | SUPT16H | 0.006167668 | down | −1.7310232 |
| NM_015384 | NIPBL | 0.003315543 | down | −1.7312564 |
| NM_001164277 | SLC37A4 | 0.006502778 | down | −1.7312629 |
| NR_027261 | POLE3 | 0.032116402 | down | −1.7315081 |
| NM_001142964 | C22orf46 | 0.01600121 | down | −1.7315156 |
| NM_001098802 | CEP78 | 0.039259084 | down | −1.7373378 |
| NM_001195193 | NASP | 0.007625648 | down | −1.7393696 |
| NM_015904 | EIF5B | 0.004889318 | down | −1.7410238 |
| NM_014727.2 | KMT2B | 0.04217389 | down | −1.7417526 |
| NM_024725 | CCDC82 | 0.028559262 | down | −1.7418975 |
| NM_138450 | ARL11 | 0.03240957 | down | −1.7445707 |
| NM_019555 | ARHGEF3 | 0.01057721 | down | −1.7473708 |
| NM_000017 | ACADS | 0.003895655 | down | −1.7502544 |
| NM_002717 | PPP2R2A | 0.004950345 | down | −1.7516084 |
| NM_016625 | RSRC1 | 0.009259853 | down | −1.7562021 |
| NM_053025 | MYLK | 0.020995026 | down | −1.7589457 |
| NM_004225 | MFHAS1 | 0.010966295 | down | −1.7592223 |
| NM_001024628 | NRP1 | 0.043072492 | down | −1.7609047 |
| NM_005591.3 | MRE11 | 0.025059672 | down | −1.7627048 |
| NM_006838 | METAP2 | 0.004823584 | down | −1.7637707 |
| NM_001001569.1 | PDE9A | 0.048554912 | down | −1.7649329 |
| NM_001191022 | CTCF | 0.014850582 | down | −1.765009 |
| NM_007065 | CDC37 | 0.015753672 | down | −1.7652181 |
| NM_181575 | AUP1 | 0.03484934 | down | −1.7656367 |
| NM_002810 | PSMD4 | 0.025265217 | down | −1.765929 |
| NM_001145316 | DSN1 | 0.0182891 | down | −1.7665137 |
| NM_017780 | CHD7 | 0.016338587 | down | −1.7671039 |
| NM_032860 | LTV1 | 0.027220141 | down | −1.7705661 |
| NM_201444 | DGKA | 0.043521367 | down | −1.771234 |
| NM_015172 | PRRC2C | 0.024093835 | down | −1.7733394 |
| NM_018129 | PNPO | 0.014787058 | down | −1.7742643 |
| NM_001237 | CCNA2 | 0.016695583 | down | −1.7746786 |
| NM_016423 | ZNF219 | 0.017848339 | down | −1.77726 |
| NR_046360 | FAM49B | 0.037655722 | down | −1.782281 |
| NM_005926 | MFAP1 | 0.003898229 | down | −1.78247 |
| NM_015005.2 | CEP170B | 0.0305339 | down | −1.783447 |
| NM_001282672.1 | FAAP20 | 0.03996587 | down | −1.784309 |
| NM_002212 | EIF6 | 0.03292249 | down | −1.7873994 |
| NM_002818 | PSME2 | 0.021827964 | down | −1.7921827 |
| NM_001017989 | OPA3 | 0.031237299 | down | −1.7934369 |
| NM_001164315 | ANKRD36 | 0.04020767 | down | 1.7940307 |
| NM_002806 | PSMC6 | 0.008055674 | down | 1.7948132 |
| NM_014972 | TCF25 | 0.002998077 | down | −1.7956157 |
| NM_002270 | TNPO1 | 0.003315543 | down | 1.7958835 |
| NM_022782 | MPHOSPH9 | 0.024098136 | down | −1.7973164 |
| NM_017895 | DDX27 | 0.004210975 | down | −1.798345 |
| NM_014904 | RAB11FIP2 | 0.019239979 | down | −1.8010197 |
| NM_033018 | CDK16 | 0.037655722 | down | 1.8024555 |
| NM_001100412 | DROSHA | 0.004210975 | down | −1.8045162 |
| NM_006656 | NEU3 | 0.049744274 | down | −1.8049009 |
| NM_003621 | PPFIBP2 | 0.012795187 | down | −1.8060932 |
| NM_001130688 | HMGB2 | 0.033025254 | down | 1.8098401 |
| NR_040711 | KCNQ1 | 0.014902733 | down | −1.8105872 |
| NM_006320 | PGRMC2 | 6.40E−04 | down | −1.8123336 |
| NM_018639 | WSB2 | 0.02740049 | down | −1.8188856 |
| NM_003703 | NOP14 | 0.013801443 | down | −1.8200778 |
| NM_001037533 | GON4L | 0.004950345 | down | −1.8220265 |
| NM_003441 | ZNF141 | 0.03402427 | down | −1.8244197 |
| NM_003104 | SORD | 0.025869396 | down | −1.8264697 |
| NM_018051 | WDR60 | 0.030463208 | down | −1.8268582 |
| NM_001256476 | WDR92 | 0.027154233 | down | −1.8282392 |
| NM_014623 | MEA1 | 0.021146182 | down | −1.8292835 |
| NM_004863 | SPTLC2 | 0.00230133 | down | −1.8301853 |
| NM_005379 | MYO1A | 0.044616915 | down | −1.8306665 |
| NM_021239 | RBM25 | 0.017128667 | down | −1.830708 |
| NM_018061 | PRPF38B | 0.018865926 | down | −1.8311852 |
| NM_004398 | DDX10 | 0.040868662 | down | 1.8316561 |
| NM_001135592 | RPS27A | 0.017327527 | down | −1.8338839 |
| NM_020710 | LRRC47 | 0.027494337 | down | −1.8391333 |
| NM_022362 | MMS19 | 0.046243005 | down | −1.8394747 |
| NM_199423 | WWP2 | 0.004933076 | down | −1.8395078 |
| NM_001077199 | SREK1 | 0.009178896 | down | −1.8418868 |
| NM_005347 | HSPA5 | 0.027220141 | down | −1.8433986 |
| NM_001134709 | DEK | 0.008473022 | down | −1.8433989 |
| NM_001199885.1 | HYPK | 0.027220141 | down | −1.8435805 |
| NM_001282806.1 | MARCH7 | 0.007154981 | down | −1.844014 |
| NM_001375 | DNASE2 | 0.03891258 | down | −1.8445545 |
| NM_006938 | SNRPD1 | 0.019239979 | down | −1.8474283 |
| NM_032492 | JAGN1 | 0.003742307 | down | −1.8483722 |
| NM_001015001 | CKMT1A | 0.012087019 | down | −1.8511674 |
| NM_006577 | B3GNT2 | 0.049744274 | down | −1.8513234 |
| NM_001026 | RPS24 | 0.023523752 | down | −1.8523924 |
| NM_003657 | BCAS1 | 0.013272148 | down | −1.8541763 |
| NM_001032730 | USP48 | 0.003742307 | down | −1.8547659 |
| NM_001067 | TOP2A | 0.02822031 | down | −1.8549732 |
| NM_018115 | SDAD1 | 0.009884885 | down | −1.8600004 |
| NM_001164407 | TLCD2 | 0.016122842 | down | −1.8607192 |
| NM_001199120 | RPP21 | 0.044112798 | down | −1.8639053 |
| NM_022731 | NUCKS1 | 0.005709389 | down | −1.8639247 |
| NM_001161533 | RBM34 | 0.047335915 | down | −1.8660119 |
| NM_013974 | DDAH2 | 0.04398274 | down | −1.8673489 |
| NM_014878.4 | PUM3 | 0.035008974 | down | −1.8679883 |
| NM_018429 | BDP1 | 0.03067039 | down | −1.8717215 |
| NM_018304 | PRR11 | 0.04413763 | down | −1.8726535 |
| NM_001020819 | MYADM | 0.023851642 | down | −1.8733776 |
| NM_001012632 | IL32 | 0.021827964 | down | −1.8759776 |
| NM_006825 | CKAP4 | 0.007027982 | down | −1.8785464 |
| NM_001136153 | ATF6B | 0.03788989 | down | −1.885114 |
| NM_019606 | MEPCE | 0.017527176 | down | −1.8854225 |
| NM_001164446 | C6orf132 | 0.031489152 | down | −1.885912 |
| NM_021915 | ZNF69 | 0.045422226 | down | −1.8871384 |
| NM_001040440 | CCDC112 | 0.016433196 | down | −1.8872321 |
| NM_016403 | CWC15 | 0.003315543 | down | −1.888155 |
| NM_181514 | MRPL21 | 0.03519735 | down | −1.8909903 |
| NM_001001503 | NDUFV3 | 0.030463208 | down | 1.894879 |
| NM_015131 | WDR43 | 0.007312382 | down | −1.8950089 |
| NM_002308 | LGALS9 | 0.032116402 | down | −1.896425 |
| NM_013433 | TNPO2 | 0.027224611 | down | −1.9006695 |
| NM_001099661 | EIF3CL | 0.038096014 | down | −1.9014058 |
| NM_201538 | NDRG2 | 0.034291364 | down | −1.9075458 |
| NM_001204090 | COX16 | 0.017599188 | down | −1.9270235 |
| NM_152434 | CWF19L2 | 0.015763555 | down | −1.9276371 |
| NM_018009 | TAPBPL | 0.025869396 | down | −1.9302168 |
| NM_001258331 | EPB41L1 | 0.03772432 | down | −1.932483 |
| NM_007298 | BRCA1 | 0.025869396 | down | −1.9346664 |
| NM_006401 | ANP32B | 0.003957412 | down | −1.9347495 |
| NM_018471 | ZC3H15 | 0.00325619 | down | −1.9373347 |
| NM_003629 | PIK3R3 | 0.021242654 | down | −1.9438714 |
| NM_001167941 | WDR76 | 0.026586808 | down | −1.9492041 |
| NM_004890 | SPAG7 | 0.02740049 | down | −1.9525083 |
| NM_001030006 | AP2B1 | 0.030542143 | down | −1.9569894 |
| NM_014109 | ATAD2 | 0.01691245 | down | −1.9578521 |
| NM_006579 | EBP | 0.025708964 | down | −1.9633696 |
| NM_014046 | MRPS18B | 0.003742307 | down | −1.9639891 |
| NM_001256666 | FAM193A | 0.030008137 | down | −1.9692286 |
| NM_017782 | FAM208B | 0.03951824 | down | −1.9740738 |
| NM_001002877 | THOC5 | 0.027220141 | down | −1.9861796 |
| NM_001005476 | PKP4 | 0.001188409 | down | −1.9864517 |
| NM_014704 | CEP104 | 0.009178896 | down | −1.9906662 |
| NM_014508 | APOBEC3C | 0.01691245 | down | −1.99686 |
| NM_013285 | GNL2 | 0.04202552 | down | −1.9993088 |
| NM_002714 | PPP1R10 | 0.014574828 | down | −1.9997517 |
| NM_001194998 | CEP152 | 0.011271159 | down | −2.0112448 |
| NM_014287 | NOMO1 | 0.01657466 | down | −2.0123777 |
| NM_032302 | PSMG3 | 0.033331167 | down | −2.0129387 |
| NM_015036 | ENDOD1 | 0.011682549 | down | −2.0187235 |
| NM_013293 | TRA2A | 0.011421336 | down | −2.0226877 |
| NM_020382.4 | KMT5A | 0.013992935 | down | −2.0227282 |
| NM_017816 | LYAR | 0.041725997 | down | −2.0235846 |
| NM_001172638 | ZFP62 | 0.020598654 | down | −2.024434 |
| NM_024108 | TRAPPC6A | 0.0239216 | down | −2.026981 |
| NM_001077186 | MYH14 | 0.045422226 | down | −2.0272028 |
| NM_144626 | TMEM125 | 0.013350707 | down | −2.031033 |
| NM_017645 | HAUS6 | 0.007037839 | down | −2.0397317 |
| NM_015510 | DHRS7B | 0.01864376 | down | −2.0456774 |
| NM_020198 | CCDC47 | 0.004210975 | down | −2.0559921 |
| NM_001135564 | HSF2 | 0.03546597 | down | −2.0642316 |
| NM_030928 | CDT1 | 0.039349694 | down | −2.0647488 |
| NM_016207 | CPSF3 | 0.003713761 | down | −2.0691328 |
| NM_014077 | FAM32A | 0.007625648 | down | −2.0705957 |
| NM_001174068 | SYTL4 | 0.030880967 | down | −2.0708818 |
| NM_152546 | SRFBP1 | 0.048054714 | down | −2.0755675 |
| NM_138287 | DTX3L | 0.033524778 | down | −2.081305 |
| NM_020137 | GRIPAP1 | 0.006167668 | down | −2.0846534 |
| NM_016426 | GTSE1 | 0.023821704 | down | −2.0847561 |
| NM_003600 | AURKA | 0.003742307 | down | −2.0889087 |
| NM_001322141.1 | TANGO2 | 0.035339225 | down | −2.09069 |
| NM_000175 | GPI | 0.010470299 | down | −2.0961623 |
| NM_001098416 | HDAC7 | 0.00230133 | down | −2.1058626 |
| NM_007002 | ADRM1 | 0.047972605 | down | −2.1059482 |
| NM_001543 | NDST1 | 0.04346288 | down | −2.1164773 |
| NM_023010 | UPF3B | 0.009026866 | down | −2.1168072 |
| NM_001164380 | STAU2 | 0.006167668 | down | −2.117224 |
| NM_001127181 | CENPL | 0.013851468 | down | −2.1177623 |
| NM_024299 | PPDPF | 0.004210975 | down | −2.1200857 |
| NM_001130057 | EEF1D* | 0.014787058 | down | −2.120689 |
| NM_000166 | GJB1 | 0.025059672 | down | −2.124312 |
| NM_001159770 | SLC39A11 | 0.021042481 | down | −2.1246893 |
| NM_020886 | USP28 | 0.02436869 | down | −2.1259046 |
| NM_016334 | GPR89B | 0.03464142 | down | −2.126268 |
| NM_207343 | RNF214 | 0.036685612 | down | −2.1314788 |
| NM_020242 | KIF15 | 0.04568518 | down | −2.1351426 |
| NM_001243225 | PES1 | 0.031489152 | down | −2.1386645 |
| NM_138421 | SAAL1 | 0.006167668 | down | −2.1468813 |
| NM_001145966 | MKI67 | 0.016695583 | down | −2.1474903 |
| NM_152362 | TNFAIP8L1 | 0.003742307 | down | −2.153528 |
| NM_030629 | CMIP | 0.012671394 | down | −2.1536067 |
| NM_001177948 | SYCP3 | 0.008473022 | down | −2.1703491 |
| NM_003685 | KHSRP | 0.017927054 | down | −2.187061 |
| NM_001376 | DYNC1H1 | 0.003177543 | down | −2.1970005 |
| NM_016399 | TRIAP1 | 0.013066018 | down | −2.2082975 |
| NM_001243224 | RAD9A | 0.008473022 | down | −2.2084272 |
| NM_001193285 | SIRT6 | 0.008405923 | down | −2.2218795 |
| NM_001166691 | TTK | 0.021737874 | down | −2.2266738 |
| NM_001077516 | SLC39A7 | 0.012005926 | down | −2.230839 |
| NM_198098 | AQP1 | 0.03287586 | down | −2.233865 |
| NM_017841 | SDHAF2 | 0.010399488 | down | −2.2364204 |
| NM_005381 | NCL | 0.004932866 | down | −2.2600253 |
| NM_002587 | PCDH1 | 0.031724196 | down | −2.2621543 |
| NM_001083893 | STRN3 | 0.011421336 | down | −2.265027 |
| NM_005792 | MPHOSPH6 | 0.003315543 | down | −2.266536 |
| NM_144982 | ZFC3H1 | 0.014574828 | down | −2.2701051 |
| NM_006306 | SMC1A | 0.003315543 | down | −2.3004236 |
| NM_003902 | FUBP1 | 0.006318725 | down | −2.3013449 |
| NM_000945 | PPP3R1 | 0.006167668 | down | −2.309041 |
| NM_152515 | CKAP2L | 0.033178218 | down | −2.31789 |
| NM_005960.1 | MUC3A | 0.030544128 | down | −2.33021 |
| NM_014657 | TTI1 | 0.010966295 | down | −2.3351223 |
| NM_024819 | DCAKD | 0.03963494 | down | −2.3411264 |
| NM_133443 | GPT2 | 0.019868482 | down | −2.3501973 |
| NM_003249 | THOP1 | 0.017848339 | down | −2.3525672 |
| NM_001114395 | CNTLN | 0.04522721 | down | −2.3606458 |
| NM_001099285 | PTMA | 0.004296454 | down | −2.3880048 |
| NM_032704 | TUBA1C | 0.02199174 | down | −2.393255 |
| NM_006389 | HYOU1* | 0.010996858 | down | −2.3978121 |
| NM_014784 | ARHGEF11 | 0.030757058 | down | −2.4103 |
| NM_022346 | NCAPG | 0.0378355 | down | −2.417053 |
| NM_001166159 | NDUFS2 | 0.014546518 | down | −2.4246175 |
| NR_002162 | ATP5EP2 | 0.008055674 | down | −2.4329066 |
| NM_001039112 | FER1L6 | 0.040550947 | down | −2.4552534 |
| NM_007072 | HHLA2 | 0.025571894 | down | −2.455378 |
| NM_030763 | HMGN5 | 0.011856554 | down | −2.4579012 |
| NM_007365 | PADI2 | 0.009178896 | down | −2.4632423 |
| NM_002804 | PSMC3 | 0.004091352 | down | −2.472983 |
| NM_000417 | IL2RA | 0.021827964 | down | −2.4985712 |
| NM_001007468 | SMARCB1 | 0.011266685 | down | −2.5146255 |
| NM_001024212 | S100A13 | 0.013335419 | down | −2.5629876 |
| NM_001031712 | TRMT11 | 0.011151491 | down | −2.5908039 |
| NM_001253861 | MND1 | 0.04397283 | down | −2.6010153 |
| NM_001040011 | SWI5 | 0.041577 | down | −2.604326 |
| NM_145233.4 | ZNF625 | 0.041577 | down | −2.6173143 |
| NM_145236 | B3GNT7 | 0.009026866 | down | −2.6183298 |
| NM_017613 | DONSON | 0.02403732 | down | −2.6804373 |
| NM_001014839 | NCDN | 0.027220141 | down | −2.7379856 |
| NM_004365 | CETN3 | 0.003742307 | down | −2.7603252 |
| NM_078488 | VNN2* | 0.028559262 | down | −2.7931726 |
| NM_001142784 | IL11RA | 0.009861059 | down | −2.8303916 |
| NM_006907 | PYCR1 | 0.04552335 | down | −2.9068618 |
| NM_001161580 | POC1A | 0.003710429 | down | −2.9079642 |
| NM_006142 | SFN | 0.009178896 | down | −2.9141273 |
| NM_017906 | PAK1IP1 | 0.020355955 | down | −3.7688813 |
| NM_001242920 | PLTP | 0.030513493 | down | −3.8500047 |
| NM_007052 | NOX1 | 0.02300072 | down | −4.2817006 |
| NM_001166131 | EME1 | 0.01146189 | down | −6.1623173 |
| NM_001042432.2 | AC073610.2 | 0.011421336 | down | −8.3613405 |
| NM_013330 | NME7 | 0.001782755 | down | −9.525431 |
| NM_194249 | DND1 | 0.003177543 | down | −13.645517 |
| NM_001017390.3 | SULT1A4 | 3.08E−04 | down | −13.920308 |

### REFERENCES

1. Rothenberg M E. Eosinophilic gastrointestinal disorders (EGID). J Allergy Clin Immunol 2004; 113:11-28.
2. Jensen E T, Martin C F, Kappelman M D, et al. Prevalence of eosinophilic gastritis, gastroenteritis, and colitis: estimates from a national administrative database. J Pediatr Gastroenterol Nutr 2016; 62:36-42.
3. Mansoor E, Saleh M A, Cooper G S. Prevalence of eosinophilic gastroenteritis and colitis in a population based study, from 2012 to 2017. Clin Gastroenterol Hepatol 2017; 15:1733-1741.
4. DiTommaso L A, Rosenberg C E, Eby M D, et al. Prevalence of eosinophilic colitis and the diagnoses associated with colonic eosinophilia. J Allergy Clin Immunol 2019; 143:1928-1930.
5. Jensen E T, Aceves S S, Bonis P A, et al. High patient disease burden in a cross-sectional, multicenter contact registry study of eosinophilic gastrointestinal diseases. J Pediatr Gastroenterol Nutr 2020; 71:524-529.
6. Turner K O, Sinkre R A, Neumann W L, et al. Primary colonic eosinophilia and eosinophilic colitis in adults. Am J Surg Pathol 2017; 41:225-233.
7. Mark J, Fernando S D, Masterson J C, et al. Clinical implications of pediatric colonic eosinophilia. J Pediatr Gastroenterol Nutr 2018; 66:760-766.
8. Blanchard C, Wang N, Stringer K F, et al. Eotaxin-3 and a uniquely conserved gene-expression profile in eosinophilic esophagitis. J Clin Invest 2006; 116:536-547.
9. Sherrill J D, Kiran K C, Blanchard C, et al. Analysis and expansion of the eosinophilic esophagitis transcriptome by RNA sequencing. Genes Immun 2014; 15:361-369.
10. Shoda T, Matsuda A, Nomura I, et al. Eosinophilic esophagitis versus proton pump inhibitor-responsive esophageal eosinophilia: Transcriptome analysis. J Allergy Clin Immunol 2017; 139:2010-2013.
11. Shoda T, Wen T, Caldwell J M, et al. Loss of endothelial TSPAN12 promotes fibrostenotic eosinophilic esophagitis via endothelial cell-fibroblast crosstalk. Gastroenterology 2022; 162:439-453.
12. Shoda T, Kaufman K M, Wen T, et al. Desmoplakin and periplakin genetically and functionally contribute to eosinophilic esophagitis. Nat Commun 2021; 12:6795.
13. Caldwell J M, Collins M H, Stucke E M, et al. Histologic eosinophilic gastritis is a systemic disorder associated with blood and extragastric eosinophilia, TH2 immunity, and a unique gastric transcriptome. J Allergy Clin Immunol 2014; 134:1114-1124.
14. Sato M, Shoda T, Shimizu H, et al. Gene expression patterns in distinct endoscopic findings for eosinophilic gastritis in children. J Allergy Clin Immunol Pract 2017; 5:1639-1649.
15. Shoda T, Wen T, Caldwell J M, et al. Molecular, endoscopic, histologic, and circulating biomarker-based diagnosis of eosinophilic gastritis: multi-site study. J Allergy Clin Immunol 2020; 145:255-269.
16. Collins M H, Capocelli K, Yang G Y. Eosinophilic gastrointestinal disorders pathology. Front Med (Lausanne) 2017; 4:261.
17. O'Shea K M, Aceves S S, Dellon E S, et al. Pathophysiology of eosinophilic esophagitis. Gastroenterology 2018; 154:333-345.
18. Dunn J L M, Shoda T, Caldwell J M, et al. Esophageal type 2 cytokine expression heterogeneity in eosinophilic esophagitis in a multisite cohort. J Allergy Clin Immunol 2020; 145:1629-1640.
19. Lyles J L, Martin L J, Shoda T, et al. Very early onset eosinophilic esophagitis is common, responds to standard therapy, and demonstrates enrichment for CAPN14 genetic variants. J Allergy Clin Immunol 2021; 147:244-254.
20. DeBrosse C W, Case J W, Putnam P E, et al. Quantity and distribution of eosinophils in the gastrointestinal tract of children. Pediatr Dev Pathol 2006; 9:210-218.
21. Sonnenberg A, Turner K O, Singhal A, et al. Prevalence and concordant occurrence of esophageal, gastric, duodenal, and colonic eosinophilia. Dis Esophagus 2020; 33:doaa064.
22. Pesek R D, Rothenberg M E. Eosinophilic gastrointestinal disease below the belt. J Allergy Clin Immunol 2020; 145:87-89.
23. Gupta S K, Falk G W, Aceves S S, et al. Consortium of Eosinophilic Gastrointestinal Disease Researchers: advancing the field of eosinophilic G I disorders through collaboration. Gastroenterology 2019; 156:838-842.
24. Collins M H. Histopathologic features of eosinophilic esophagitis and eosinophilic gastrointestinal diseases. Gastroenterol Clin North Am 2014; 43:257-268.
25. Pesek R D, Greuter T, Lopez-Nunez O, et al. Clinicopathologic correlations in eosinophilic gastrointestinal disorders. J Allergy Clin Immunol Pract 2021; 9:3258-3266.
26. Bousvaros A, Antonioli D A, Colletti R B, et al. Differentiating ulcerative colitis from Crohn disease in children and young adults: report of a working group of the North
American Society for Pediatric Gastroenterology, Hepatology, and Nutrition and the Crohn's and Colitis Foundation of America. J Pediatr Gastroenterol Nutr 2007; 44:653-674.
27. Bindea G, Mlecnik B, Hackl H, et al. ClueGO: a Cytoscape plug-in to decipher functionally grouped gene ontology and pathway annotation networks. Bioinformatics 2009; 25:1091-1093.
28. Chen J, Bardes E E, Aronow B J, et al. ToppGene Suite for gene list enrichment analysis and candidate gene prioritization. Nucleic Acids Res 2009; 37:W305-W311.
29. Aran D, Hu Z, Butte A J. xCell: digitally portraying the tissue cellular heterogeneity landscape. Genome Biol 2017; 18:220.
30. Wen T, Stucke E M, Grotjan T M, et al. Molecular diagnosis of eosinophilic esophagitis by gene expression profiling. Gastroenterology 2013; 145:1289-1299.
31. Haberman Y, Karns R, Dexheimer P J, et al. Ulcerative colitis mucosal transcriptomes reveal mitochondriopathy and personalized mechanisms underlying disease severity and treatment response. Nat Commun 2019; 10:38.
32. Travers J, Rochman M, Caldwell J M, et al. IL-33 is induced in undifferentiated, non-dividing esophageal epithelial cells in eosinophilic esophagitis. Sci Rep 2017; 7:17563.
33. Walker M M, Potter M D, Talley N J. Eosinophilic colitis and colonic eosinophilia. Curr Opin Gastroenterol 2019; 35:42-50.
34. Pinero J, Ramirez-Anguita J M, Sauch-Pitarch J, et al. The DisGeNET knowledge platform for disease genomics: 2019 update. Nucleic Acids Res 2020; 48:D845-D855.
35. Klion A D, Ackerman S J, Bochner B S. Contributions of eosinophils to human health and disease. Annu Rev Pathol 2020; 15:179-209.
36. Persson E K, Verstraete K, Heyndrickx I, et al. Protein crystallization promotes type 2 immunity and is reversible by antibody treatment. Science 2019; 364:eaaw4295.
37. Cello J P. Eosinophilic gastroenteritis—a complex disease entity. Am J Med 1979; 67:1097-1104.
38. Moore D, Lichtman S, Lentz J, et al. Eosinophilic gastroenteritis presenting in an adolescent with isolated colonic involvement. Gut 1986; 27:1219-1222.
39. Uppal V, Kreiger P, Kutsch E. Eosinophilic gastroenteritis and colitis: a comprehensive review. Clin Rev Allergy Immunol 2016; 50:175-188.
40. Hogan S P, Mishra A, Brandt E B, et al. A critical role for eotaxin in experimental oral antigen-induced eosinophilic gastrointestinal allergy. Proc Natl Acad Sci USA 2000; 97:6681-6686.
41. Ahrens R, Waddell A, Seidu L, et al. Intestinal macrophage/epithelial cell-derived CCL11/eotaxin-1 mediates eosinophil recruitment and function in pediatric ulcerative colitis. J Immunol 2008; 181:7390-7399.
42. Coburn L A, Horst S N, Chaturvedi R, et al. Highthroughput multi-analyte Luminex profiling implicates eotaxin-1 in ulcerative colitis. PLoS One 2013; 8:e82300.
43. Juhasz A, Markel S, Gaur S, et al. NADPH oxidase 1 supports proliferation of colon cancer cells by modulating reactive oxygen species-dependent signal transduction. J Biol Chem 2017; 292:7866-7887.
44. Trocme C, Deffert C, Cachat J, et al. Macrophage-specific NOX2 contributes to the development of lung emphysema through modulation of SIRT1/MMP-9 pathways. J Pathol 2015; 235:65-78.
45. Shiba-Ishii A, Noguchi M. Aberrant stratifin overexpression is regulated by tumor-associated CpG demethylation in lung adenocarcinoma. Am J Pathol 2012; 180:1653-1662.
46. Moon S J, Min C K, Lee D G, et al. Pseudomembranous colitis following bortezomib therapy in a myeloma patient. Acta Haematol 2007; 117:211-214.
47. Nogales Rincón O, Huerta Madrigal A, Merino Rodriguez B, et al. Rectal bleeding and diarrhea caused by bortezomib-induced colitis. Gastroenterol Hepatol 2010; 33:753-754.
48. Kumagai H, Masuda T, Maisawa S, et al. Apoptotic epithelial cells in biopsy specimens from infants with streaked rectal bleeding. J Pediatr Gastroenterol Nutr 2001; 32:428-433.
49. Kiss Z, Béres N J, Sziksz E, et al. Specific microRNA pattern in colon tissue of young children with eosinophilic colitis. Int J Mol Sci 2017; 18:1050.
50. Brusilovsky M, Rochman M, Rochman Y, et al. Environmental allergens trigger type 2 inflammation through ripoptosome activation. Nat Immunol 2021; 22:1316-1326.
51. Li J, Mao R, Kurada S, et al. Pathogenesis of fibrostenosing Crohn's disease. Transl Res 2019; 209:39-54.
52. Gupta S K, Falk G W, Aceves S S, et al. Consortium of Eosinophilic Gastrointestinal Disease Researchers: Advancing the Field of Eosinophilic G I Disorders Through Collaboration. Gastroenterology 2019; 156:838-842.
53. Raffaele A, Vatta F, Votto M, et al. Eosinophilic colitis in children: a new and elusive enemy?Pediatr Surg Int 2021; 37:485-490.
54. Kupershmidt I, Su Q J, Grewal A, et al. Ontology-based meta-analysis of global collections of high-throughput public data. PLoS One 2010; 5.
13. Travers J, Rochman M, Caldwell J M, et al. IL-33 is induced in undifferentiated, nondividing esophageal epithelial cells in eosinophilic esophagitis. Sci Rep 2017; 7:17563.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

1. A method of treating active eosinophilic colitis (EoC) in an individual, the method comprising
a) assaying a tissue sample obtained from a colon of said individual for expression of at least one gene of an EoC transcriptome gene set;
b) calculating an EoC score based on said expression of EoC transcriptome gene set;
c) selecting a tissue sample that exhibits an EoC score indicative of active EoC; and
d) administering an anti-inflammatory therapy to the individual whose tissue sample was selected in (c).

2. (canceled)

3. The method of claim 1, wherein said EoC transcriptome gene set comprises each EoC transcriptome gene set of Table 12.

4. The method of claim 1, wherein said EoC transcriptome gene set comprises the EoC transcriptome genes of Table 12 having at least a 3-fold change compared to a normal control.

5. The method of claim 1, wherein said EoC transcriptome gene set comprises the EoC transcriptome genes of Table 12 having at least a 5-fold change compared to a normal control.

6. The method of claim 1, wherein said tissue sample is obtained from a site selected from ascending colon, descending colon, sigmoid colon, or a combination thereof.

7. The method of claim 1, wherein said EoC score is calculated by summing the normalized expression values of genes dysregulated in the EoC transcriptome set.

8. The method of claim 1, which is performed prior to treatment with a therapy for active EoC.

9. The method of claim 1, which is performed after treatment with a therapy for active EoC.

10. The method of claim 1, which is performed during disease progression or clinical relapse on a therapy for active EoC.

11. The method of claim 1, which is performed after suspension of a therapy for active EoC.

12. The method of claim 1, wherein said anti-inflammatory therapy is selected from a glucocorticoid, food restriction, leukotriene inhibitor, mast cell stabilizer, mast cell depleting drug, an anti-TNF agent, interleukin-5 inhibitor, an eosinophil depleting drug, anti-eotaxin agent, anti-CCR3 agent, an immunosuppressive drug, a biological therapy, and combinations thereof.

13. A method of treating inflammatory bowel disease (IBD) with high colonic eosinophilia in an individual, the method comprising
a) assaying a colonic tissue sample obtained from said individual for expression of at least one gene, or at least two genes, or at least two genes, or at least two genes, or at least three genes, or at least four genes, or at least five genes, or at least six genes, or at least seven genes, or at least eight genes, or at least nine genes, or at least ten genes, or at least 11 genes, or at least 12 genes, or at least 13 genes, or at least 14 genes, or at least 15 genes, or at least 16 genes, or all genes of the EoC transcriptome of Table 11;
b) calculating an EoC-IBD differential score;
c) selecting a sample that exhibits an EoC-IBD differential score indicative of IBD; and
d) administering a therapy for IBD to the individual whose sample was selected in (c).

14. The method of claim 13 wherein said assaying comprises detection of each of the genes in the EoC transcriptome of Table 11.

15. The method of claim 13, further comprising assaying for one or more pathologic changes selected from the presence of eosinophil sheets, cryptitis, crypt abscesses, muscular involvement, and combinations thereof, wherein one or more pathological change weighs in favor of a diagnosis of EoC.

16. The method of claim 13, further comprising assaying for one or more pathologic changes selected from a lack of acute inflammation and cryptitis, wherein a lack of said features is indicative of EoC.

17. The method of claim 13, wherein the assaying comprises whole transcriptome sequencing, antibody-based protein quantifications, mass spectrometry based protein quantification, targeted mRNA sequencing, targeted mRNA quantification, nanostring determination, real-time RT-PCR, or a combination thereof.

18. The method of claim 13, wherein the assaying comprises Sanger sequencing, targeted sequencing, whole exome/genome sequencing, whole exome/genome quantification, or a combination thereof.

19. A method of diagnosing an individual with active EoC, said method comprising
a) assaying a tissue sample obtained from a colon of said individual for expression of at least one gene of an EoC transcriptome gene set;
b) calculating an EoC score based on said expression of EoC transcriptome gene set;
c) selecting a tissue sample that exhibits an EoC score indicative of active EoC; and
d) diagnosing the individual whose tissue sample was selected in (c) with active EoC.

20. The method of claim 19, wherein said EoC transcriptome gene set comprises at least 70% or at least 80%, or at least 90% of said EoC transcriptome gene set.

21. The method of claim 19, wherein said EoC transcriptome gene set comprises each of the EoC transcriptome gene set of Table 12.

22. The method of claim 20, wherein said EoC transcriptome gene set comprises the EoC transcriptome genes of Table 12 having at least a 3-fold change compared to a normal control or at least a 5-fold change compared to a normal control.

23. (canceled)

24. The method of claim 19, wherein said tissue sample is obtained from a site selected from ascending colon, descending colon, sigmoid colon, or a combination thereof.

25. The method of claim 19, wherein said EoC score is calculated by summing the normalized expression values of genes dysregulated in the EoC transcriptome set.

26. (canceled)

27. (canceled)

28. (canceled)

29. (canceled)

30. A method of diagnosing inflammatory bowel disease (IBD) with high colonic eosinophilia in an individual, the method comprising
a) assaying a colonic tissue sample obtained from said individual for expression of at least one gene, or at least two genes, or at least two genes, or at least two genes, or at least three genes, or at least four genes, or at least five genes, or at least six genes, or at least seven genes, or at least eight genes, or at least nine genes, or at least ten genes, or at least 11 genes, or at least 12 genes, or at least 13 genes, or at least 14 genes, or at least 15 genes, or at least 16 genes, or all genes of the EoC transcriptome as listed in Table 11;
b) calculating an EoC-IBD differential score;
c) selecting a sample that exhibits an EoC-IBD differential score indicative of IBD; and
d) diagnosing the individual whose sample was selected in (c) with IBD.

31. The method of claim 30 wherein said assaying comprises detecting expression of each gene of the EoC transcriptome of Table 11.

32. (canceled)

33. (canceled)
